(19) United States
(12) Patent Application Publication
(10) Pub. No.: US 2024/0377400 A1
(43) Pub. Date: Nov. 14, 2024

(54) METHODS FOR TREATING A SUBTYPE OF COLORECTAL CANCER

(71) Applicants: Jorge MOSCAT-GUILLEN, New York, NY (US); Maria T. DIAZ-MECO CONDE, New York, NY (US); Maria Angeles DURAN-MOLINA, New York, NY (US); Anxo MARTINEZ-ORDONEZ, New York, NY (US); Cornell University, Ithaca, NY (US); Sanford Burnham Prebys Medical Discovery Institute, La Jolla, CA (US)

(72) Inventors: Jorge Moscat-Guillen, New York, NY (US); Maria T. Diaz-Meco Conde, New York, NY (US); Maria Angeles Duran-Molina, New York, NY (US); Anxo Martinez-Ordonez, New York, NY (US)

(21) Appl. No.: 18/692,253

(22) PCT Filed: Sep. 20, 2022
(86) PCT No.: PCT/US2022/076717

§ 371 Date: Mar. 14, 2024

Related U.S. Application Data
(60) Provisional application No. 63/246,190, filed on Sep. 20, 2021.

(51) Int. Cl. G01N 33/574; C12Q 1/6886

(52) U.S. Cl. CPC G01N 33/57419; C12Q 1/6886; C12Q 2600/112; G01N 2333/912; G01N 2400/40; G01N 2800/52

(57) ABSTRACT

This invention relates generally to methods for determining a subject having or is suspected of having a subtype of colorectal cancer and methods for treating the subject.

### CROSS-REFERENCE TO RELATED APPLICATIONS

This application claims the benefit of the filing date of U.S. application No. 63/246,190, filed on Sep. 20, 2021, the disclosure of which is incorporated by reference herein.

### STATEMENT AS TO FEDERALLY SPONSORED RESEARCH

This invention was made with government support under Contract number CA207177 awarded by the National Institutes of Health. The government has certain rights in the invention.

### BACKGROUND

Colorectal cancer (CRC) is one of the leading causes of cancer-related deaths worldwide. The consensus molecular subtypes (CMS) classification of CRCs is based on new genetic and transcriptomics classification, resulting in four consensus molecular subtypes (CMSs) with distinguishing features: CMS1 (microsatellite instability immune, 14%), CMS2 (canonical, 37%), CMS3 (metabolic, 13%), and CMS4 (mesenchymal, 23%). (Guinney et al., 2015). The CMS4 subtype is characterized by signatures indicative of EMT and a stromal-enriched immune microenvironment. The CMS4 CRC is the subgroup with the poorest prognosis. (Nakanishi et al., 2019). Thus, there is a need for accurate methods for diagnosing CMS4 CRC patients and effective methods for treating CMS4 CRC patients.

Colorectal cancer (CRC) is a major cause of cancer mortality worldwide (Siegel et al., 2020). Despite improvements in systemic treatment, the metastatic disease shows a dismal 5-year survival rate of only 12%-14% (Martini et al., 2020). CRCs can develop from any region of the colon or the rectum. However, it has become increasingly clear that primary tumor locations show distinct pathophysiological and clinical characteristics (Huyghe et al., 2021; Missiaglia et al., 2014). Thus, in the distal (left-sided) colon, tumors typically initiate from tubular adenomas, whereas in the proximal (right-sided) colon, tumors initiate from sessile serrated lesions (SSL). These SSL-derived proximal CRCs can evolve through a transcriptional pathway termed CMS1, are often associated with BRAF mutations, are characterized by microsatellite instability (MSI-H) with a CpG island methylator phenotype (CIMP), are immune active, present with a good prognosis, and are relatively sensitive to immune checkpoint blockade therapy (ICB) (Mandal et al., 2019; Overman et al., 2017). In contrast, distal CRCs are microsatellite stable (MSS), often chromosomal instable, belonging to the CMS2/3 transcriptional category, initiated by mutations in APC, TP53, KRAS, and the TGFβ pathway, have an intermediate prognosis, and account for about 50% of all CRCs (Dienstmann et al., 2017; Guinney et al., 2015). The transcriptional classification of CRC tumors also revealed a CMS4-enriched subtype, which accounts for about 35% of all CRCs and displays the highest risk of distant relapse and the worst prognosis (De Sousa et al., 2013; Fessler and Medema, 2016). CMS4 tumors could evolve from SSL or tubular adenomas, are microsatellite stable (MSS), and resistant to ICB therapy (Calon et al., 2015). In contrast to CMS2/3 tumors, which are immunologically deserted, CMS4 tumors are inflamed with CD8+ T cells accumulating in the stromal periphery and excluded from the cancer epithelial core (Kasashima et al., 2021; Nakanishi et al., 2019; Nakanishi et al., 2018).

Other distinctive features of the CMS4-enriched tumors are their high content in activated cancer-associated fibroblasts (CAFs) and the acquisition of a mesenchymal phenotype that predicts adverse outcomes in CRC patients better than the presence of prevalent mutations (Calon et al., 2015; De Sousa et al., 2013; Guinney et al., 2015). This agrees with the increasingly accepted notion that an activated desmoplastic stroma drives the resistance of CRC cells to conventional and targeted therapies (Moscat et al., 2022). However, the molecular and cellular mechanisms that drive mesenchymal tumorigenesis are still far from clear. Given the relatively scarce information on the fundamental players controlling the development of this type of intestinal tumor, it is critical to find new regulators of these pathways beyond the known mutated tumor suppressors and oncogenes already identified in cancers from CMS2/3 distal tubular lesions or CMS1 proximal SSL. In this regard, we have recently reported that low levels of the atypical PKCs (aPKCs; PKCQ and PKCX/1) are drivers for the initiation and progression of mesenchymal CRC (mCRC), as well as for their resistance to ICB (Nakanishi et al., 2018; Reina-Campos et al., 2019). Thus, the simultaneous inactivation of both aPKCs in the mouse intestinal epithelium results in the generation of aggressive MSS mesenchymal intestinal tumors with serrated and signet ring carcinoma features and a reactive desmoplastic and immunosuppressive tumor microenvironment (Moscat et al., 2022; Nakanishi et al., 2018). aPKC-deficient tumors excluded CD8+ T cells to the stromal periphery but were infiltrated with myeloid-derived suppressor cells (MDSCs) expressing PD-L1 and were resistant to anti-PD-L1 treatment (Nakanishi et al., 2018).

Because mCRC tumors were populated by CAFs and enriched in a TGFβ signaling gene expression signature, several studies explored the possibility of treating these tumors by inhibiting TGFβ function. Surprisingly, this approach was not sufficient to reduce the tumor load or to reactivate the immune system, although it decreased the desmoplastic reaction and enabled response to anti-PD-L1 therapy (Mariathasan et al., 2018; Nakanishi et al., 2018; Tauriello et al., 2018). Therefore, it could be argued that targeting CAFs, while inefficient as monotherapy, at least at the TGFβ level, might be a new potential venue to promote immunotherapy in mCRC. However, the design of therapeutics aimed at selectively de-reprograming CAFs is extremely challenging due to the heterogeneity of the stromal fibroblasts in several cancers, including CRC, and a limited understanding of the master regulators of the different CAF subtypes.

The difficulty of targeting CAFs impelled the search for alternative ways to modulate the tumor stroma. Thus, several laboratories have focused their interest on targeting not the cellular components but different molecules of the TME extracellular matrix, like collagens and the glycosaminoglycan hyaluronan (HA) (De Jaeghere et al., 2019; Jacobetz et al., 2013; Levental et al., 2009; Provenzano et al., 2012). The rationale behind this approach is that reducing the extracellular matrix barrier purportedly protecting the tumor would result in better access to chemotherapeutics and ICB treatments. However, genetically targeting collagen I, the major fibrillar collagen type, in αSMA⁺ pancreatic and intestinal CAFs did not reduce but enhanced tumor growth by either decreasing the barrier effect, which allows the tumor to expand free of its stromal boundaries, or by promoting the recruitment of MDSCs, which stimulates a tumor microenvironment conducive to immunosuppression and enhanced tumorigenesis (Bhattacharjee et al., 2021; Chen et al., 2021b). Therefore, a better understanding of the mesenchymal TME ecosystem will unveil the mechanistic cross-talks that define their malignancy and resistance to immunotherapy, leading to identifying new potential vulnerabilities to be exploited therapeutically.

### SUMMARY

Mesenchymal colorectal cancer (mCRC) is microsatellite stable (MSS), highly desmoplastic, with CD8+ T cells excluded to the stromal periphery, resistant to immunotherapy, and is driven by low levels of the atypical PKCs (aPKCs). Herein it is shown that a salient feature of these tumors is the accumulation of hyaluronan (HA), which along with reduced aPKC levels, predict poor survival. HA promotes epithelial heterogeneity, and the emergence of a tumors fetal metaplastic cell population (TFSC) endowed with invasive cancer features through a network of interactions with activated fibroblasts and components of the immune system. TFSCs are sensitive to HA deposition in vivo and organoids, and their gene expression signature has prognostic value. It was demonstrated that in vivo HA degradation with a clinical dose of hyaluronidase impairs mCRC tumorigenesis and enables immune checkpoint blockade therapy by promoting the recruitment of B and CD8⁺ T cells, including a proportion with resident memory features.

A detailed characterization of the epithelial compartment and microenvironment of mCRC and its response to HA-depleting treatment is provided. It was found that low aPKC levels are a driver of mCRC not only through the serrated but also through the tubular histological pathways. HA depletion is sufficient to inhibit tumorigenesis and reactivate an anti-tumor immune response as monotherapy. The crosstalk between epithelial cancer cells, the stromal fibroblasts, and the immune system that dictate mCRC dependency on stromal HA and their response to hyaluronidase treatment was defined.

Thus, reduced aPKC drives HA deposition, the mCRC phenotype, and predicts poor survival, HA enhances malignancy and promotes CAF and epithelial heterogeneity in mCRC, immunosuppression is maintained by cross-compartment interactions driven by HA and HA degradation inhibits mCRC tumorigenesis and enables PD-L1 immunotherapy.

In an aspect, provided herein are methods for determining that a subject has or is at risk of having a subtype of colorectal cancer, comprising (a) obtaining an expression level or an amount of (i) hyaluronic acid (HA) and (ii) at least two atypical protein kinase Cs (PKCs) comprising PKCζ and PKCλ/ι in a biological sample from the subject; (b) outputting a report indicative of said subject having or being at risk of having said subtype of the colorectal cancer at least based in part on (a); and (c) administering a therapeutically effective measurement, e.g., a therapeutic compound including an enzyme or antibody or fragment thereof, to the subject.

In another aspect, provided herein are methods for determining that a subject has or is at risk of having a subtype of colorectal cancer, comprising: (a) obtaining an expression level or amount of (i) at least one HA receptor and (ii) at least two atypical PKCs comprising PKCζ and PKCλ/ι in a biological sample from the subject; (b) outputting a report indicative of said subject having or being at risk of having said subtype of the colorectal cancer at least based in part on (a); and (c) administering a therapeutically effective measurement to the subject.

In another aspect, provided herein are methods for determining a subject having or is suspect of having a subtype of colorectal cancer, comprising: (a) obtaining an expression level or an amount of (i) HA, (ii) at least one HA receptor, and (iii) at least two atypical PKCs comprising PKCζ and PKCλ/ι in a biological sample from the subject; (b) determining the subtype of the colorectal cancer at least based in part on (a); and (c) administering a therapeutically effective measurement to the subject.

In some embodiments, the therapeutically effective measurement comprises administering a HA inhibitor. In some embodiments, the HA inhibitor is selected from the group consisting of pegvorhyaluronidase alfa (PVHA), PEGPH20, a hyaluronidase, a HA synthase inhibitor, a HA receptor inhibitor, a CD44-ligant inhibitor, or a combination thereof. In some embodiments, the hyaluronidase is a recombinant hyaluronidase. In some embodiments, the subtype of colorectal cancer is CMS4. In some embodiments, the obtaining step comprises assaying the expression level or the amount of HA, the at least one HA receptor, or the at least two atypical PKCs comprising PKCζ and PKCλ/ι. In some embodiments, the assaying comprises performing immunohistochemistry (IHC), enzyme-linked immunosorbent assay (ELISA), single-molecule array (Simoa), hyaluronidase activity assay, or a combination thereof. In some embodiments, assaying further comprises determining the biological sample has an elevated expression level of the HA as compared to a reference sample that does not have the subtype of colorectal cancer. In some embodiments, the method further comprises assaying an expression level or amount of at least one HA synthase. In some embodiments, assaying comprises determining the biological sample has an elevated expression level of the at least one HA synthase as compared to a reference sample that does not have the subtype of colorectal cancer. In some embodiments, the at least one HA synthase is HAS1, HAS2, or HAS3. In some embodiments, assaying further comprises determining the biological sample has an elevated expression level of the at least one HA receptor as compared to a reference sample that does not have the subtype of colorectal cancer. In some embodiments, the at least one HA receptor comprises CD44. In some embodiments, the method further comprises assaying an expression level or amount of at least one CD44-ligand. In some embodiments, assaying further comprises determining the colorectal cancer has an elevated expression level of the at least one CD44-ligand as compared to a reference sample that does not have the subtype of colorectal cancer. In some embodiments, the at least one CD44-ligand comprises osteopontin (OPN/Spp1). In some embodiments, the method further comprises assaying an expression level or amount of at least one stromal cell marker. In some embodiments, assaying further comprises determining the biological sample has an elevated expression level of the at least one stromal cell marker as compared to a reference sample that does not have the subtype of colorectal cancer. In some embodiments, the at least one stromal cell marker is selected from the group consisting of GREM1, SFRP1, SFRP2, SFRP4, CXCL14, MMP3, IGF1, and cell proliferation makers. In some embodiments, the at least one stromal cell marker comprises SFRP2 or SFRP4. In some embodiments, the at least one stromal cell marker comprises SFRP2 and SFRP4. In some embodiments, assaying further comprises determining the biological sample has a lower expression level of the at least two atypical PKCs as compared to a reference sample that does not have the subtype of colorectal cancer.

In another aspect, provided herein are methods for determining that a subject has or is at risk of having a subtype of colorectal cancer, comprising: (a) obtaining an expression level or amount of at least one stromal cell marker in a biological sample from the subject, wherein the at least one stromal cell marker comprises SFRP2 or SFRP4; (b) outputting a report indicative of said subject having or being at risk of having the subtype of the colorectal cancer at least based on (a); and (c) administering a therapeutically effective measurement to the subject.

In some embodiments, the biological sample has an elevated expression level or amount of the at least one stromal cell marker as compared to a reference sample that does not have the subtype of colorectal cancer. In some embodiments, the at least one stromal cell marker is a fibroblast marker. In some embodiments, the at least one stromal cell marker further comprises GREM1, SFRP1, CXCL14, MMP3, IGF1, or a cell proliferation maker. In some embodiments, the at least one stromal cell marker comprises SFRP2 and SFRP4. In some embodiments, the therapeutically effective measurement comprises administering a HA inhibitor. In some embodiments, the HA inhibitor is selected from the group consisting of pegvorhyaluronidase alfa (PVHA), PEGPH20, a hyaluronidase, a HA synthase inhibitor, a HA receptor inhibitor, a CD44-ligant inhibitor, or a combination thereof. In some embodiments, the hyaluronidase is a recombinant hyaluronidase. In some embodiments, the subtype of colorectal cancer is CMS4. In some embodiments, the obtaining step comprises assaying the expressive level or the amount of the at least one stromal cell marker. In some embodiments, the assaying comprises performing PCR, RT-PCR, DNA sequencing, RNA sequencing, genotyping assay, immunohistochemistry (IHC), enzyme-linked immunosorbent assay (ELISA), single-molecule array (Simoa), or a combination thereof. In some embodiments, the method further comprises assaying an expression level or amount of hyaluronic acid (HA). In some embodiments, assaying further comprises determining the biological sample has an elevated expression level of the HA as compared to a reference sample that does not have the subtype of colorectal cancer. In some embodiments, the method further comprises assaying an expression level or amount of at least one HA synthase. In some embodiments, assaying further comprises determining the biological sample has an elevated expression level of the at least one HA synthase as compared to a reference sample that does not have the subtype of colorectal cancer. In some embodiments, the at least one HA synthase is HAS1, HAS2, or HAS3. In some embodiments, the method further comprises assaying an expression level or amount of at least one HA receptor. In some embodiments, assaying further comprises determining the biological sample has an elevated expression level of the at least one HA receptor as compared to a reference sample that does not have the subtype of colorectal cancer. In some embodiments, the at least one HA receptor is CD44. In some embodiments, the method further comprises assaying an expression level or amount of at least one CD44-ligand. In some embodiments, assaying further comprises determining the colorectal cancer has an elevated expression level of the at least one CD44-ligand as compared to a reference sample that does not have the subtype of colorectal cancer. In some embodiments, the at least one CD44-ligand comprises osteopontin (OPN/Spp1).

In some embodiments, the method further comprises assaying an expression level or amount of at least two atypical PKCs comprising PKCζ and PKCλ/t. In some embodiments, assaying further comprises determining the biological sample has a lower expression level of the at least two atypical PKCs as compared to a reference sample that does not have the subtype of colorectal cancer.

Further provided herein are methods for treating a subject having or is suspected of having a CMS4 colorectal cancer, comprising administering an effective amount of hyaluronic acid (HA) inhibitor to the subject.

Further provided herein are methods for treating a subject having or is suspected of having a CMS4 colorectal cancer, comprising administering an effective amount of hyaluronic acid (HA) inhibitor to the subject, wherein the CMS4 colorectal cancer has an elevated expression level of HA as compared to a reference sample that does not have the CMS4 colorectal cancer.

Further provided herein are methods for treating a subject having or is suspected of having a CMS4 colorectal cancer, comprising administering an effective amount of hyaluronic acid (HA) inhibitor to the subject, wherein the CMS4 colorectal cancer has an elevated expression level of at least one HA receptor as compared to a reference sample that does not have the CMS4 colorectal cancer.

Further provided herein are methods for treating a subject having or is suspected of having a CMS4 colorectal cancer, comprising administering an effective amount of hyaluronic acid (HA) inhibitor to the subject, wherein the CMS4 colorectal cancer has a lower expression level of at least two atypical PKCs comprising PKCζ and PKCλ/ι as compared to a reference sample that does not have the colorectal cancer.

In some embodiments, the CMS4 colorectal cancer has an elevated expression level of at least one HA receptor as compared to the reference sample. In some embodiments, the CMS4 colorectal cancer has a lower expression level of at least two atypical PKCs comprising PKCζ and PKCλ/ι as compared to the reference sample.

Further provided herein are methods for treating a subject having or is suspected of having a CMS4 colorectal cancer, comprising administering an effective amount of hyaluronic acid (HA) inhibitor to the subject, wherein the CMS4 colorectal cancer has an elevated expression level of HA as compared to a reference sample that does not have the CMS4 colorectal cancer, wherein the CMS4 colorectal cancer has an elevated expression level of at least one HA receptor as compared to the reference sample, and wherein the CMS4 colorectal cancer has a lower expression level of at least two atypical PKCs comprising PKCζ and PKCλ/ι as compared to the reference sample.

In some embodiments, the HA inhibitor is pegvorhyaluronidase alfa (PVHA), PEGPH20, a hyaluronidase, a HA synthase inhibitor, a HA receptor inhibitor, or a CD44-ligant inhibitor, or a combination thereof. In some embodiments, the hyaluronidase is a recombinant hyaluronidase. In some embodiments, the CMS4 colorectal cancer has an elevated expression level of at least one HA synthase as compared to a reference sample that does not have the CMS4 colorectal cancer. In some embodiments, the at least one HA synthase is HAS1, HAS2, or HAS3. In some embodiments, the at least one HA receptor is CD44. In some embodiments, the colorectal cancer has an elevated expression level of at least one CD44-ligand as compared to a reference sample that does not have the CMS4 colorectal cancer. In some embodiments, the at least one CD44-ligand comprises osteopontin (OPN/Spp1). In some embodiments, the CMS4 colorectal cancer has an elevated expression level of at least one stromal cell marker as compared to a reference sample that does not have the CMS4 colorectal cancer. In some embodiments, the at least one stromal cell marker is a fibroblast marker. In some embodiments, the at least one stromal marker is selected from the group consisting of GREM1, SFRP1, SFRP2, SFRP4, CXCL14, MMP3, IGF1, and cell proliferation makers. In some embodiments, the at least one stromal cell marker comprises SFRP2 or SFRP4. In some embodiments, the at least one stromal cell marker comprises SFRP2 and SFRP4.

In some embodiments, the method further comprises administering another agent. In some embodiments, the another agent is a TGF-β1 inhibitor, a PD-L1 inhibitor, or a combination thereof. In some embodiments, the CMS4 colorectal cancer is resistant to a TGF-β1 inhibitor treatment.

Further provided herein are methods for treating colorectal cancer, comprising preparing a biological sample taken from a patient in potential need of treatment for colorectal cancer; assaying an expression level or amount of hyaluronic acid (HA) or expression level of at least one HA receptor and at least two atypical PKCs comprising PKCζ and PKCλ/ι in the prepared biological sample; determining a subtype of colorectal cancer based at least in part on the results of the assay; and treating the subject by administering at least one therapeutically effective measurement to the subject based at least in part on the determined subtype.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The following detailed description of the embodiments of the invention will be better understood when read in conjunction with the appended drawings. For the purpose of illustrating the invention, there are shown in the drawing embodiments, which are presently exemplified. It should be understood, however, that the invention is not limited to the precise arrangement and instrumentalities of the embodiments shown in the drawings.

FIGS. 1A-1F demonstrate a reduced expression of atypical PKCs (aPKCs) in human CMS4 tumors.

FIG. 1A illustrates TGFβ-related and EMT-related signatures from GSEA of PRKCI^{low}PRKCZ^{low} (n=31) versus PRKCI^{high}PRKCZ^{high} (n=24) CRC patients from TCGA. (EMT: epithelial-mesenchymal transition).

FIG. 1B illustrates GSEA plots of enrichment in Hallmark EMT and TGFβ signaling for PRKCI^{low} PRKCZ^{low} versus PRKCI^{high}PRKCZ^{high} CRC patients from TCGA. The top 25% of samples were classified as the high group and the bottom 25% of samples as the low group.

FIG. 1C demonstratres a pie chart of TCGA COAD and READ samples showing percentages of each CMS subtype according to the aPKCs expression. CMS annotation was estimated using the CMScaller R package.

FIG. 1D illustrates HAS1, HAS2, HAS3, CD44, SPP1 and SFRP2 levels in CRC patients stratified based on aPKCs expression as in FIG. 1B. *p<0.05, **p<0.01, ***p<0.001, NS, not significant.

FIG. 1E illustrates GSEA plots of enrichment in Hallmark EMT and TGFβ signaling for patients with high HAS score versus with low HAS score. HAS score for each patient was calculated using the GSVA R package and reflects the mean expression for HAS1, HAS2, and HAS3.

FIG. 1F illustrates pie charts of TCGA COAD and READ samples showing percentages of each CMS subtype according to the HAS score.

FIGS. 2A-2K demonstrate that SFRP2 and SFRP4 expression in CRC stroma correlates with CMS4.

FIG. 2A demonstrates an analysis of single-cell RNA-seq data (GSE132465) from human CRC stroma. Clustering results for n=2736 cells are visualized on a UMAP (Uniform manifold approximation and projection) plot. Violin plots show the gene expression levels per single cell in each cluster for key stromal marker genes.

FIGS. 2B-2C illustrate UMAP with clustering results color-coded by tumor stromal types (FIG. 2B) and tissue CMS class of the tumor samples (FIG. 2C). (VSMC: Vascular smooth muscle cells).

FIG. 2D illustrates the proportion and average cell number of tumor stromal cell types for each tissue CMS class.

FIG. 2E illustrates UMAP plot with re-clustering of tumoral fibroblast (n=981). Violin plots showing gene expression levels per single cell in each cluster for top gene markers.

FIG. 2F demonstrates a heatmap of top 25 tumor fibroblast marker genes.

FIG. 2G demonstrates UMAP with fibroblast clustering results divided by tissue CMS class and color-coded by cluster subtype.

FIG. 2H illustrates the proportion and average cell number of tumor fibroblast clusters cell for each tissue CMS class.

FIG. 2I demonstrates pie charts of TCGA COAD and READ samples showing percentages of each CMS subtype according to according to the SFRP2 expression (top) or SFRP4 (bottom). CMS annotation was estimated using the CMScaller R package. The top 25% of samples were classified as the high group and the bottom 25% of samples as the low group.

FIG. 2J illustrates Kaplan-Meier of TCGA COAD and READ samples according to SFRP2 (left) and SFRP4 (right).

FIG. 2K illustrates UMAP of whole tumor cells (n=47285) with clustering results color-coded by tumor cell types (left) and expression of SFRP2 and SFRP4 in whole tumor cells (right).

FIGS. 3A-3K demonstrate scRNAseq of Prkci^{fl/fl}Prkcz^{fl/fl} tumors identifying PVHA as a promising therapy in CMS4 CRC.

FIG. 3A illustrates the experimental design: Prkci^{fl/fl}Prkcz^{fl/fl} (DKO) mice were treated either with PVHA (0.0375 mg/Kg twice per week) or Galunisertib (10 mg twice daily) as described. Intestinal tumors were analyzed by scRNAseq.

FIG. 3B illustrates UMAP of whole cells recovered (n=158661) with clustering results color-coded by each experimental group.

FIG. 3C illustrates UMAP of whole cells with clustering results divided by group and color-coded by cell compartment.

FIG. 3D illustrates the proportion and average cell number of cells for each experimental group.

FIGS. 3E-3F illustrate UMAP of epithelial cells recovered (n=21949) with clustering results color-coded by each experimental group (FIG. 3E) or color-coded by cluster (FIG. 3F). Violin plots showing gene expression levels per single cell in each cluster for top gene markers.

FIG. 3G illustrates expression of DKO tumor signature (TOP 10 DEG from bulk RNAseq), TGFβ signaling and EMT from H compilation (MSigDB), and signatures of gene markers for Revival Stem Cells, Fetal Stem, and YAP/TAZ targets.

FIG. 3H illustrates UMAP of epithelial cells with clustering results divided by group and color-coded by cell-type.

FIG. 3I illustrates the proportion and average cell number of cells for each experimental group.

FIGS. 3J-3K illustrate Olfm4 staining in a non-tumor area of Prkci^{fl/fl}Prkcz^{fl/fl} mice vehicle or treated with PVHA (FIG. 3J) and Clu staining in a tumor area (FIG. 3K).

FIGS. 4A-4G demonstrate the goblet cell analysis identifying a goblet subtype reminiscent of DKO tumor cells.

FIGS. 4A-4B illustrate UMAP of goblet cells (n=3566) indicating goblet cell compartment (FIG. 4A) and UMAP of goblet cells with clustering results color-coded by cell-type (FIG. 4B). Violin plots showing gene expression levels per single cell in each cluster for top gene markers.

FIG. 4C illustrates the expression of differentiating genes found in both vehicle and treatments in goblet cells and YAP/TAZ targets signature.

FIG. 4D illustrates a heatmap of top 10 goblet cells marker genes.

FIG. 4E illustrates UMAP of goblet cells with clustering results divided by group and color-coded by cell-type.

FIG. 4F illustrates the proportion and average cell number of cells for each experimental group.

FIG. 4G illustrates the alcian blue (AB) and Lyz1 double staining in a tumor area of Prkci^{fl/fl}Prkcz^{fl/fl} mice vehicle or treated with PVHA or GAL (left) and their quantification (right). *p<0.05, **p<0.01, ***p<0.001.

FIGS. 5A-5J demonstrate a protective role of a fibroblast population for stem cells induced by PVHA.

FIG. 5A illustrates UMAP of stromal cells (n=17282) clusters with clustering results color-coded by cluster number. Violin plots showing gene expression levels per single cell in each cluster for top gene markers.

FIG. 5B illustrates UMAP of stromal cells with clustering results color-coded by cell-type (left) and proportion and average cell number of cells for each experimental group (right).

FIG. 5C illustrates UMAP plot with re-clustering of tumoral fibroblast (n=1932). Violin plots showing gene expression levels per single cell in each cluster for top gene markers.

FIG. 5D illustrates a heatmap of top 25 tumor fibroblast marker genes.

FIG. 5E illustrates UMAP with fibroblast clustering results divided by tissue group and color-coded by cluster subtype.

FIG. 5F illustrates a heatmap of relative expression of genes involved in proliferation, Immune cell activation, or ECM remodeling process for each fibroblast cluster.

FIG. 5G illustrates UMAP plots showing expression of Pdgfra, Cd81, and Cd34 at the single-cell level.

FIG. 5H illustrates images of Prkci^{fl/fl}Prkcz^{fl/fl} intestine immunostained for Cd81 (red), Pdgfra (green), and DAPI (blue).

FIG. 5I illustrates a heatmap of relative expression of secreted factors resulting in stem cell maintenance and secreted factors resulting in epithelial differentiation.

FIG. 5J illustrates UMAP plots showing expression of TGFβ response, SOX2 targets, and KLF4 target genes signatures at single-cell resolution (top) and violin plots for the mean expression of each fibroblast cluster (bottom).

FIGS. 6A-6N demonstrate the immune landscape of DKO tumors modulated by PVHA and GAL treatment.

FIG. 6A illustrates UMAP of immune cells (n=101543) clustering results color-coded by group.

FIG. 6B illustrates UMAP plots showing expression of well-known immune cell-type markers. Cd3g, Cd8a, and Cd4 for T cells; Cd79 and Jchain for B and plasma cells; S100a8, Apoe, Plac8 and Siglech for myeloid, Hba-a1 for erythrocytes and Mcpt1 for Mast cells.

FIG. 6C illustrates UMAP with immune clustering results divided by group and color-coded by immune cell-type.

FIG. 6D illustrates the proportion and average cell number of immune cells for each experimental group.

FIG. 6E illustrates UMAP plot with re-clustering of myeloid cells (n=33507) color-coded by group.

FIG. 6F illustrates UMAP plots showing expression of myeloid subtypes markers. S100a8 and Icam1 for Neutrophils; Chil3 for Tumor-associated macrophages; C1qa for inflammatory monocytes, and Cst3 and Siglech for dendritic cells.

FIG. 6G illustrates UMAP with myeloid clustering results divided by group and color-coded by myeloid cell-type.

FIG. 6H illustrates the proportion and average cell number of myeloid cells for each experimental group.

FIG. 6I illustrates UMAP plot with re-clustering of T cells (n=26667) color-coded by group.

FIG. 6J illustrates UMAP plots showing expression of T cells subtypes markers: Cd3g and Cd8a for Cd8 T Cells; Cd4 for Cd4 T cells; Klrb1b for NK (natural killer) cells; Stmn1 for proliferating T cells; Foxp3 for Treg and I117a for Th17 cells.

FIG. 6K illustrates UMAP with T cells clustering results divided by group and color-coded by T cell-type.

FIG. 6L illustrates the proportion and average cell number of T cells for each experimental group.

FIG. 6M illustrates the number of cell-cell (tumor epithelium-immune) communications in each treatment condition.

FIG. 6N illustrates a heatmap showing number of communications targeting tumor cells from Inflammatory monocytes (I. Monocytes), Tumor-associated neutrophils (TAN1 and TAN2), dendritic cells (DCs), Tumor-associated macrophages (TAM), CD8 T cells (CD8) and CD4 T cells (CD4) to tumor epithelial cells for each experimental group.

FIGS. 7A-7P illustrate aPKC-low levels correlate with HA-deposition and poor prognosis in human CRC.

FIG. 7A illustrates GSEA of transcriptomic data from TCGA CRC patients according to PRKCI/PRKCZ expression.

FIG. 7B illustrates GSEA of the indicated gene signatures from TCGA CRC patients according to PRKCI/PRKCZ expression.

FIG. 7C illustrates GSEA of transcriptomic data from TCGA CRC patients according to PRKCI/PRKCZ expression using stroma-related signatures.

FIG. 7D illustrates GSEA plots of the indicated gene signatures in TCGA CRC patients according to PRKCI/PRKCZ expression.

FIG. 7E illustrates MCP-counter scores for monocytic lineage, endothelium, and fibroblast according to PRKCI/PRKCZ expression. Box and whiskers graphs indicate the median and the 25th and 75^{th} percentiles, with minimum and maximum values at the extremes of the whiskers.

FIG. 7F illustrates experimental design of PRKCI and PRKCZ editing in patient-derived organoids (PDOs) from CRC.

FIG. 7G illustrates immunoblot of indicated proteins in sgPRKCI/PRKCZ and sgC PDOs (n=3 biological replicates).

FIG. 7H illustrates qPCR of HAS2 in sgPRKCI/PRKCZ and sgC PDOs (n=3 biological replicates).

FIG. 7I illustrates IF for HA (yellow) in sgPRKCI/PRKCZ and sgC PDOs. Scale bars, 50 μm.

FIG. 7J illustrates IF for aPKCs (red) and HA (green) in a human cohort of CRC samples (n=390). Scale bars, 100 μm.

FIG. 7K and FIG. 7L illustrate Kaplan-Meier curve for overall survival of CRC patients according to aPKCs (K) and HA. (L) expression.

FIG. 7M and FIG. 7N illustrate Pie chart of relative distribution of CRC patients according to aPKCs/HA expression. (M) and Kaplan-Meier curve for overall survival (N).

FIG. 7O and FIG. 7 (illustrate IF for aPKCs (red) and HA (green) (O) and quantification (P) in primary CRC samples and metastatic counterparts (n=21, paired). Scale bars, 100 μm. Results are shown as mean±SEM *p<0.05, **p<0.01, ***p<0.001, ****p<0.0001. See also FIG. 14.

FIGS. 8A-8M illustrate targeting HA disrupts the desmoplastic response and impairs mesenchymal tumorigenesis.

FIG. 8A illustrates experimental design of Prkci and Prkcz editing in mouse tumor organoids (MTOs).

FIG. 8B illustrates immunoblot analysis of indicated proteins in sgPrkci/Prkcz and sgC MTOs (n=3 biological replicates).

FIG. 8C illustrates qPCR of indicated genes in sgPrkci/Prkcz and sgC MTOs (n=3 biological replicates).

FIG. 8D illustrates IF for HA (yellow) in sgPrkci/Prkcz and sgC MTOs. Scale bars, 50 μm.

FIG. 8E illustrates GSEA plots of the indicated gene set signatures for sgPrkci/Prkcz versus sgC MTOs (n=3 biological replicates).

FIGS. 8F-8J illustrate subcutaneous injection of sgPrkci/Prkcz and sgC MTOs in WT mice. Mice were treated twice a week with Veh or PEGPH20 0.0375 mg/kg for 3 weeks (MTOs: sgC n=10, sgC PEGPH20-treated n=10, sgPrkci/Prkcz n=10, and sgPrkci/Prkcz PEGPH20-treated n=8). Experimental design (F); Immunohistochemistry (IHC) for HA, Masson's trichrome, and αSMA (G) staining quantification (H); tumor volume (I) and tumor weight (J). Scale bars, 100 μm.

FIG. 8K and FIG. 8L illustrate GSEA from Quant-seq on sgPrkci/Prkcz MTOs PEGPH20-treated tumors (n=4) versus Veh (n=3) using compilation H (MSigDB) (K) and stroma-related signatures (L).

FIG. 8M illustrates GSEA of the indicated gene signatures for sgPrkci/Prkcz MTOs PEGPH20-treated tumors (n=4) versus Veh (n=3). Results are shown as mean±SEM *p<0.05, **p<0.01, ***p<0.001, ****p<0.0001. See also FIG. 15.

FIGS. 9A-9O illustrate targeting HA represses mesenchymal intestinal CRC.

FIG. 9A illustrates experimental design for tamoxifen treatment in organoids from Prkci^{f/f}Prkcz^{f/f}; Villin-CreER mice.

FIG. 9B illustrates qPCR of indicated genes in tamoxifen- or Veh-treated organoids (n=3 biological replicates).

FIG. 9C illustrates IF for HA (yellow) of Veh- or tamoxifen-treated organoids. Scale bars, 50 μm.

FIG. 9D illustrates IHC for HA of small-intestinal sections from Prkci^{f/f}Prkcz^{f/f} and Prkci^{f/f}Prkcz^{f/f}; Villin-Cre mice. Scale bars, 50 μm.

FIGS. 9E-9O illustrate Prkci^{f/f}Prkcz^{f/f}; Villin-Cre mice (mOle and female, 11-week-old) treated with Veh (n=10) or PEGPH20 (n=12), 0.0375 mg/kg for 3 weeks. Experimental design (E); IHC HA, Masson's trichrome, and αSMA(F); staining quantification (n=5) (G); macroscopic images of tumors (H); total tumor number, tumor size, and tumor load (I); quantification of cancer incidence (J); and sessile serrated lesions (SSL) and carcinoma quantification of tumors (K). Scale bars, 50 μm (F), 10 mm (H). The red arrow denotes tumors>5 mm (H). (L-N) GSEA of transcriptomic data from Quant-seq on PEGPH20-treated tumors versus Veh (n=3) using compilation H (MSigDB) (L), stroma-related signatures (M) and serrated-related signatures (N). (O) GSEA plots of the indicated gene signatures for PEGPH20-treated tumors versus Veh (n=3). Results are shown as mean±SEM *p<0.05, **p<0.01, ***p<0.001, ****p<0.0001. See also FIG. 16.

FIGS. 10A-10S illustrate remodeling of the mesenchymal intestinal tumor stroma by PEGPH20 treatment.

FIG. 10A illustrates experimental design and workflow of scRNAseq. Tumors from Veh (n=5) and PEGPH20-treated mice (n=3) were dissected and digested into single-cell suspensions for sequencing.

FIGS. 10B and 10C illustrate uniform manifold approximation and projection (UMAP) of tumor cells colored by treatment (B) and by the major cellular compartments (C).

FIGS. 10D and 10E illustrate UMAP of stromal cells colored by treatment (D) and by the major stromal cell type (E).

FIG. 10F illustrates stromal-cell-type percentage relative to the total stromal cells count per treatment.

FIGS. 10G and 10H illustrate UMAP of endothelial cells colored by treatment (G) and by endothelial cell type (H).

FIG. 10I illustrates endothelial-cell-type percentage relative to the total stromal cells count per treatment.

FIGS. 10J and 10K illustrate UMAP of fibroblast colored by treatment (J) and by fibroblast cell type (K).

FIG. 10L illustrates violin plots for the indicated gene signatures in the fibroblast cell types.

FIGS. 10M, 10N and 10O illustrate UMAP of fibroblast colored by fibroblast cell type (M) and fibroblast-cell-type percentage relative to the total fibroblast count per treatment (N). (O) Significantly enriched extracellular matrix (ECM)-related signatures in intermediate fibroblast treated with PEGPH20 versus Veh.

FIGS. 10P and 10Q illustrate PDGFRA staining (red) with NRG1 (green) and panCK (white) (P) and quantification (Q) in Veh- and PEGPH20-treated tumors (n=5).

FIGS. 10R and 10Q illustrate CD34 staining (green) with PDFGRA (red) and CD31 (white) (R) and quantification (S) in Veh- and PEGPH20-treated tumors (n=5). Scale bars, 100 μm. Results are shown as mean±SEM *p<0.05, **p<0.01, ***p<0.001, ****p<0.0001, See also FIG. 17.

FIGS. 11A-11Q illustrate scRNA-seq reveals a complex heterogeneity and hierarchy maintained by HA in tumor epithelial cells.

FIGS. 11A and 11B illustrate UMAP of epithelial cells colored by treatment (A) and by epithelial cell type (B).

FIG. 11C illustrates RNA velocities visualized on the UMAP projection in (B).

FIG. 11D illustrates violin plots for the indicated gene signatures in cycling transient amplifying (cTAs), tumor cTAs (TcTAs), tumor revival stem cells (TRSCs), and tumor fetal stem cells (TFSCs).

FIG. 11E illustrates scheme showing that cTAs can differentiate to TcTA and conserve epithelial cancer cell hierarchical heterogeneity.

FIG. 11F illustrates violin plots for the indicated gene signatures in immature goblet cells, mature goblet cells, and tumor goblet cells (TGC).

FIG. 11G illustrates RNA velocities in tumor epithelial cells for each treatment (left) and tumor-cell-type percentage relative to the total tumoral cells count per treatment (right).

FIG. 11H illustrates IHC for ANXA10 and CLU in PEGPH20-treated tumors (n=5). Scale bars, 50 μm.

FIGS. 111 and 11J illustrate UMAP of goblet cells colored by goblet cell type (I) and goblet-cell-type percentage relative to the total goblet cells count per treatment (J).

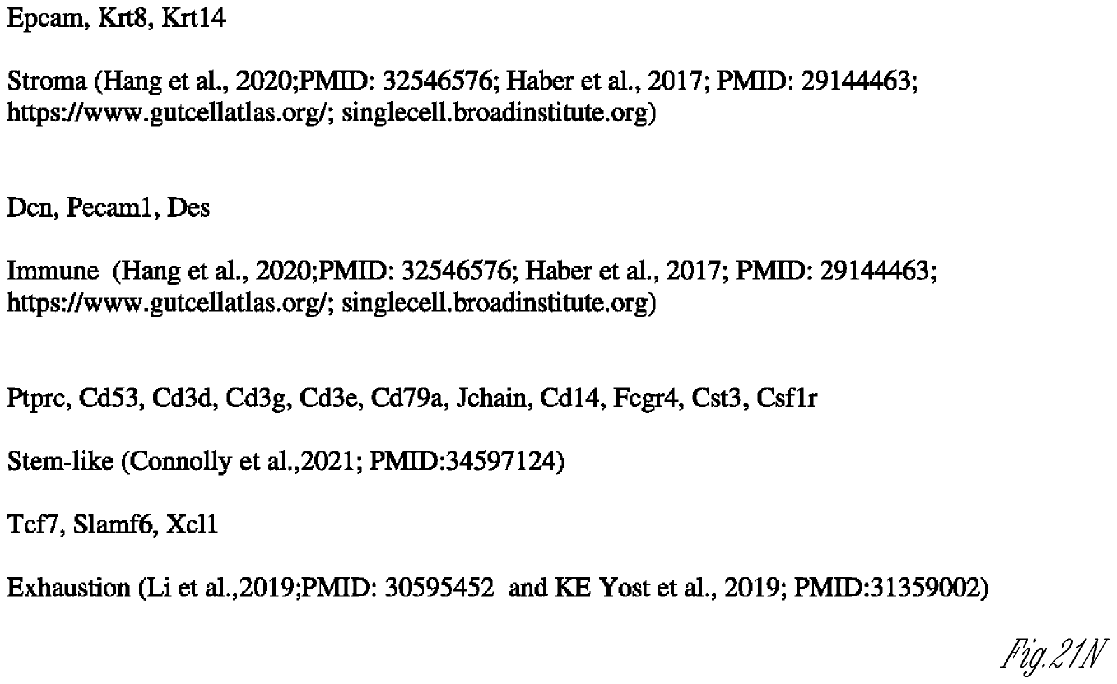

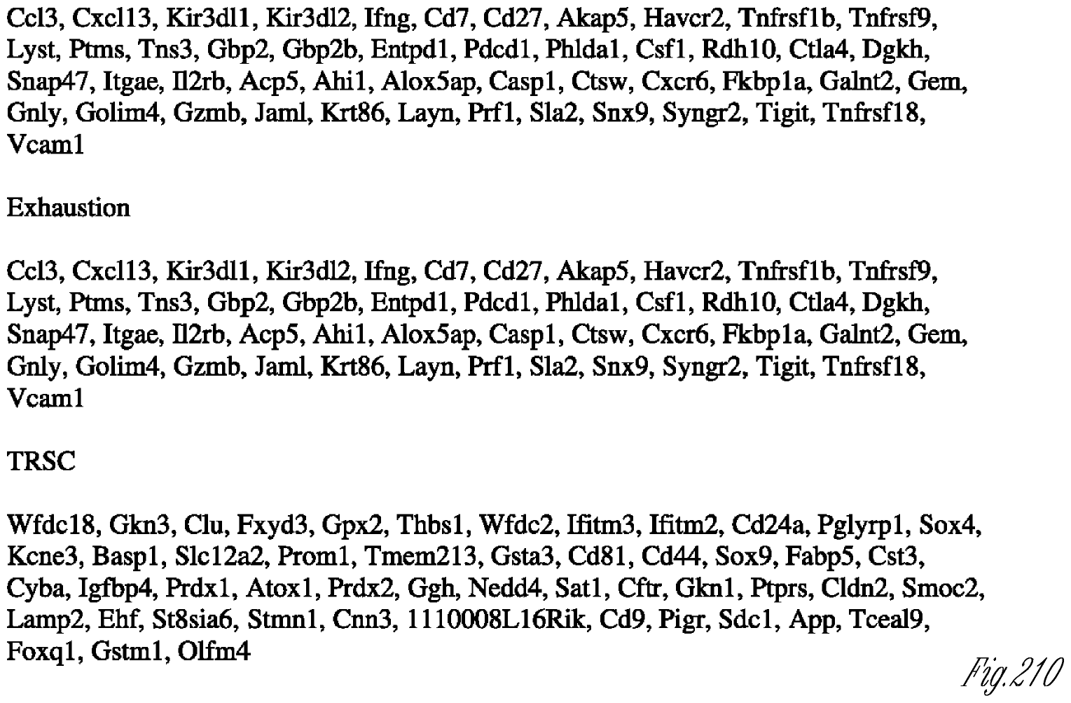

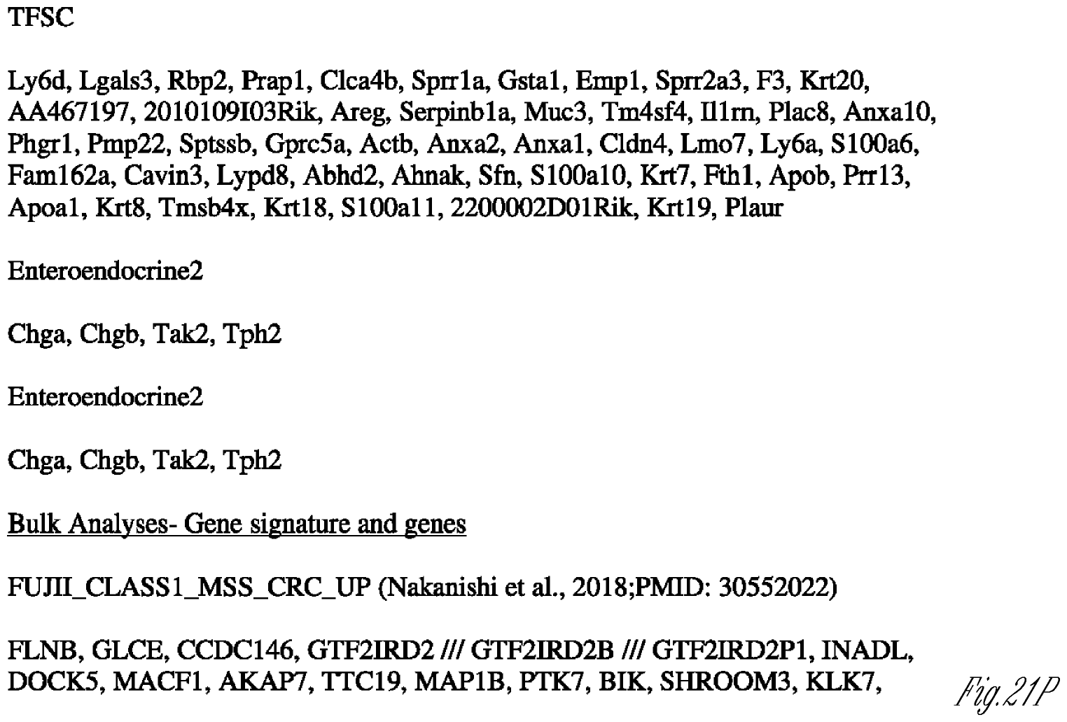

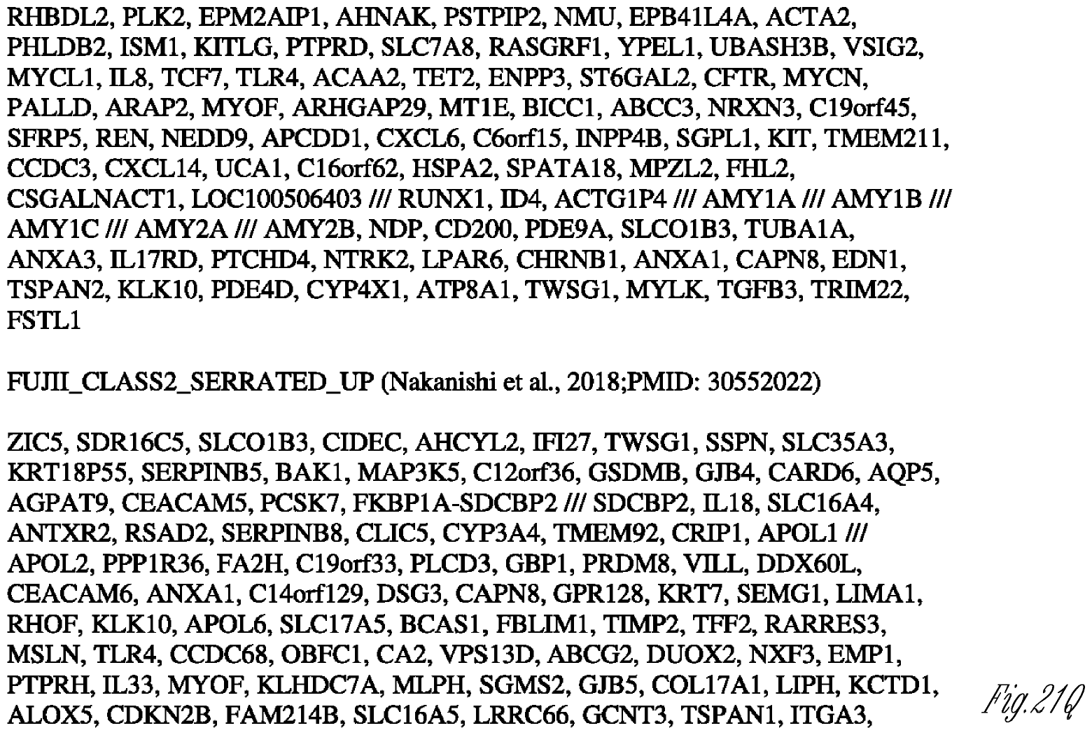

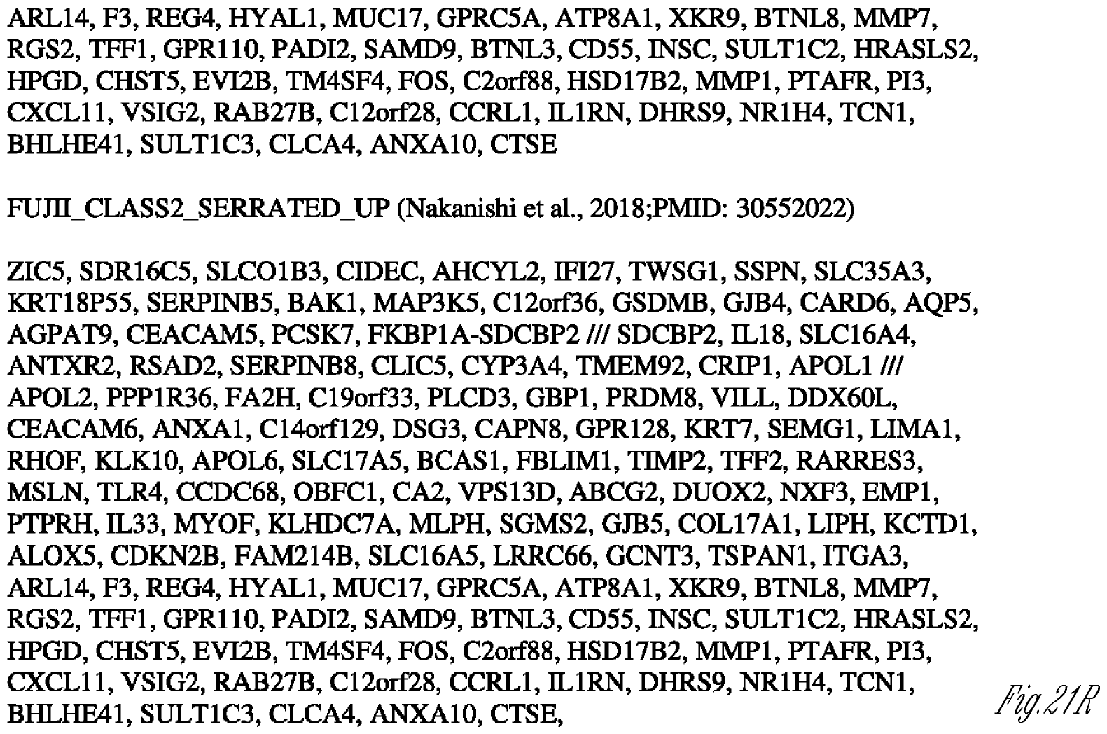

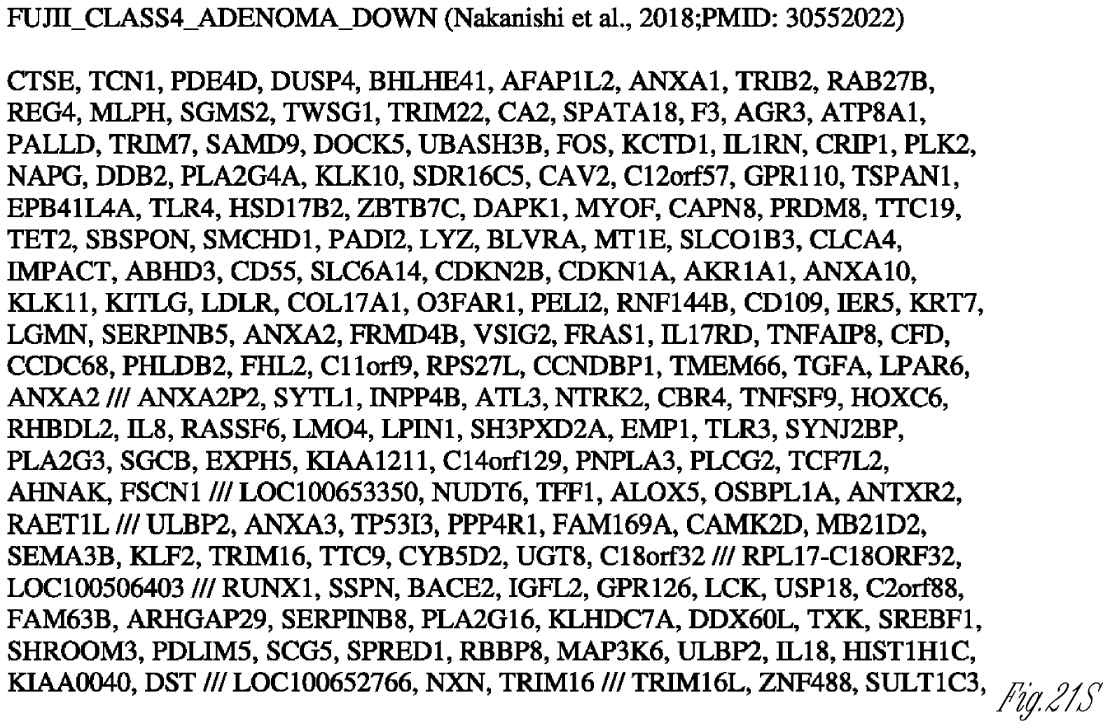

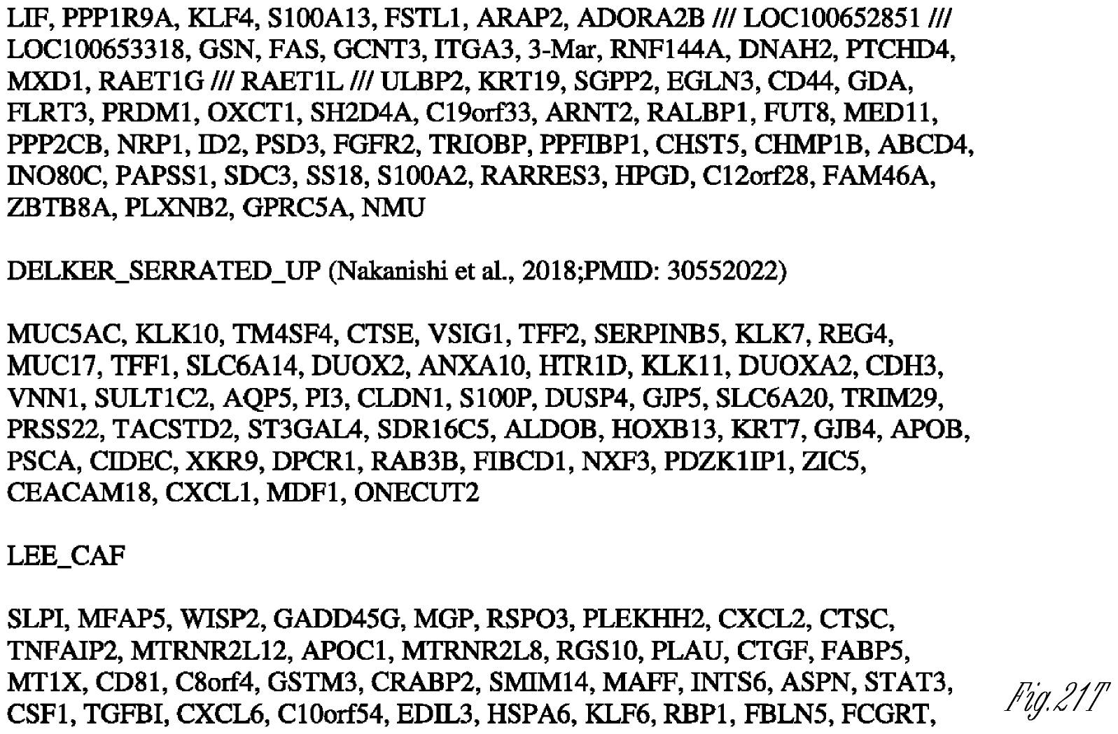

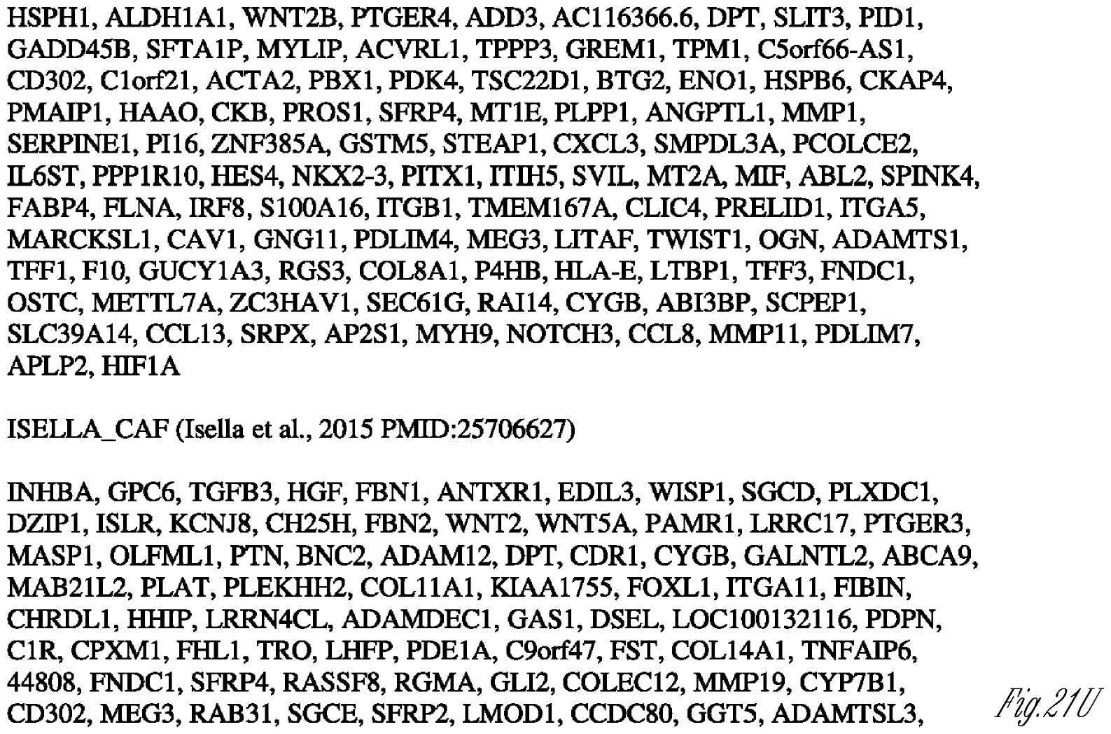

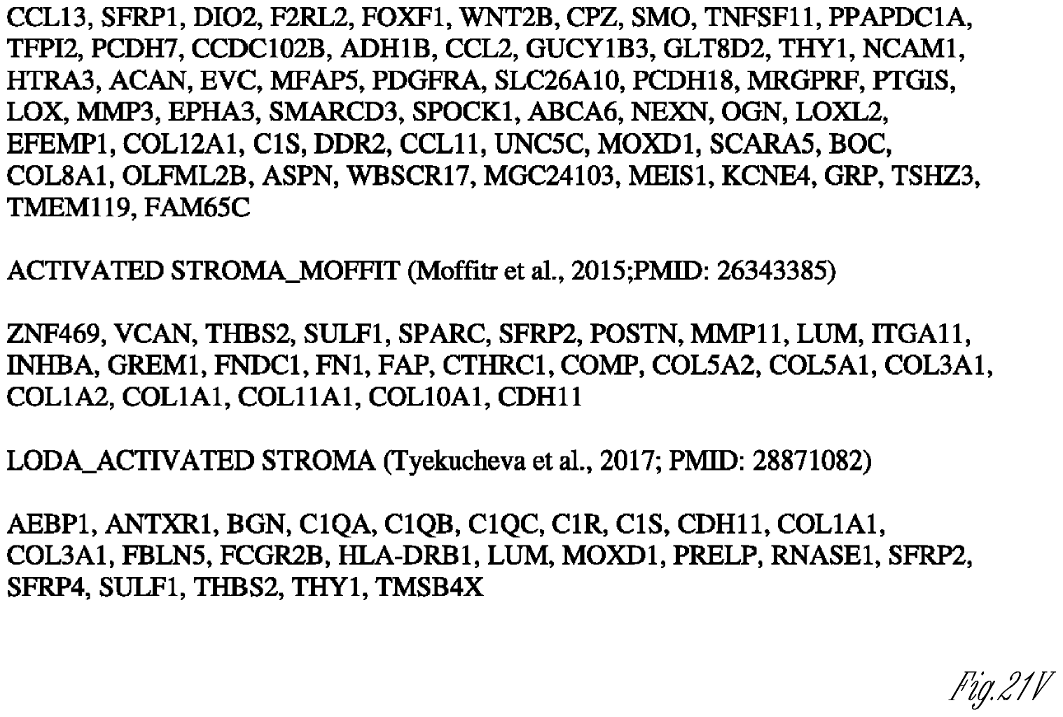

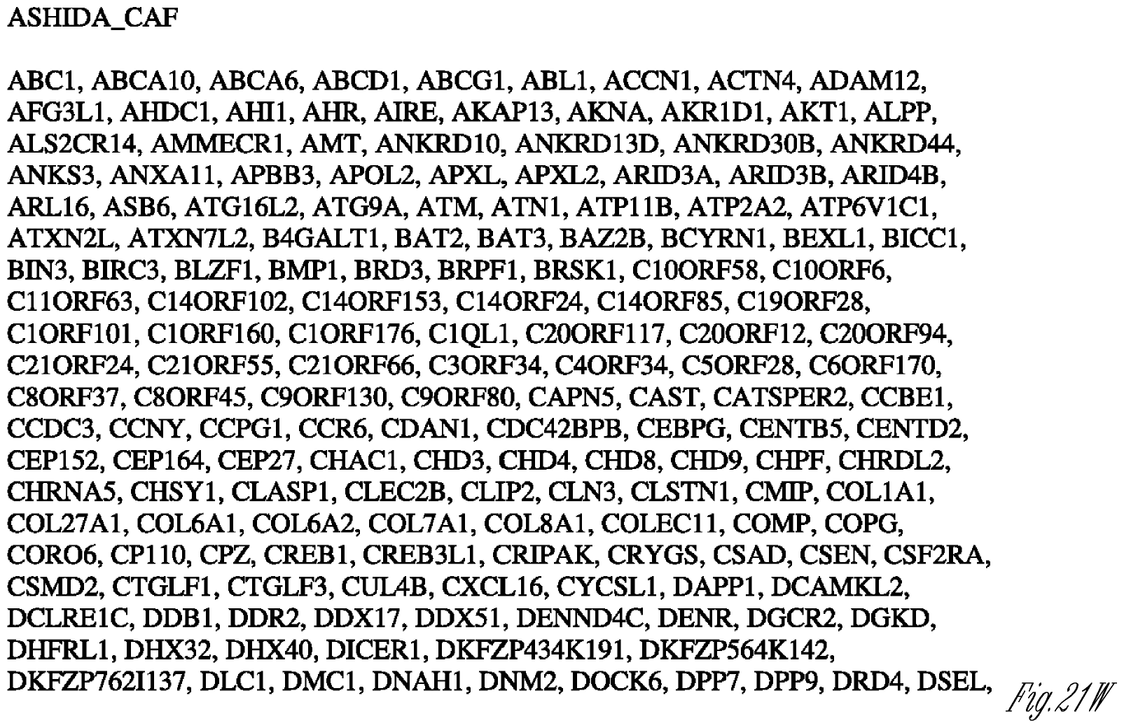

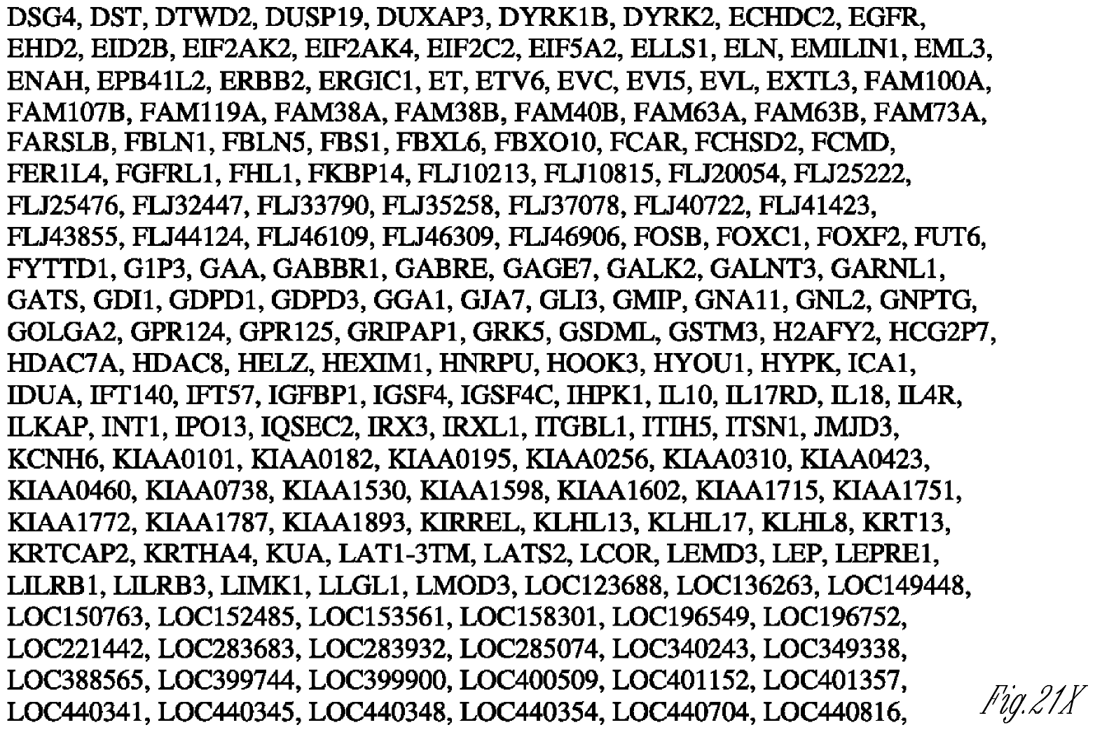

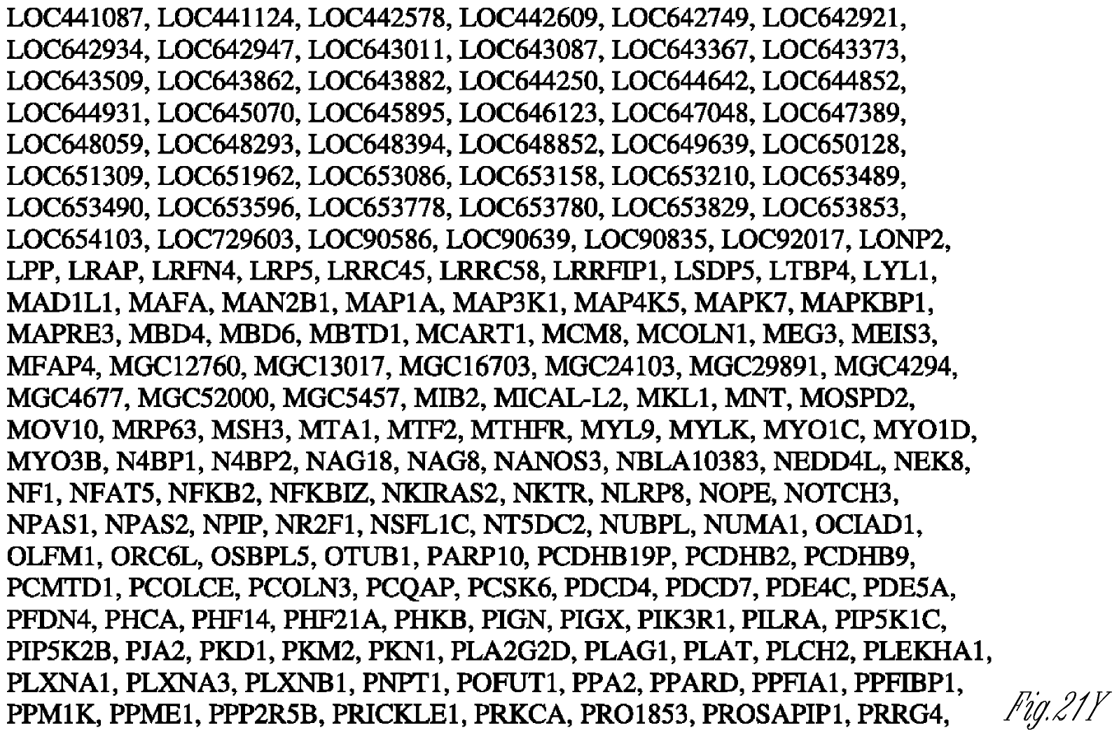

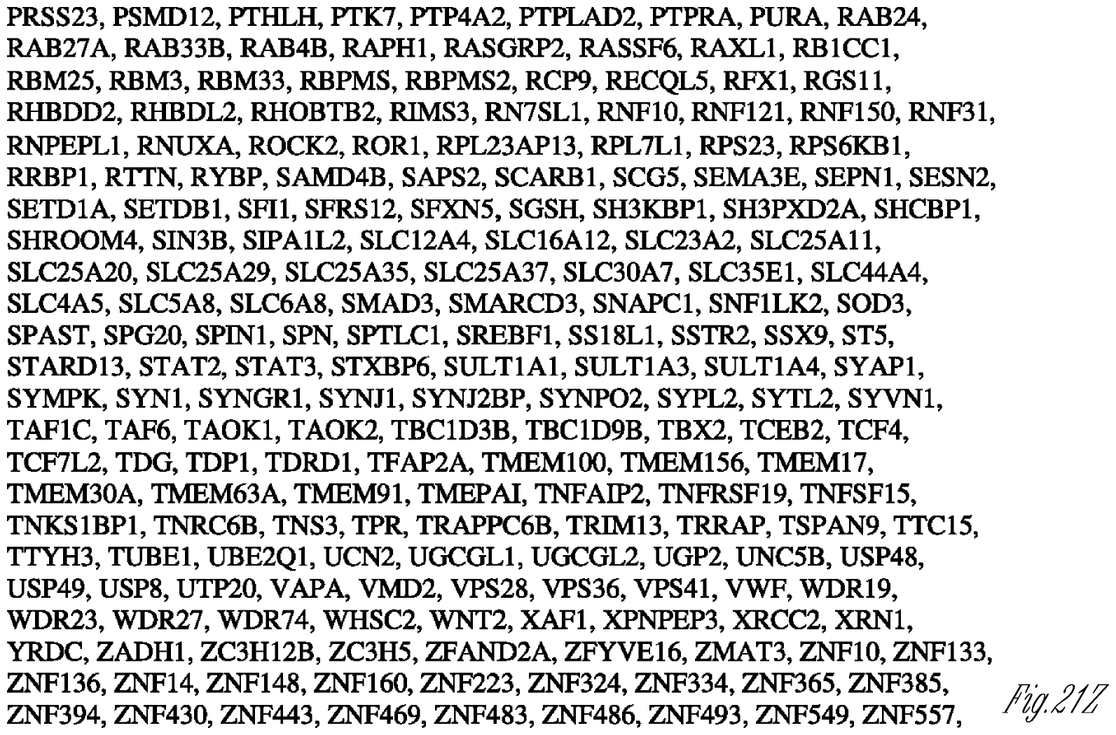

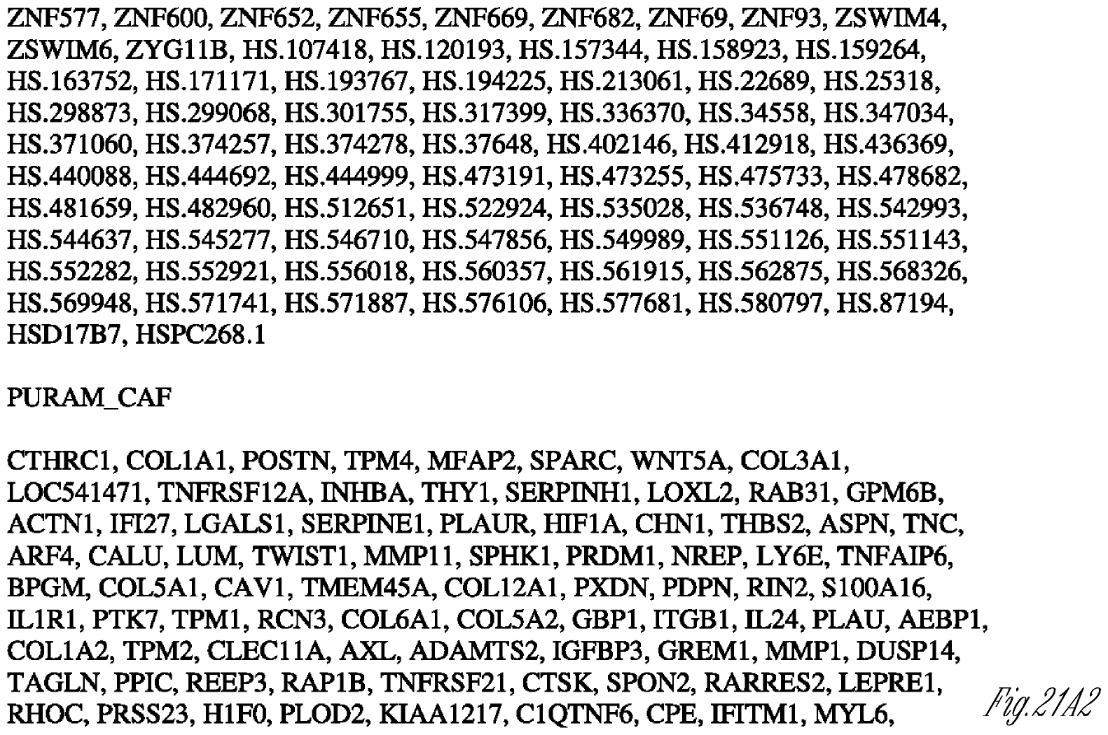

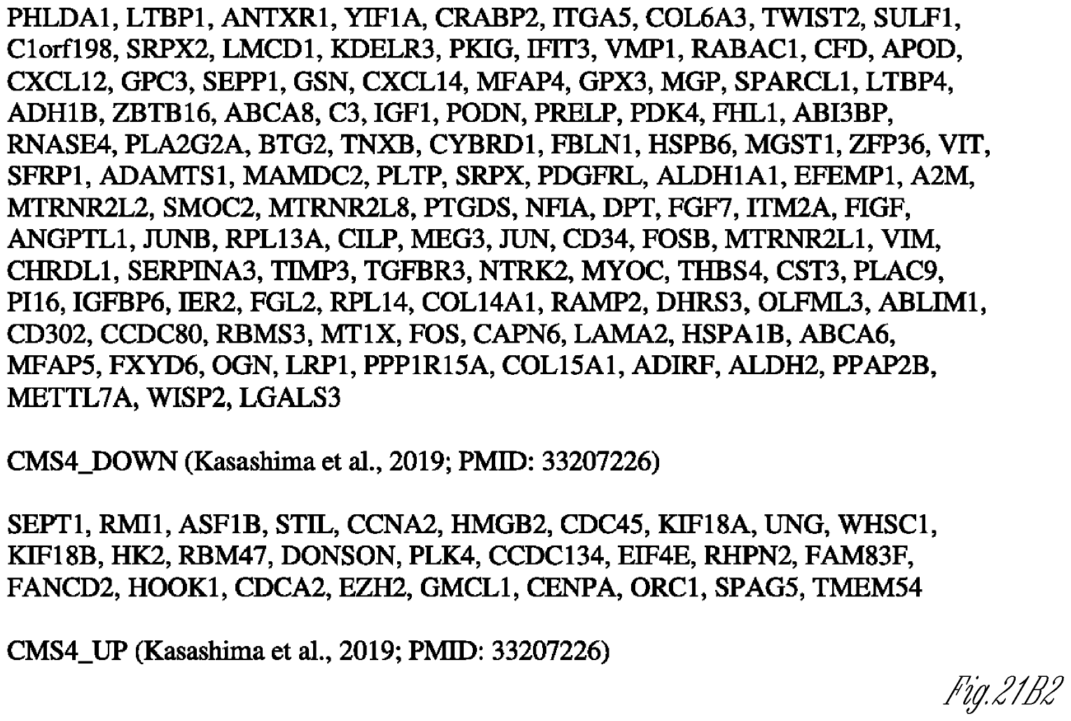

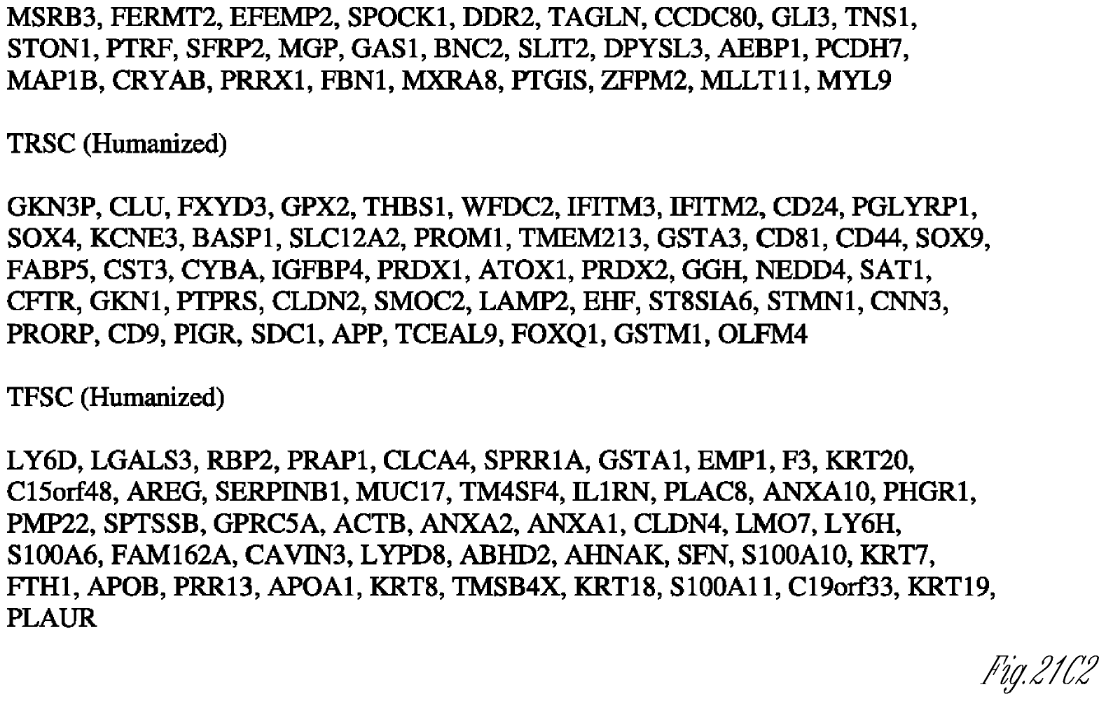

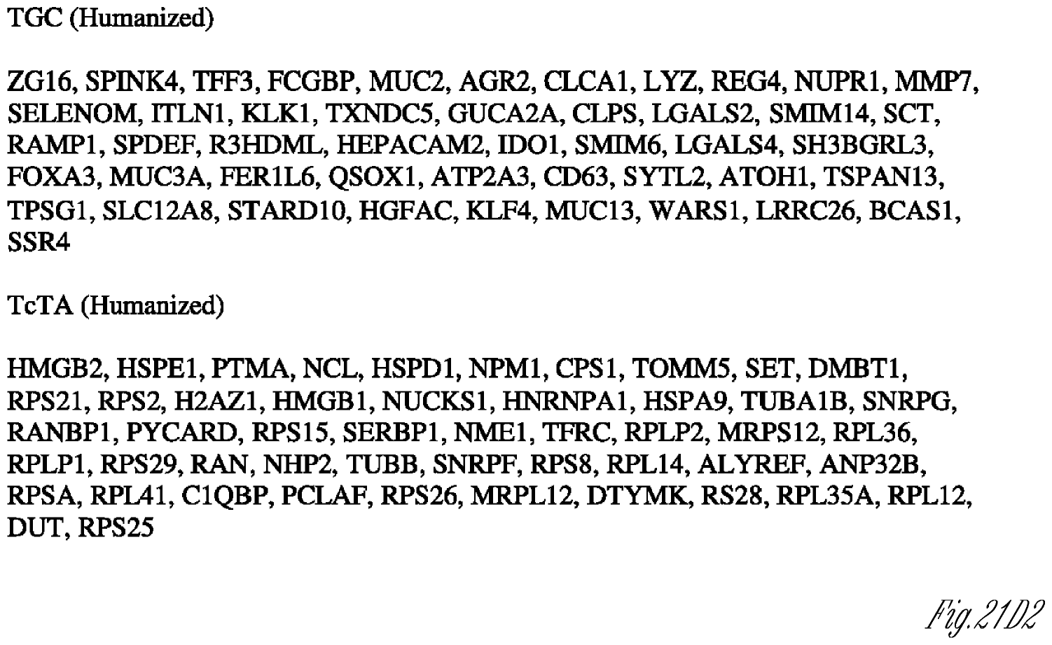

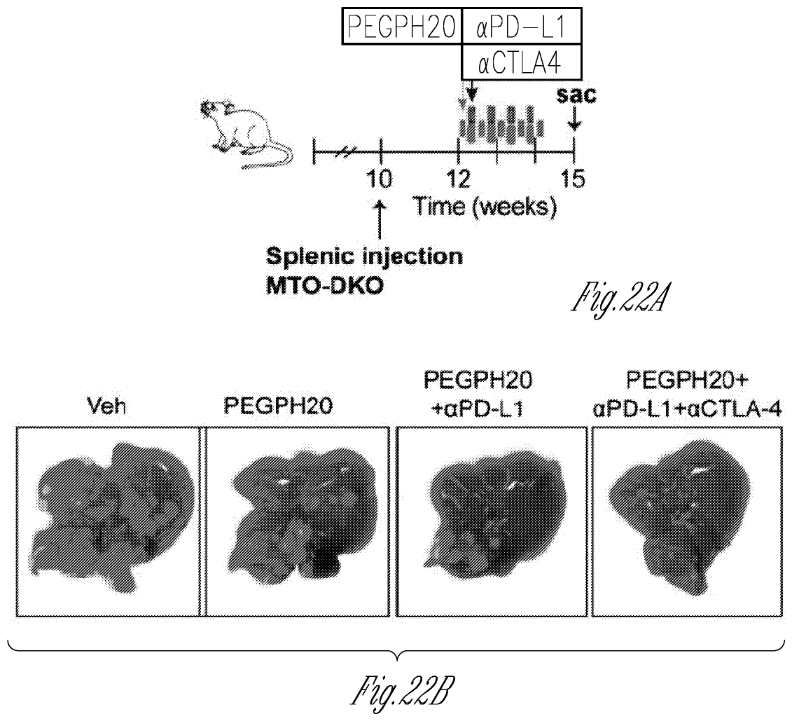

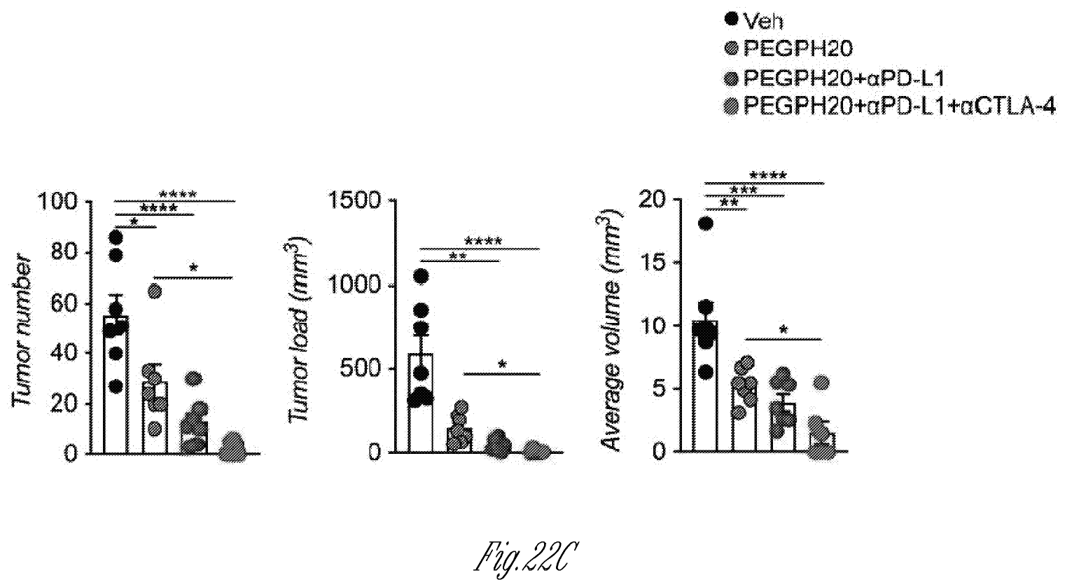

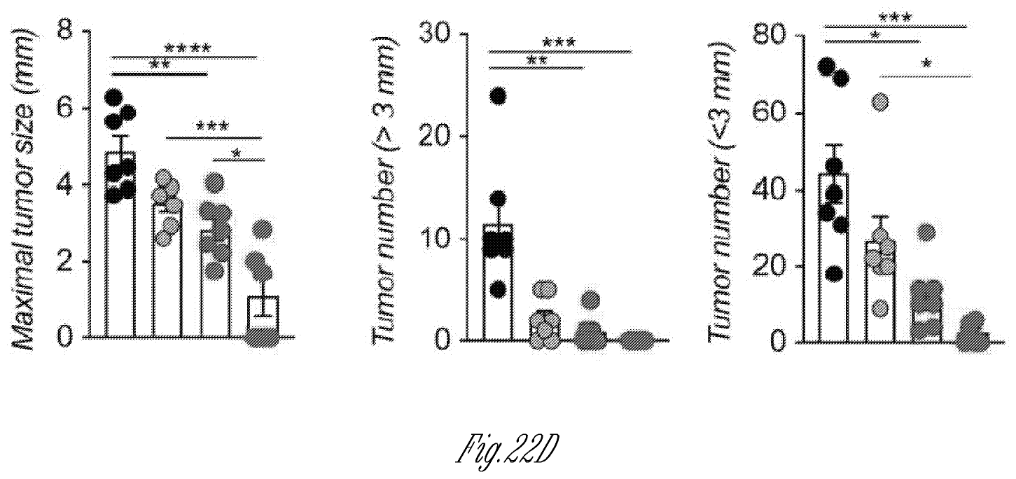

FIGS. 11K and 11L illustrate IHC for ANXA10 with alcian blue (K) and IF for ANXA10 (red) and MUC2 (green) (L) in Veh- and PEGPH20-treated tumors (n=5). Scale bars, 25 μm.

FIG. 11M illustrates Kaplan-Meier curve for disease-free survival of TCGA CRC patients according to TFSC score.

FIGS. 11N and 11O illustrate CellphoneDB analysis showing the number of ligand-receptor interactions between tumor epithelial cells and fibroblast (N) and Dot plot for ligand-receptor pairs of growth factors between telocytes and tumor epithelial in Veh-treated tumors (O).

FIGS. 11P and 11Q illustrate CellphoneDB analysis showing the number of ligand-receptor interactions between tumor epithelial cells and fibroblast cell types (N) and Dot plot for ligand-receptor pairs of Lgr5 and Rspo co-factors between trophocyte and tumor epithelial in Veh- and PEGPH20-treated tumors (Q). Results are shown as mean±SEM *p<0.05, **p<0.01, ***p<0.001, ****p<0.0001. See also FIG. 18.

FIGS. 12A-12M illustrate HA induces immunosuppression and impairs immunosurveillance in mesenchymal intestinal tumors.

FIG. 12A illustrates GSEA of transcriptomic data from Quant-seq on Prkci^{f/f} Prkcz^{f/f}; Villin-Cre PEGPH20-treated tumors versus Veh (n=3) (top), and sgPrkci/Prkcz PEGPH20-treated tumors (n=4) versus Veh (n=3) (bottom) using compilation H (MSigDB).

FIG. 12B illustrates violin plots for the indicated gene signatures in TcTAs, TRSCs, TFSCs and TGCs treated with Veh or PEGPH20.

FIGS. 12C and 12D illustrate UMAP of all immune cells colored by the major immune cell type (C) and immune-cell-type percentage relative to the total immune cells count per treatment (D).

FIGS. 12E and 12F illustrate UMAP of all T cells colored by the T cell type (E) and canonical lineage marker expression for CD8+ T cell and CD4+Treg (left) showing the percentage of CD4+Treg, CD8+T, and CD8+T/CD4+Treg ratio per treatment (right) (F).

FIG. 12G illustrates T-cell-type percentage relative to the total T cells count per treatment.

FIG. 12H illustrates violin plots for the indicated gene signatures in CD8+ T cells treated with Veh or PEGPH20.

FIG. 12I illustrates seven-color overlay image for the indicated genes in Veh- and PEGPH20-treated tumors (n=3). Scale bars, 100 m. The white arrows denote CD4+ Treg and myeloid cells; the yellow arrows mark CD8+ T cells and the red arrows point to B cells.

FIG. 12J illustrates cellphoneDB analysis showing dot plot for ligand-receptor pairs of cytokines between telocytes, intermediate and myeloid cells in Veh- or PEGPH20-treated tumors.

FIGS. 12K and 12L illustrate Dot plot for ligand-receptor pairs of Ccl27a/Ccl28-Ccr10 (K) and Ccl25-Ccr9 and XcIl-XcrI (L) between tumor epithelial cells and immune cells in Veh- or PEGPH20-treated tumors.

FIG. 12M illustrates predicted regulatory crosstalk between tumor epithelial cells, fibroblasts, and the immune system in Veh- or PEGPH20-treated tumors. Results are shown as mean±SEM *p<0.05, **p<0.01, ***p<0.001, ****p<0.0001. See also FIG. 19.

FIGS. 13A-13F illustrate the combination therapy of PEGPH20 with anti-PDL1 improves the response of mesenchymal CRC tumors.

FIG. 13A illustrates IF for PDL1 (green) and CD45 (red) of tumor-draining lymph nodes (TDLNs) in sgC and sgPrkci/Prkcz MTOs. Scale bars, 100 μm.

FIGS. 13B-13F illustrate Subcutaneous injection of sgPrkci/Prkcz MTOs in WT mice treated twice a week with PEGPH20 (0.0375 mg/kg) and αPD-L1 (5 mg/kg) for 4 weeks (n:Veh=14, PEGPH20-treated=15 and PEGPH20-treated with αPD-L1=15). Experimental design (B); tumor volume (C); tumor weight (D); IHC for HA, αSMA, CD8, FOXP3, S100a8, and CD19 of tumors (E) and quantification (F). Scale bars, 100 μm. Results are shown as mean±SEM *p<0.05, **p<0.01, ***p<0.001, ****p<0.0001.

FIGS. 14A-14F illustrate aPKCs expression correlates with mesenchymal features and HA deposition in human CRC, related to FIG. 7.

FIG. 14A illustrates a pie chart of CMS relative distribution of CRC TCGA samples according to aPKCs expression (n=31).

FIGS. 14B and 14C illustrate volcano plot showing HAS1 and HAS2 as differentially expressed genes (B) and HAS1 and H-AS2 mRNA levels.

FIGS. 14D and 14E illustrate PRKCI^{low}PRKCZ^{low} (n=31) versus PRKCL^{high}PRKCZ^{high} (n=24) in CRC patients. (D) GSEA plots of enrichment in the indicated gene signatures in CRC patient samples from TCGA according to HAS score (n=301). (E) Baseline clinical and pathological characteristics in four groups classified by tumoral aPKC expression and stromal HA deposition.

FIGS. 14F and 14G illustrate Multivariable logistic regression analyses for the factors associated with positive HA deposition (F) and high aPKC expression (G).

FIG. 14H illustrates multivariable Cox proportional hazards regression analysis for overall survival. aPKC-high/HA-positive group was used as a reference to estimate the relative risks of the other three groups. 95% CI: 95% confidence intervals, HR: hazard ratios. Box and whiskers graphs indicate the median and the 25th and 75^{th} percentiles, with minimum and maximum values at the extremes of the whiskers. *p<0.05, **p<0.01, ***p<0.001, ****p<0.0001.

FIGS. 15A-15E illustrate aPKC promotes HA deposition in vivo in orthotopic MTO transplantation, related to FIG. 8. Colonoscopy-guided orthotopic transplantation of sgPrkci/Prkcz (n=6) and sgC (n=6) MTOs in WT mice. Experimental design (A), colonoscopy (top) and colonic macroscopic images (bottom) of tumors generated in the colon submucosa by injection of sgPrkci/Prkcz and sgC MTOs (B), tumor volume (C), Hematoxylin and eosin (H&E) (D) and IHC for HA (E) of colon sections from sgPrkci/Prkcz and sgC MTOs. Scale bars, 50 μm. Results are shown as mean±SEM. *p<0.05, **p<0.01, ***p<0.001, ****p<0.0001.

FIGS. 16A-16E illustrate degradation of hyaluronan by PEGPH20 treatment reduces Prkci^{f/f}Prkcz^{f/f}; Villin-Cre intestinal and colon tumors, related to FIG. 9.

FIG. 16A illustrates H&E staining, red lines mark tumor and black lines SSL of small intestine sections from Prkci^{f/f}Prkcz^{f/f}; Villin-Cre mice treated with Veh (n=10) or PEGPH20 (n=12). Scale bars, 2 mm.

FIG. 16B illustrates representative H&E images of SSL, poorly differentiated adenocarcinoma, and invasive carcinoma of small intestine sections from Prkci^{f/f}Prkcz^{f/f}; Villin-Cre mice treated with Veh (n=10) or PEGPH20 (n=12). Scale bars, 100 μm (B).

FIGS. 16C-16E illustrate H&E staining, black lines mark tumor (T). Scale bars, 200 μm. (C). Total tumor number, tumor load (D), and quantification of cancer incidence (E) from small intestine of Prkci^{f/f}Prkcz^{f/f}; Villin-Cre mice treated with Veh (n=10) or PEGPH20 (n=12). Results are shown as mean±SEM *p<0.05, **p<0.01, ***p<0.001, ****p<0.0001.

FIGS. 17A-17L illustrate scRNA-seq of Prkci^{f/f}Prkcz^{f/f}; Villin-Cre tumors treated with PEGPH20,

FIGS. 17A-17C illustrate UMAP feature plots colored by the expression of indicated gene signatures (A) or by the expression of indicated genes (B) and dot plot of indicated gene expression by stromal cell type in all Prkci^{f/f}Prkcz^{f/f}; Villin-Cre stromal cells (C).

FIGS. 17D-17G illustrate UMAP feature plots colored by the expression of indicated genes (D), dot plot of indicated gene expression by endothelial cell type (E), UMAP feature plots colored by the expression of indicated signatures (F), and violin plots for the indicated gene signatures (G) in Prkci^{f/f}Prkcz^{f/f}; Villin-Cre endothelial cells.

FIGS. 17H and 17I illustrate violin plots for endothelial normalization and disorganization signature (H) and IFNγ, IFNα, angiogenesis, glycolysis, and hypoxia from H compilation (MSigDB) (I) in endothelial cells re-clustered and splitted by treatment, Veh or PEGPH20.

FIGS. 17J-17M illustrate UMAP feature plots colored by the expression of indicated genes (J), dot plot of indicated gene expression by fibroblast cell type (K), and heatmap showing average expression levels of the indicated genes (L) in Prkci^{f/f}Prkcz^{f/f}; Villin-Cre fibroblasts. (M) Volcano plot of differentially expressed genes in intermediate fibroblast treated with PEGPH20 versus untreated intermediate fibroblast. Results are shown as mean±SEM *p<0.05, **p<0.01, ***p<0.001, ****p<0.0001.

FIGS. 18A-18O illustrate tumor epithelial cell annotation and subpopulation characterization, related to FIG. 11.

FIG. 18A illustrates UMAP feature plots colored by the expression of indicated signatures in Prkci^{f/f}Prkcz^{f/f}; Villin-Cre epithelial cells.

FIG. 18B illustrates dot plot of indicated marker gene expression by epithelial cell type.

FIG. 18C illustrates inferred large-scale copy number variations (CNVs) identifying TcTA, TRSC, TFSC, TGC and non-cancer cells. Epithelial and spike in control cells are included on the y-axis and chromosomal regions on the x-axis. Amplifications (red) or deletions (blue) were inferred by averaging expression over 100-gene stretches on the respective chromosome.

FIGS. 18D and 18E illustrate UMAP of epithelial cells colored by inferred cell malignancy identity (D) and malignant epithelial cell percentage relative to the total epithelial cells count per treatment (E) in Prkci^{f/f}Prkcz^{f/f}; Villin-Cre tumors.

FIG. 18F illustrates UMAP tumor epithelial cells colored by tumor epithelial cell type in all Prkci^{f/f}Prkcz^{f/f}; Villin-Cre tumors.

FIG. 18G illustrates dot plot of indicated gene expression by tumor epithelial cell type.

FIG. 18H illustrates UMAP feature plots colored by the expression of indicated signatures in Prkci^{f/f}Prkcz^{f/f}; Villin-Cre tumor epithelial cells.

FIGS. 18I and 18J illustrate UMAP goblet cells colored by goblet cell type (I) and colored by the expression of indicated genes (J) in all Prkci^{f/f}Prkcz^{f/f}; Villin-Cre tumors.

FIG. 18K illustrates dot plot of indicated gene expression by goblet cell type.

FIG. 18L illustrates UMAP feature plots colored by the expression of indicated signatures in Prkci^{f/f}Prkcz^{f/f}; Villin-Cre goblet cells.

FIG. 18M illustrates schematic representation of PEGPH20 treatment in mouse tumor organoids (MTOs).

FIG. 18N illustrates qPCR of indicated genes in Veh- and PEGPH20-treated MTOs (n=3 biological replicates).

FIG. 18O illustrates Kaplan-Meier curve for disease-free survival (DFS) of TCGA CRC patients according to TcTA, TRSC and TGC scores. Results are shown as mean±SEM *p<0.05, **p<0.01, ***p<0.001, ****p<0.0001.

FIGS. 19A-19M illustrate PEGPH20 alleviates immunosuppression and allows CD8+ T cell infiltration, related to FIG. 12.

FIG. 19A illustrates UMAP of immune cells colored by treatment in Prkci^{f/f}Prkcz^{f/f}; Villin-Cre tumors.

FIG. 19B illustrates eatmap showing average expression of B and plasma cells, tumor-associated neutrophils (TANs), T and NK cells, tumor-associated macrophages (TAMs), dendritic cells (DCs), and inflammatory monocytes (IMs) marker genes across immune cells subset in scRNA-seq data.

FIG. 19C illustrates UMAP of T cells colored by treatment in Prkci^{f/f}Prkcz^{f/f}; Villin-Cre tumors.

FIG. 19D illustrates heatmap showing average expression of CD8⁺ Tem, CD8⁺ Trm, CD8⁺ Tex, resting CD4⁺ T, CD4⁺ Treg, CD4⁺ Th17, CD4⁺ Th2-like, proliferative, gd and NK marker genes across T cell subset in scRNA-seq data.

FIG. 19E illustrates violin plots for the indicated gene signatures in CD8⁺ T cells in CD8⁺ Tem, CD8⁺ Trm, and CD8⁺ Tex.

FIG. 19F illustrates CD138 staining from Prkci^{f/f}Prkcz^{f/f}; Villin-Cre Veh- or PEGPH20-treated tumors (n=3).

FIGS. 19G and 19H illustrate CellphoneDB analysis showing the number of ligand-receptor interactions between fibroblast and immune cells (G) and dot plot of HA receptors for fibroblast and immune cells (H) in Prkci^{f/f}Prkcz^{f/f}; Villin-Cre Veh-treated tumors.

FIG. 19I illustrates CellphoneDB analysis dot plot for ligand-receptor pairs of cytokines between telocytes, intermediate and CD4+ Treg cells in Prkci^{f/f}Prkcz^{f/f}; Villin-Cre Veh- and PEGPH20-treated tumors.

FIGS. 19J and 19K illustrate CellphoneDB analysis dot plot for ligand-receptor pairs of PDGFR/TGFb signaling between telocytes, intermediate with myeloid cells (J) or CD4+ Treg (K) in Prkci^{f/f}Prkcz^{f/f}; Villin-Cre Veh- and PEGPH20-treated tumors.

FIGS. 19L and 19M illustrate CellphoneDB analysis showing dot plot for ligand-receptor pairs of growth factors between telocytes, intermediate with myeloid cells (L) or CD4+ Treg (M) in Prkci^{f/f}Prkcz^{f/f}; Villin-Cre Veh- and PEGPH20-treated tumors. Results are shown as mean±SEM *p<0.05, **p<0.01, ***p<0.001, ****p<0.0001.

FIGS. 20A-20I illustrate hyaluronan depletion renders aPKC-deficient tumors sensitive to immune checkpoint blockade therapy.

FIG. 20 A illustrates dot plot for ligand-receptor pairs of immune checkpoint inhibitors between immune cell compartments in Veh- and PEGPH20-treated tumors.

FIG. 20B illustrates violin plot for Ctla4 expression in CD4+ Treg treated with Veh or PEGPH20.

FIGS. 20C and 20D illustrate subcutaneous injection of sgPrkci/Prkcz MTOs in WT mice. Mice were treated twice a week with Veh or PEGPH20 0.0375 mg/kg and aCTLA4 (100 ug/dose) for 4 weeks (Veh n=8, and PEGPH20-treated with aCTLA4 n=8). Experimental design (C) and tumor volume (D).

FIGS. 20E and 20F illustrate subcutaneous injection of sgPrkci/Prkcz MTOs in WT mice. Mice were treated twice a week with Veh or aCTLA4 (100 ug/dose) for 4 weeks (Veh n=8, and treated with aCTLA4 n=8). Experimental design (E) and tumor volume (F).

FIGS. 20G and 20H illustrate dot plot for novel immune checkpoint inhibitors expressed by tumoral epithelial cells (G) and their cognate receptors expressed by fibroblasts and immune populations in Veh-treated tumors.

FIG. 20I illustrates bot plot for Cd47-SIRPa immune checkpoint inhibitor pair between immune cell compartments in Veh- and PEGPH20-treated tumors.

FIG. 21 lists gene signatures and genes for scRNA and bulk analyses.

FIGS. 22A-22D illustrate that hyaluronan depletion renders liver metastasis sensitive to immune checkpoint blockade therapy.

FIG. 22A illustrates experimental design.

FIG. 22B illustrates macroscopic images of liver metastasis tumors.

FIG. 22C illustrates total tumor number, tumor load, and average tumor volume. Results are shown as mean±SEM *p<0.05, **p<0.01, ***p<0.001, ****p<0.0001.

FIG. 22D illustrates quantification of maximal tumor size, and the number of tumors>3 mm and <3 mm. Results are shown as mean±SEM *p<0.05, **p<0.01, ***p<0.001, ****p<0.0001.

### DETAILED DESCRIPTION

The CMS4 CRC subtype is characterized by strong stromal infiltration, TGFβ activation, and poor responses to chemotherapy and targeted therapies. There is a need for more accurate methods for diagnosing CMS4 CRC patients and better methods for treating CMS4 CRC in patients.

This disclosure relates generally to methods for using biomarkers to determine a subject having or is suspected of having a subtype of colorectal cancer and methods for treating a subject having or is suspected of having a CMS4 colorectal cancer.

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

The terminology used herein is for the purpose of describing particular cases only and is not intended to be limiting. As used herein, the singular forms “a”, “an” and “the” are intended to include the plural forms as well, unless the context clearly indicates otherwise. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

### Definitions

The “amount” or “level” of a biomarker associated with an increased clinical benefit to an individual is a detectable level in a biological sample. These can be measured by methods known to one skilled in the art and also disclosed herein. The expression level or amount of biomarker assessed can be used to determine the response to the treatment.

The term “biomarker” as used herein refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features. In some embodiments, a biomarker is a gene. Biomarkers include, but are not limited to, polynucleotides (e.g., DNA, and/or RNA), polypeptides, polypeptide and polynucleotide modifications (e.g. posttranslational modifications), carbohydrates, and/or glycolipid-based molecular markers.

The terms “biomarker signature” or “signature” are used interchangeably herein and refer to one or a combination of biomarkers whose expression is an indicator, e.g., predictive, diagnostic, and/or prognostic. The biomarker signature may serve as an indicator of a particular subtype of a disease or disorder (e.g., cancer) characterized by certain molecular, pathological, histological, and/or clinical features. In some embodiments, the biomarker signature is a “gene signature.” The term “gene signature” is used interchangeably with “gene expression signature” and refers to one or a combination of polynucleotides whose expression is an indicator, e.g., predictive, diagnostic, and/or prognostic. In some embodiments, the biomarker signature is a “protein signature.” The term “protein signature” is used interchangeably with “protein expression signature” and refers to one or a combination of polypeptides whose expression is an indicator, e.g., predictive, diagnostic, and/or prognostic.

The term “sample” refers to a composition that is obtained or derived from a subject and/or individual of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. Samples include, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

A “reference sample”, “reference cell”, “reference tissue”, “control sample”, “control cell”, or “control tissue”, as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual. For example, healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual. In another embodiment, a reference sample, reference cell, reference tissue, control sample, control cell, or control tissue is obtained from a part of the body (e.g., tissue or cells) of a subject or individual that does not have the specific subtype of cancer (e.g., CMS4 CRC). For example, CMS1 CRC cells or tissue.

The term “CRC” refers to colorectal cancer. The term “CMS4” refers to a subtype of colorectal cancer. The term “CMS” refers to the consensus molecular subtypes classification. CMS classifies CRC into four molecular subtypes with distinct biological characteristics, which may be used for subtype-based targeted treatment. The CMS4 subtype is characterized by epithelial mesenchymal transformation (EMT) accompanied by prominent stromal invasion and angiogenesis, hallmarked by transforming growth factor-β (TGFβ) activation. (Menter, D et al. Back to the colorectal cancer consensus molecular subtype future, Curr Gastroenterol Rep, 2019 Jan. 30; 21(2): 5).

The term “diagnosis” is used herein to refer to the identification or classification of a molecular or pathological state, disease or condition (e.g., cancer). For example, “diagnosis” may refer to identification of a particular type of cancer. “Diagnosis” may also refer to the classification of a particular subtype of cancer, e.g., by histopathological criteria, or by molecular features (e.g., a subtype characterized by expression of one or a combination of biomarkers (e.g., particular genes or proteins encoded by the genes)).

The term “aiding diagnosis” is used herein to refer to methods that assist in making a clinical determination regarding the presence, or nature, of a particular type of symptom or condition of a disease or disorder (e.g., cancer). For example, a method of aiding diagnosis of a disease or condition (e.g., cancer) can comprise measuring certain biomarkers in a biological sample from an individual.

The terms “level of expression” or “expression level” in general are used interchangeably and generally refer to the amount of a biomarker in a biological sample. “Expression” generally refers to the process by which information (e.g., gene-encoded and/or epigenetic) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

“Elevated expression,” “elevated expression levels,” or “elevated levels” refers to an increased expression or increased levels of a biomarker in an individual relative to a control, such as an individual or individuals who are not suffering from the disease or disorder (e.g., cancer) or an internal control (e.g., housekeeping biomarker).

“Reduced expression,” “reduced expression levels,” or “reduced levels” refers to a decrease expression or decreased levels of a biomarker in an individual relative to a control, such as an individual or individuals who are not suffering from the disease or disorder (e.g., cancer) or an internal control (e.g., housekeeping biomarker). In some embodiments, reduced expression is little or no expression.

An “effective amount” of an agent refers to an amount effective, at dosages and for periods of time necessary, to achieve the desired therapeutic or prophylactic result.

A “therapeutically effective amount” of a substance/molecule, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects.

The terms “treat,” “treating,” or “treatment” as used herein, include reducing, alleviating, abating, ameliorating, relieving, or lessening the symptoms associated with a disease, disease sate, or indication (e.g., addiction, such as opioid addiction, or pain) in either a chronic or acute therapeutic scenario. Also, treatment of a disease or disease state described herein includes the disclosure of use of such compound or composition for the treatment of such disease, disease state, or indication.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “anti-cancer therapy” refers to a therapy useful in treating cancer. Examples of anti-cancer therapeutic agents include, but are limited to, e.g., chemotherapeutic agents, growth inhibitory agents, cytotoxic agents, agents used in radiation therapy, anti-angiogenesis agents, apoptotic agents, anti-tubulin agents, and other agents to treat cancer, anti-CD20 antibodies, platelet derived growth factor inhibitors (e.g., Gleevec™ (Imatinib Mesylate)), a COX-2 inhibitor (e.g., celecoxib), interferons, cytokines, antagonists (e.g., neutralizing antibodies) that bind to one or more of the following targets PDGFR-beta, BlyS, APRIL, BCMA receptor(s), TRAIL/Apo2, and other bioactive and organic chemical agents, etc. Combinations thereof are also included in the invention.

The term “cytotoxic agent” as used herein refers to a substance that inhibits or prevents the function of cells and/or causes destruction of cells. The term is intended to include radioactive isotopes (e.g., At¹¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents e.g., methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

A “chemotherapeutic agent” refers to a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Nicolaou et al., Angew. Chem Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®), albumin-engineered nanoparticle formulation of paclitaxel (ABRAXANE™), and docetaxel (TAXOTERE®); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; to FGFR3somerase inhibitor RFS 2000; difluoromethylornithine (DMF®); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; to FGFR3somerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g., celecoxib or etoricoxib), proteosome inhibitor (e.g., PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors (see definition below); tyrosine kinase inhibitors (see definition below); serine-threonine kinase inhibitors such as rapamycin (sirolimus, RAPAMUNE®); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents as defined herein include “anti-hormonal agents” or “endocrine therapeutics” which act to regulate, reduce, block, or inhibit the effects of hormones that can promote the growth of cancer. They may be hormones themselves, including, but not limited to: anti-estrogens with mixed agonist/antagonist profile, including, tamoxifen (NOLVADEX®), 4-hydroxytamoxifen, toremifene (FARESTON®), idoxifene, droloxifene, raloxifene (EVISTA®), trioxifene, keoxifene, and selective estrogen receptor modulators (SERMs) such as SERM3; pure anti-estrogens without agonist properties, such as fulvestrant (FASLODEX®), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (AROMASIN®), and nonsteroidal aromatase inhibitors such as anastrazole (ARIMIDEX®), letrozole (FEMARA®) and aminoglutethimide, and other aromatase inhibitors include vorozole (RIVISOR®), megestrol acetate (MEGASE®), fadrozole, and 4(5)-imidazoles; lutenizing hormone-releaseing hormone agonists, including leuprolide (LUPRON® and ELIGARD®), goserelin, buserelin, and tripterelin; sex steroids, including progestines such as megestrol acetate and medroxyprogesterone acetate, estrogens such as diethylstilbestrol and premarin, and androgens/retinoids such as fluoxymesterone, all transretionic acid and fenretinide; onapristone; anti-progesterones; estrogen receptor down-regulators (ERDs); anti-androgens such as flutamide, nilutamide and bicalutamide; and pharmaceutically acceptable salts, acids or derivatives of any of the above; as well as combinations of two or more of the above.

The term “prodrug” as used in this application refers to a precursor or derivative form of a pharmaceutically active substance that is less cytotoxic to tumor cells compared to the parent drug and is capable of being enzymatically activated or converted into the more active parent form. See, e.g., Wilman, Prodrugs in Cancer Chemotherapy, Biochemical Society Transactions, 14, pp. 375-382, 615th Meeting Belfast (1986) and Stella et al., Prodrugs: A Chemical Approach to Targeted Drug Delivery, Directed Drug Delivery, Borchardt et al., (ed.), pp. 247-267, Humana Press (1985). The prodrugs may include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use may include, but are not limited to, those chemotherapeutic agents described above.

A “growth inhibitory agent” when used herein refers to a compound or composition which inhibits growth of a cell (e.g., a cell whose growth is dependent upon FGFR3 expression either in vitro or in vivo). Examples of growth inhibitory agents include agents that block cell cycle progression (at a place other than S phase), such as agents that induce G1 arrest and M-phase arrest. Classical M-phase blockers include the vincas (vincristine and vinblastine), taxanes, topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995), especially p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

“Radiation therapy” refers to the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one-time administration and typical dosages range from 10 to 200 units (Grays) per day.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

By “reduce or inhibit” is meant the ability to cause an overall decrease of 20%, 30%, 40%, 50%, 60%, 70%, 75%, 80%, 85%, 90%, 95%, or greater. Reduce or inhibit can refer to the symptoms of the disorder being treated, the presence or size of metastases, or the size of the primary tumor.

The phrase “based on” when used herein means that the information about one or more biomarkers is used to inform a diagnosis decision, treatment decision, information provided on a package insert, or marketing/promotional guidance, etc.

The term “hyaluronic acid inhibitor” or “HA inhibitor” refers to a compound or a molecule that inhibit hyaluronic acid synthesis and/or deposition. In some embodiment, an HA inhibitor can degrade HA.

### Methods for Diagnosing CRC Subtype in a Subject

Provided herein are methods for diagnosing or adding diagnosis of a subject having or is suspected of having a subtype of colorectal cancer. In particular, provided herein are methods for determining a subject having or is suspected of having a subtype of colorectal cancer, comprising (a) assaying a level of hyaluronic acid (HA) in a biological sample from the subject; and (b) determining a subtype of the colorectal cancer at least based on a result from (a). In some embodiments, the subtype of the colorectal cancer is CMS4 subtype. Further provided herein are methods for determining a subject having or is suspected of having a subtype of colorectal cancer, comprising (a) assaying a level of HA and at least two atypical PKCs comprising PKCζ and PKCλ/ι in a biological sample from the subject; and (b) determining a subtype of the colorectal cancer at least based on a result from (a). Further provided herein are methods for determining a subject having or is suspected of having a subtype of colorectal cancer, comprising (a) assaying a level of HA, at least two atypical PKCs comprising PKCζ and PKCλ/ι, and at least one HA receptor in a biological sample from the subject; and (b) determining a subtype of the colorectal cancer at least based on a result from (a). Further provided herein are methods for determining a subject having or is suspected of having a subtype of colorectal cancer, comprising (a) assaying a level of at least two atypical PKCs comprising PKCζ and PKCλ/ι and at least one HA receptor in a biological sample from the subject; and (b) determining a subtype of the colorectal cancer at least based on a result from (a).

### Biomarkers Used in the Diagnosis and Prognosis of CRC Subtype

Atypical protein kinase C (aPKC) ζ (and λ/ι belong to a subfamily of PKCs. PKCζ is encoded by the gene, PRKCZ (Entrez Gene ID No. 5584). PKC λ/ι is encoded by the gene, PRKC1 (Entrez Gene ID No. 5590). The present disclosure herein provides that simultaneous deletions of PKCζ and PKCλ/ι in intestinal epithelium cells results in the development of a CMS4 CRC with a strongly reactive and immunosuppressive stroma. The present disclosure herein further provides that increased expression of HA and its receptor, CD44—a growth factor implicated in CRC tumorigenesis, in CMS4 CRC tumors. The present disclosure provides that PKCζ, PKCλ/ι, HA and/or CD44 may be useful biomarkers/signatures for identifying subjects suffering from, or who will develop, CMS4 CRC. Subjects with CMS4 CRC lacking PKCζ and PKCλ/ι, and increased expression of HA and/or CD44, are resistant to many immunotherapy-based strategies. Thus, the biomarkers disclosed herein are useful to identify subjects suffering from, or susceptible to developing, the CMS4 subtype of CRC, and enable the discovery and improvement of alternative therapeutic solutions.

Provided herein are uses of biomarker aPKC comprising PKCζ and PKCλ/ι for CRC subtype diagnosis and prognosis (e.g., CMS4 CRC). Further provided herein are uses of biomarker hyaluronic acid (HA) for CRC subtype diagnosis and prognosis (e.g., CMS4 CRC). Further provided herein are uses of biomarker HA receptor such as CD44 for CRC subtype diagnosis and prognosis (e.g., CMS4 CRC). Further provided here are uses of biomarker HA synthase (HAS) such as HAS1, HAS2, or HAS3 for CRC subtype diagnosis and prognosis (e.g., CMS4 CRC). Further provided herein are uses of biomarker CD44 ligand such as osteopontin (OPN/Spp1) for CRC subtype diagnosis and prognosis (e.g., CMS4 CRC). Further provided herein are uses of biomarker stromal cell marker such as GREM1, SFRP1, SFRP2, SFRP4, CXCL14, MMP3, IGF1, or cell proliferation makers for CRC subtype diagnosis and prognosis (e.g., CMS4 CRC). In some embodiments, the stromal cell marker comprises SFRP2 or SFRP4. In some embodiments, the stromal cell marker comprises SFRP2 and SFRP4.

In some embodiments, a biomarker signature is used for CRC subtype diagnosis and prognosis (e.g., CMS4 CRC). In some embodiments, the biomarker signature comprises at least two biomarker selected from the group consisting of HA, aPKCs comprising PKCζ and PKCλ/ι, HA receptor such as CD44, HA synthesase (HAS1, HAS2, HAS3), CD44 ligand such as osteopontin (OPN/Spp1), and stromal cell marker disclosed herein. In some embodiments, the biomarker signature comprises HA and aPKCs comprising PKCζ and PKCλ/ι. In some embodiments, the biomarker signature comprises HA and HA receptor such as CD44. In some embodiments, the biomarker signature comprises HA and HA synthesase (HAS1, HAS2, or HAS3). In some embodiments, the biomarker signature comprises HA and CD44 ligand such as osteopontin (OPN/Spp1). In some embodiments, the biomarker signature comprises HA and at least one of the stromal cell markers disclosed herein. In some embodiments, the biomarker signature comprises HA and SFRP2. In some embodiments, the biomarker signature comprises HA and SFRP4. In some embodiments, the biomarker signature comprises HA and SFRP2 and SFRP4. In some embodiments, the biomarker signature comprises aPKCs comprising PKCζ and PKCλ/ι and HA receptor such as CD44. In some embodiments, the biomarker signature comprises aPKCs comprising PKCζ and PKCλ/ι and HA synthesase (HAS1, HAS2, or HAS3). In some embodiments, the biomarker signature comprises aPKCs comprising PKCζ and PKCλ/ι and CD44 ligand such as osteopontin (OPN/Spp1). In some embodiments, the biomarker signature comprises aPKCs comprising PKCζ and PKCλ/ι and at least one of the stromal cell markers disclosed herein. In some embodiments, the biomarker signature comprises aPKCs comprising PKCζ and PKCλ/ι and SFRP2. In some embodiments, the biomarker signature comprises aPKCs comprising PKCζ and PKCλ/ι and SFRP4. In some embodiments, the biomarker signature comprises aPKCs comprising PKCζ and PKCλ/ι and SFRP2 and SFRP4. In some embodiments, the biomarker signature comprises HA synthesase (HAS1, HAS2, or HAS3) and CD44 ligand such as osteopontin (OPN/Spp1). In some embodiments, the biomarker signature comprises HA synthesase (HAS1, HAS2, or HAS3) at least one of the stromal cell markers disclosed herein. In some embodiments, the biomarker signature comprises HA synthesase and SFRP2. In some embodiments, the biomarker signature comprises HA synthesase and SFRP4. In some embodiments, the biomarker signature comprises HA synthesase and SFRP2 and SFRP4. In some embodiments, the biomarker signature comprises CD44 ligand such as osteopontin (OPN/Spp1), and stromal cell marker disclosed herein. In some embodiments, the biomarker signature comprises CD44 ligand and SFRP2. In some embodiments, the biomarker signature comprises CD44 ligand and SFRP4. In some embodiments, the biomarker signature comprises CD44 ligand and SFRP2 and SFRP4. In some embodiments, the biomarker signature comprises HA, aPKCs comprising PKCζ and PKCλ/ι, and HA receptor such as CD44. In some embodiments, the biomarker signature comprises HA, aPKCs comprising PKCζ and PKCλ/ι, HA receptor, and HA synthesase (HAS1, HAS2, HAS3). In some embodiments, the biomarker signature comprises HA, aPKCs comprising PKCζ and PKCλ/ι, HA receptor, HA synthesase (HAS1, HAS2, HAS3), and CD44 ligand such as osteopontin (OPN/Spp1). In some embodiments, the biomarker signature comprises HA, aPKCs comprising PKCζ and PKCλ/ι, HA receptor, and HA synthesase (HAS1, HAS2, HAS3), CD44 ligand, and at least one of the stromal cell markers disclosed herein. In some embodiments, the biomarker signature comprises HA, aPKCs comprising PKCζ and PKCλ/ι, HA receptor, and HA synthesase (HAS1, HAS2, HAS3), CD44 ligand, and SFRP2. In some embodiments, the biomarker signature comprises HA, aPKCs comprising PKCζ and PKCλ/ι, HA receptor, and HA synthesase (HAS1, HAS2, HAS3), CD44 ligand, and SFRP4. In some embodiments, the biomarker signature comprises HA, aPKCs comprising PKCζ and PKCλ/ι, HA receptor, and HA synthesase (HAS1, HAS2, HAS3), CD44 ligand, and SFRP2 and SFRP4.

Methods for Diagnosing a Subject with a Subtype of CRC

Aspects disclosed herein provide methods of diagnosing a subtype of colorectal cancer (CRC) in a subject in need thereof, the method comprising: (a) assaying a level of hyaluronic acid (HA) in a biological sample from the subject; and (b) determining a subtype of the colorectal cancer at least based on a result from (a). Further provided herein are methods for determining a subject having or is suspected of having a subtype of colorectal cancer, comprising (a) assaying a level of HA and at least two atypical PKCs comprising PKCζ and PKCλ/ι in a biological sample from the subject; and (b) determining a subtype of the colorectal cancer at least based on a result from (a). Further provided herein are methods for determining a subject having or is suspected of having a subtype of colorectal cancer, comprising (a) assaying a level of HA, at least two atypical PKCs comprising PKCζ and PKCλ/ι, and at least one HA receptor in a biological sample from the subject; and (b) determining a subtype of the colorectal cancer at least based on a result from (a). Further provided herein are methods for determining a subject having or is suspected of having a subtype of colorectal cancer, comprising (a) assaying a level of at least two atypical PKCs comprising PKCζ and PKCλ/ι and at least one HA receptor in a biological sample from the subject; and (b) determining a subtype of the colorectal cancer at least based on a result from (a). In some embodiments, the subtype of the colorectal cancer is CMS4 subtype.

The biological sample may be obtained directly, or indirectly from the subject. In some embodiments, the biological sample comprises a tissue biopsy from the intestine, tumor, or both. In some instances, the tissue biopsy is a needle biopsy, a surgical biopsy or an aspiration biopsy. In some instances, the fine needle biopsy comprises a fine need aspiration (FNA). In some embodiments, the biological sample comprises whole blood, sera or plasma. In some instances, the subject is a mammal. In some instances, the subject is a mouse, rat, monkey, or rabbit. In some instances, the subject is a human. In some embodiments, the assay used in herein may be an assay comprising polymerase chain reaction (PCR), reverse transcription PCR (RT-PCR), deoxyribonucleic acid (DNA) sequencing, ribonucleic acid (RNA) sequencing, genotyping array, immunohistochemistry, enzyme-linked immunosorbent assay (ELISA), single-molecule array (Simoa), or a combination thereof. In some embodiments, assaying comprises determining the biological sample has an elevated expression level of HA as compared to a reference sample that does not have the subtype of colorectal cancer. In some embodiments, the reference sample is obtained from a healthy and/or non-diseased part of the body (e.g., tissue or cells) of the same subject or individual. In some embodiments, the reference sample is obtained from a healthy and/or non-diseased part of the body (e.g., tissues or cells) of an individual who is not the subject or individual. In some embodiments, the reference sample is obtained from a part of the body (e.g., tissue or cells) of a subject or individual that does not have the specific subtype of cancer. For example, when the subtype of colorectal cancer is CMS4, the reference sample is obtained from a subject or individual of CMS1, CMS2, or CMS3 CRC. In some embodiments, the expression level of HA is determined by an assay comprising immunohistochemistry, enzyme-linked immunosorbent assay (ELISA), high performance liquid chromatography (HPLC), capillary electrophoresis (CE), mass spectrometry, or fluorophore-assisted carbohydrate electrophoresis, or a combination thereof.

In some embodiments, the method further comprises assaying an expression level or amount of at least one HA synthase. In some embodiments, assaying comprises determining the biological sample has an elevated expression level of at least one HA synthase as compared to a reference sample that does not have the subtype of colorectal cancer. In some embodiments, the reference sample is obtained from a healthy and/or non-diseased part of the body (e.g., tissue or cells) of the same subject or individual. In some embodiments, the reference sample is obtained from a healthy and/or non-diseased part of the body (e.g., tissues or cells) of an individual who is not the subject or individual. In some embodiments, the reference sample is obtained from a part of the body (e.g., tissue or cells) of a subject or individual that does not have the specific subtype of cancer. For example, when the subtype of colorectal cancer is CMS4, the reference sample is obtained from a subject or individual of CMS1, CMS2, or CMS3 CRC. In some embodiments, the at least one HA synthase is HA synthase 1 (HAS1), HA synthase 2 (HAS2), or HA synthase 3 (HAS3). In some embodiments, the expression level of HA synthase comprises ribonucleic acid (RNA), deoxyribonucleic acid (DNA), or protein. In some embodiments, the levels of expression of HA synthase may be detected by measuring the expression of the genes HAS1, HAS2, and/or HAS3, or gene products expressed from the genes HAS1, HAS2, and/or HAS3. In some embodiments, HA synthase expression comprises ribonucleic acid (RNA) expression. In some embodiments, HA synthase expression comprises protein expression. In some embodiments, HA synthase expression comprises deoxyribonucleic acid (DNA) expression. An “elevated” level of expression of HA synthase is a statistically significant amount of expression above the level of expression in a reference sample that does not have the specific subtype of CRC. For example, a sample obtained from a normal, or non-diseased, individual, or a subject having a different CRC subtype.

In some embodiments, assaying further comprises determining the biological sample has an elevated expression level of the at least one HA receptor as compared to a reference sample that does not have the subtype of colorectal cancer. In some embodiments, the reference sample is obtained from a healthy and/or non-diseased part of the body (e.g., tissue or cells) of the same subject or individual. In some embodiments, the reference sample is obtained from a healthy and/or non-diseased part of the body (e.g., tissues or cells) of an individual who is not the subject or individual. In some embodiments, the reference sample is obtained from a part of the body (e.g., tissue or cells) of a subject or individual that does not have the specific subtype of cancer. For example, when the subtype of colorectal cancer is CMS4, the reference sample is obtained from a subject or individual of CMS1, CMS2, or CMS3 CRC. In some embodiments, the at least one HA receptor is CD44, RHAMM, or ICAM-1. In some embodiments, the at least one HA receptor is CD44. The levels of expression of CD44 may be detected by measuring the expression of the gene CD44, or gene products expressed from the gene CD44. In some embodiments, CD44 expression comprises RNA expression. In some embodiments, CD44 expression comprises protein expression. In some embodiments, CD44 expression comprises DNA expression. An “elevated” level of expression of CD44 is a statistically significant amount of expression above the level of expression in a normal, or non-diseased, individual, or a subject having a different CRC subtype.

In some embodiments, the method further comprises assaying an expression level or amount of at least one CD44-ligand. In some embodiments, assaying comprises determining the colorectal cancer has an elevated expression level of at least one CD44-ligand as compared to a reference sample that does not have the subtype of colorectal cancer. In some embodiments, the reference sample is obtained from a healthy and/or non-diseased part of the body (e.g., tissue or cells) of the same subject or individual. In some embodiments, the reference sample is obtained from a healthy and/or non-diseased part of the body (e.g., tissues or cells) of an individual who is not the subject or individual. In some embodiments, the reference sample is obtained from a part of the body (e.g., tissue or cells) of a subject or individual that does not have the specific subtype of cancer. For example, when the subtype of colorectal cancer is CMS4, the reference sample is obtained from a subject or individual of CMS1, CMS2, or CMS3 CRC. In some embodiments, the at least one CD44-ligand comprises osteopontin (OPN/Spp1), collagens, HA, or matrix metalloproteinases (MMPs). The levels of expression of the at least one CD44-ligand may be detected by measuring the expression of the gene of the at least one CD44-ligand, or gene products expressed from the gene of the at least one CD44-ligand. In some embodiments, the at least one CD44-ligand expression comprises RNA expression. In some embodiments, the at least one CD44-ligand expression comprises protein expression. In some embodiments, the at least one CD44-ligand expression comprises DNA expression. An “elevated” level of expression of the at least one CD44-ligand is a statistically significant amount of expression above the level of expression in a normal, or non-diseased, individual, or a subject having a different CRC subtype. In some embodiments, the at least one CD44-ligand comprises osteopontin (OPN/Spp1). The levels of expression of osteopontin may be detected by measuring the expression of the gene OPN/Spp1, or gene products expressed from the gene OPN/Spp1. In some embodiments, osteopontin expression comprises RNA expression. In some embodiments, osteopontin expression comprises protein expression. In some embodiments, osteopontin expression comprises DNA expression. An “elevated” level of expression of osteopontin is a statistically significant amount of expression above the level of expression in a normal, or non-diseased, individual, or a subject having a different CRC subtype.

In some embodiments, assaying further comprises determining the biological sample has an elevated expression level of at least one stromal cell marker as compared to a reference sample that does not have the subtype of colorectal cancer. In some embodiments, the reference sample is obtained from a healthy and/or non-diseased part of the body (e.g., tissue or cells) of the same subject or individual. In some embodiments, the reference sample is obtained from a healthy and/or non-diseased part of the body (e.g., tissues or cells) of an individual who is not the subject or individual. In some embodiments, the reference sample is obtained from a part of the body (e.g., tissue or cells) of a subject or individual that does not have the specific subtype of cancer. For example, when the subtype of colorectal cancer is CMS4, the reference sample is obtained from a subject or individual of CMS1, CMS2, or CMS3 CRC. In some embodiment, the at least one stromal marker is a fibroblast marker. In some embodiments, the at least one stromal marker is selected from the group consisting of GREM1, SFRP1, SFRP2, SFRP4, CXCL14, MMP3, IGF1, and cell proliferation makers. The levels of expression of the at least one stromal cell marker may be detected by measuring the expression of the gene of the at least one cell marker, or gene products expressed from the gene. In some embodiments, the at least one stromal cell marker expression comprises RNA expression. In some embodiments, the at least one stromal cell marker expression comprises protein expression. In some embodiments, the at least one stromal cell marker expression comprises DNA expression. An “elevated” level of expression of the at least one stromal cell marker is a statistically significant amount of expression above the level of expression in a normal, or non-diseased, individual, or a subject having a different CRC subtype. In some embodiments, the at least one stromal cell marker comprises SFRP2 or SFRP4. In some embodiments, the at least one stromal cell marker comprises SFRP2 and SFRP4. The levels of expression of SFRP2 and/or SFRP4 may be detected by measuring the expression of the genes SFRP2 and/or SFRP4, or gene products expressed from the genes SFRP2 and/or SFRP4. In some embodiments, SFRP2 and/or SFRP4 expression comprises RNA expression. In some embodiments, SFRP2 and/or SFRP4 expression comprises protein expression. In some embodiments, SFRP2 and/or SFRP4 expression comprises DNA expression. An “elevated” level of expression of SFRP2 and/or SFRP4 is a statistically significant amount of expression above the level of expression in a normal, or non-diseased, individual, or a subject having a different CRC subtype.

In some embodiments, the low levels of expression of PKCζ and PKCλ/ι are indicative of an increase in expression of HA in the subject, as compared to an individual who does not have the disease or condition. In some embodiments, the subtype of colorectal cancer has a lower expression level of at least two atypical PKCs as compared to a reference sample that does not have the subtype of colorectal cancer. In some embodiments, the at least two atypical PKCs comprise PKCζ and PKCλ/ι. The levels of expression of PKCζ and/or PKCλ/ι may be detected by measuring the expression of the genes PRKCZ and/or PRKCI, or gene products expressed from the genes PRKCZ and/or PRKCI. In some embodiments, PKCζ and/or PKCλ/ι expressions comprise RNA expression. In some embodiments, PKCζ and/or PKCλ/ι expressions comprise protein expression. In some embodiments, PKCζ and/or PKCλ/ι expressions comprise DNA expression. An “lower” level of expression of PKCζ or PKCλ/ι is a statistically significant amount of expression below the level of expression in a normal, or non-diseased, individual, or a subject having a different CRC subtype.

In some embodiments, a subject diagnosed with a subtype of CRC according to the present methods is administered an HA inhibitor. An HA inhibitor can be any compound or molecule that can inhibit hyaluronic acid synthesis and/or deposition. In some embodiments, the HA inhibitor can degrade HA, partially or completely. In some embodiments, the HA inhibitor reduces and/or deactivates HA activity, partially or completely. In some embodiments, the HA inhibitor comprises pegvorhyaluronidase alfa (PVHA). In some embodiments, the HA inhibitor comprises PEGPH20—a PEGylated version of human recombinant PH20 hyaluronidase. In some embodiments, the HA inhibitor comprises an enzyme. In some embodiments, the HA inhibitor comprises a hyaluronidase. In some embodiments, the hyaluronidase comprises a puried hyaluronidase. In some embodiments, the hyaluronidase comprises a recombinant hyaluronidase. In some embodiments, the HA inhibitor comprises an HA synthase (HAS) inhibitor. In some embodiments, the HAS inhibitor reduces and/or deactivates HAS activity, partially or completely. In some embodiments, the HAS inhibitor inhibit or reduce the expression of HAS, partially or completely. In some embodiments, the HAS inhibitor comprises 4-Methylumbelliferone (MU). In some embodiments, the HA inhibitor comprises an HA receptor inhibitor. In some embodiments, the HA receptor inhibitor comprises an antibody against the HA receptor. In some embodiments, the HA receptor comprises CD44, RHAMM, or ICAM-1. In some embodiments, the HA inhibitor comprises an inhibitor of HA-CD44 binding or agonism. In some embodiments, the HA inhibitor comprises an antagonist of CD44. In some embodiments, the HA inhibitor comprises an antibody against CD44 activity or expression. In some embodiments, the HA inhibitor comprises a small molecule inhibitor of CD44 activity or expression. In some embodiments, the HA inhibitor is any of the combination of the HA inhibitors disclosed herein.

In some embodiments, the subject diagnosed with CMS4 CRC according to the methods disclosed herein is further administered a therapeutically effective amount of transforming growth factor beta 1 (TGF-β1) inhibitor. In some embodiments, the TGF-β1 inhibitor inhibits the expression of TGF-β1. In some embodiments, the TGF-β1 inhibitor reduces or deactivates the activity of TGF-β1. In some embodiments, the TGF-β1 inhibitor comprises an antibody, or antigen-binding fragment. In some embodiments, the TGF-β1 inhibitor comprises a small molecule. In some embodiments, the TGF-β1 inhibitor comprises a small molecule inhibitor of a receptor of TGF-β1. In some embodiments, the TGF-β1 inhibitor comprises galunisertib. In some embodiments, the TGF-β1 inhibitor is administered concurrently with the HA inhibitor. In some embodiments, the TGF-β1 inhibitor is administered before the HA inhibitor. In some embodiments, the TGF-β1 inhibitor is administered after the HA inhibitor. In some embodiments, the methods further comprise administering to the subject a therapeutically effective amount of a programmed death-ligand 1 (PD-L1) inhibitor. In some embodiments, the PD-L1 inhibitor inhibits the expression of PD-L1. In some embodiments, the PD-L1 inhibitor reduces or deactivates the activity of PD-L1. In some embodiments, the PD-L1 inhibitor comprises an antibody, or antigen-binding fragment. In some embodiments, the PD-L1 inhibitor comprises a small molecule. In some embodiments, the PD-L1 inhibitor comprises an antagonist of PD-L1. In some embodiments, the PD-L1 inhibitor comprises an antagonist of programmed cell death protein 1 (PD-1). In some embodiments, the PD-L1 inhibitor comprises an anti-PD-L1 antibody, e.g., atezolizumab, avelumab or durvalumab. In some embodiments, the PD-L1 inhibitor comprises an anti-PD-1 antibody, e.g., pembrolizumab, nivolumab or cemiplimab. In some embodiments, the PD-L1 inhibitor is administered concurrently with the HA inhibitor. In some embodiments, the PD-L1 inhibitor is administered before the HA inhibitor. In some embodiments, the PD-L1 inhibitor is administered after the HA inhibitor.

### Methods for Determining Whether a Subject has or Will Develop CMS4 CRC

Aspects disclosed herein provide methods of determining whether a subject has, or will develop, CMS4 CRC, the method comprising (a) assaying a level of hyaluronic acid (HA) in a biological sample from the subject; and (b) determining a subtype of the colorectal cancer at least based on a result from (a). In some embodiments, the method comprises (a) assaying a level of HA and at least two atypical PKCs comprising PKCζ and PKCλ/ι in a biological sample from the subject; and (b) determining a subtype of the colorectal cancer at least based on a result from (a). In some embodiments, the method comprises (a) assaying a level of HA, at least two atypical PKCs comprising PKCζ and PKCλ/ι, and at least one HA receptor in a biological sample from the subject; and (b) determining a subtype of the colorectal cancer at least based on a result from (a). In some embodiments, the method comprises (a) assaying a level of at least two atypical PKCs comprising PKCζ and PKCλ/ι and at least one HA receptor in a biological sample from the subject; and (b) determining a subtype of the colorectal cancer at least based on a result from (a). The biological sample may be obtained directly, or indirectly from the subject. In some embodiments, the biological sample comprises a tissue biopsy from the intestine, tumor, or both. In some instances, the tissue biopsy is a needle biopsy, a surgical biopsy or an aspiration biopsy. In some instances, the fine needle biopsy comprises a fine need aspiration (FNA). In some embodiments, the biological sample comprises whole blood, sera or plasma. In some instances, the subject is a mammal. In some instances, the subject is a mouse, rat, monkey, or rabbit. In some instances, the subject is a human. In some embodiments, the assay used in herein may be an assay comprising polymerase chain reaction (PCR), reverse transcription PCR (RT-PCR), deoxyribonucleic acid (DNA) sequencing, ribonucleic acid (RNA) sequencing, genotyping array, immunohistochemistry, enzyme-linked immunosorbent assay (ELISA), single-molecule array (Simoa), or a combination thereof.

In some embodiments, assaying comprises determining the biological sample has an elevated expression level of HA as compared to a reference sample that does not have the subtype of colorectal cancer. In some embodiments, the reference sample is obtained from a healthy and/or non-diseased part of the body (e.g., tissue or cells) of the same subject or individual. In some embodiments, the reference sample is obtained from a healthy and/or non-diseased part of the body (e.g., tissues or cells) of an individual who is not the subject or individual. In some embodiments, the reference sample is obtained from a part of the body (e.g., tissue or cells) of a subject or individual that does not have the specific subtype of cancer. For example, when the subtype of colorectal cancer is CMS4, the reference sample is obtained from a subject or individual of CMS1, CMS2, or CMS3 CRC. In some embodiments, the expression level of HA is determined by an assay comprising immunohistochemistry, enzyme-linked immunosorbent assay (ELISA), high performance liquid chromatography (HPLC), capillary electrophoresis (CE), mass spectrometry, or fluorophore-assisted carbohydrate electrophoresis, or a combination thereof.

In some embodiments, the method further comprises assaying an expression level or amount of at least one HA synthase. In some embodiments, assaying comprises determining the biological sample has an elevated expression level of at least one HA synthase as compared to a reference sample that does not have the subtype of colorectal cancer. In some embodiments, the reference sample is obtained from a healthy and/or non-diseased part of the body (e.g., tissue or cells) of the same subject or individual. In some embodiments, the reference sample is obtained from a healthy and/or non-diseased part of the body (e.g., tissues or cells) of an individual who is not the subject or individual. In some embodiments, the reference sample is obtained from a part of the body (e.g., tissue or cells) of a subject or individual that does not have the specific subtype of cancer. For example, when the subtype of colorectal cancer is CMS4, the reference sample is obtained from a subject or individual of CMS1, CMS2, or CMS3 CRC. In some embodiments, the at least one HA synthase is HA synthase 1 (HAS1), HA synthase 2 (HAS2), or HA synthase 3 (HAS3). In some embodiments, the expression level of HA synthase comprises ribonucleic acid (RNA), deoxyribonucleic acid (DNA), or protein. In some embodiments, the levels of expression of HA synthase are detected by measuring the expression of the genes HAS1, HAS2, and/or HAS3, or gene products expressed from the genes HAS1, HAS2, and/or HAS3. In some embodiments, HA synthase expression comprises ribonucleic acid (RNA) expression. In some embodiments, HA synthase expression comprises protein expression. In some embodiments, HA synthase expression comprises deoxyribonucleic acid (DNA) expression. An “elevated” level of expression of HA synthase is a statistically significant amount of expression above the level of expression in in a reference sample that does not have the specific subtype of CRC. For example, a sample obtained from a normal, or non-diseased, individual, or a subject having a different CRC subtype.

In some embodiments, assaying further comprises determining the biological sample has an elevated expression level of at least one HA receptor as compared to a reference sample that does not have the subtype of colorectal cancer. In some embodiments, the reference sample is obtained from a healthy and/or non-diseased part of the body (e.g., tissue or cells) of the same subject or individual. In some embodiments, the reference sample is obtained from a healthy and/or non-diseased part of the body (e.g., tissues or cells) of an individual who is not the subject or individual. In some embodiments, the reference sample is obtained from a part of the body (e.g., tissue or cells) of a subject or individual that does not have the specific subtype of cancer. For example, when the subtype of colorectal cancer is CMS4, the reference sample is obtained from a subject or individual of CMS1, CMS2, or CMS3 CRC. In some embodiments, the at least one HA receptor is CD44, RHAMM, or ICAM-1. In some embodiments, the at least one HA receptor is CD44. The levels of expression of CD44 may be detected by measuring the expression of the gene CD44, or gene products expressed from the gene CD44. In some embodiments, CD44 expression comprises RNA expression. In some embodiments, CD44 expression comprises protein expression. In some embodiments, CD44 expression comprises DNA expression. An “elevated” level of expression of CD44 is a statistically significant amount of expression above the level of expression in a normal, or non-diseased, individual, or a subject having a different CRC subtype.

In some embodiments, the method further comprises assaying an expression level or amount of at least one CD44-ligand. In some embodiments, assaying comprises determining the colorectal cancer has an elevated expression level of at least one CD44-ligand as compared to a reference sample that does not have the subtype of colorectal cancer. In some embodiments, the reference sample is obtained from a healthy and/or non-diseased part of the body (e.g., tissue or cells) of the same subject or individual. In some embodiments, the reference sample is obtained from a healthy and/or non-diseased part of the body (e.g., tissues or cells) of an individual who is not the subject or individual. In some embodiments, the reference sample is obtained from a part of the body (e.g., tissue or cells) of a subject or individual that does not have the specific subtype of cancer. For example, when the subtype of colorectal cancer is CMS4, the reference sample is obtained from a subject or individual of CMS1, CMS2, or CMS3 CRC. In some embodiments, the at least one CD44-ligand comprises osteopontin (OPN/Spp1), collagens, HA, or matrix metalloproteinases (MMPs). The levels of expression of the at least one CD44-ligand may be detected by measuring the expression of the gene of the at least one CD44-ligand, or gene products expressed from the gene of the at least one CD44-ligand. In some embodiments, the at least one CD44-ligand expression comprises RNA expression. In some embodiments, the at least one CD44-ligand expression comprises protein expression. In some embodiments, the at least one CD44-ligand expression comprises DNA expression. An “elevated” level of expression of the at least one CD44-ligand is a statistically significant amount of expression above the level of expression in a normal, or non-diseased, individual, or a subject having a different CRC subtype. In some embodiments, the at least one CD44-ligand comprises osteopontin (OPN/Spp1). The levels of expression of osteopontin may be detected by measuring the expression of the gene OPN/Spp1, or gene products expressed from the gene OPN/Spp1. In some embodiments, osteopontin expression comprises RNA expression. In some embodiments, osteopontin expression comprises protein expression. In some embodiments, osteopontin expression comprises DNA expression. An “elevated” level of expression of osteopontin is a statistically significant amount of expression above the level of expression in a normal, or non-diseased, individual, or a subject having a different CRC subtype.

In some embodiments, assaying further comprises determining the biological sample has an elevated expression level of at least one stromal cell marker as compared to a reference sample that does not have the subtype of colorectal cancer. In some embodiments, the reference sample is obtained from a healthy and/or non-diseased part of the body (e.g., tissue or cells) of the same subject or individual. In some embodiments, the reference sample is obtained from a healthy and/or non-diseased part of the body (e.g., tissues or cells) of an individual who is not the subject or individual. In some embodiments, the reference sample is obtained from a part of the body (e.g., tissue or cells) of a subject or individual that does not have the specific subtype of cancer. For example, when the subtype of colorectal cancer is CMS4, the reference sample is obtained from a subject or individual of CMS1, CMS2, or CMS3 CRC. In some embodiment, the at least one stromal marker is a fibroblast marker. In some embodiments, the at least one stromal marker is selected from the group consisting of GREM1, SFRP1, SFRP2, SFRP4, CXCL14, MMP3, IGF1, and cell proliferation makers. The levels of expression of the at least one stromal cell marker may be detected by measuring the expression of the gene of the at least one cell marker, or gene products expressed from the gene. In some embodiments, the at least one stromal cell marker expression comprises RNA expression. In some embodiments, the at least one stromal cell marker expression comprises protein expression. In some embodiments, the at least one stromal cell marker expression comprises DNA expression. An “elevated” level of expression of the at least one stromal cell marker is a statistically significant amount of expression above the level of expression in a normal, or non-diseased, individual, or a subject having a different CRC subtype. In some embodiments, the at least one stromal cell marker comprises SFRP2 or SFRP4. In some embodiments, the at least one stromal cell marker comprises SFRP2 and SFRP4. The levels of expression of SFRP2 and/or SFRP4 may be detected by measuring the expression of the genes SFRP2 and/or SFRP4, or gene products expressed from the genes SFRP2 and/or SFRP4. In some embodiments, SFRP2 and/or SFRP4 expression comprises RNA expression. In some embodiments, SFRP2 and/or SFRP4 expression comprises protein expression. In some embodiments, SFRP2 and/or SFRP4 expression comprises DNA expression. An “elevated” level of expression of SFRP2 and/or SFRP4 is a statistically significant amount of expression above the level of expression in a normal, or non-diseased, individual, or a subject having a different CRC subtype.

In some embodiments, the low levels of expression of PKCζ and PKCλ/ι are indicative of an increase in expression of HA in the subject, as compared to an individual who does not have the disease or condition. In some embodiments, the subtype of colorectal cancer has a lower expression level of at least two atypical PKCs as compared to a reference sample that does not have the subtype of colorectal cancer. In some embodiments, the at least two atypical PKCs comprise PKCζ and PKCλ/ι. The levels of expression of PKCζ and/or PKCλ/ι may be detected by measuring the expression of the genes PRKCZ and/or PRKCI, or gene products expressed from the genes PRKCZ and/or PRKCI. In some embodiments, PKCζ and/or PKCλ/ι expressions comprise RNA expression. In some embodiments, PKCζ and/or PKCλ/ι expressions comprise protein expression. In some embodiments, PKCζ and/or PKCλ/ι expressions comprise DNA expression. An “lower” level of expression of PKCζ or PKCλ/ι is a statistically significant amount of expression below the level of expression in a normal, or non-diseased, individual, or a subject having a different CRC subtype.

Aspects disclosed herein provide methods of evaluating a biological sample obtained from the subject for the presence, absence, and/or quantity of a nucleic acid sequence from a gene of the biomarker disclosed herein, or a gene product expressed from the gene of the biomarker disclosed herein. In some cases, the nucleic acid sequence comprises deoxyribonucleic acid (DNA). In some instances, the nucleic acid sequence comprises a denatured DNA molecule or fragment thereof. In some instances, the nucleic acid sequence comprises DNA selected from: genomic DNA, viral DNA, mitochondrial DNA, plasmid DNA, amplified DNA, circular DNA, circulating DNA, cell-free DNA, or exosomal DNA. In some instances, the DNA is single-stranded DNA (ssDNA), double-stranded DNA, denaturing double-stranded DNA, synthetic DNA, and combinations thereof. The circular DNA may be cleaved or fragmented. In some instances, the nucleic acid sequence comprises ribonucleic acid (RNA). In some instances, the nucleic acid sequence comprises fragmented RNA. In some instances, the nucleic acid sequence comprises partially degraded RNA. In some instances, the nucleic acid sequence comprises a microRNA or portion thereof. In some instances, the nucleic acid sequence comprises an RNA molecule or a fragmented RNA molecule (RNA fragments) selected from: a microRNA (miRNA), a pre-miRNA, a pri-miRNA, a mRNA, a pre-mRNA, a viral RNA, a viroid RNA, a virusoid RNA, circular RNA (circRNA), a ribosomal RNA (rRNA), a transfer RNA (tRNA), a pre-tRNA, a long non-coding RNA (lncRNA), a small nuclear RNA (snRNA), a circulating RNA, a cell-free RNA, an exosomal RNA, a vector-expressed RNA, an RNA transcript, a synthetic RNA, and combinations thereof.

Disclosed herein, in some embodiments, are nucleic acid-based detection assays useful for the detection of a presence, absence, and/or quantity of a nucleic acid sequence from a gene of the biomarker disclosed herein, or gene product expressed from the gene of the biomarker disclosed herein. In some instances, the nucleic acid-based detection assay comprises quantitative polymerase chain reaction (qPCR), gel electrophoresis (including for e.g., Northern or Southern blot), immunochemistry, in situ hybridization such as fluorescent in situ hybridization (FISH), cytochemistry, or sequencing. In some embodiments, the sequencing technique comprises next generation sequencing. In some embodiments, the methods involve a hybridization assay such as fluorogenic qPCR (e.g., TaqMan™ or SYBR green), which involves a nucleic acid amplification reaction with a specific primer pair, and hybridization of the amplified nucleic acid probes comprising a detectable moiety or molecule that is specific to a target nucleic acid sequence. An additional exemplary nucleic acid-based detection assay comprises the use of nucleic acid probes conjugated or otherwise immobilized on a bead, multi-well plate, or other substrate, wherein the nucleic acid probes are configured to hybridize with a target nucleic acid sequence. In some instances, the nucleic acid probe is specific to one or more genes disclosed herein is used. In some instances, the nucleic acid probe is specific to the gene of the biomarker disclosed herein, or gene product expressed from the gene of the biomarker disclosed herein. Disclosed herein, in some embodiments, are methods of detecting a gene of an individual by subject a sample obtained from the individual to a nucleic acid amplification assay. In some instances, the amplification assay comprises polymerase chain reaction (PCR), qPCR, self-sustained sequence replication, transcriptional amplification system, Q-Beta Replicase, rolling circle replication, or any suitable other nucleic acid amplification technique. A suitable nucleic acid amplification technique is configured to amplify a region of a nucleic acid sequence comprising one or more genes, or gene expression products thereof, disclosed herein. In some instances, the amplification assay requires primers. The nucleic acid sequence for the gene, or gene expression products thereof, known or provided herein is sufficient to enable one of skill in the art to select primers to amplify any portion of the gene or genetic variants. A DNA sample suitable as a primer may be obtained, e.g., by polymerase chain reaction (PCR) amplification of genomic DNA, fragments of genomic DNA, fragments of genomic DNA ligated to adaptor sequences or cloned sequences. A person of skill in the art would utilize computer programs to design of primers with the desired specificity and optimal amplification properties, such as Oligo version 7.0 (National Biosciences). It will be apparent to one skilled in the art that controlled robotic systems are useful for isolating and amplifying nucleic acids and can be used.

In some embodiments, detecting the presence or absence and/or quantity of a gene of the biomarker disclosed herein, or gene expression produced expressed from the gene of the biomarker disclosed herein, comprises sequencing genetic material obtained from a biological sample from the subject. Sequencing can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modern sequencing technologies such as Illumina sequencing (e.g., Solexa), Roche 454 sequencing, Ion torrent sequencing, and SOLiD sequencing. In some cases, next-generation sequencing involves high-throughput sequencing methods. Additional sequencing methods available to one of skill in the art may also be employed.

In some instances, a number of nucleotides that are sequenced are at least 5, 10, 15, 20, 25, 30, 35, 40, 45, 50, 100, 150, 200, 300, 400, 500, 2000, 4000, 6000, 8000, 10000, 20000, 50000, 100000, or more than 100000 nucleotides. In some instances, the number of nucleotides sequenced is in a range of about 1 to about 100000 nucleotides, about 1 to about 10000 nucleotides, about 1 to about 1000 nucleotides, about 1 to about 500 nucleotides, about 1 to about 300 nucleotides, about 1 to about 200 nucleotides, about 1 to about 100 nucleotides, about 5 to about 100000 nucleotides, about 5 to about 10000 nucleotides, about 5 to about 1000 nucleotides, about 5 to about 500 nucleotides, about 5 to about 300 nucleotides, about 5 to about 200 nucleotides, about 5 to about 100 nucleotides, about 10 to about 100000 nucleotides, about 10 to about 10000 nucleotides, about 10 to about 1000 nucleotides, about 10 to about 500 nucleotides, about 10 to about 300 nucleotides, about 10 to about 200 nucleotides, about 10 to about 100 nucleotides, about 20 to about 100000 nucleotides, about 20 to about 10000 nucleotides, about 20 to about 1000 nucleotides, about 20 to about 500 nucleotides, about 20 to about 300 nucleotides, about 20 to about 200 nucleotides, about 20 to about 100 nucleotides, about 30 to about 100000 nucleotides, about 30 to about 10000 nucleotides, about 30 to about 1000 nucleotides, about 30 to about 500 nucleotides, about 30 to about 300 nucleotides, about 30 to about 200 nucleotides, about 30 to about 100 nucleotides, about 50 to about 100000 nucleotides, about 50 to about 10000 nucleotides, about 50 to about 1000 nucleotides, about 50 to about 500 nucleotides, about 50 to about 300 nucleotides, about 50 to about 200 nucleotides, or about 50 to about 100 nucleotides.

In some embodiments, detecting the presence or absence and/or quantity of a gene of the biomarker disclosed herein, or gene expression produced expressed from the gene of the biomarker disclosed herein, comprises hybridizing a probe or reporting sequence to a target nucleic acid described herein. Examples of molecules that are utilized as probes include, but are not limited to, RNA and DNA. In some embodiments, the term “probe” with regards to nucleic acids, refers to any molecule that is capable of selectively binding to a specifically intended target nucleic acid sequence. In some instances, probes are specifically designed to be labeled, for example, with a radioactive label, a fluorescent label, an enzyme, a chemiluminescent tag, a colorimetric tag, or other labels or tags that are known in the art. In some instances, the fluorescent label comprises a fluorophore. In some instances, the fluorophore is an aromatic or heteroaromatic compound. In some instances, the fluorophore is a pyrene, anthracene, naphthalene, acridine, stilbene, benzoxaazole, indole, benzindole, oxazole, thiazole, benzothiazole, canine, carbocyanine, salicylate, anthranilate, xanthenes dye, coumarin. Exemplary xanthene dyes include, e.g., fluorescein and rhodamine dyes. Fluorescein and rhodamine dyes include, but are not limited to 6-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N; N′-tetramethyl-6-carboxyrhodamine (TAMRA), 6-carboxy-X-rhodamine (ROX). Suitable fluorescent probes also include the naphthylamine dyes that have an amino group in the alpha or beta position. For example, naphthylamino compounds include 1-dimethylaminonaphthyl-5-sulfonate, 1-anilino-8-naphthalene sulfonate and 2-p-toluidiny1-6-naphthalene sulfonate, 5-(2′-aminoethyl)aminonaphthalene-1-sulfonic acid (EDANS). Exemplary coumarins include, e.g., 3-phenyl-7-isocyanatocoumarin; acridines, such as 9-isothiocyanatoacridine and acridine orange; N-(p-(2-benzoxazolyl)phenyl) maleimide; cyanines, such as, e.g., indodicarbocyanine 3 (Cy3), indodicarbocyanine 5 (Cy5), indodicarbocyanine 5.5 (Cy5.5), 34-carboxy-pentyl)-3′-ethyl-5,51-dimethyloxacarbocyanine (CyA); 1H, 5H, 11H, 15H-Xantheno[2,3,4-ij:5,6,7-i′j′]diquinolizin-18-ium, 9-[2 (or 4)-[[[6-(2,5-dioxo-1-pyrrolidinyl)oxy]-6-oxohexyl]amino]sulfony1]-4 (or 2)-sulfophenyl]-2,3,6,7,12,13,16,17-octahydro-inner salt (TR or Texas Red); or BODIPY™ dyes. In some cases, the probe comprises FAM as the dye label.

In some instances, primers and/or probes described herein for detecting a target nucleic acid are used in an amplification reaction. In some instances, the amplification reaction is qPCR. An exemplary qPCR is a method employing a TaqMan™ assay.

In some instances, qPCR comprises using an intercalating dye. Examples of intercalating dyes include SYBR green I, SYBR green II, SYBR gold, ethidium bromide, methylene blue, Pyronin Y, DAPI, acridine orange, Blue View or phycoerythrin. In some instances, the intercalating dye is SYBR.

In some instances, a number of amplification cycles for detecting a target nucleic acid in an amplification assay is about 5 to about 30 cycles. In some instances, the number of amplification cycles for detecting a target nucleic acid is at least about 5 cycles. In some instances, the number of amplification cycles for detecting a target nucleic acid is at most about 30 cycles. In some instances, the number of amplification cycles for detecting a target nucleic acid is about 5 to about 10, about 5 to about 15, about 5 to about 20, about 5 to about 25, about 5 to about 30, about 10 to about 15, about 10 to about 20, about 10 to about 25, about 10 to about 30, about 15 to about 20, about 15 to about 25, about 15 to about 30, about 20 to about 25, about 20 to about 30, or about 25 to about 30 cycles.

In one aspect, the methods provided herein for determining the presence, absence, and/or quantity of a nucleic acid sequence from a particular genotype comprise an amplification reaction such as qPCR. In an exemplary method, genetic material is obtained from a sample of a subject, e.g., a sample of blood or serum. In certain embodiments where nucleic acids are extracted, the nucleic acids are extracted using any technique that does not interfere with subsequent analysis. In certain embodiments, this technique uses alcohol precipitation using ethanol, methanol or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example would be the GenElute Bacterial Genomic DNA Kit available from Sigma Aldrich. In certain embodiments, after extraction the nucleic acid is stored in water, Tris buffer, or Tris-EDTA buffer before subsequent analysis. In an exemplary embodiment, the nucleic acid material is extracted in water. In some cases, extraction does not comprise nucleic acid purification.

In the exemplary qPCR assay, the nucleic acid sample is combined with primers and probes specific for a target nucleic acid that may or may not be present in the sample, and a DNA polymerase. An amplification reaction is performed with a thermal cycler that heats and cools the sample for nucleic acid amplification, and illuminates the sample at a specific wavelength to excite a fluorophore on the probe and detect the emitted fluorescence. For TaqMan™ methods, the probe may be a hydrolysable probe comprising a fluorophore and quencher that is hydrolyzed by DNA polymerase when hybridized to a target nucleic acid. In some cases, the presence of a target nucleic acid is determined when the number of amplification cycles to reach a threshold value is less than 30, 29, 28, 27, 26, 25, 24, 23, 22, 21, or 20 cycles.

In some embodiments, detecting and quantifying soluble protein levels of a biomarker disclosed herein in a subject by detecting and quantifying the levels from a biological sample obtained from the subject are provided. The biomarker may be detected by use of an antibody-based assay, where an antibody specific to a biomarker disclosed herein (e.g., anti-HA, anti-CD44, anti-PKCζ and/or anti-PKCλ/ι antibodies) is utilized. For antibody-based detection methods, the antibody may bind to any region of the biomarker disclosed herein. An exemplary method of analysis comprises performing an enzyme-linked immunosorbent assay (ELISA). The ELISA assay may be a sandwich ELISA or a direct ELISA. Another exemplary method of analysis comprises a single molecule array, e.g., Simoa. Other exemplary methods of detection include immunohistochemistry and lateral flow assay.

In some embodiments, HA and CD44 protein may be detected by detecting binding between HA and/or CD44 and other binding partners of HA and/or CD44. Methods of analysis of binding between HA and/or CD44, as well as with other binding partners comprise performing an assay in vivo or in vitro, or ex vivo. In some instances, the assay may comprise co-immunoprecipitation (co-IP), pull-down, crosslinking protein interaction analysis, labeled transfer protein interaction analysis, or Far-western blot analysis, FRET based assay, including, for example FRET-FLIM, a yeast two-hybrid assay, BiFC, or split luciferase assay.

In some embodiments, HA can be detected or a level of HA can be determined using one of a variety of well known assays based on HA-binding proteins or anti-HA antibodies, or by quantitation of purified HA. HA-binding proteins, for example, can be useful in detecting HA; a radiometric assay for HA based on 125I-labelled HA-binding protein is available from Pharmacia (Guechot et al., Clin. Chem. 42:558-563 (1996). Other commercial assays based on HA-binding proteins are available, for example, from Corgenix (Westminster, Conn.; kit 029001). In addition, HA can be detected or a level of HA can be determined using hyaluronectin as described in Maingonnat and Delpech, Ann. Clin. Biochem. 28:305-306 (1991), or using the kit available from Nalgenunc International (Rochester, N.Y.; Delpech and Bertrand, Anal. Biochem. 149:555-565 (1985)). Assays for detecting HA or determining a level of HA include a variety of competitive and non-competitive binding assays, for example, competitive binding assays using 125I-labeled HA binding protein; competitive binding assays based on alkaline phosphatase labeled-hyaluronectin (HN); and non-competitive binding assays based on peroxidase-labeled proteoglycan or peroxidase-labeled HA-binding protein, among others (Lindquist et al., Clin. Chem. 38:127-132 (1992)). See, also, Delpech and Bertrand, supra, 1985; Engstrom-Laurent et al., Scand. J. Clin. Lab. Invest. 45:497-504 (1985); Brandt et al., Acta Otolaryn. 442 (Suppl.):31-35 (1987); Goldberg, Anal. Biochem. 174:448-458 (1988); Chichibu et al., Clin. Chim. Acta 181:317-324 (1989); Li et al., Conn. Tissue Res. 19:243-254 (1989); Poole et al., Arth. Rheum. 33:790-799 (1990); Poole et al., J. Biol. Chem. 260:6020-6025 (1985); and Laurent and Tengblad, Anal. Biochem. 109:386-394 (1980)). Assays for detecting HA or determining a level of HA in a sample can be performed using a variety of immunoassay formats, including radioimmunoassays and enzyme-linked immunoassays. Anti-HA antiserum useful in immunoassays can be, for example, affinity purified sheep anti-HA antiserum available from Biotrend (Cologne, Germany; #5029-9990).

A level of HA also can be determined by purifying HA from a sample, and quantifying the amount of purified polysaccharide. High performance liquid chromatography can be used alone or in conjunction with mass spectrophotometry. As an example, HPLC can be used to determine HA levels after digestion of samples containing an internal standard with hyaluronidase, separation by a reversed phase octadecylsilyl column and elution with 0.01 M tetrabutylammonium phosphate-acetonitrile (83:17, v/v) at pH 7.35 (Payan et al., J. Chromatogr. 566:9-18 (1991)). HA levels have been shown to correlate with hyaluronidase levels (Bray et al., Am. Rev. Respir. Dis. 3:284-288 (1991)). Thus, HA can be detected or a level of HA can be determined indirectly by assaying for hyaluronidase activity. Assays for hyaluronidase activity are known in the art, as described in Bray et al., supra, 1991. One skilled in the art understands that these and other routine assays for determining hyaluronidase or HA levels are encompassed by the phrases “assaying an expression level or amount of HA” and “determining the level of HA” and can be useful in diagnosing the presence of CMS4 CRC according to methods disclosed herein.

### Methods for Treating CRC Subtype

The present disclosure provides that treatment of subjects diagnosed with or suspect of having CMS4 CRC with an HA inhibitor. An HA inhibitor can be any compound or molecule that can inhibit hyaluronic acid synthesis and/or deposition. In some embodiments, the HA inhibitor is an indirect inhibitor. In some embodiments, the HA inhibitor is a direct inhibitor. In some embodiments, the HA inhibitor comprises an antibody, or antibody fragment. In some embodiments, the antibody comprises a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, or a combination thereof. In some embodiments, the antibody or antigen-binding fragment comprises an IgG antibody. In some instances, the antibody or antibody fragment binds to HA. In some embodiments, the antibody or antibody fragment binds to 70%, 80%, 90%, or 100% of the HA molecule.

In some embodiments, the HA inhibitor comprises a small molecule. In some embodiments, the small molecule specifically binds to an HA. In some embodiments, the HA inhibitor comprises a small molecule effective to inhibit binding and/or agonism between HA and CD44. In some embodiments, the small molecule specifically binds to CD44. Non-limiting examples of HA inhibitors include 4-methylumbelliferone (4-MU), hyaluronidase PH2O (rHuPH2O), recombinant human hyaluronidase, PEGylated recombinant human hyaluronidase.

In some instances, the indirect HA inhibitor comprises a direct inhibitor of CD44. In some instances, the direct inhibitor of CD44 comprises an anti-CD44 antibody or antibody fragment. In some instances, the direct inhibitor of CD44 comprises a DNA vaccine. In some instances, the direct inhibitor of CD44 comprises CD44siRNA. In some instances, the indirect inhibitor of hyaluronan comprises a direct inhibitor of CD44. In some instances, the direct inhibitor of CD44 comprises an anti-CD44 conjugated cell therapy. The Non-limiting examples of inhibitors of CD44 activity or expression include AMC303, and RG7356.

In some embodiments, the HA inhibitor can degrade HA, partially or completely. In some embodiments, the HA inhibitor reduces and/or deactivates HA activity, partially or completely. In some embodiments, the HA inhibitor comprises pegvorhyaluronidase alfa (PVHA). In some embodiments, the HA inhibitor comprises PEGPH20. In some embodiments, the HA inhibitor comprises an enzyme. In some embodiments, the HA inhibitor comprises a hyaluronidase. In some embodiments, the hyaluronidase comprises a puried hyaluronidase. In some embodiments, the hyaluronidase comprises a recombinant hyaluronidase.

In some embodiments, the HA inhibitor comprises an HA synthase (HAS) inhibitor. In some embodiments, the HAS inhibitor reduces and/or deactivates an HA synthase activity, partially or completely. In some embodiments, the HAS inhibitor inhibit or reduce the expression of HAS, partially or completely. In some embodiments, the HAS inhibitor comprises an antibody, or antibody fragment. In some embodiments, the antibody comprises a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, or a combination thereof. In some embodiments, the antibody or antigen-binding fragment comprises an IgG antibody. In some instances, the antibody or antibody fragment binds to the HAS. In some embodiments, the HAS inhibitor comprises a small molecule. In some embodiments, the small molecule specifically binds to an HAS. In some embodiments, the HAS inhibitor comprises 4-Methylumbelliferone (MU).

In some embodiments, the HA inhibitor comprises an HA receptor inhibitor. In some embodiments, the HA receptor inhibitor comprises an antibody against the HA receptor. In some embodiments, the HA receptor comprises CD44, RHAMM, or ICAM-1. In some embodiments, the HA inhibitor comprises an inhibitor of HA-CD44 binding or agonism. In some embodiments, the HA inhibitor comprises an antagonist of CD44. In some embodiments, the HA inhibitor comprises an antibody against CD44 activity or expression. In some embodiments, the HA inhibitor comprises a small molecule inhibitor of CD44 activity or expression. In some embodiments, the HA receptor inhibitor comprises an antibody, or antibody fragment against the HA receptor. In some embodiments, the antibody comprises a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, or a combination thereof. In some embodiments, the antibody or antigen-binding fragment comprises an IgG antibody. In some instances, the antibody or antibody fragment binds to the HA receptor. In some embodiments, the HA receptor inhibitor comprises a small molecule. In some embodiments, the small molecule specifically binds to an HA receptor. In some embodiments, the HA inhibitor is any of the combination of the HA inhibitors disclosed herein.

### Pharmaceutical Compositions

Pharmaceutical compositions containing an HA inhibitor disclosed herein, a TGF-β1 inhibitor disclosed herein, a PD-L1 inhibitor disclosed herein, and/or another therapeutic agent, or any combinations thereof, are provided in some embodiments of the present disclosure for treating a subject diagnosed with or suspect of having a subtype of CRC. In some embodiment, the subtype of CRC is CMS4 CRC. In some embodiments, the pharmaceutical composition comprises an HA inhibitor disclosed herein. In some embodiments, the pharmaceutical composition comprises a TGF-β1 inhibitor disclosed herein. In some embodiments, the pharmaceutical composition comprises an HA inhibitor disclosed herein and a TGF-β1 inhibitor. In some embodiments, the pharmaceutical composition comprises a PD-L1 inhibitor disclosed herein. In some embodiments, the pharmaceutical composition comprises an HA inhibitor disclosed herein and a PD-L1 inhibitor disclosed herein. In some embodiments, the pharmaceutical composition comprises an HA inhibitor disclosed herein, a TGF-β1 inhibitor disclosed herein, and a PD-L1 inhibitor disclosed herein. In some embodiments, the pharmaceutical composition further comprises a cytotoxic agent. In some embodiments, the pharmaceutical composition further comprises a chemotherapeutic agent. In some embodiments, the pharmaceutical composition comprises a prodrug. In some embodiments, the pharmaceutical composition comprises a growth inhibitory agent. In some embodiments, the pharmaceutical compositions of this disclosure are prepared as solutions, dispersions in glycerol, liquid polyethylene glycols, and any combinations thereof in oils, in solid dosage forms, as inhalable dosage forms, as intranasal dosage forms, as liposomal formulations, dosage forms comprising nanoparticles, dosage forms comprising microparticles, polymeric dosage forms, or any combinations thereof. In some embodiments, the pharmaceutical composition as described herein comprises a pharmaceutical acceptable carrier. In some embodiments, the pharmaceutical acceptable carrier is an excipient. An excipient is, in some examples, an excipient described in the Handbook of Pharmaceutical Excipients, American Pharmaceutical Association (1986). Non-limiting examples of suitable excipients include a buffering agent, a preservative, a stabilizer, a binder, a compaction agent, a lubricant, a chelator, a dispersion enhancer, a disintegration agent, a flavoring agent, a sweetener, a coloring agent.

In some embodiments an excipient is a buffering agent. Non-limiting examples of suitable buffering agents include histidine, sodium citrate, magnesium carbonate, magnesium bicarbonate, calcium carbonate, and calcium bicarbonate. As a buffering agent, histidine, sodium bicarbonate, potassium bicarbonate, magnesium hydroxide, magnesium lactate, magnesium gluconate, aluminium hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts or combinations thereof is used, in some embodiments, in a pharmaceutical composition of the present disclosure.

In some embodiments an excipient comprises a preservative. Non-limiting examples of suitable preservatives include antioxidants, such as alpha-tocopherol and ascorbate, and antimicrobials, such as parabens, chlorobutanol, and phenol. In some examples, antioxidants further include but are not limited to EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some instances preservatives include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitor.

In some embodiments a pharmaceutical composition as described herein comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof. The binders used in a pharmaceutical formulation are, in some examples, selected from starches such as potato starch, corn starch, wheat starch; sugars such as sucrose, glucose, dextrose, lactose, maltodextrin; natural and synthetic gums; gelatine; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol and water or any combinations thereof.

In some embodiments a pharmaceutical composition as described herein comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil. The lubricants that are used in a pharmaceutical formulation, in some embodiments, are be selected from metallic stearates (such as magnesium stearate, calcium stearate, aluminium stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate and talc or a combination thereof.

In some embodiments a pharmaceutical formulation comprises a dispersion enhancer as an excipient. Non-limiting examples of suitable dispersants include, in some examples, starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments a pharmaceutical composition as described herein comprises a disintegrant as an excipient. In some embodiments a disintegrant is a non-effervescent disintegrant. Non-limiting examples of suitable non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. In some embodiments a disintegrant is an effervescent disintegrant. Non-limiting examples of suitable effervescent disintegrants include sodium bicarbonate in combination with citric acid, and sodium bicarbonate in combination with tartaric acid.

In some embodiments an excipient comprises a flavoring agent. Flavoring agents incorporated into an outer layer are, in some examples, chosen from synthetic flavor oils and flavoring aromatics; natural oils; extracts from plants, leaves, flowers, and fruits; and combinations thereof. In some embodiments a flavoring agent can be selected from the group consisting of cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiments an excipient comprises a sweetener. Non-limiting examples of suitable sweeteners include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like.

In some instances, a pharmaceutical composition as described herein comprises a coloring agent. Non-limiting examples of suitable color agents include food, drug and cosmetic colors (FD&C), drug and cosmetic colors (D&C), and external drug and cosmetic colors (Ext. D&C). A coloring agent can be used as dyes or their corresponding lakes.

In some instances, a pharmaceutical composition as described herein comprises a chelator. In some cases, a chelator is a fungicidal chelator. Examples include, but are not limited to: ethylenediamine-N,N,N′,N′-tetraacetic acid (EDTA); a disodium, trisodium, tetrasodium, dipotassium, tripotassium, dilithium and diammonium salt of EDTA; a barium, calcium, cobalt, copper, dysprosium, europium, iron, indium, lanthanum, magnesium, manganese, nickel, samarium, strontium, or zinc chelate of EDTA; trans-1,2-diaminocyclohexane-N,N,N′,N′-tetraaceticacid monohydrate; N,N-bis(2-hydroxyethyl)glycine; 1,3-diamino-2-hydroxypropane-N,N,N′,N′-tetraacetic acid; 1,3-diaminopropane-N,N,N′,N′-tetraacetic acid; ethylenediamine-N,N′-diacetic acid; ethylenediamine-N,N′-dipropionic acid dihydrochloride; ethylenediamine-N,N′-bis(methylenephosphonic acid) hemihydrate; N-(2-hydroxyethyl)ethylenediamine-N,N′,N′-triacetic acid; ethylenediamine-N,N,N′,N′-tetrakis(methylenephosponic acid); 0,0′-bis(2-aminoethyl)ethyleneglycol-N,N,N′,N′-tetraacetic acid; N,N-bis(2-hydroxybenzyl)ethylenediamine-N,N-diacetic acid; 1,6-hexamethylenediamine-N,N,N′,N′-tetraacetic acid; N-(2-hydroxyethyl)iminodiacetic acid; iminodiacetic acid; 1,2-diaminopropane-N,N,N′,N′-tetraacetic acid; nitrilotriacetic acid; nitrilotripropionic acid; the trisodium salt of nitrilotris(methylenephosphoric acid); 7,19,30-trioxa-1,4,10,13,16,22,27,33-octaazabicyclo[11,11,11]pentatriacontane hexahydrobromide; or triethylenetetramine-N,N,N′,N″,N′″,N′″-hexaacetic acid.

Also contemplated are combination products that include one or more immunotherapeutic agents disclosed herein and one or more other antimicrobial or antifungal agents, for example, polyenes such as amphotericin B, amphotericin B lipid complex (ABCD), liposomal amphotericin B (L-AMB), and liposomal nystatin, azoles and triazoles such as voriconazole, fluconazole, ketoconazole, itraconazole, pozaconazole and the like; glucan synthase inhibitors such as caspofungin, micafungin (FK463), and V-echinocandin (LY303366); griseofulvin; allylamines such as terbinafine; flucytosine or other antifungal agents, including those described herein. In addition, it is contemplated that a peptide can be combined with topical antifungal agents such as ciclopirox olamine, haloprogin, tolnaftate, undecylenate, topical nysatin, amorolfine, butenafine, naftifine, terbinafine, and other topical agents. In some instances, a pharmaceutical composition comprises an additional agent. In some cases, an additional agent is present in a therapeutically effective amount in a pharmaceutical composition.

Under ordinary conditions of storage and use, the pharmaceutical compositions as described herein comprise a preservative to prevent the growth of microorganisms. In certain examples, the pharmaceutical compositions as described herein do not comprise a preservative. The pharmaceutical forms suitable for injectable use include sterile aqueous solutions or dispersions and sterile powders for the extemporaneous preparation of sterile injectable solutions or dispersions. The pharmaceutical compositions comprise a carrier which is a solvent or a dispersion medium containing, for example, water, ethanol, polyol (e.g., glycerol, propylene glycol, and liquid polyethylene glycol, and the like), and/or vegetable oils, or any combinations thereof. Proper fluidity is maintained, for example, by the use of a coating, such as lecithin, by the maintenance of the required particle size in the case of dispersion and by the use of surfactants. The prevention of the action of microorganisms is brought about by various antibacterial and antifungal agents, for example, parabens, chlorobutanol, phenol, sorbic acid, thimerosal, and the like. In many cases, isotonic agents are included, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the liquid dosage form is suitably buffered if necessary and the liquid diluent rendered isotonic with sufficient saline or glucose. The liquid dosage forms are especially suitable for intravenous, intramuscular, subcutaneous, intratumoral, and intraperitoneal administration. In this connection, sterile aqueous media that can be employed will be known to those of skill in the art in light of the present disclosure. For example, one dosage is dissolved, in certain cases, in 1 mL to 20 mL of isotonic NaCl solution and either added to 100 mL to 1000 mL of a fluid, e.g., sodium-bicarbonate buffered saline, or injected at the proposed site of infusion.

In certain embodiments, sterile injectable solutions are prepared by incorporating a therapeutic agent, in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. The compositions disclosed herein are, in some instances, formulated in a neutral or salt form. Pharmaceutically acceptable salts include, for example, the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups are, in some cases, derived from inorganic bases such as, for example, sodium, potassium, ammonium, calcium, or ferric hydroxides, and such organic bases as isopropylamine, trimethylamine, histidine, procaine and the like. Upon formulation, the pharmaceutical compositions are administered, in some embodiments, in a manner compatible with the dosage formulation and in such amount as is therapeutically effective.

In certain embodiments, a pharmaceutical composition of this disclosure comprises an effective amount of a therapeutic agent, as disclosed herein, combined with a pharmaceutically acceptable carrier. “Pharmaceutically acceptable,” as used herein, includes any carrier which does not interfere with the effectiveness of the biological activity of the active ingredients and/or that is not toxic to the patient to whom it is administered. Non-limiting examples of suitable pharmaceutical carriers include phosphate buffered saline solutions, water, emulsions, such as oil/water emulsions, various types of wetting agents and sterile solutions. Additional non-limiting examples of pharmaceutically compatible carriers can include gels, bioadsorbable matrix materials, implantation elements containing the immunotherapeutic agents or any other suitable vehicle, delivery or dispensing means or material. Such carriers are formulated, for example, by conventional methods and administered to the subject at an effective amount.

In some embodiments, the pharmaceutical composition is a formulation comprising an immunotherapy agent (e.g., an immune check point inhibitor, regulator, or activator) and a buffering agent. In some embodiments, the immunotherapy agent is present at a concentration of about 10 to about 50 mg/mL, about 15 to about 50 mg/mL, about 20 to about 45 mg/mL, about 25 to about 40 mg/mL, about 30 to about 35 mg/mL, about 25 to about 35 mg/mL, or about 30 to about 40 mg/mL, about 15 mg/mL, about 20 mg/mL, about 25 mg/mL, about 30 mg/mL, about 33.3 mg/mL, about 35 mg/mL, about 40 mg/mL, about 45 mg/mL, or about 50 mg/mL. In some embodiments, the formulation comprises a buffering agent comprising histidine (e.g., a histidine buffer). In certain embodiments, the buffering agent (e.g., histidine buffer) is present at a concentration of about 1 mM to about 20 mM, about 2 mM to about 15 mM, about 3 mM to about 10 mM, about 4 mM to about 9 mM, about 5 mM to about 8 mM, or about 6 mM to about 7 mM, about 1 mM, about 2 mM, about 3 mM, about 4 mM, about 5 mM, about 6 mM, about 6.7 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, or about 20 mM. In some embodiments, the buffering agent (e.g., histidine buffer) is present at a concentration of about 6 mM to about 7 mM, about 6.7 mM. In other embodiments, the buffering agent (e.g., a histidine buffer) has a pH of about 4 to about 7, about 5 to about 6, about 5.5, or about 6.

In some embodiments, the formulation further comprises a carbohydrate. In certain embodiments, the carbohydrate is sucrose. In some embodiments, the carbohydrate (e.g., sucrose) is present at a concentration of about 50 mM to about 150 mM, about 25 mM to about 150 mM, about 50 mM to about 100 mM, about 60 mM to about 90 mM, about 70 mM to about 80 mM, or about 70 mM to about 75 mM, about 25 mM, about 50 mM, about 60 mM, about 70 mM, about 80 mM, about 90 mM, about 100 mM, or about 150 mM.

In some embodiments, the formulation further comprises a surfactant. In certain embodiments, the surfactant is polysorbate 20. In some embodiments, the surfactant or polysorbate 20 is present at a concentration of about 0.005% to about 0.025% (w/w), about 0.0075% to about 0.02% or about 0.01% to 0.015% (w/w), about 0.005%, about 0.0075%, about 0.01%, about 0.013%, about 0.015%, or about 0.02% (w/w). In certain embodiments, the formulation is a reconstituted formulation. For example, a reconstituted formulation is prepared, in some instances, by dissolving a lyophilized formulation in a diluent such that the immunotherapy agent is dispersed in the reconstituted formulation. In some embodiments, the lyophilized formulation is reconstituted with about 0.5 mL to about 2 mL, such as about 1 mL, of water or buffer for injection. In certain embodiments, the lyophilized formulation is reconstituted with 1 mL of water for injection at a clinical site.

### Combination Therapies

In certain embodiments, the methods of this disclosure comprise administering a therapeutic agent as disclosed herein, followed by, and preceded by or in combination with one or more further therapy. Examples of the further therapy can include, but are not limited to, anti-cancer therapy, chemotherapy, radiation therapy, or any combinations thereof. The further therapy can be administered concurrently or sequentially with respect to administration of any of the inhibitors (e.g., HA inhibitors) or the pharmaceutical compostions disclosed herein. In certain embodiments, the methods of this disclosure comprise administering an inhibitor such as HA inhibitor or pharmaceutical composition as disclosed herein, followed by, preceded by, or in combination with one or more anti-cancer agents or cancer therapies. Anti-cancer agents include, but are not limited to, chemotherapeutic agents, radiotherapeutic agents, cytokines, immune checkpoint inhibitors, anti-angiogenic agents, apoptosis-inducing agents, anti-cancer antibodies and/or anti-cyclin-dependent kinase agents. In certain embodiments, the cancer therapies include chemotherapy, biological therapy, radiotherapy, immunotherapy, cell therapy, hormone therapy, anti-vascular therapy, cryotherapy, toxin therapy and/or surgery or combinations thereof. In certain embodiments, the methods of this disclosure include administering an inhibitor or pharmaceutical composition, as disclosed herein, followed by, preceded by or in combination with one or more immunomodulatory agents. An immunomodulatory agent includes, in some examples, any compound, molecule or substance capable of stimulating anti-tumor immunity. Non-limiting examples of the immunomodulatory agents include, but not limited to, checkpoint inhibitors such as Atezolizumab (Tecentriq®), Avelumab (Bavencio®), Cemiplimab (Libtayo*), Dostarlimab (Jemperli™), Durvalumab (Imfinzi®), Ipilimumab (Yervoy®), Nivolumab (Opdivo®), Pembrolizumab (Keytruda®); cytokines including Aldesleukin (Proleukin®), Granulocyte-macrophage colony-stimulating factor (GM-CSF), Interferon α-2a (IFNα-2a), Interferon α-2b (Intron® A, IFNα-2b), Peginterferon α-2b (Sylatron®/PEG-Intron®); adjuvants including Imiquimod, Poly ICLC (Hiltonol®); and other immunomodulators such as Pexidartinib (Turalio®).

In certain examples, where the further therapy is cell therapy, exemplary cell therapies include without limitation immune effector cell therapy, chimeric antigen receptor T-cell (CAR-T) therapy, natural killer cell therapy and chimeric antigen receptor natural killer (NK) cell therapy. Either NK cells, or CAR-NK cells, or a combination of both NK cells and CAR-NK cells can be used in combination with the methods disclosed herein. In some embodiments, the NK cells and CAR-NK cells are derived from human induced pluripotent stem cells (iPSC), umbilical cord blood, or a cell line. In some embodiments, the NK cells and CAR-NK cells comprise a cytokine receptor and a suicide gene. In some embodiments, the NK cells and CAR-NK cells are characterized by the presence of CD56 and the absence of CD3 surface markers. In some embodiments, the NK cells and CAR-NK cells target tumor cells (including solid tumors) and/or cells harboring viruses. In some embodiments, the NK cells and CAR-NK cells are administered in combination with an inhibitor (e.g., HA inhibitor) or a pharmaceutical composition disclosed herein.

In certain examples, where the further therapy is radiation, exemplary doses are 5,000 Rads (50 Gy) to 100,000 Rads (1000 Gy), or 50,000 Rads (500 Gy), or other appropriate doses within the recited ranges. Alternatively, the radiation doses are about 30 to 60 Gy, about 40 to about 50 Gy, about 40 to 48 Gy, or about 44 Gy, or other appropriate doses within the recited ranges, with the dose determined, example, by means of a dosimetry study as described above. “Gy” as used herein can refer to a unit for a specific absorbed dose of radiation equal to 100 Rads. Gy is the abbreviation for “Gray.”

In certain examples, where the further therapy is chemotherapy, exemplary chemotherapeutic agents include without limitation alkylating agents (e.g., nitrogen mustard derivatives, ethylenimines, alkylsulfonates, hydrazines and triazines, nitrosureas, and metal salts), plant alkaloids (e.g., vinca alkaloids, taxanes, podophyllotoxins, and camptothecan analogs), antitumor antibiotics (e.g., anthracyclines, chromomycins, and the like), antimetabolites (e.g., folic acid antagonists, pyrimidine antagonists, purine antagonists, and adenosine deaminase inhibitors), topoisomerase I inhibitors, topoisomerase II inhibitors, and miscellaneous antineoplastics (e.g., ribonucleotide reductase inhibitors, adrenocortical steroid inhibitors, enzymes, antimicrotubule agents, and retinoids). Exemplary chemotherapeutic agents can include, without limitation, anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®), Ibrutinib, idelalisib, and brentuximab vedotin.

Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine*, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Exemplary anthracyclines can include, without limitation, e.g., doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin.

Exemplary vinca alkaloids include, but are not limited to, vinorelbine tartrate (Navelbine®), Vincristine (Oncoving), and Vindesine (Eldisine®); vinblastine (also known as vinblastine sulfate, vincaleukoblastine and VLB, Alkaban-AQ® and Velban®); and vinorelbine (Navelbine®).

Exemplary proteosome inhibitors can, but are not limited to, bortezomib (Velcade®); carfilzomib (PX-171-007, (S)-4-Methyl-N—((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoac etamido)-4-phenylbutanamido)-pentanamide); marizomib (NPI-0052); ixazomib citrate (MLN-9708); delanzomib (CEP-18770); and 0-Methyl-N-[(2-methyl-5-thiazolyl)carbonyl]-L-seryl-0-methyl-N—R1S)-2-[(2R)-2-methyl-2-oxiranyl]-2-oxo-1-(phenylmethyl)ethyl]-L-serinamide (ONX-0912).

“In combination with,” as used herein, means that the therapeutic agent and the further therapy are administered to a subject as part of a treatment regimen or plan. In certain embodiments, being used in combination does not require that the HA inhibitors or the pharmaceutical compositions disclosed herein and the further therapy are physically combined prior to administration or that they be administered over the same time frame. For example, and not by way of limitation, the HA inhibitors or the pharmaceutical compositions and the one or more agents are administered concurrently to the subject being treated, or are administered at the same time or sequentially in any order or at different points in time.

The further therapy is administered, in various embodiments, in a liquid dosage form, a solid dosage form, a suppository, an inhalable dosage form, an intranasal dosage form, in a liposomal formulation, a dosage form comprising nanoparticles, a dosage form comprising microparticles, a polymeric dosage form, or any combinations thereof. In certain embodiments, the further therapy is administered over a period of about 1 week to about 2 weeks, about 2 weeks to about 3 weeks, about 3 weeks to about 4 weeks, about 4 weeks to about 5 weeks, about 6 weeks to about 7 weeks, about 7 weeks to about 8 weeks, about 8 weeks to about 9 weeks, about 9 weeks to about 10 weeks, about 10 weeks to about 11 weeks, about 11 weeks to about 12 weeks, about 12 weeks to about 24 weeks, about 24 weeks to about 48 weeks, about 48 weeks or about 52 weeks, or longer. The frequency of administration of the further therapy is, in certain instances, once daily, twice daily, once every week, once every three weeks, once every four weeks (or once a month), once every 8 weeks (or once every 2 months), once every 12 weeks (or once every 3 months), or once every 24 weeks (once every 6 months).

All references and publications cited herein are hereby incorporated by reference.

The following examples are provided to further illustrate some embodiments of the present invention, but are not intended to limit the scope of the invention; it will be understood by their exemplary nature that other procedures, methodologies, or techniques known to those skilled in the art can alternatively be used.

### EXAMPLES

### Example 1: Biomarker Identification of CMS4 CRC

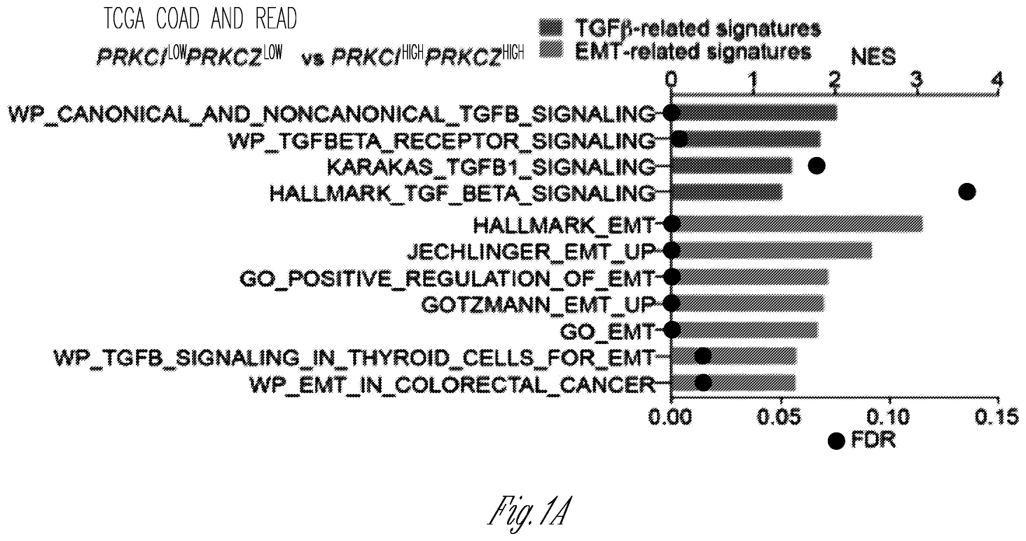

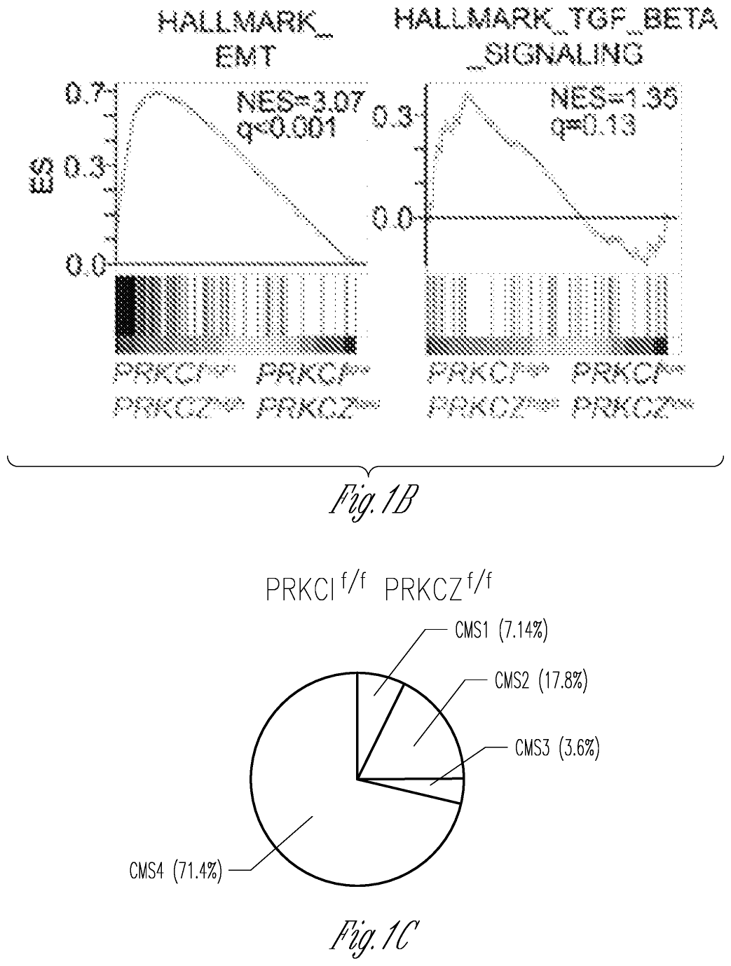

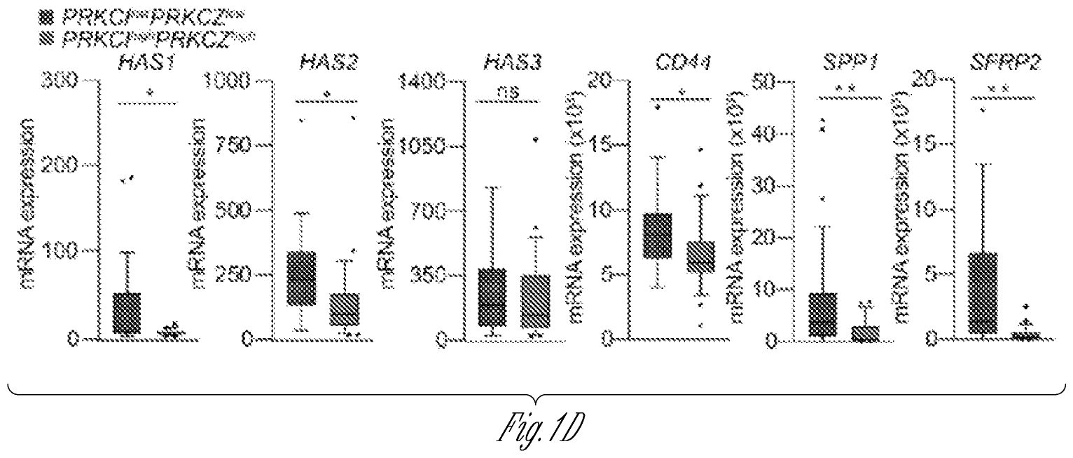

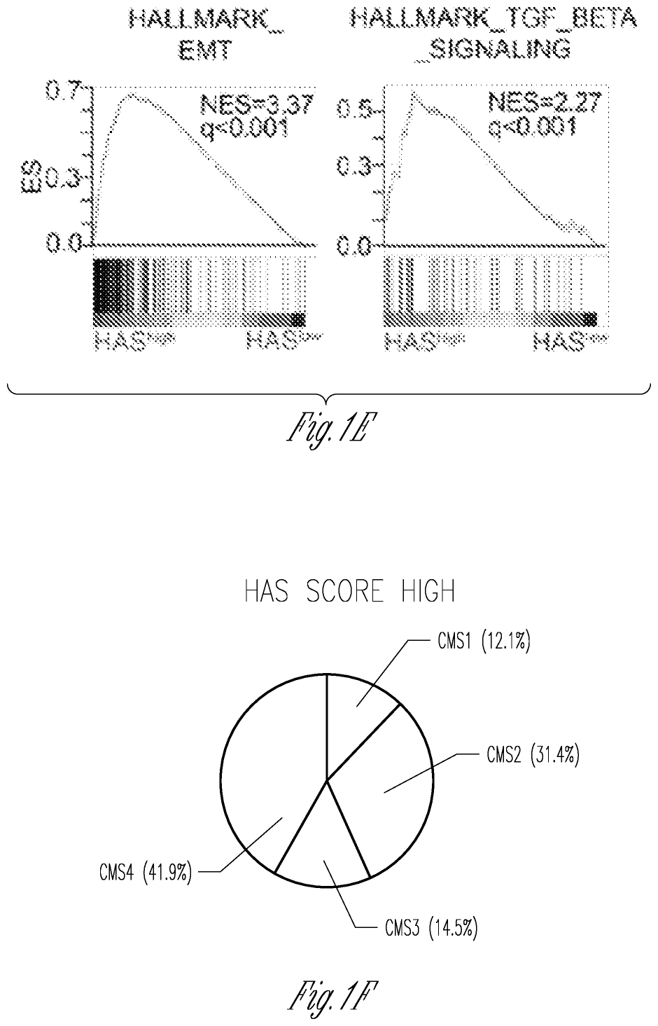

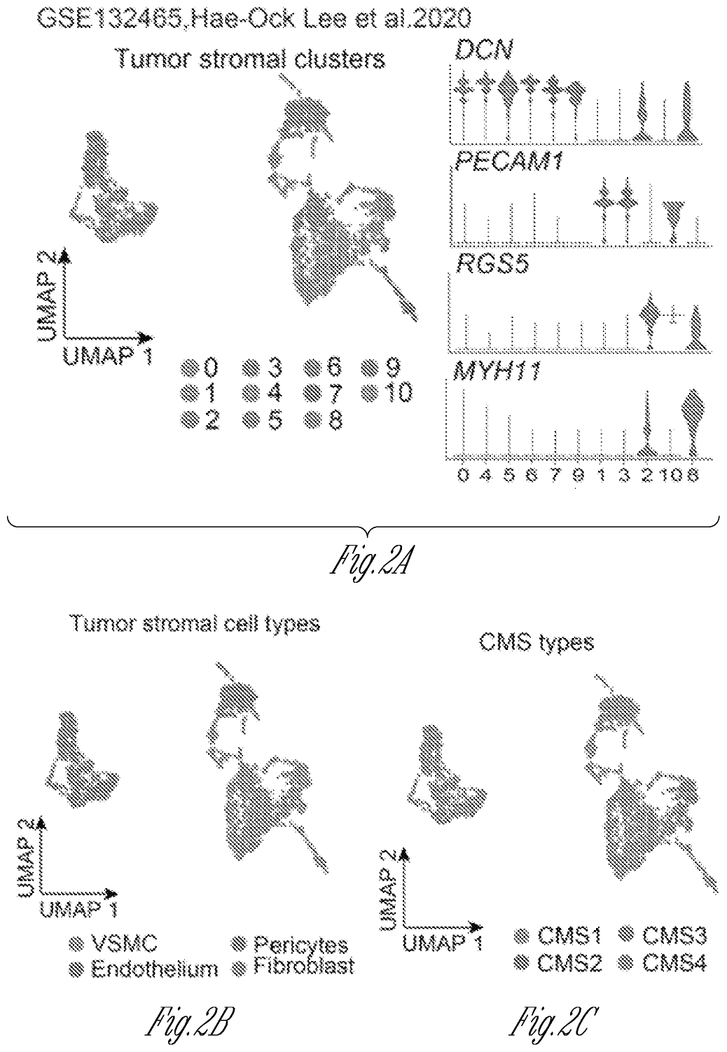

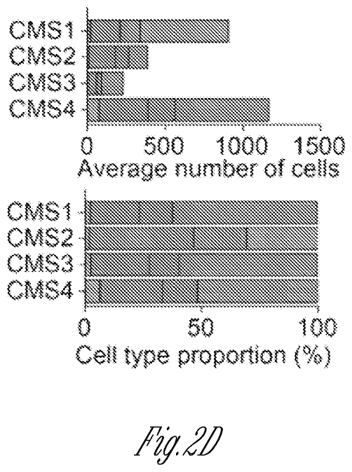

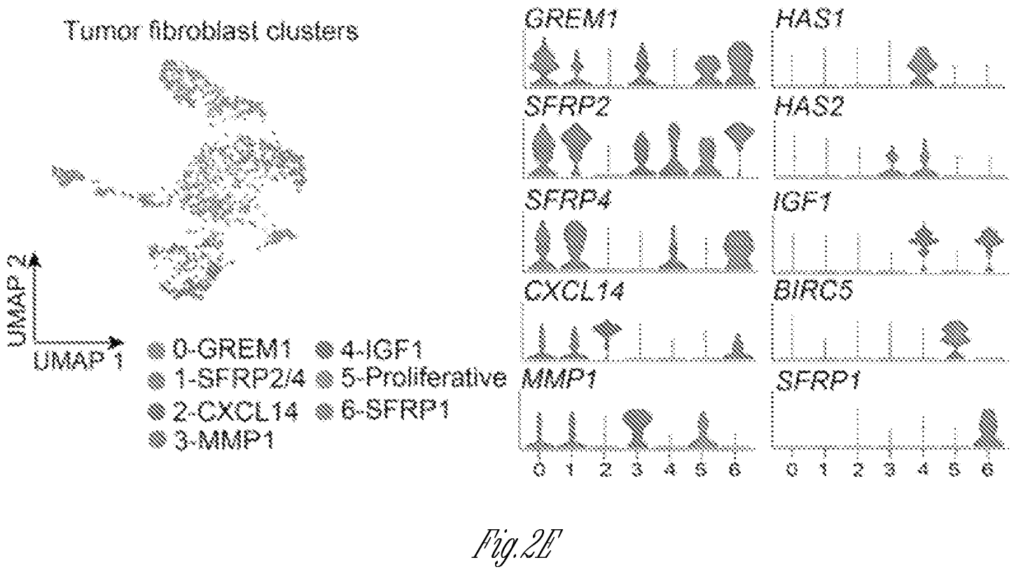

It has been demonstrated that the atypical PKCs (PKCζ and PKCλ/ι, encoded by PRKCZ and PRKCI genes) are two critical players in the progression of CRC tumors to the CMS4 category. This Example demonstrates that the phenotypic characteristics of Prkci^{f/f}Prkcz^{f/f} DKO mouse tumors in which PRKCI and PRKCZ were selectively knock-out in the intestinal epithelium fall into this most aggressive, mesenchymal, and immunosuppressed CMS4 group and constitutes a faithful endogenous mouse model of this poor prognosis CRC subtype (Nakanishi et al., Simultaneous Loss of Both Atypical Protein Kinase C Genes in the Intestinal Epithelium Drives Serrated Intestinal Cancer by Impairing Immunosurveillance. Immunity 49, 1132-1147 e1137, 2018). Intestinal tumors of DKO mice showed a highly desmoplastic stroma, which is rich in Hyaluronan (HA), a non-sulfated glycosaminoglycan and is a major component of the extracellular matrix (ECM). To support the human relevance of these observations, the colorectal adenocarcinoma set of The Cancer Genome Atlas (TCGA) was interrogated by stratifying patients based on aPKC mRNA expression. GSEA showed a significant positive enrichment of TGFβ-related and EMT-related signatures in the PRKCI^{low}PRKCZ^{low} group as compared with the PRKCI^{high}PRKCZ^{high} group (FIGS. 1A-1B). Importantly, classification based on the consensus molecular subtype signatures (CMS) revealed that the CMS4 group was predominantly (71.4%) enriched in the PRKCI^{low} PRKCZ^{low} patients (FIG. 1C).

Next, the potential of HA deposition as a biomarker for CMS4 patients was interrogated. Since HA accumulation is a result of the balance between their synthesis by HA-synthases (HAS1, HAS2, and HAS3) and their degradation by hyaluronidases, HAS levels was first evaluated in the PRKCI^{low} PRKCZ^{low} and PRKCl^{high}PRKCZ^{high} groups. mRNA expression of HAS1 and HAS2 was significantly upregulated in the PRKCI^{low} PRKCZ^{low} group (FIG. 1D), consistent with the enrichment of HA in DKO stroma. Furthermore, a significant upregulation of the HA receptor, CD44 was found in the PRKCI^{low}PRKCZ^{low} as compared to the PRKCI^{high} PRKCZ^{high} patient group (FIG. 1D). Concomitant with the upregulation of CD44, increased expression of osteopontin (OPN/Spp1), another well-known CD44-ligand, was also identified. It was noted that PRKCI^{low} PRKCZ^{low} patients had also significantly higher levels of SFRP2 (FIG. 1D), a recently identified biomarker of CMS4 stroma (Kasashima et al., 2020). GSEA analysis of CRC patients based on a HAS score calculated according to their HAS1, HAS2, and HAS3 levels showed a positive enrichment in TGFβ signaling and EMT signatures in CRC with a high HAS score (FIG. 1E). Patients with high HAS score also showed a positive correlation (41.9%) with CMS4 CRC (FIG. 1F).

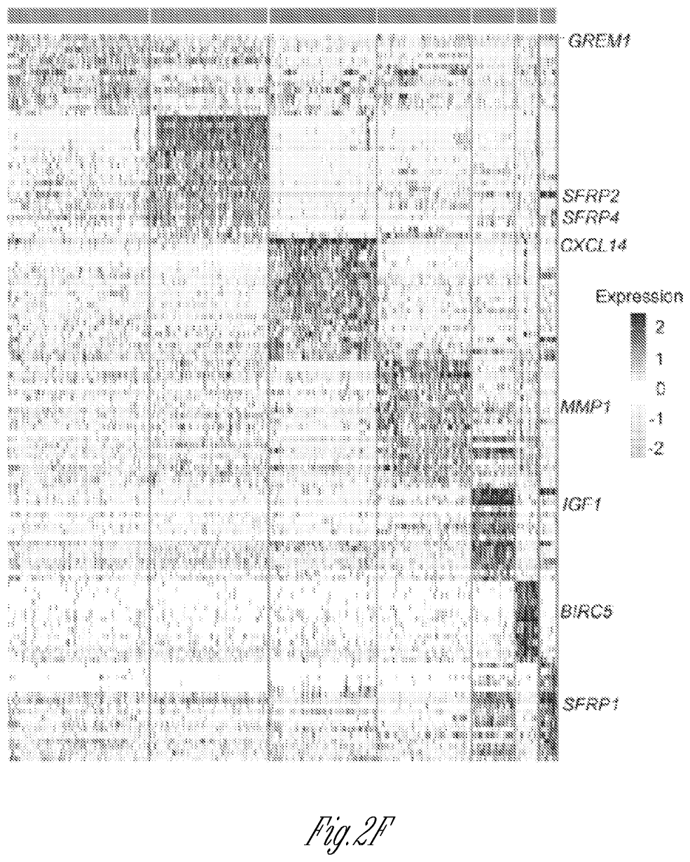

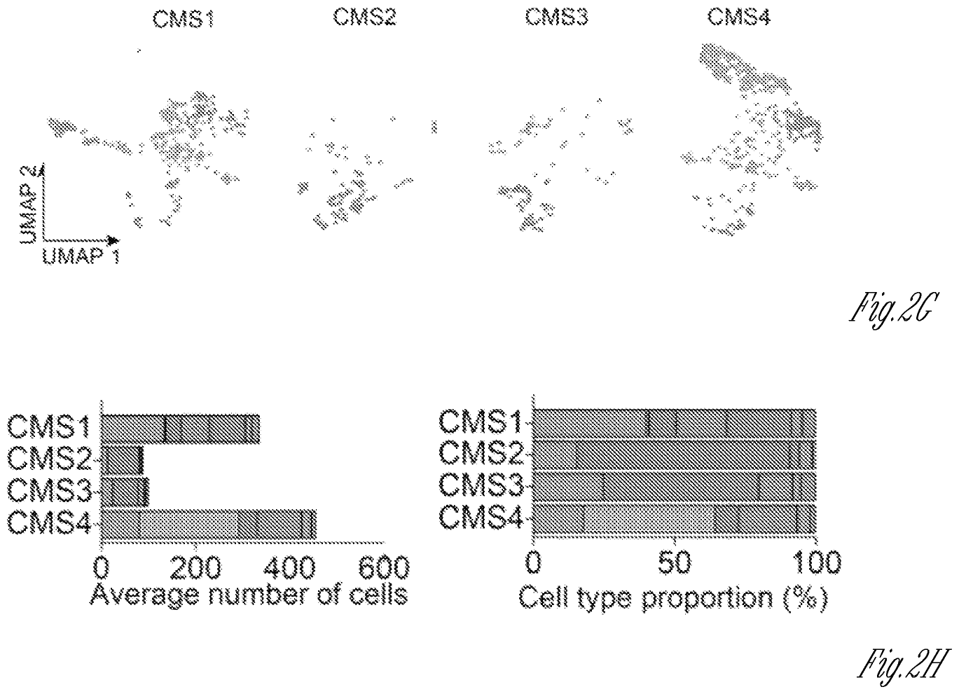

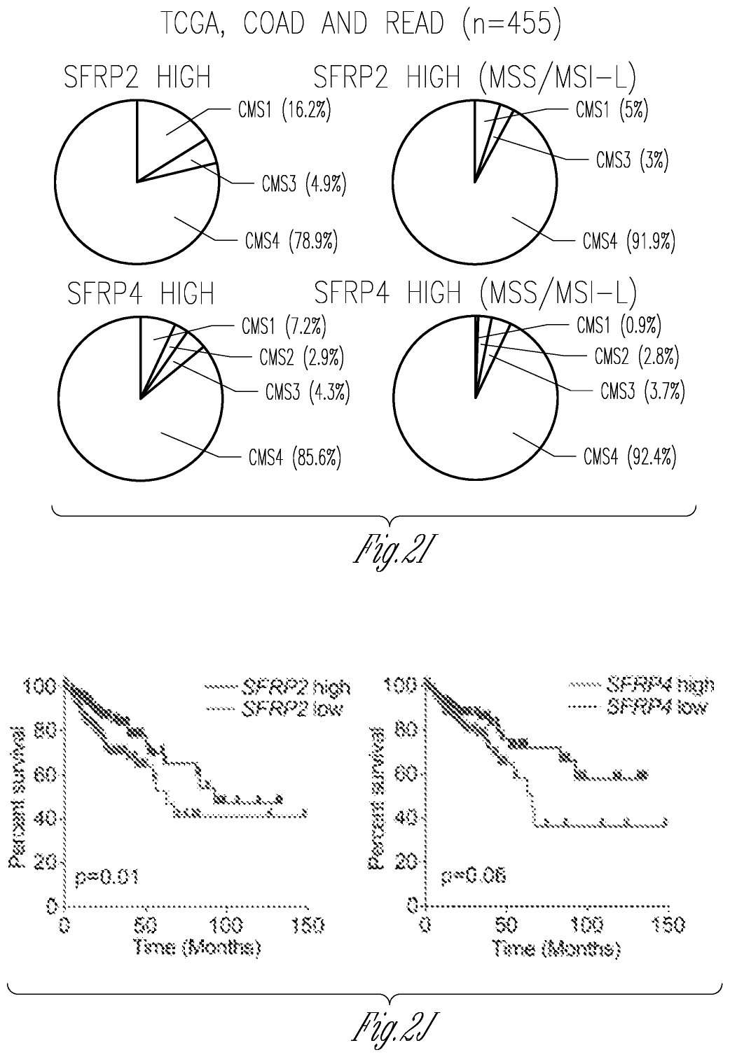

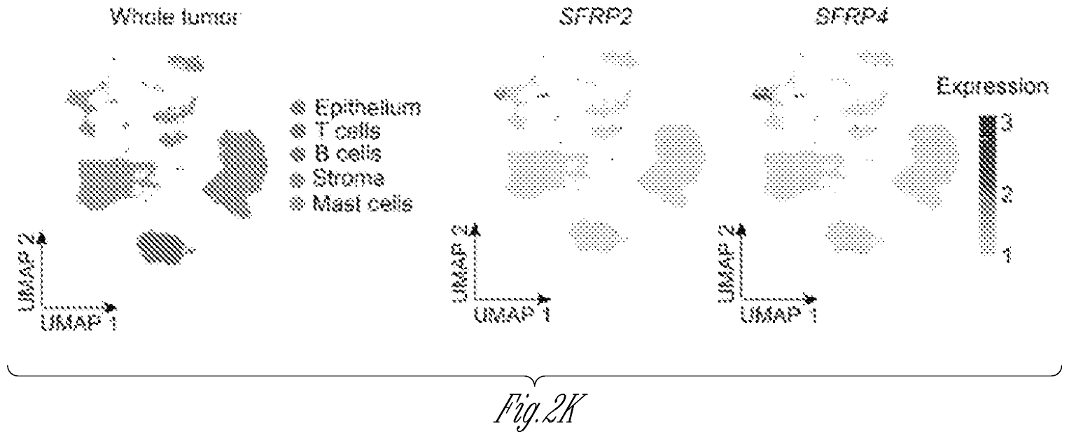

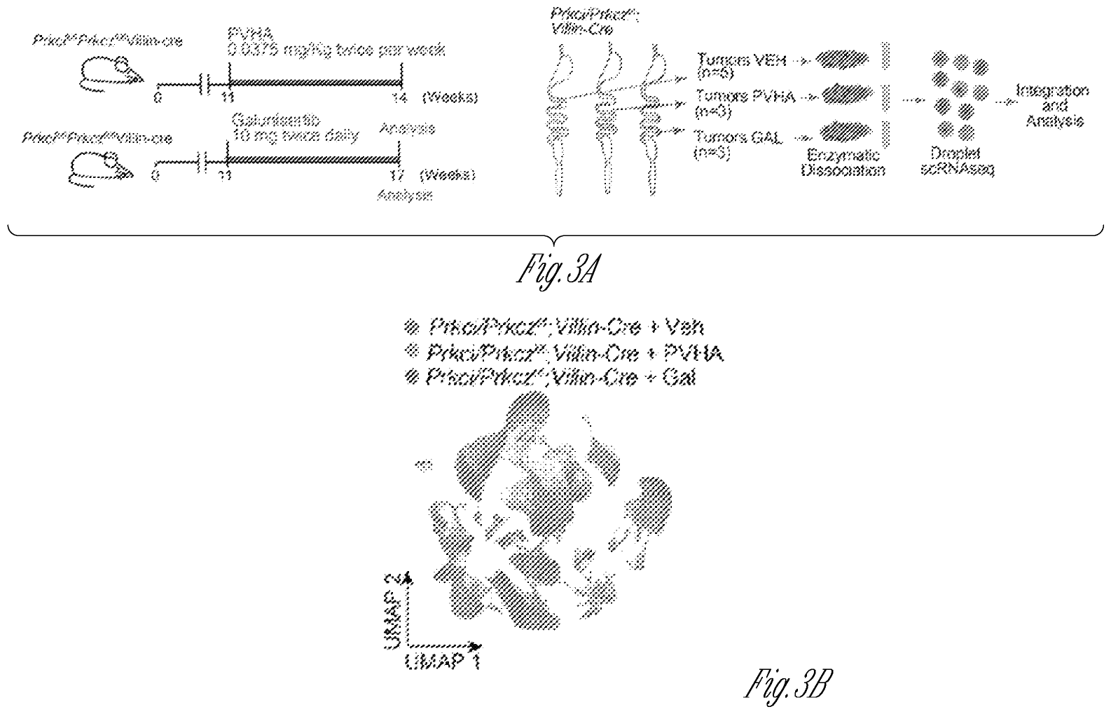

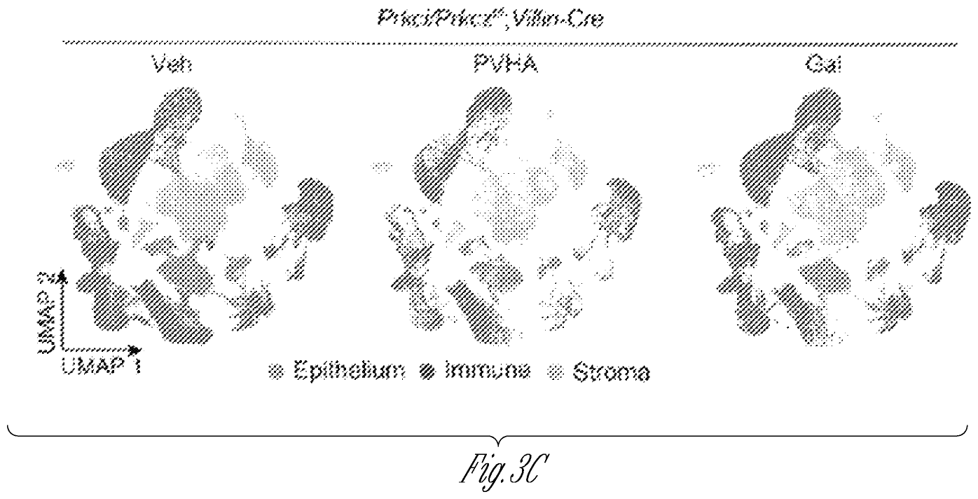

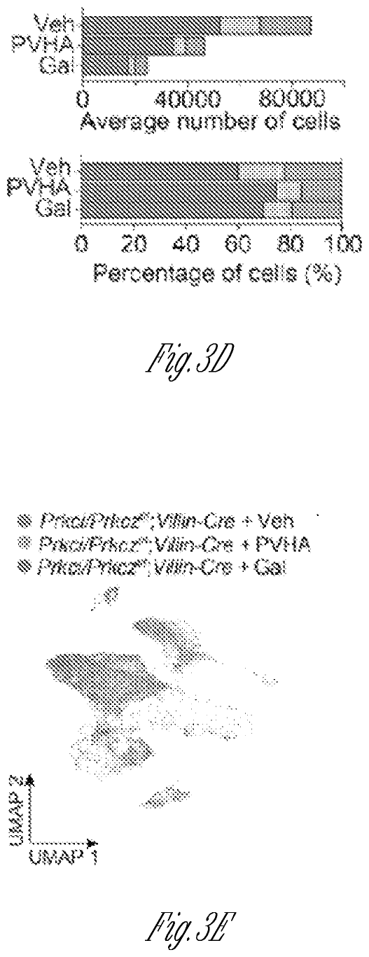

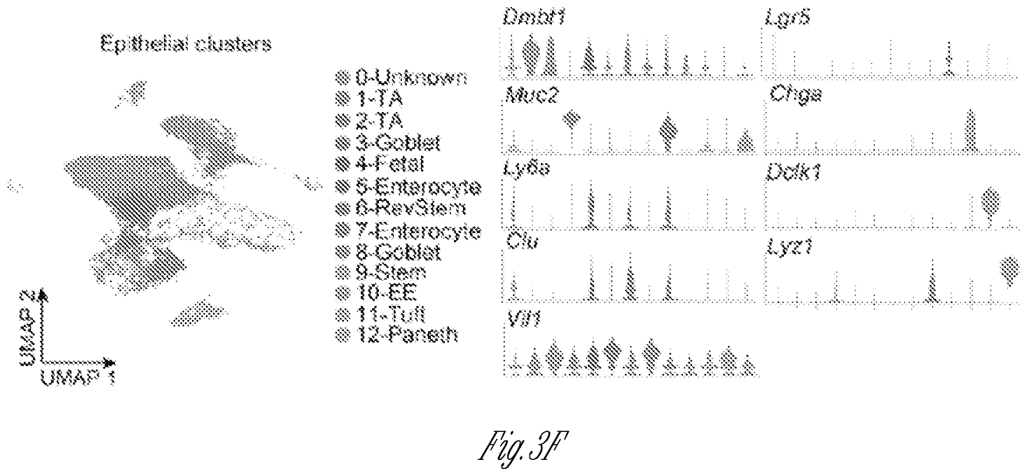

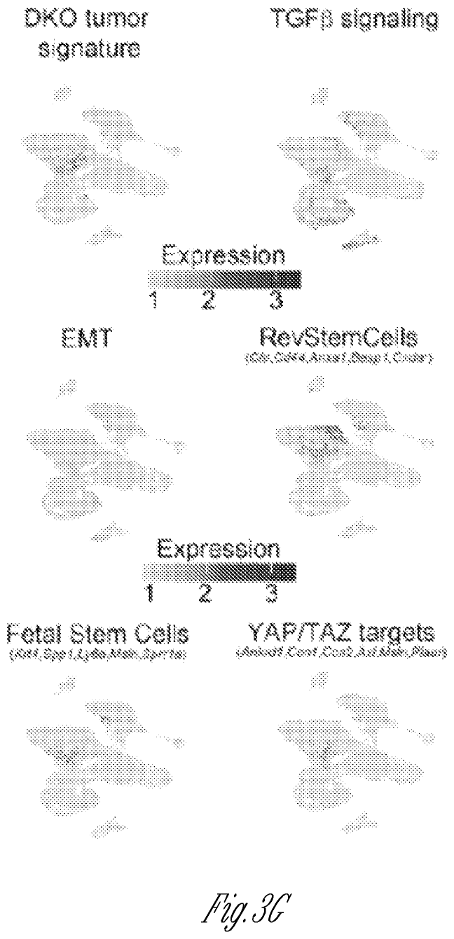

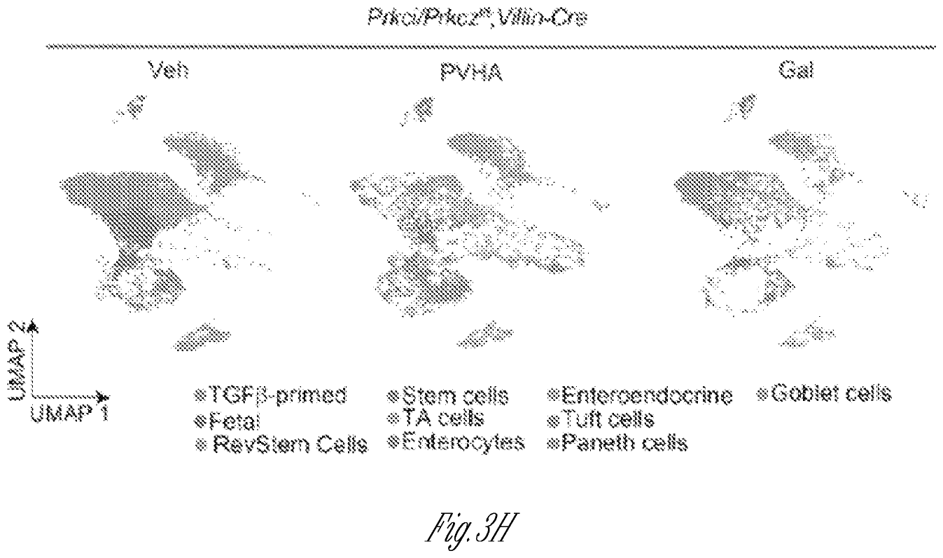

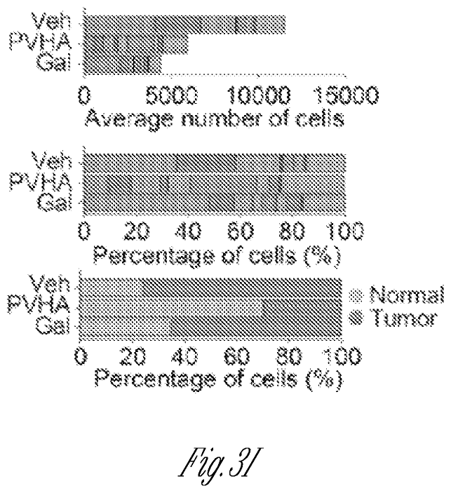

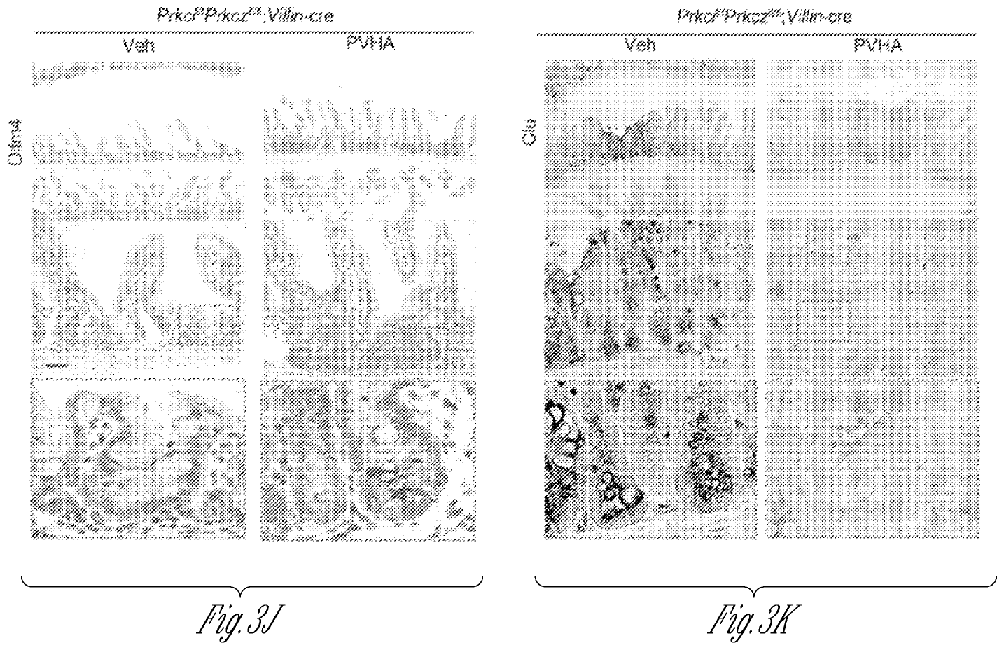

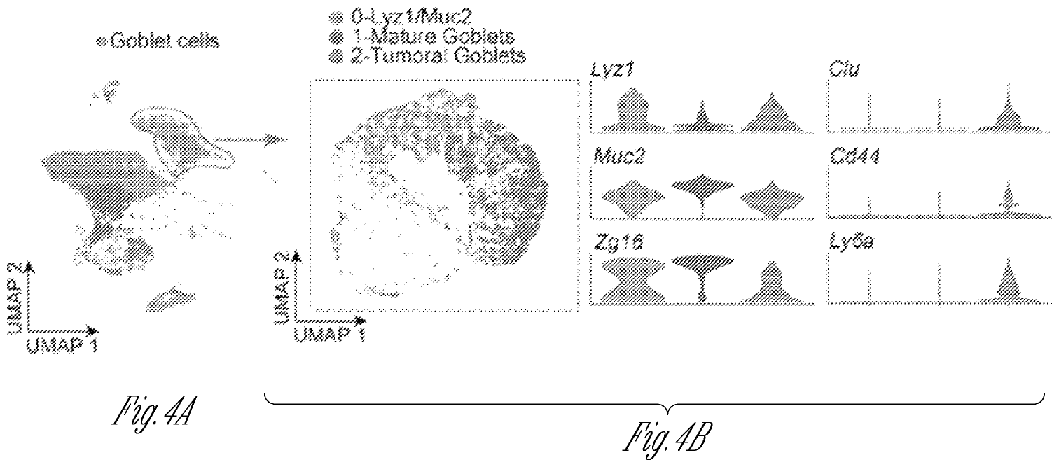

A previously published dataset of single-cell RNA-seq from human CRC patients (GSE132465) (Lee et al., Lineage-dependent gene expression programs influence the immune landscape of colorectal cancer. Nat Genet 52, 594-603, 2020) was analyzed to identify novel biomarkers that could better identify CMS4 tumors. The bioinformatic analysis of the stroma compartment of this dataset demonstrated the existence of four major tumor stomal cell types based on known markers for the different cell type populations: fibroblast (DCN), Endothelium (PECAM1), Pericytes (RGS5), and Vascular smooth muscle cells (MYH11) (FIGS. 2A-2B). The number of stromal cells was enriched in CMS4 and CMS1 groups, although all four subtypes contained stromal populations with similar proportions in CMS1, CSM3 and CMS4 types (FIGS. 2C-2D). Given that among stromal cell clusters fibroblasts were the main cell type, the fibroblast compartment was reclustered, which resulted in the identification of 7 distinct subpopulations (FIG. 2E). Gene expression analysis showed that cluster 0 was characterized by GREM1 expression, cluster 1 by SFRP2 and SFRP4, cluster 2 by CXCL14, cluster 3 by MMP3, cluster 4 by IGF1, cluster 5 by expression of genes involved in cell proliferation, and cluster 6 by SFRP1 expression (FIGS. 2E-2F).

Analysis of clusters across CMS subtypes identified cluster 1 with biomarkers SFRP2 and SFPR4 as exclusively presented in CMS4 patients (FIGS. 2G-2H and Table 1). To further validated these data, the TCGA CRC dataset was interrogated. High expression of SFRP2 or SFRP4 was strongly correlated with the CMS4 subtype (FIG. 2I). Since CMS4 is comprised mainly of MSS tumors, analysis excluding MSI-H patients showed a higher proportion of CMS4 cases correlating with high expression of SFRP2 and SFRP4 (FIG. 2I) was increased. Patients with higher levels of SFRP2 or SFRP4 gene expression showed worse overall survival (FIG. 2J), in keeping with the relevance of this fibroblast population in human CRC SFRP2 and SFRP4 were exclusively expressed in the stroma compartment as shown by analysis of the whole tumor population by scRNAseq (FIG. 2K). These results suggest that fibroblasts expressing SFRP2/SFRP4 might play a role in CMS4 cancer progression and can constitute a biomarker to selectively identify this type of tumors and predict clinical outcome in CRC.

TABLE 1
Fibroblast biomarkers selective of CMS4 tumors

| Gene symbol | Avg.logFC | p_val | p_val_adj |
|---|---|---|---|
| IGLC7 | 0.8669929 | 2.71E−173 | 4.28E−169 |
| SPINK4 | 0.801027 | 1.62E−133 | 2.55E−129 |
| TFF3 | 0.7809959 | 1.44E−100 | 2.27E−96 |
| TFF1 | 0.7726675 | 5.58E−128 | 8.79E−124 |
| IGFL2 | 0.7009532 | 2.63E−76 | 4.15E−72 |
| EPYC | 0.6827575 | 4.57E−57 | 7.20E−53 |
| REG4 | 0.6648143 | 1.23E−123 | 1.94E−119 |
| C1QTNF3 | 0.6255566 | 1.66E−51 | 2.62E−47 |
| GJB2 | 0.5935408 | 1.95E−47 | 3.07E−43 |
| SDC1 | 0.5901631 | 2.85E−64 | 4.49E−60 |
| ITLN1 | 0.588943 | 2.38E−128 | 3.75E−124 |
| MMP13 | 0.577397 | 2.86E−42 | 4.51E−38 |
| SFRP2 | 0.536741 | 3.44E−37 | 5.42E−33 |
| CPE | 0.5180354 | 1.95E−55 | 3.07E−51 |
| SFRP4 | 0.4974997 | 4.59E−26 | 7.23E−22 |
| TACSTD2 | 0.4873165 | 2.36E−84 | 3.72E−80 |
| AGR2 | 0.476894 | 2.30E−54 | 3.63E−50 |
| COL10A1 | 0.462467 | 8.76E−35 | 1.38E−30 |
| ASPN | 0.4547545 | 3.00E−29 | 4.73E−25 |
| CLDN4 | 0.4413463 | 1.01E−48 | 1.60E−44 |
| COL11A1 | 0.4399753 | 4.04E−33 | 6.36E−29 |
| MFAP5 | 0.4274909 | 2.91E−35 | 4.59E−31 |
| IGHG3 | 0.3980844 | 3.91E−42 | 6.16E−38 |
| CLDN3 | 0.3803987 | 1.63E−44 | 2.57E−40 |
| THBS2 | 0.3564331 | 2.92E−30 | 4.60E−26 |
| SCUBE3 | 0.3452036 | 1.15E−49 | 1.82E−45 |
| CD9 | 0.3327381 | 8.94E−21 | 1.41E−16 |
| COMP | 0.3286033 | 6.01E−22 | 9.47E−18 |
| HTRA1 | 0.3219267 | 9.23E−29 | 1.45E−24 |
| APOD | 0.3213093 | 1.48E−19 | 2.33E−15 |
| MMP11 | 0.3157726 | 2.33E−13 | 3.66E−09 |
| MGP | 0.3098747 | 3.87E−14 | 6.11E−10 |
| FBLN2 | 0.3066644 | 1.38E−22 | 2.18E−18 |
| GALNS | 0.3039461 | 3.16E−22 | 4.98E−18 |
| CD55 | 0.3029155 | 1.21E−20 | 1.91E−16 |
| FIBIN | 0.300431 | 2.60E−20 | 4.10E−16 |

### Example 2: Mechanism of Action of PVHA/PEGPH20 Treatment of CMS4 Tumors as Compared to TGFb Inhibition by Galunisertib

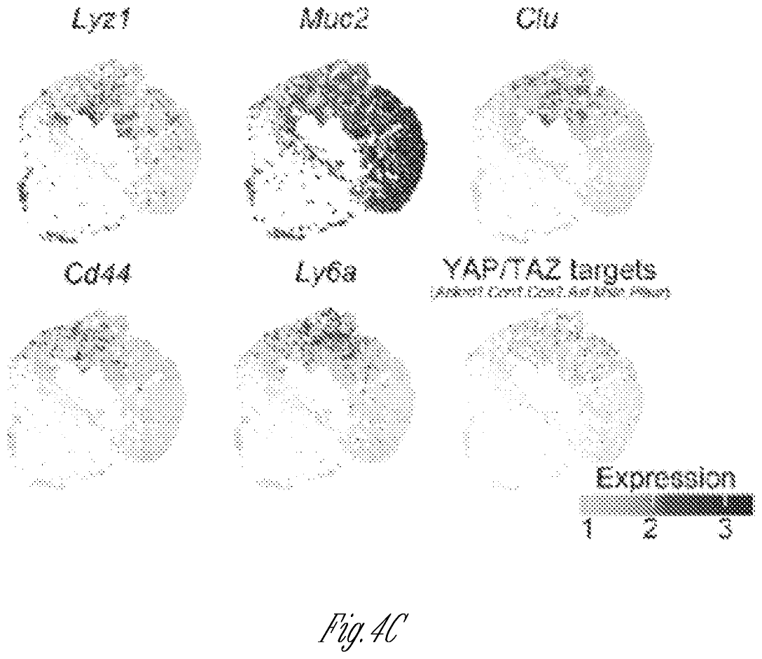

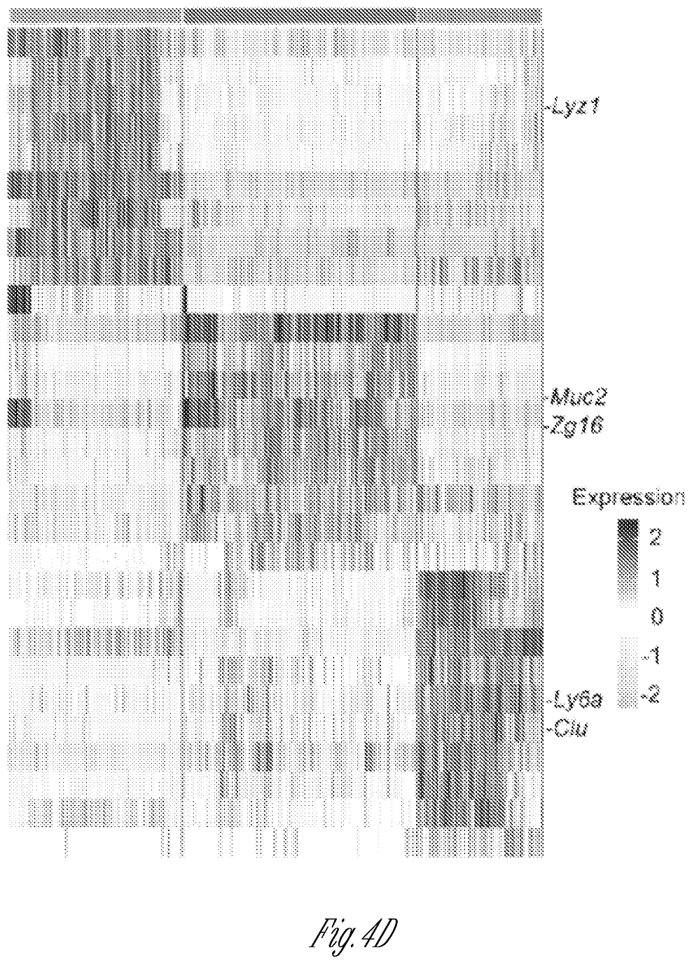

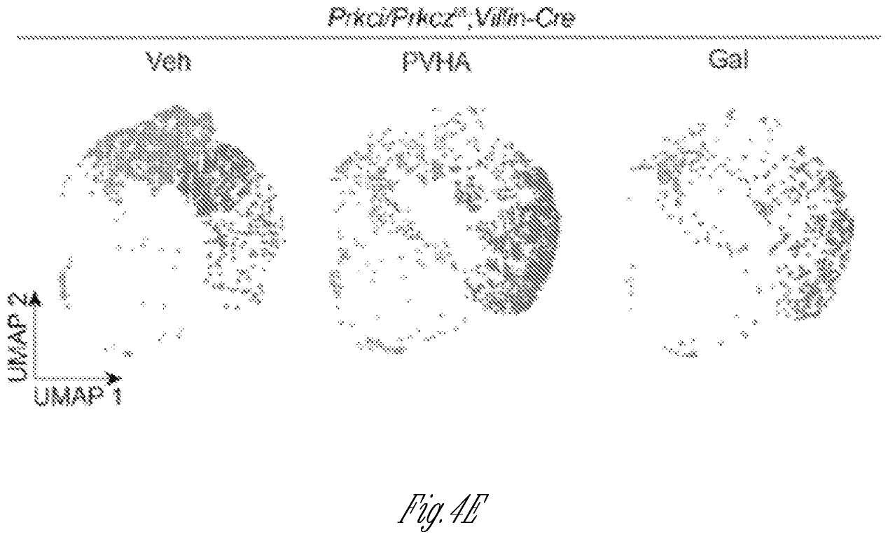

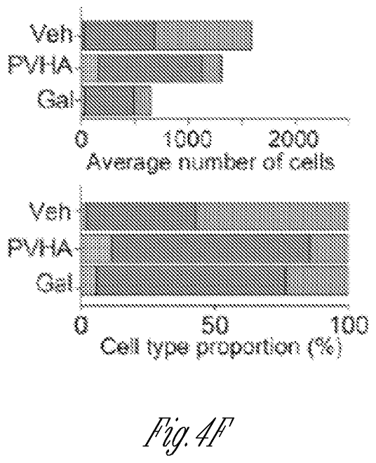

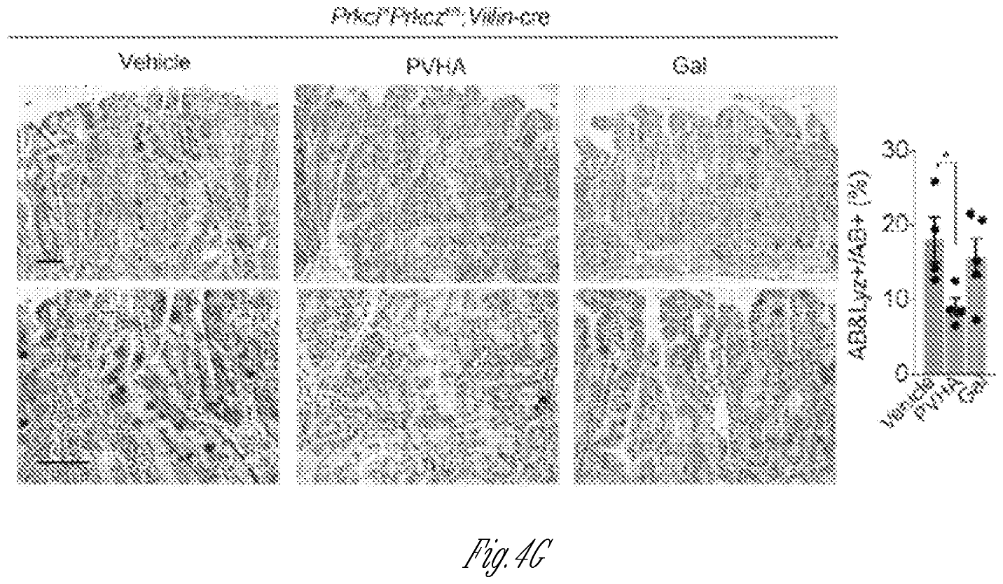

To identify the mechanism of action of PVHA in the treatment of CMS4 tumors, scRNAseq was performed using 10× Genomics Chromium platform on tumors from Prkci^{fl/fl} Prkcz^{fl/fl} Villin-cre (DKO) mice (n=5) treated with PVHA for 14 weeks (n=3), as compared to those treated with Galunisertib (GAL) for 17 weeks (n=3), as shown in experimental design (FIG. 3A). Unsupervised clustering identified tumor and normal epithelial cells as well as stromal and immune cells with selective markers for each population (FIGS. 3B-3C). The percentage of cells for each group showed a decreased in stromal and immune cells upon both treatments (FIG. 3D). After re-clustering epithelial cells, mapping of marker gene expression across 13 identified cell clusters (clusters 0-12) revealed multiple distinct populations, including Lgr5 stem cells, enterocytes, goblet, Paneth, and enteroendocrine cells (FIGS. 3E-3F). Among tumor clusters (0,4,6), a cell population with transcriptomic similarities to the recently identified “revival stem cells” (RSC; high Clu) in the vehicle treated tumors was identified (FIGS. 3F-3G).

Gene expression analysis also showed the expansion of other two different tumoral clusters. Despite cluster 4 having some overlap with the RSC cluster, these cells are characterized by elevation of the Fetal Stem Cell signature and YAP/TAZ targets. Furthermore, Fetal Stem signature mostly overlap at the transcriptome level with DKO tumor (from bulk RNAseq), Hallmark EMT, and TGFβ signaling signatures (FIG. 3G). Finally, a third cluster was identified by lacking specific gene markers and the loss of TGFβ receptors and TGFβ signaling, which anticipates resistance to GAL treatment (FIG. 3G). Cell percentage analysis showed not only a clear reduction of tumor cells upon PVHA treatment as compared to vehicle or GAL-treated but also a recovered Lgr5 Stem Cells population lost in the DKO intestine (FIGS. 3H-3I). In good agreement with these observations, immunohistochemical (IHC) analysis showed an increase of Olfm4 (Stem cell marker) and a decrease of Clu upon PVHA treatment (FIGS. 3J-3K).

In addition, it was noted that some Goblet cells showed an overlap with signatures related to tumoral clusters (FIG. 3G), therefore this cell population was analyzed and re-clustered (FIG. 4A). After re-clustering, three major Goblet cell populations were obtained (FIG. 4B). Based on gene expression markers, cluster 0 was annotated as Lyz1/Muc2, suggesting a phenotype of secretory progenitor or goblet immature. Cluster 1 was annotated as a mature goblet, characterized by high Muc2 and Zg16 expression. Finally, cluster 3 was annotated as a tumoral goblet with a transcriptional profile overlapping with that of Revival Stem cells and Fetal Stem cells found in tumors (FIGS. 4B and 4D). Both PVHA and GAL treatments showed a reduction in the tumoral goblet population but PVHA increased mature goblets proportion as compared to GAL (FIGS. 4E-4F). Furthermore, IHC double staining for Alcian blue and Lyz1 showed a reduction of immature goblets upon PVHA treatment (FIG. 4H). Without beding limited to any specific theory, this analysis of tumor epithelial cells suggests that HA degradation was more effective reducing tumor cell populations and restoring stem cell homeostasis than TGFβ inhibition in a model of CMS4 CRC.

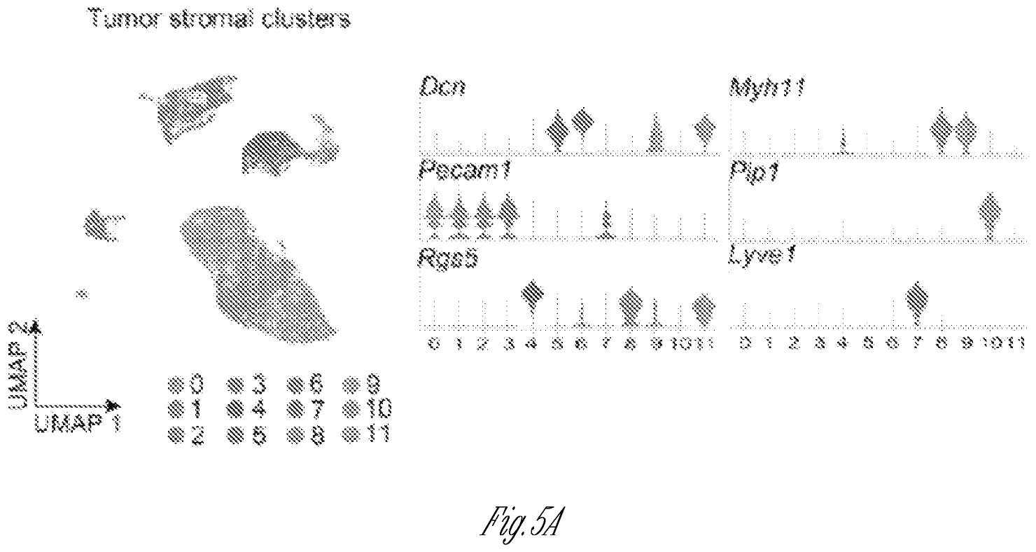

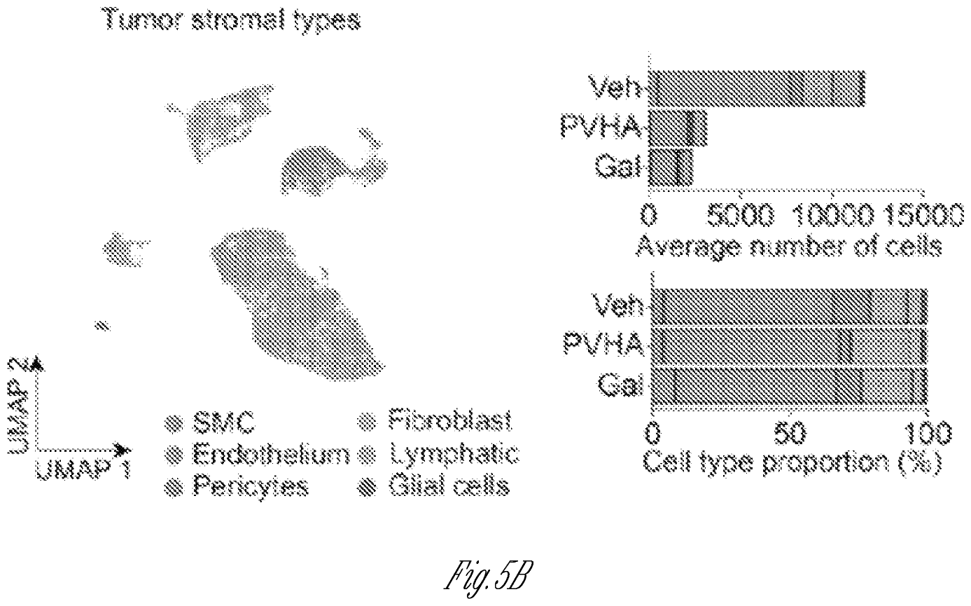

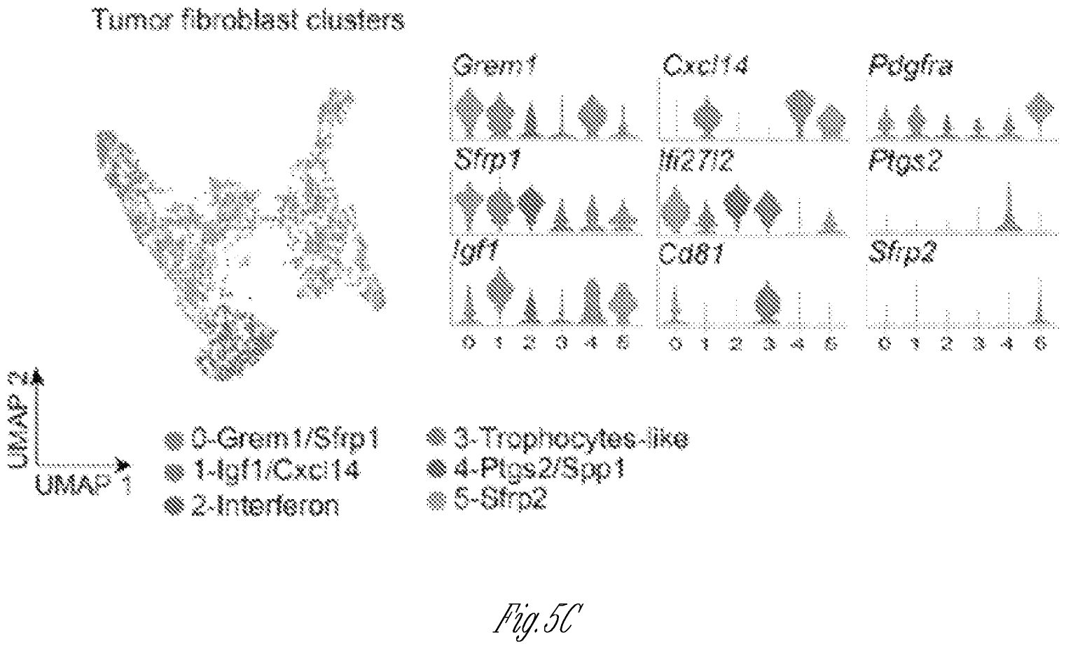

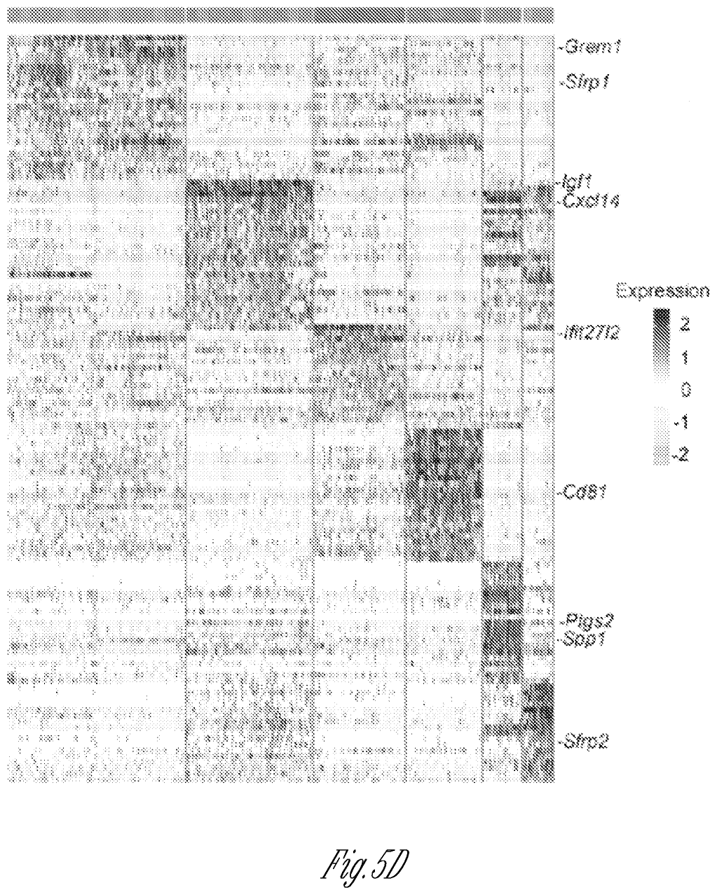

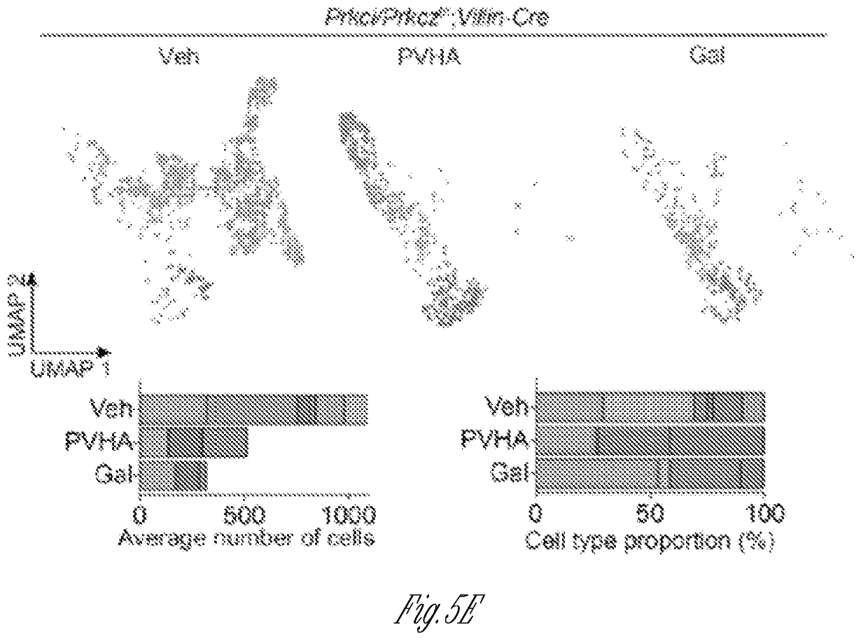

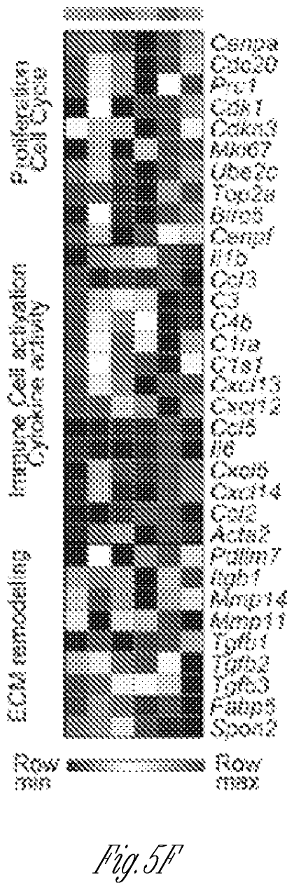

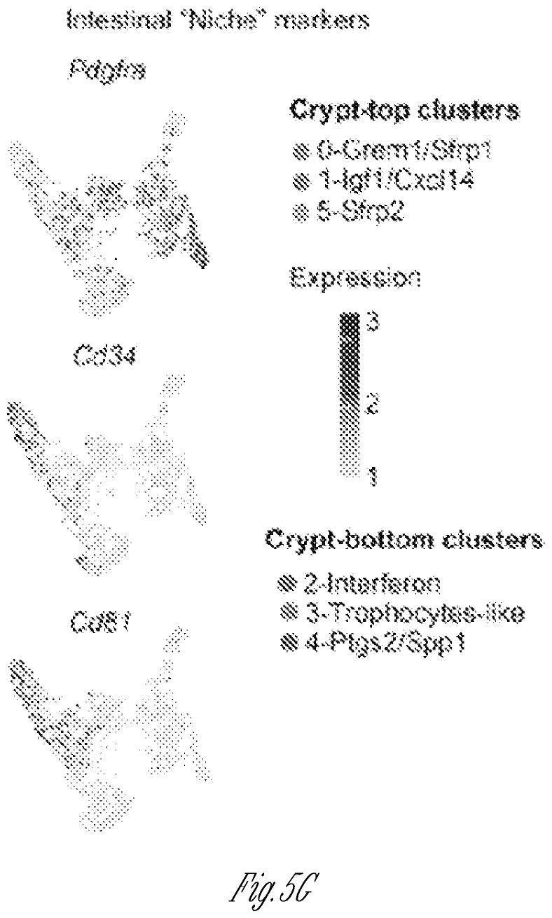

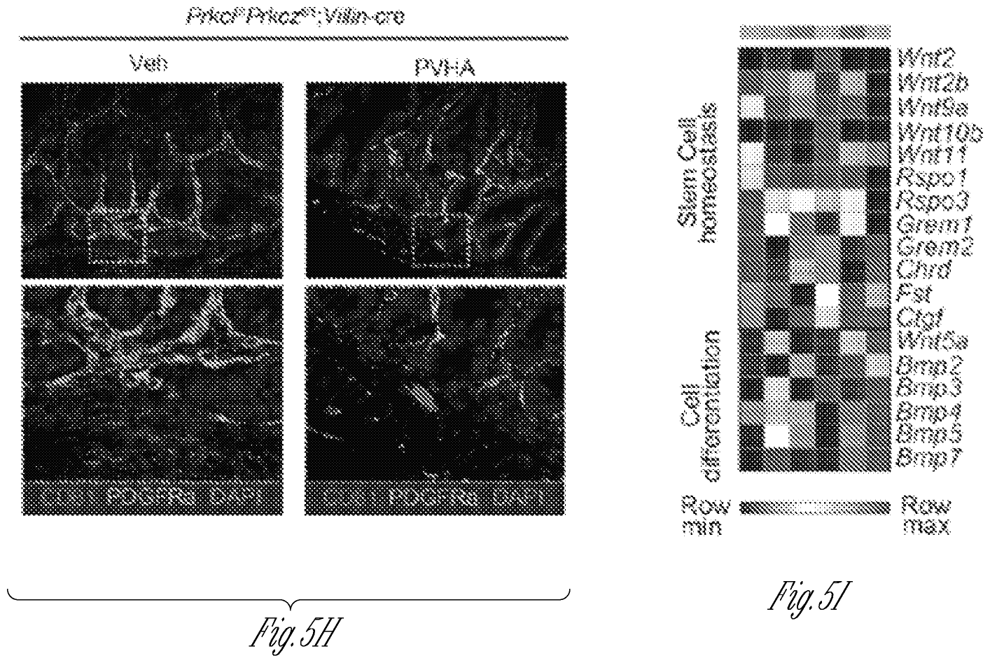

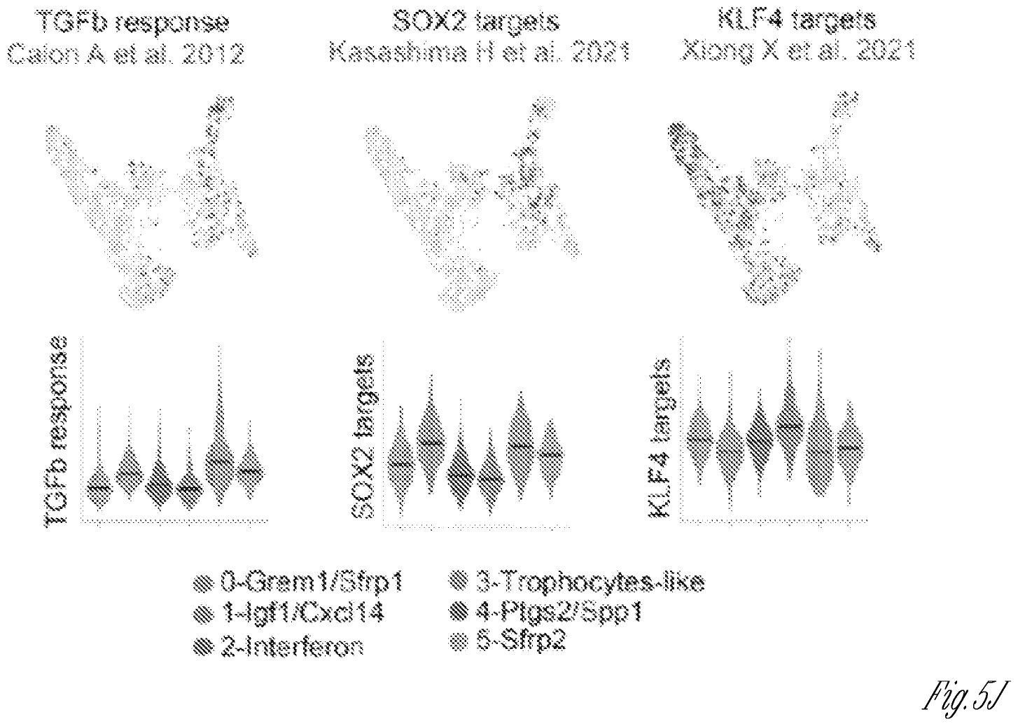

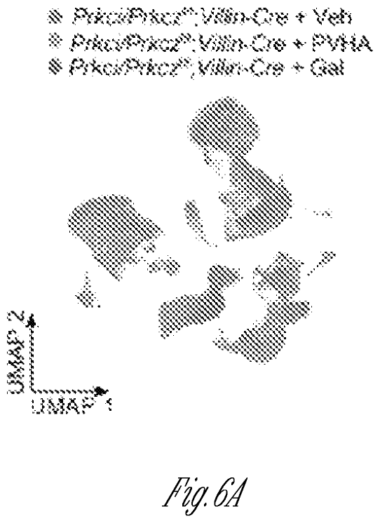

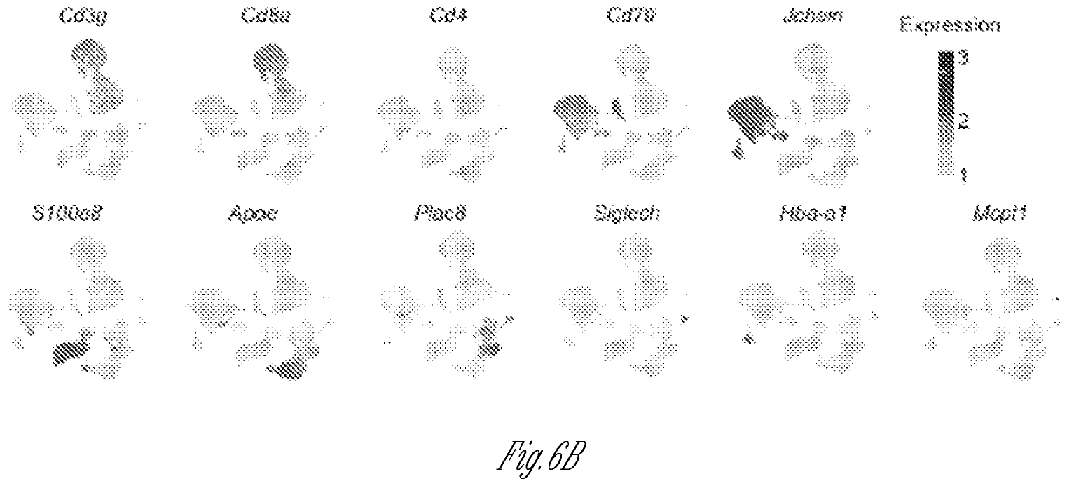

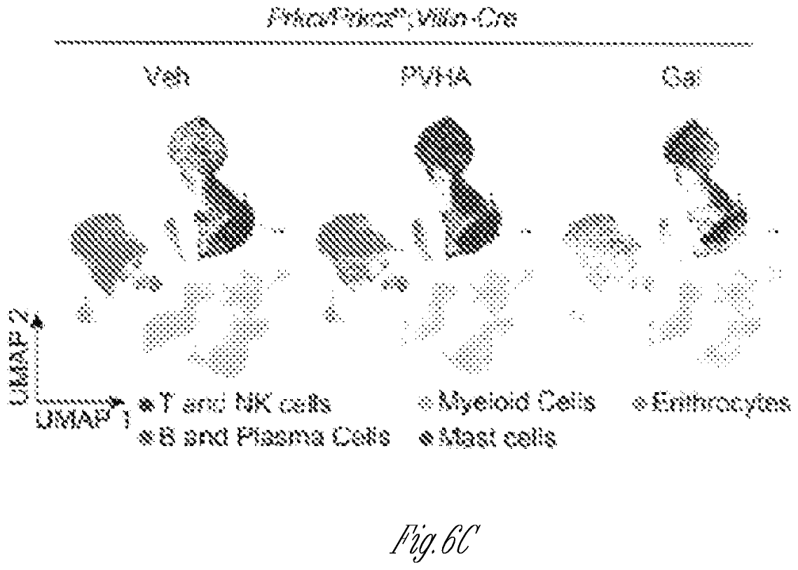

To investigate the potential mechanism of action of PVHA, the stromal and immune compartment were analyzed upon both treatments. Analysis of the stroma identified 12 clusters (Cluster 0-11) classified by using known markers (FIG. 5A). Among all stromal clusters, six major tumor stromal cell types were identified including fibroblast (Dcn), Endothelium (Pecam1), Pericytes (Rgs5), Smooth muscle cells (Myh11), Glial cells (Plp1), and Lymphatic-endothelial (Lyve1) (FIG. 5B). Cell count analysis revealed that both PVHA and GAL treatment reduced the number of stromal cells without apparent changes in cell-type proportions (FIG. 5B). Next, the fibroblast was isolated and six distinct populations were identified (FIG. 5C). Analysis of gene expression showed similar profiles to those obtained from human CRC patients (FIGS. 2A-2K). In addition, an interferon-enriched cluster was identified that was not present in the human CRC fibroblasts (FIGS. 2A-2K, 5C and 5D). This cluster shared features with a fibroblast type recently described (McCarthy et al., Distinct Mesenchymal Cell Populations Generate the Essential Intestinal BMP Signaling Gradient. Cell stem cell 26, 391-402 e395, 2020) as the key component of the Stem Cell niche (Cd34, Cd81) and with high expression of Ptgs2 and Spp1 (Cd44 ligand), the signaling hub of tumor initiating cells (FIGS. 5C and 5D). Cell-type proportion analysis showed complete elimination of clusters associated with human CRC (Grem1/Sfrp1, Igf1/Cxcl14), CMS4 subtype (Sfrp2), and tumor initiation (Ptgs2/Spp1) upon PVHA and GAL treatments. However, PVHA, but not GAL, treatment strongly increased the Trophocyte-like cluster (Cd34, Cd81), a key population to maintain the Stem Cell homeostasis (FIG. 5E). Further pathway analysis confirmed that eliminated clusters were highly proliferative and high ECM remodeling (FIG. 5F). The expression of Pdgfra (Crypt-top) and Cd81/Cd34 (Crypt-bottom) are well-known zonation markers in the small intestine (McCarthy et al., 2020). Gene expression analysis of these markers suggested a Peri-cryptal distribution of the Trophocyte-like and the Ptgs2/Spp1 fibroblast (FIG. 5G). The position of Trophocytes-like fibroblast was confirmed by IHC, which demonstrated the induction of Cd81+Pdgfra− fibroblast upon PVHA around the Crypt in DKO intestine mice (FIG. 5H).

Moreover, analysis of secreted factors involved in Stem Cell homeostasis such as Wnt/Rspo1 and Cell differentiation (Bmp genes) demonstrated the functionality of these fibroblast populations modulated by PVHA treatment (FIG. 5I). Next, the upstream regulators that control the expression of this fibroblast population was identified. The data revealed enrichment in TGFβ response and Sox2 target signatures in fibroblast eliminated by PVHA and GAL (FIG. 5J). Klf4 targets signature was increased in those fibroblasts induced by PVHA.

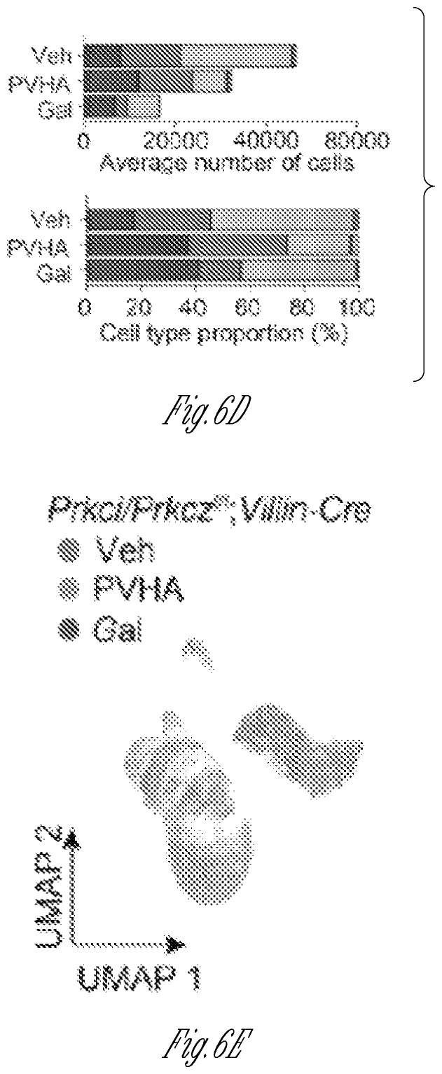

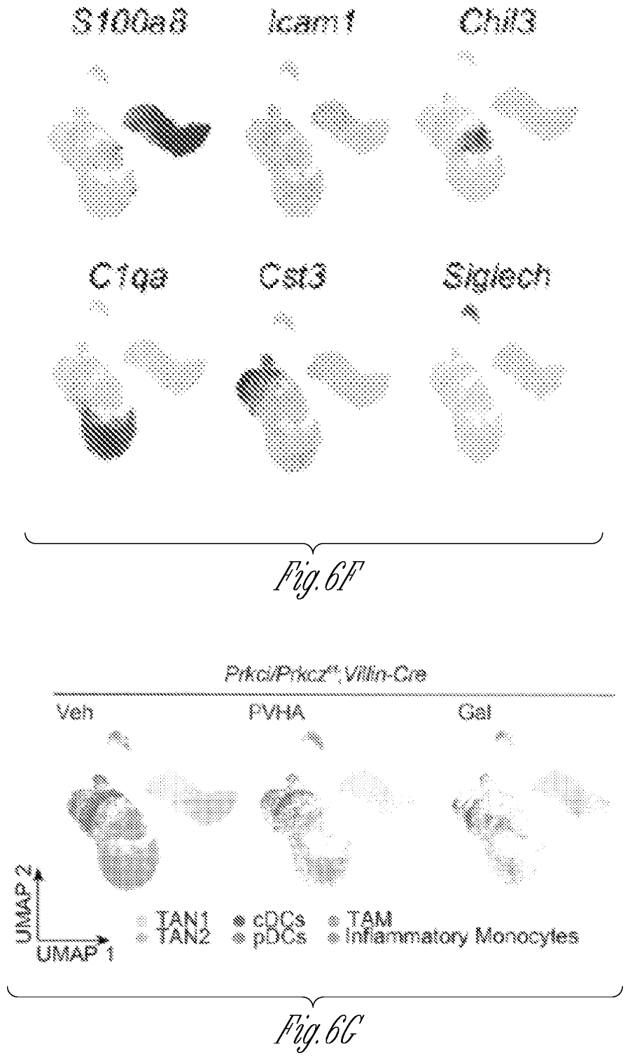

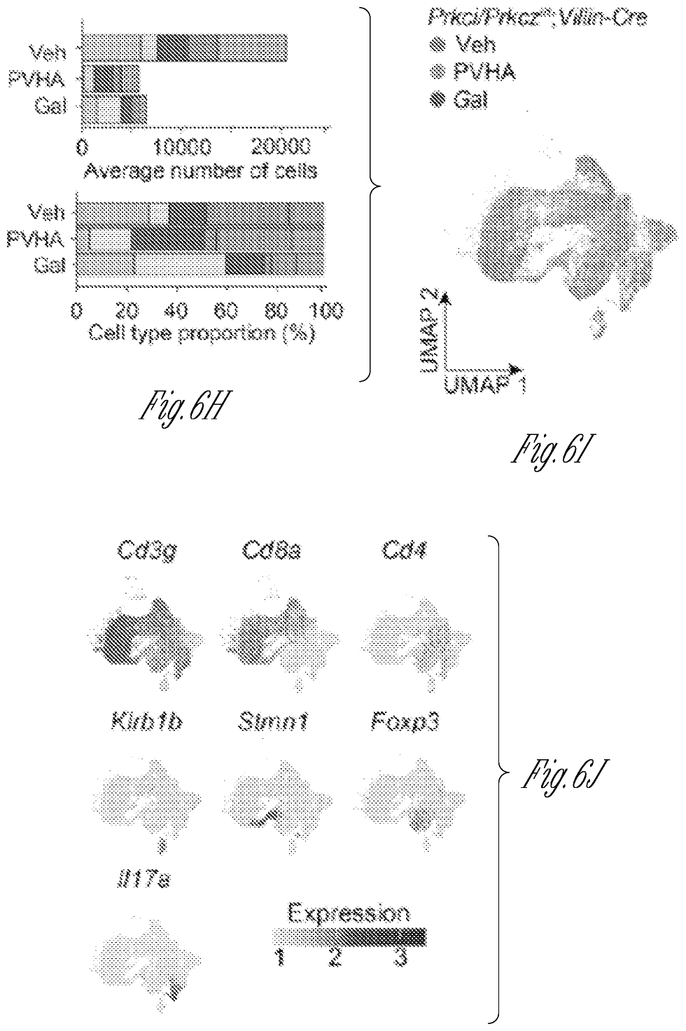

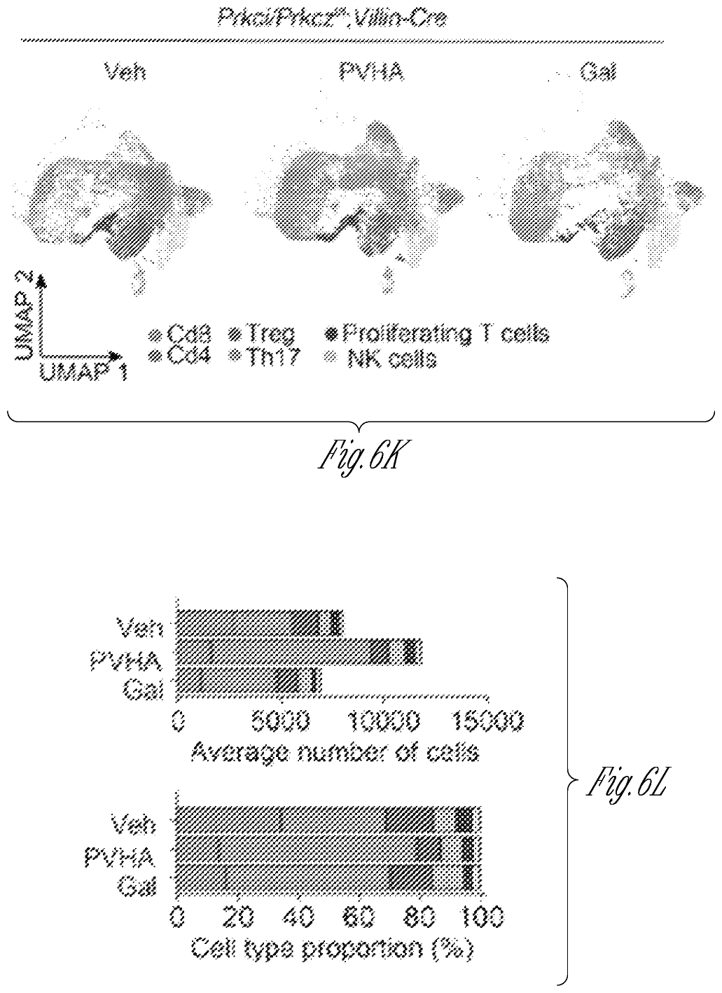

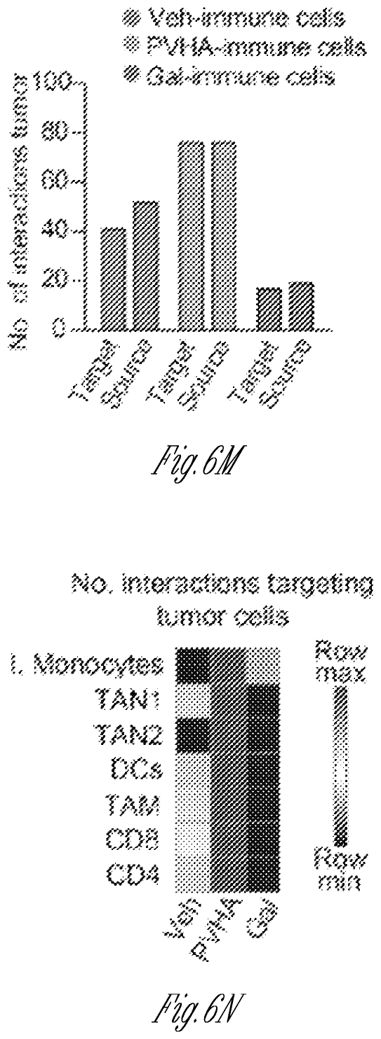

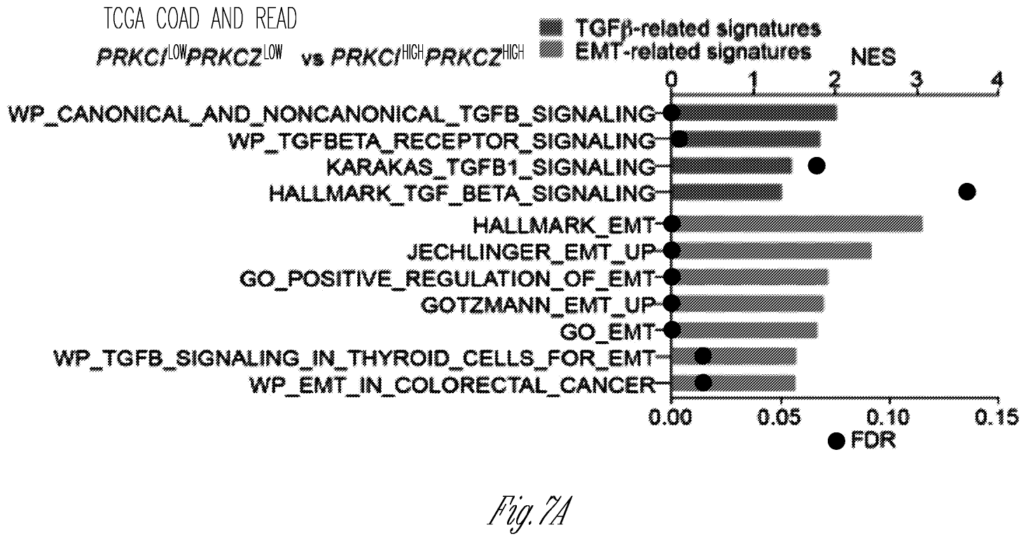

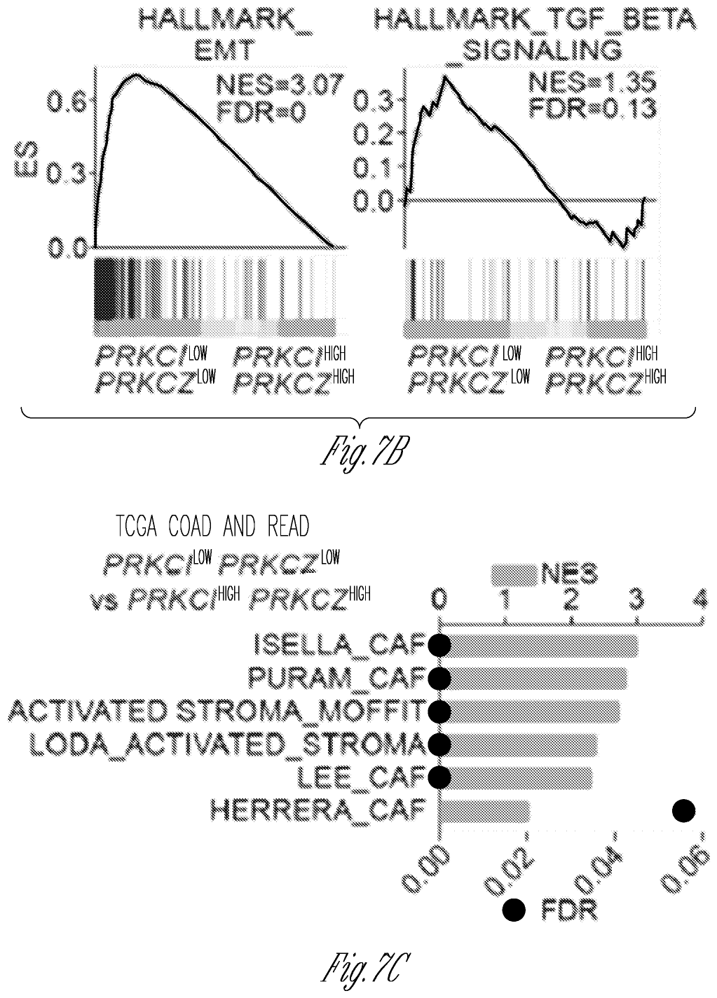

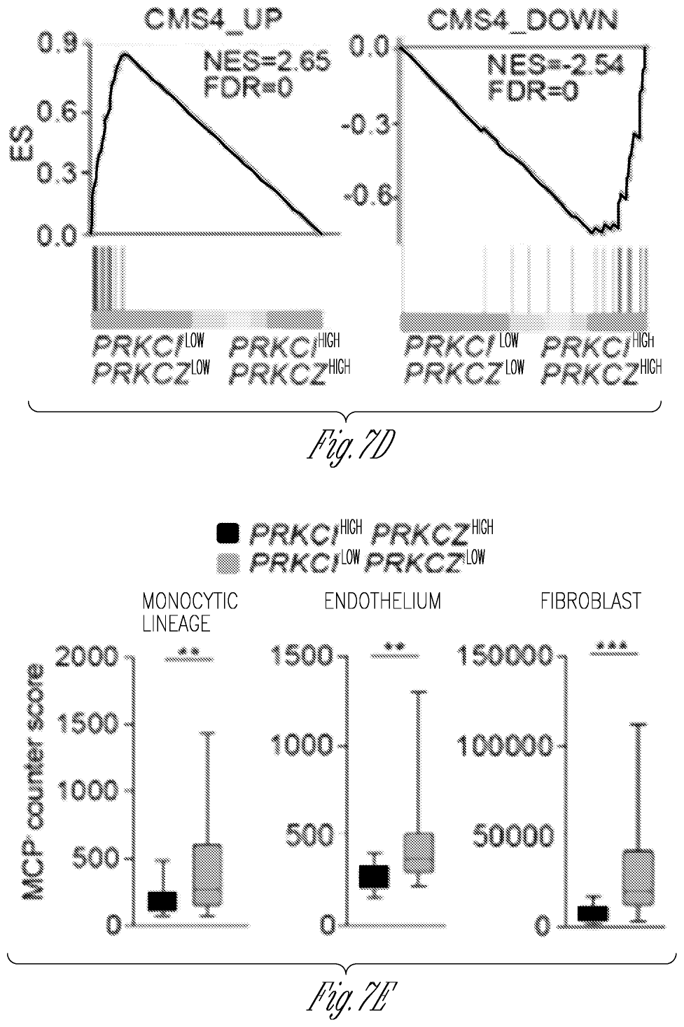

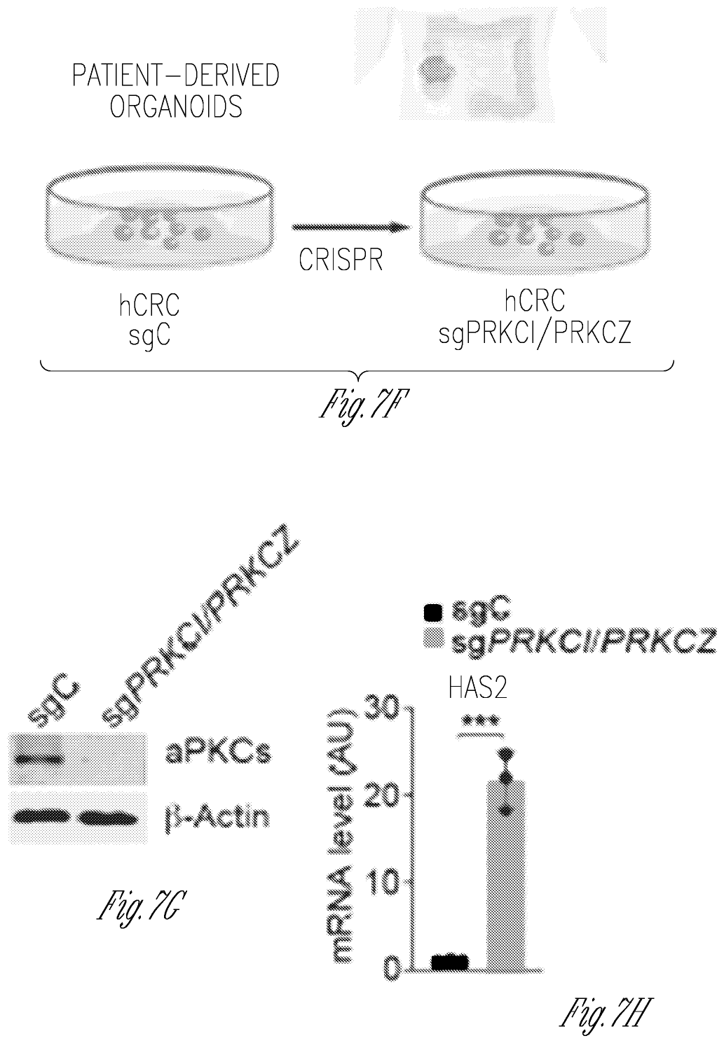

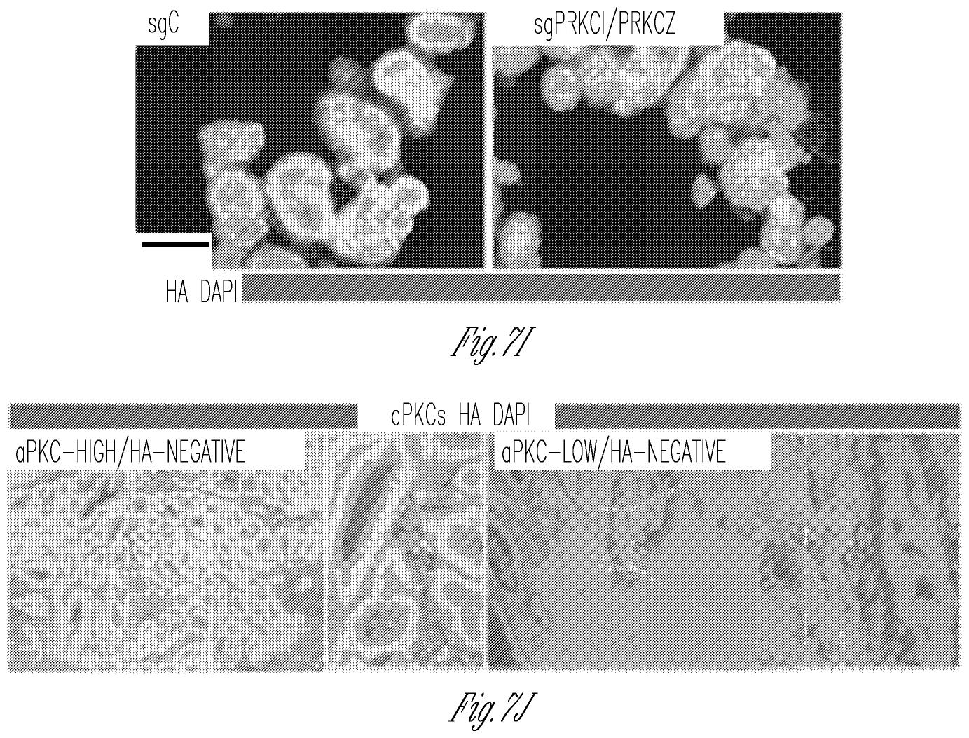

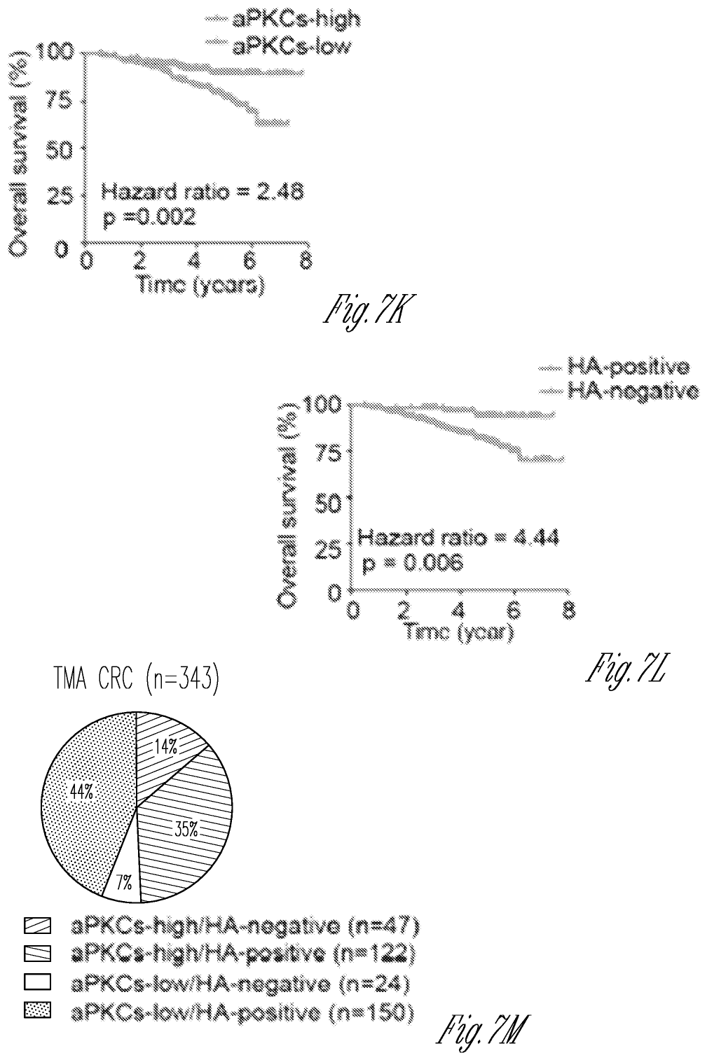

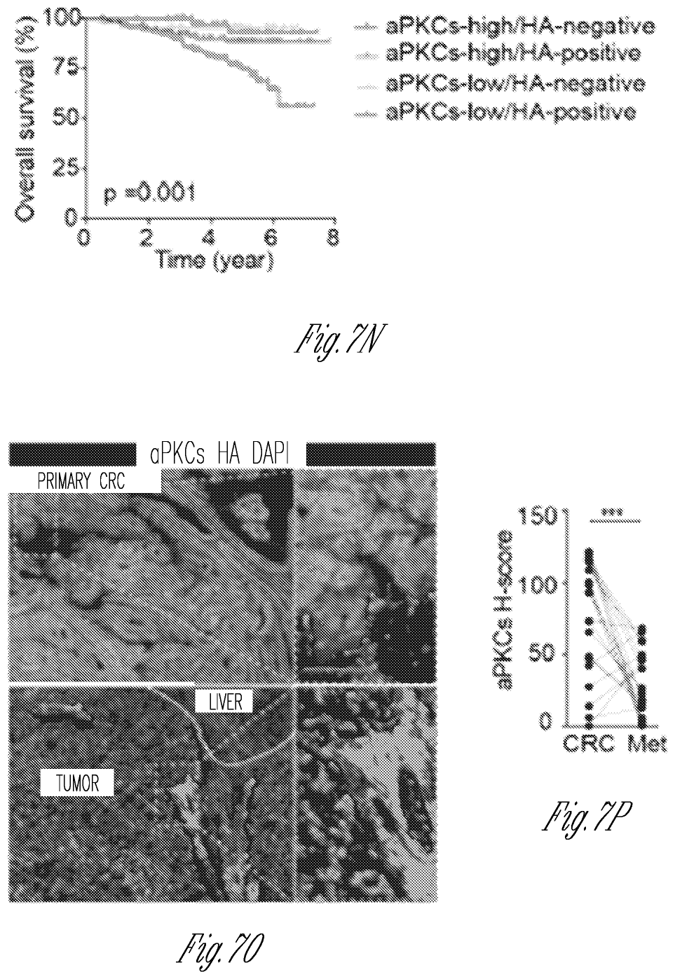

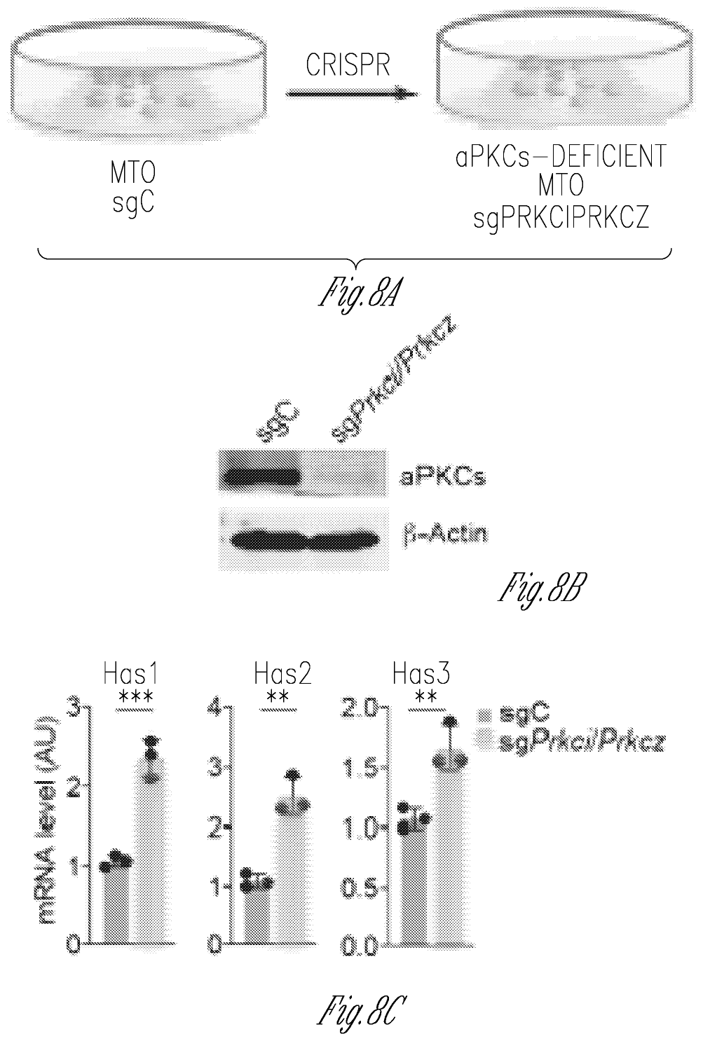

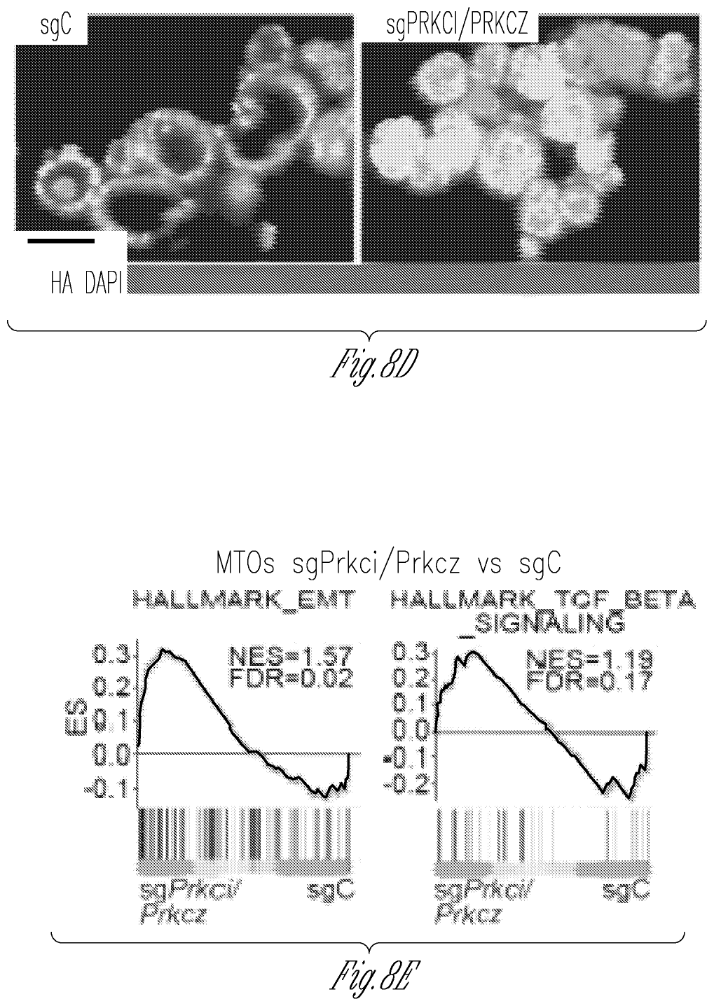

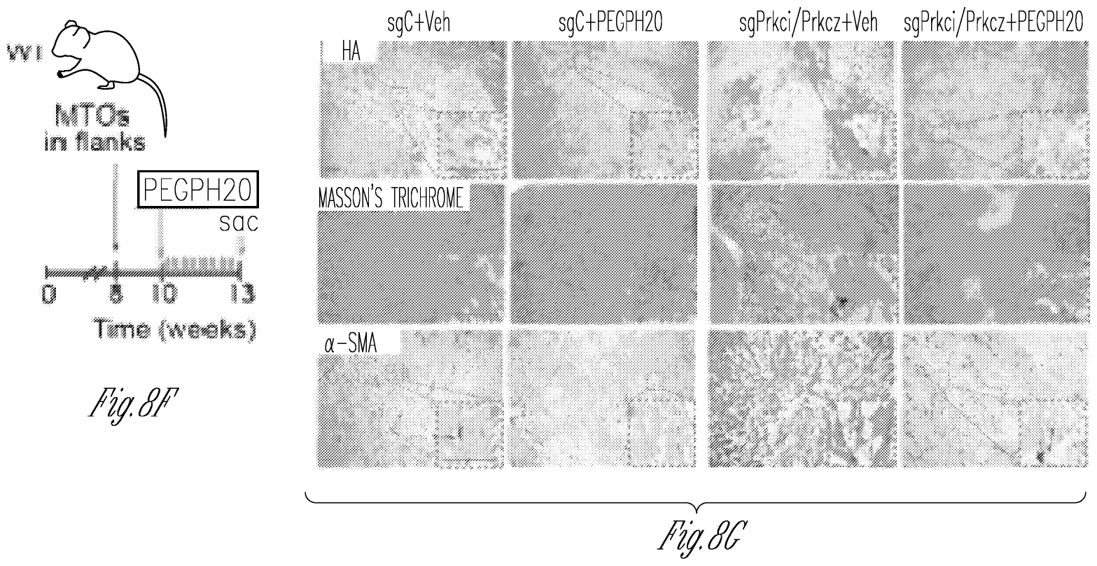

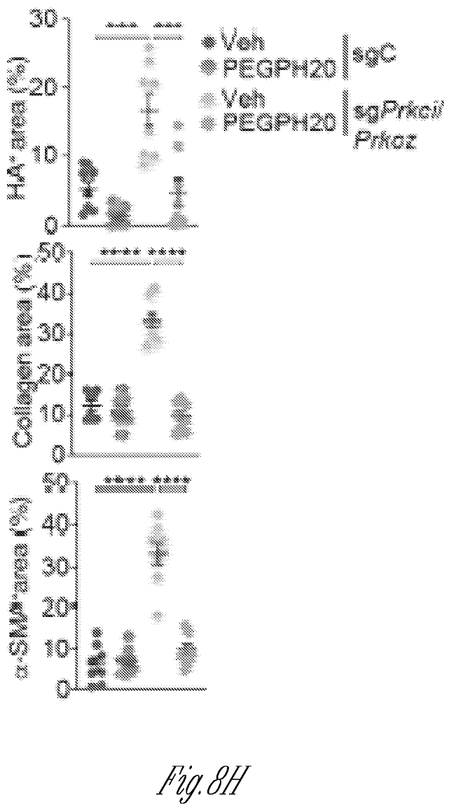

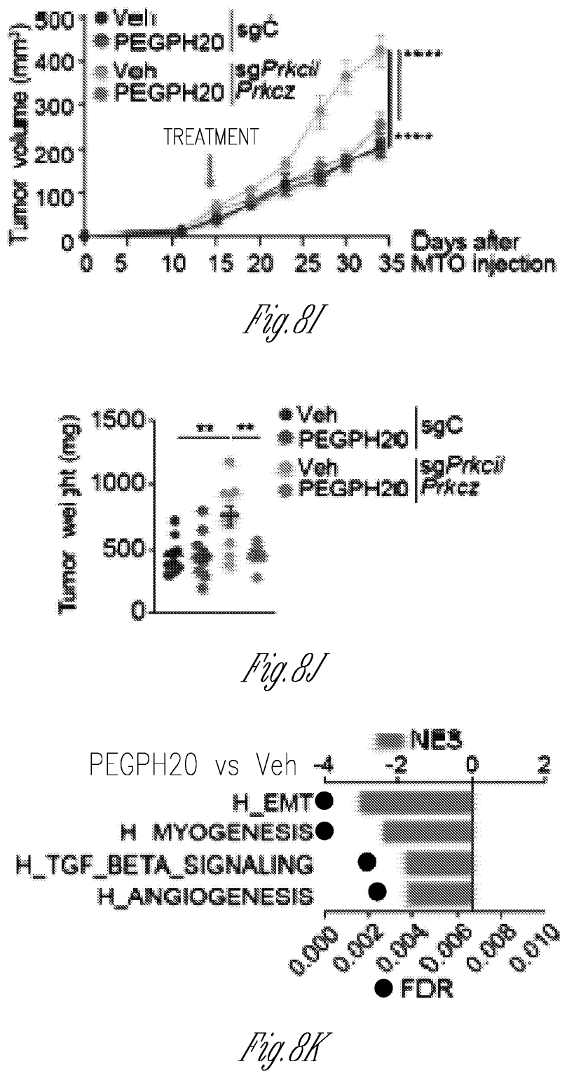

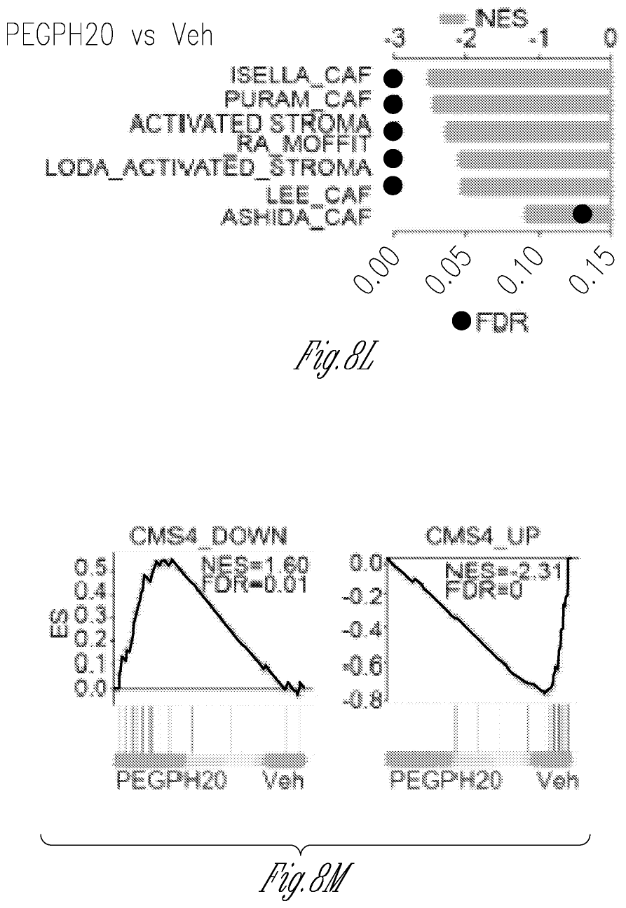

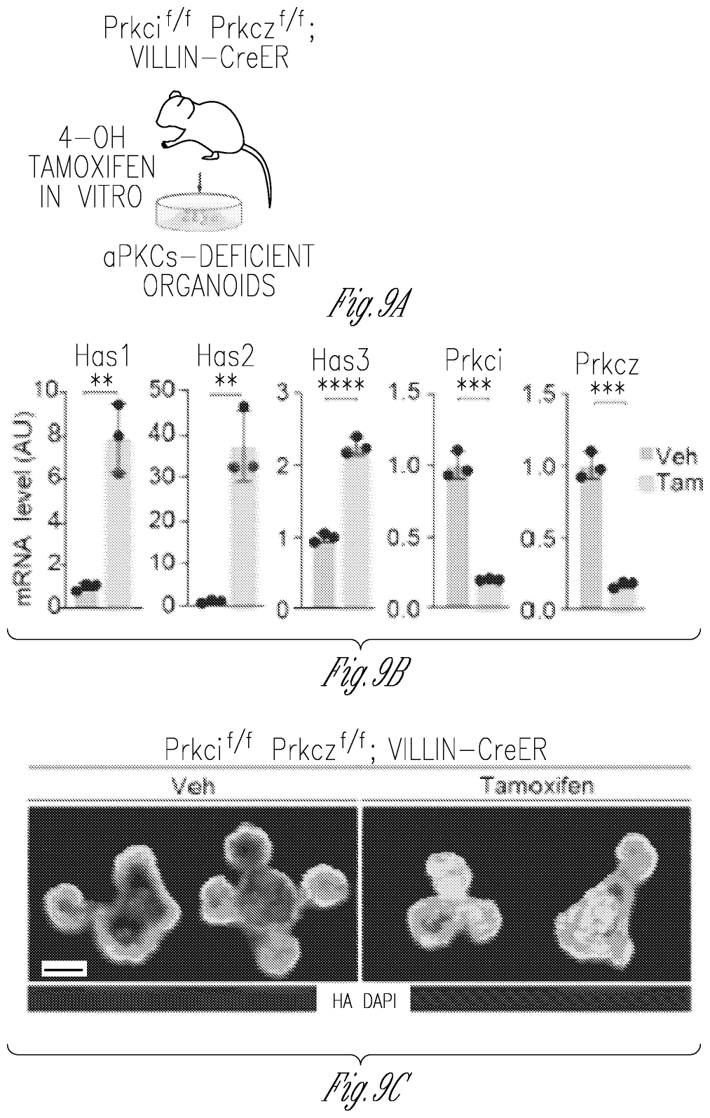

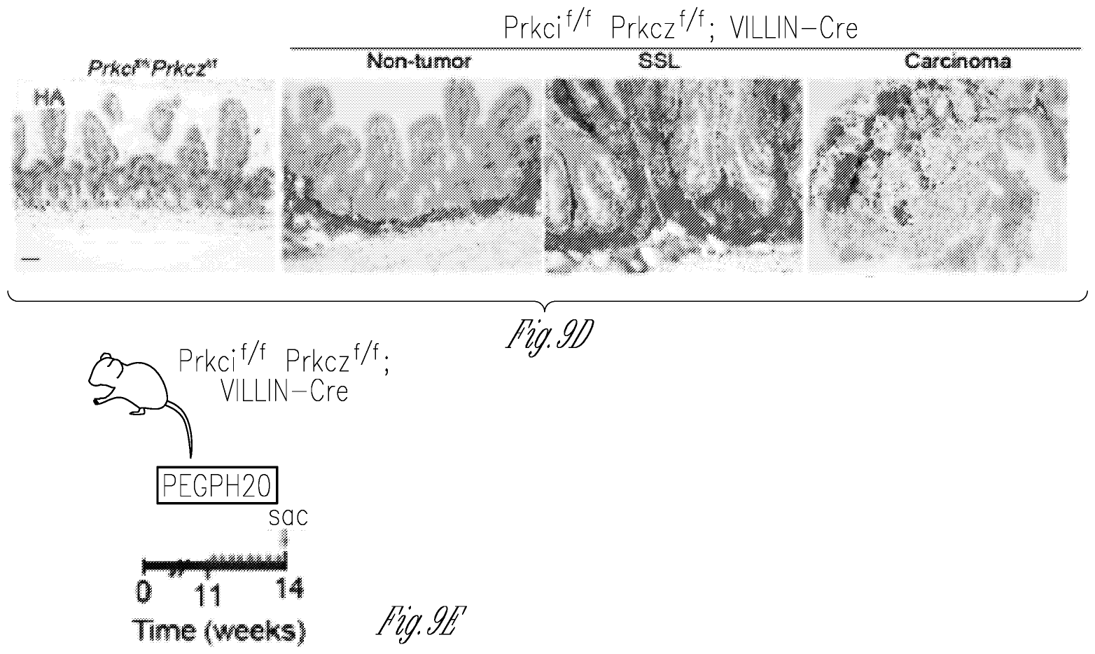

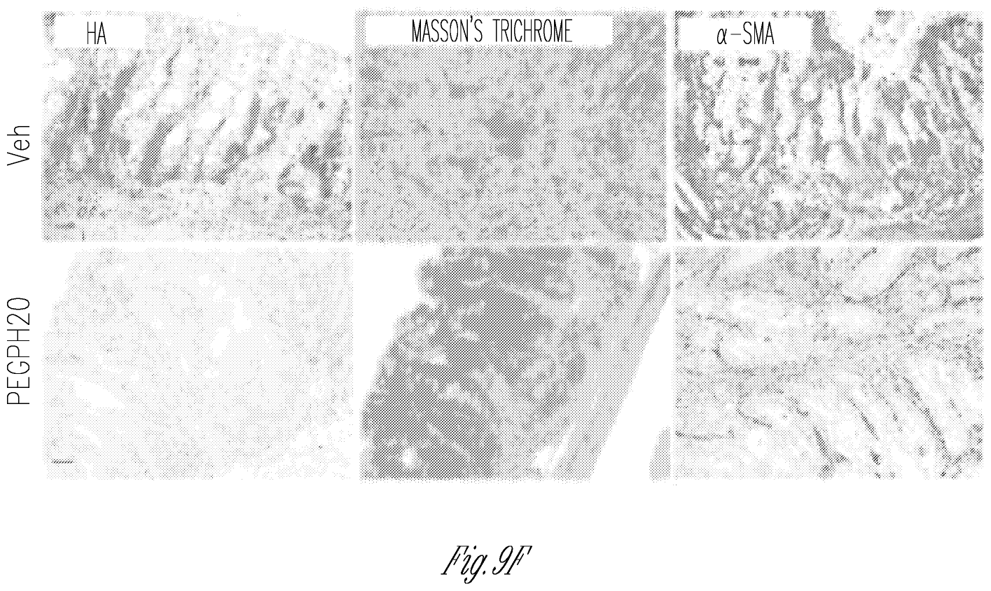

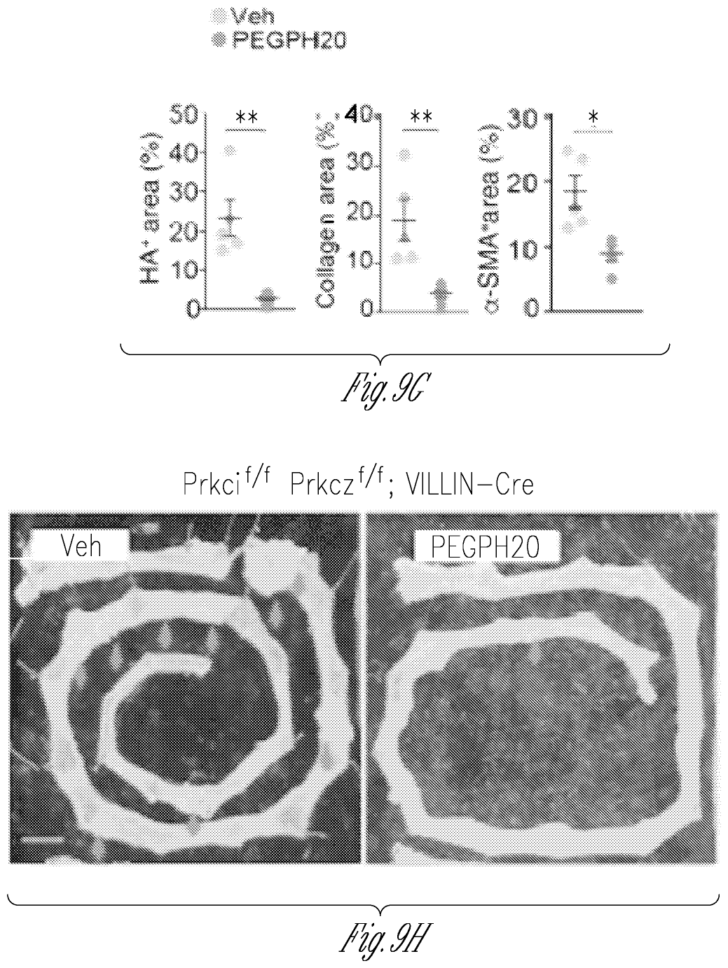

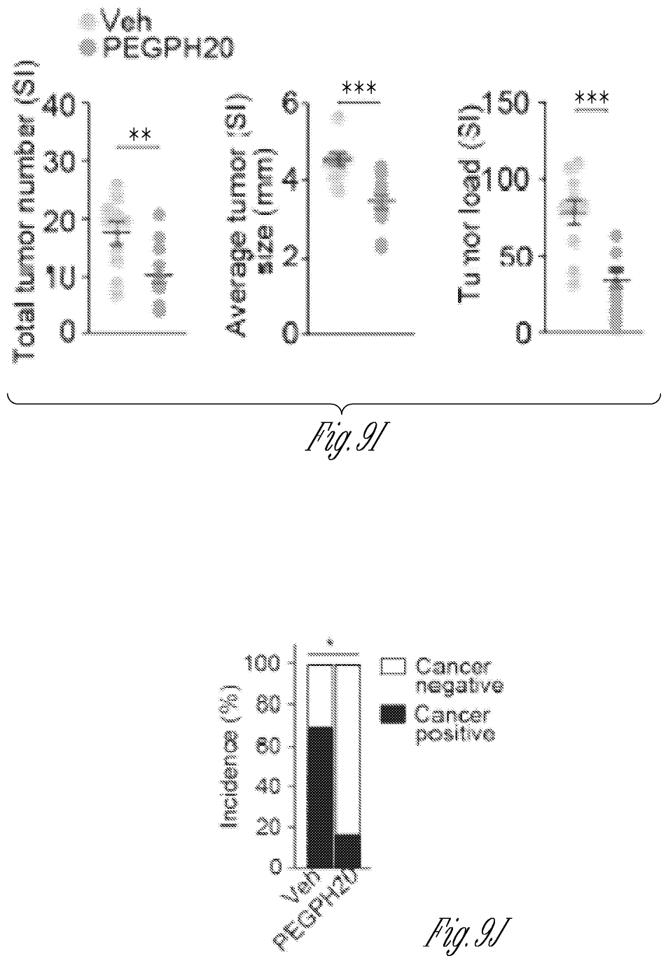

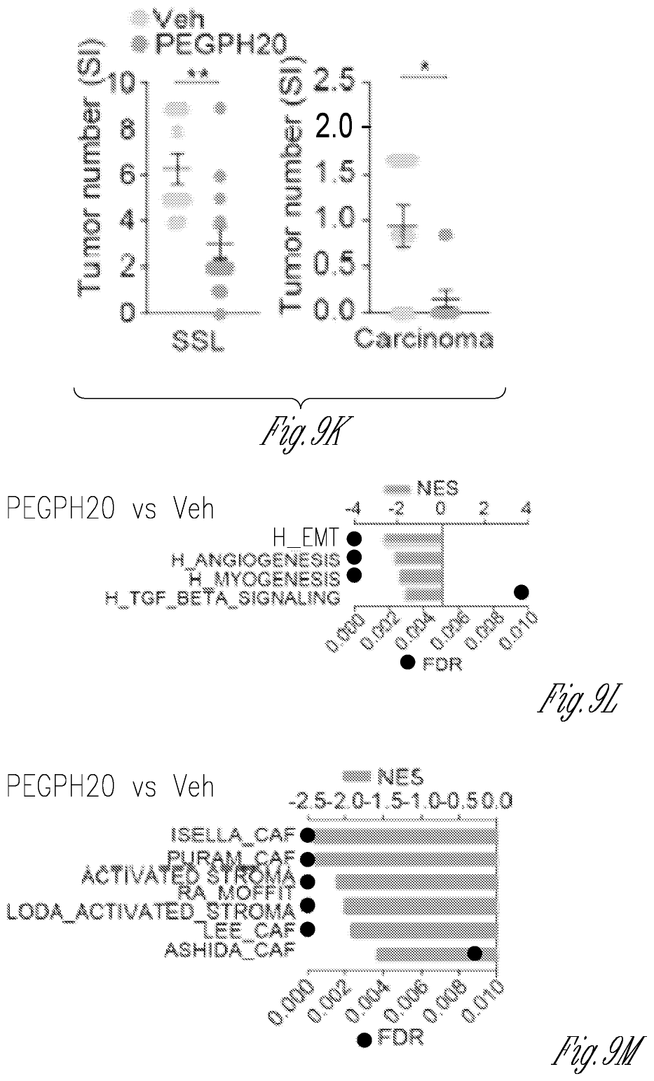

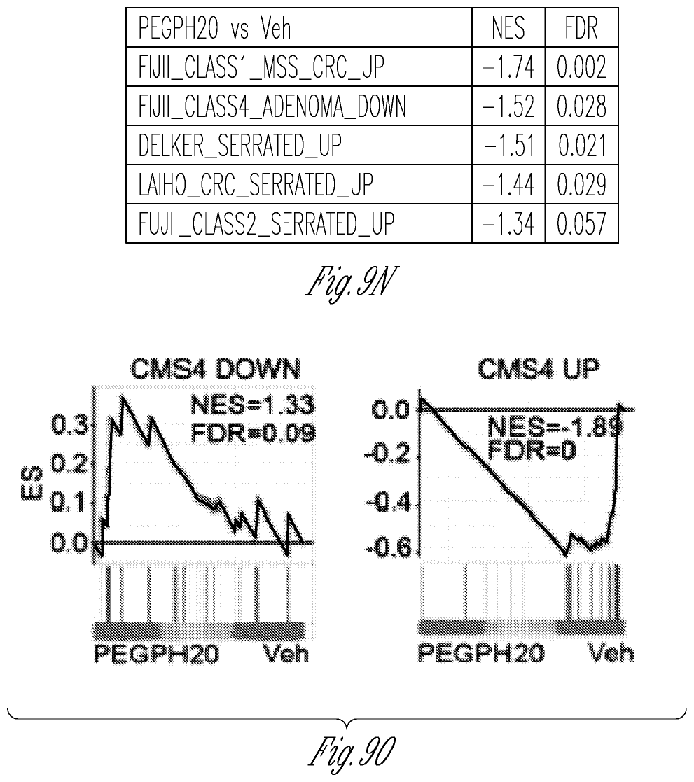

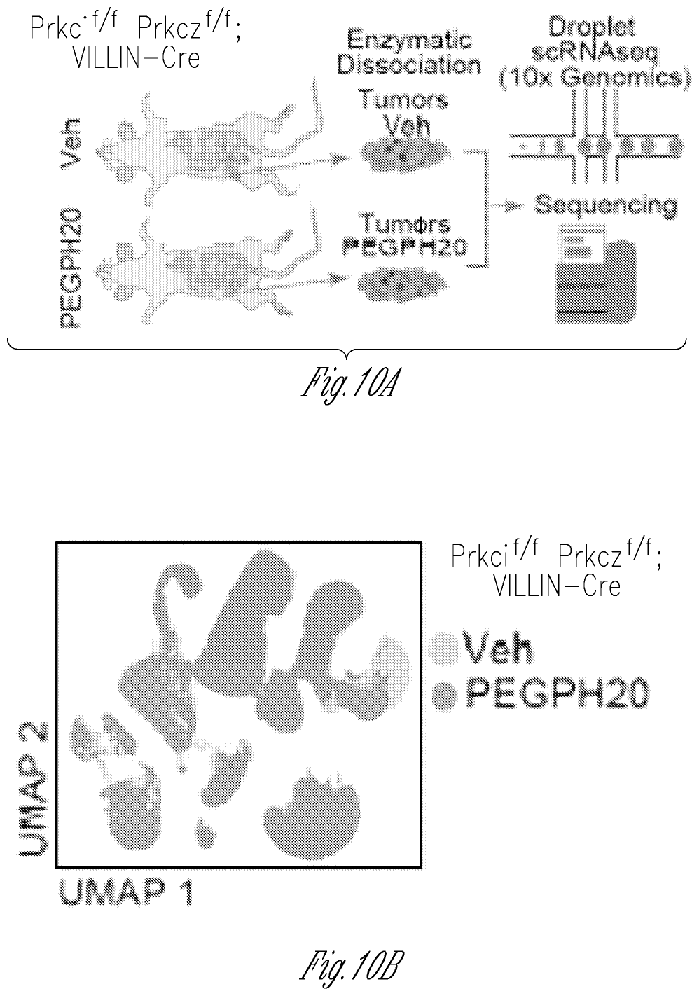

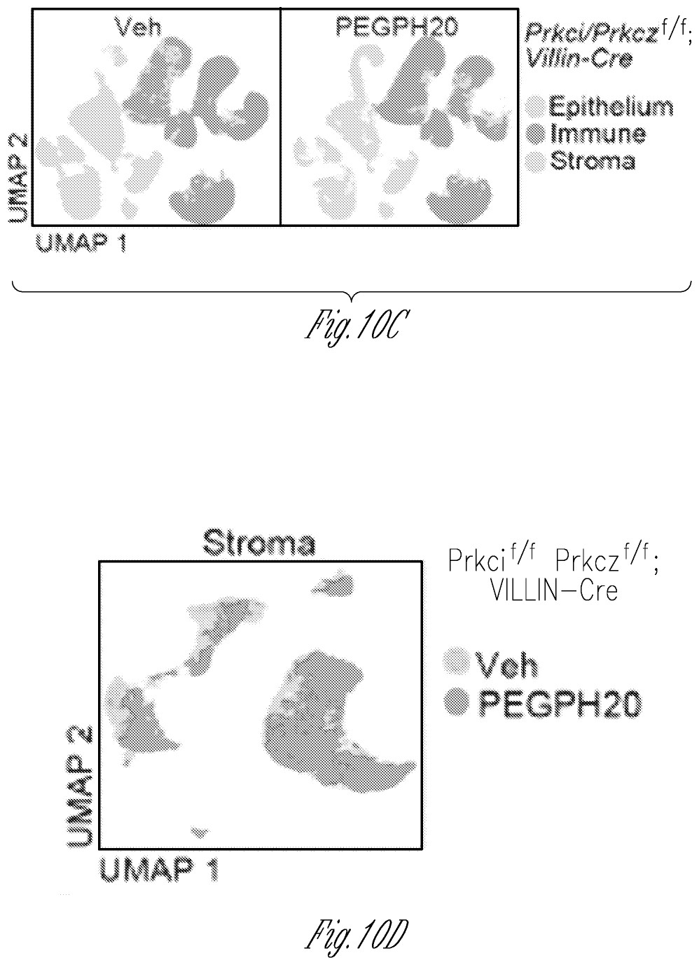

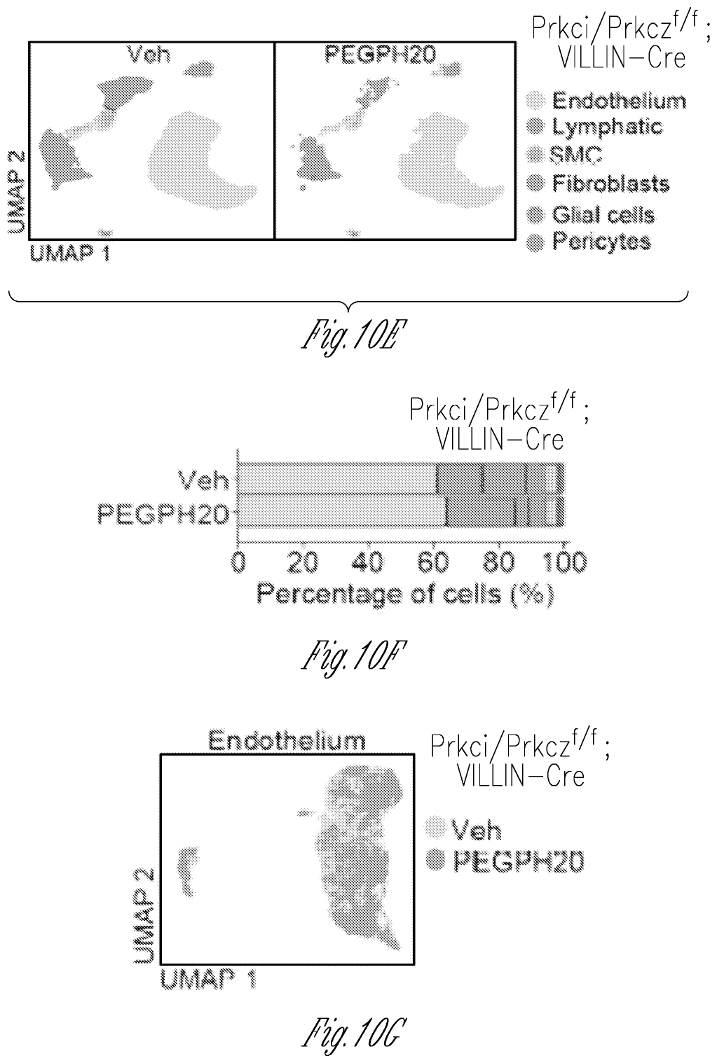

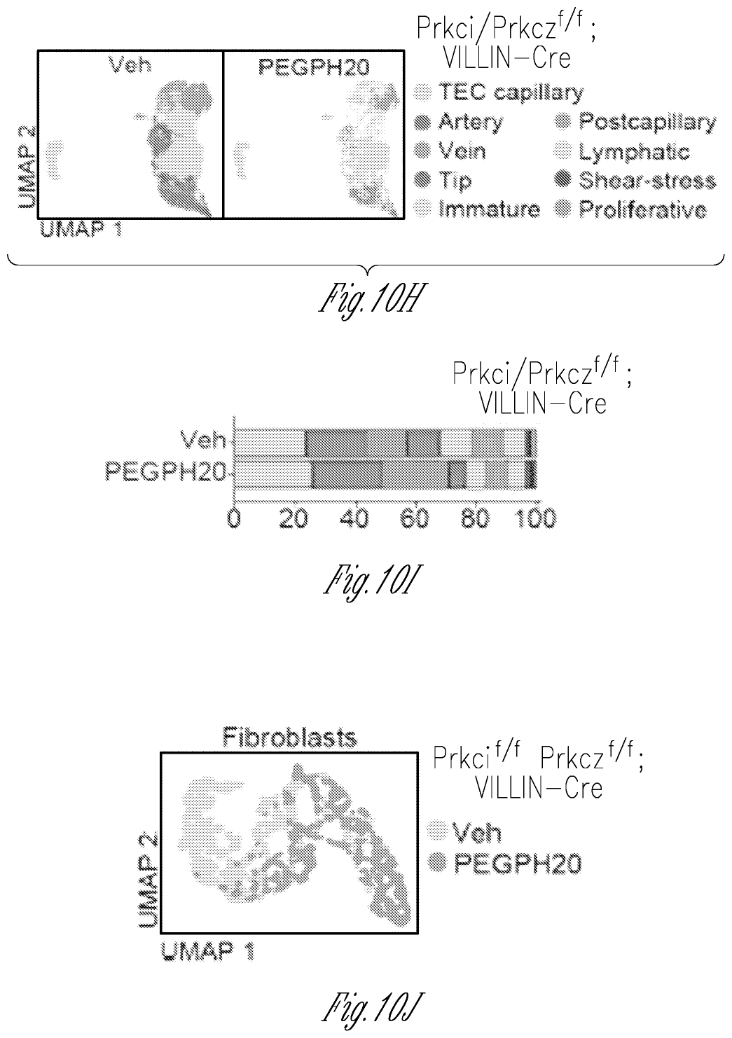

Next, the immune compartment was analyzed. Ptprc+ (CD45) cells from PVHA and GAL-treated DKO tumors were isolated and analyzed (FIG. 6A). After clustering, four major immune cell types components were identified based on well-known gene expression markers: Cd3g, Cd8a, and Cd4 for T cells; Cd79 and Jchain for B and plasma cells; S100a8, Apoe, Plac8, and Siglech for Myeloids and Mcpt1 for Mast cells (FIG. 6B). Erythrocytes were also identified but removed for the analysis.

Analysis of the cell-type proportions showed an increase of T cells and a decrease of myeloid cells upon PVHA and GAL treatment. However, the effect in myeloid cells was more remarkable upon PVHA treatment as compared to control and GAL-treated tumors (FIGS. 6C-6D). To test further whether both treatments impact myeloid and T cells composition, both compartments were isolated and analyzed. Based on well-known gene expression markers, six different myeloid populations were identified and annotated as Tumor-associated neutrophils (TAN1 and TAN2), dendritic cells (cDCs and pDCs), Tumor-associated macrophages (TAM), and inflammatory monocytes (FIGS. 6E-6F). Proportion analysis for myeloid cell subtypes showed a switch in TANs in PVHA-treated tumors, that is TAN1 (anti-tumoral neutrophils) are increased whereas TAN2 (pro-tumoral neutrophils) are reduced. However, GAL-treated tumors only increased the number of TAN1 without affecting TAN2 proportion (FIGS. 6G and 6H). Interestingly, PVHA but not GAL increased the proportion of dendritic cells. For T cells, six major subpopulations were identified (Cd8, Cd4, Treg, Th17, NK cells, and Proliferating T cells) according to the expression of well-known markers (FIGS. 6I and 6J). Cell type proportion showed an increase in Cd8 T cells upon PVHA and GAL treatment (FIGS. 6K and 6L). However, PVHA but not GAL was also able to reduce the proportion of Treg, which has been linked with immunosuppressive function. Next, cell-cell interactions of immune cells with tumor epithelial cells with CellphoneDB was investigated. PVHA-treated tumors have higher numbers of inferred interactions with immune cells as compared to GAL treatment (FIGS. 6M and 6N). Without being limited to any specific theory, these results indicate that PVHA, but not GAL, effectively remodeled the stroma and immune landscape to block CMS4 tumors.

### Example 3: Hyaluronan Driven by Epithelial aPKC Deficiency Remodels the Microenvironment and Creates a Therapeutic Vulnerability in Mesenchymal Colorectal Cancer

### Experimental Model and Subject Details

### Mice

Animal handling and experimental procedures conformed to institutional guidelines and were approved by the Sanford-Burnham-Prebys Medical Discovery Institute Institutional Animal Care and Use Committee, and by the Weill Cornell Medicine Institutional Animal Care and Use Committee. Prkci^{f/f}Prkcz^{f/f}, Villin-Cre, Prkci^{f/f}Prkcz^{f/f}; Villin-Cre and Villin-CreERT2 mice were previously described (el Marjou et al., 2004; Madison et al., 2002; Nakanishi et al., 2018) Prkci^{f/f}Prkcz^{f/f}; Villin-CreERT2 mice were generated by breeding Prkci^{f/f}Prkcz^{f/f} mice with Villin-CreERT2 (Jackson Laboratory, stock number 020282) mice. All mouse strains were generated in a C57BL/6 background and were born and maintained under pathogen-free conditions. For PEGPH20 treatment, 11-week-old Prkci^{f/f}Prkcz^{f/f}; Villin-Cre were retro-orbitally injected twice a week for 3 weeks with a dose of 0.0375 mg/kg of PEGPH20 (Halozyme Therapeutics), until they were sacrificed. Control mice were treated with vehicle. For xenograft experiments, 6-7 week-old male C57BL/6 were purchased from Charles River Labs (Wilmington, MA, USA). All mice were maintained on food and water ad libitum and were age-matched and co-housed for all experiments. Mice were sacrificed and small intestine, colon, tumors or other organs were collected for analysis. All genotyping was done by PCR. Age- and sex-matched mice were used for all experiments

### Human Samples

For CRC samples, a total of 343 CRC patients (male, n=176; female, n=167) who had undergone a resection of the primary tumor at Osaka City University Hospital were analyzed. Baseline characteristics are shown in FIG. 15. CRC tissues were obtained from each patient and none of the patients had undergone preoperative radiation or chemotherapy. This study was approved by the Osaka City University Ethics Committee and written informed consent was obtained from patients. De-identified human samples were sent to Weill Cornell Medicine and used for histological analyses. The study was approved by the IRB Committee of Weill Cornell Medicine.

### Cell Lines

Patient-derived organoids (PDOs) were isolated from patient-derived xenografts (PDXs). First, surgically resected colorectal cancer tissues were transplanted into NSG mice to generate PDXs. After engraftment, PDOs were isolated and cultured. Briefly, PDX tissues were washed vigorously with ice-cold phosphate-buffered saline (PBS) and minced into 1 mm³ fragments using surgical scalpel blades. The fragments were digested with digestion buffer containing 25 mg/ml Collagenase A (Sigma), 25 mg/ml Dispase II (Sigma), and 500 μ/ml DNase I (Sigma) for 10 minutes at 37° C. The supernatant was filtered through a 70 μm cell strainer and centrifuged at 400G for 5 minutes. The pellet was embedded in Matrigel and applied onto 12-well plates as 20 μl droplets. Organoids were cultured in advanced Dulbecco's Modified Eagle's Medium/F12 (Thermo Fisher Scientific) supplemented with 50% Wnt-3A conditioned medium, 50 ng/ml recombinant human EGF (Peprotech), 0.25% Noggin-FC conditioned medium, 10% mouse R-spondin 1-FC conditioned medium, 500 nM A83-01, 100 ng/ml recombinant human IGF-1 (BioLegend), 50 ng/ml recombinant human FGF-2 (Peprotech), 10 μM Y-27632, 1× B27 supplement (Life Technologies), 10 nM human [Leu15]-Gastrin I (Sigma), 1× Glutamax (Life Technolgies), 1×HEPES (Life Technologies), and 1× Penicillin/Streptomycin (Corning). After the first passage, Wnt-3a and R-spondin 1 were removed from the media. Mouse tumor organoids (MTOs) were obtained from Dr. Eduard Batlle (Institute for Research in Biomedicine, Barcelona, Spain) and previously described (Tauriello et al., 2018). MTOs were cultured in advanced DMEM/F12 medium supplemented with 10 mM HEPES, Glutamax, B-27 (all Life Technologies), 50 ng/ml recombinant human EGF (Peprotech). Normal intestinal organoids were cultured in Advanced DMEM/F12 containing 10 mM HEPES, 1× Glutamax, 1× N2 supplement, 1× B27 supplement, 50 ng/ml EGF, 1000 ng/ml R-spondin 1, 100 ng/ml Noggin, and 10 μM Y-2763L in an atmosphere of 95% air and 5% CO2. Cultures were tested monthly for mycoplasma contamination.

### Method Details

### Colonoscopy-Guided Injection and Xenograft Experiments

For colonoscopy-guided orthotopic injection in the colon submucosa using mouse tumor organoids (MTOs), a 10 μl organoid suspension of equivalent to 40-50 MTOs admixed with 10% Matrigel (Corning) was directly injected into the colonic submucosa of 7 weeks old C57BL/6 mice. 4 weeks after implantation, tumors were collected and analyzed histologically. Orthotopic tumor sizes were measured by caliper after mice were sacrificed. For xenografts using MTOs, a 100 μl cell suspension of equivalent to 0.5×106 cells of sgPrkci/Prkcz or sgC MTOs admixed with 30% Matrigel (Corning) was directly injected subcutaneously into the right flank of 7 weeks old C57BL/6 mice. Tumors were allowed to grow for 14 days, and mice were randomly divided to receive vehicle, n=20, or PEGPH20 (0.0375 mg/kg, 2 days each week) via retro-orbital injection, n=18. Tumors were measured twice a week. Mice were euthanized three weeks after treatment and tumors were analyzed histologically. For the combination therapy of anti-PD-L1 (Clone B7-H1, Bioxcell) antibody and PEGPH20, mice were injected with a dose of 5 mg/kg (body mass) of anti-PD-L1 antibody in combination with PEGPH20 (n=15) twice a week for 4 weeks until they were sacrificed.

### Cell Culture Experiments

To perform PRKCI and PRKCZ editing in PDOs, single-guide RNA sequences targeting PRKCI and PRKCZ (Table 3), were purchased from Synthego and transduced into PDOs with recombinant Streptococcus pyogenes Cas9 protein (Truecut Cas9 Protein v2, Thermo. To knock out Prkcz in mouse sgPrcki MTOs (Linares et al., 2021), single-guide RNA sequence targeting Prkcz (Table 3) was purchased from Synthego and transduced into sgPrcki MTOs as described above. PRKCI, PRKCZ, and Prkcz were knocked out using the Neon Transfection System 1 (Invitrogen) following the manufacturer's protocol and single clones were expanded and screened by protein immunoblotting. For the deletion of Prkci and Prkcz in normal organoids, 500 nM 4-OH-tamoxifen was added the second day after organoids were passaged. For PEGPH20 treatment in vitro using MTOs, 300 organoids per Matrigel dome were seeded in a p12 well plate (Thermo Fisher Scientific) and cultured as described above. PEGPH20 at 2.5 μg/ml was added every other day for 6 days for a total of three doses.

### Histology, Immunohistochemistry, and Immunofluorescence

Tissues from indicated mice were isolated, rinsed in ice-cold PBS, fixed in 10% neutral buffered formalin overnight at 4° C., dehydrated, and embedded in paraffin. Sections (5 μm) were stained with hematoxylin and eosin (H&E). For immunohistochemistry, sections were deparaffinized, rehydrated, and then treated for antigen retrieval. After blocking in Protein Block Serum-Free solutions (DAKO), tissues were incubated with primary antibody overnight at 4° C. followed by incubation with biotinylated secondary antibody. Endogenous peroxidase was quenched in 3% H2O2 in water for 10 minutes at room temperature. Antibodies were visualized with avidin-biotin complex (Vectastain Elite; Vector Laboratories) using diaminobenzidine as the chromogen. Stained sections were analyzed with a Zeiss light microscope supplemented with Zen 3.3 Bule edition software. For immunofluorescence, the signal was visualized using the OPAL™ 4-Color Manual IHC Kit (Akoya Biosciences, MA), which allows simultaneous detection of multiple targets in the same image. Fluorophores Opal 520, Opal 570, and TSA Plus Cyanine 5 were used, and the sections were counterstained with Spectral DAPI.

### VECTRA Staining

Primary antibody dilutions were optimized individually at H220 retrieval using the Leica BondRx Automated IHC stainer and evaluated by a board-certified pathologist for specificity. Multiplexed staining was optimized and then performed using the Automated Opal 7-Color IHC Kit (NEL821001KT) from Akoya Biosciences. A library for spectral separation was generated by staining control tissue with each opal fluor conjugated to CD20. Slides were imaged in the Vectra Polaris Automated Quantitative Pathology Multispectral Imaging System. Exposure times were maximized under the constraint that no pixel saturates the detector (Patel and Rodig, 2020; Patel et al., 2019).

### Tissue Microarray Analyses

Image analysis was processed in QuPath v.0.1.3 (Queen's University, Belfast, Northern Ireland). Scanned TMA slides were dearrayed and preprocessed as previously described (Bankhead et al., 2017). After dearraying, all cores were examined and those with either no tumor represented or with artefacts (tissue broken, for example) were excluded. Cell-detection was determined for each core, and the total number of positive cells using DAPI staining as a cell reference was assessed. H-score, calculated based on the extent and intensity of the staining, was used for aPKC scoring. CRC patients were categorized into high and low aPKC expression groups using 90 H-score as the cut-off selected by an experienced pathologist. For HA expression, CRC samples were considered positive when at least 1%-30% of stromal cells were stained.

### Whole-Mount Organoid Staining

Organoid staining was performed as previously described (van Ineveld et al., 2020). Briefly, the organoid suspension was washed once in PBS before adding 500 μl of ice-cold cell recovery solution (Corning) per well. Organoids were incubated for 30-60 minutes at 4° C. on a horizontal shaker until Matrigel was dissolved. Organoids were resuspended 5-10 times transferred to a pre-coated 15 ml falcon tube (1% BSA-PBS) and centrifuged for 3 minutes at 70 g, 4° C. The organoid pellet was resuspended in 1 ml of ice-cold PFA (4%) and organoids were fixed for 45 minutes. 10 ml of ice-cold PBS with 0.1% Tween-20 was added, incubated for 10 minutes, and centrifuged at 70 g for 3 minutes at 4° C. The organoid pellet was resuspended in organoid wash buffer and transferred to a 24-well plate for blocking and overnight antibody incubation. Organoids were washed for 2 hours three times and then incubated overnight with secondary antibodies in the wash buffer with 1 μg/ml DAPI. The next day, the washing steps were repeated, and the organoids were mounted onto 35 mm glass-bottom dishes and imaged under confocal microscopy.

### Immunoblotting Analysis

Cells for protein analysis were lysed in RIPA buffer (20 mM Tris-HCl, 37 mM NaCl2, 2 mM EDTA, 1% Triton-X, 10% glycerol, 0.1% SDS, and 0.5% sodium deoxycholate) with phosphatase and protease inhibitors. Protein concentration in lysates were determined by using Protein Assay Kit (Bio-Rad). Cell extracts were denatured, subjected to SDS-PAGE, transferred to PVDF membranes (GE Healthcare). After blocking with 5% nonfat dry milk in Tris-buffered saline and 0.1% Tween (TBS-T), the membranes were incubated with specific antibodies overnight at 4° C. After 2 hours incubation with the appropriate fluorochrome-conjugated antibodies, the immune complexes were detected by Near-infrared fluorescence (LI-COR).

### RNA Extraction and Analysis

Total RNA from cultured organoids was extracted using TRIZOL reagent (Invitrogen) and purified by the RNeasy Mini Kit (QIAGEN), followed by DNase treatment. After quantification using a Nanodrop 1000 spectrophotometer (Thermo Scientific), RNA was either processed for RNA-seq or reverse-transcribed using random primers and MultiScribe Reverse Transcriptase (Applied Biosystems). Gene expression was analyzed by amplifying 500 ng of the complementary DNA using the CFX96 Real Time PCR Detection System with SYBR Green Master Mix (BioRad) and primers described in Table 2. The amplification parameters were set at 95° C. for 30 seconds, 58° C. for 30 seconds, and 72° C. for 30 seconds (40 cycles total). Gene expression values for each sample were normalized to the 18S RNA.

### 3′ RNA-Seq Preparation and Sequencing

Total RNA was extracted using Quick-RNA MiniPrep kit (Zymo Research). Libraries were prepared from 200 ng of total RNA using the QuantSeq 3′ mRNA-Seq Library Prep Kit FWD for Illumina from Lexogen, and optional UMIs (Vienna, Austria). Barcoded libraries were pooled, and single end sequenced (1×75) on the Illumina NextSeq 500 using the High output V2.5 kit (Illumina Inc., San Diego CA).

### 10× Library Preparation and Sequencing

Tumors were minced thoroughly and digested by 0.5 mg/ml Liberase TH (Sigma) for 30 minutes at 37° C. Dead cells were removed by Annexin V (STEMCELL technologies). scRNAseq libraries were generated using the Chromium Single Cell 30 Reagent Kit v2 (10× Genomics). Cells were loaded onto the 10× Chromium Single Cell Platform (10× Genomics) at a concentration of 2,000 cells per μl (Single Cell 3′ library and Gel Bead Kit v.2) as described in the manufacturer's protocol (10× User Guide, Revision B). Generation of gel beads in emulsion (GEMs), barcoding, GEM-RT clean-up, complementary DNA amplification and library construction were all performed as per the manufacturer's protocol. Individual sample quality was checked using a Bioanalyzer Tapestation (Agilent). Qubit was used for library quantification before pooling. The final library pool was sequenced on an Illumina NovaSeq6000 instrument using a S1 flow cell. Average cell recovery for Prkci^{f/f}Prkcz^{f/f}; Villin-Cre tumors was 104,954 cells with a total of 209,907 cells captured at a mean depth of 14,568 read per cell and 895 mean genes per cell.

Analysis of scRNA Sequencing Data

For scRNA-seq, raw sequence reads were quality-checked using FastQC software. The Cell Ranger version 2.1.1 software suite from 10× Genomics (https://support.10× genomics.com/single-cell-gene-expression/software/downloads/latest) was used to process, align, and summarize unique molecular identifier (UMI) counts against the mouse mm10 assembly reference genome analysis set, obtained from the University of California Santa Cruz (UCSC). Raw, unfiltered count matrices were imported into R for further processing. Raw UMI count matrices were filtered using the Seurat v 3.0 R package (Butler et al., 2018) to remove: barcodes with very low (less than 200, empty wells) and very high (more than 3000, probably doublets) total UMI counts; matrices for which a high percentage of UMIs originated from mitochondrial features (more than 12%); and matrices for which fewer than 250 genes were expressed. Subsequently, the data were normalized using the SCTransform function, regressing out the following variables: total number of UMIs per cell and percentage of mitochondrial UMIs. Following normalization, the principal components were computed. The top principal components were identified using the ElbowPlot function and used for the UMAP dimensionality reduction. For clustering, Seurat was used in combination with Harmony software (Korsunsky et al., 2019) to correct the potential effects of technical differences between sequencing batches. The RunHarmonyo, RunUMAP( ), FindNeighbors( ), and FindClusters( ) functions were run for clustering and the percentage of mitochondrial features was considered to be a source of unwanted variation and was regressed out using the Seurat package. Genes specifically expressed in each cluster were identified with the FindAllMarkers( ) function and the Wilcoxon test labeling the different populations using the genes differentially up-regulated in each population. For the differential gene expression analysis, the FindMarker( ) function with default parameters was run for comparison between populations. The cell groups were annotated based on the marker gene analysis and canonical markers from the literature. The gene set enrichment analysis was performed using the results of the differentially expressed gene analysis between populations using Preranked GSEA analysis with default parameters (permutations=1000, enrichment score=weighted) and MSigDB H.all.v7.0.symbols (H), c2.all.v7.0.symbols (C2) and c5.all.v7.0.symbols (C5) collections. The scoring for the indicated signatures was performed using the AddModuleScore function in Seurat with default parameters. Single-cell RNA-seq data of CRC patients were obtained from GSE132465 and processed as described in Linares et al 2021 to generate CRC TEC and hCRC CAF signatures based on a list of differentially expressed genes (upregulated genes, pdj<0.05) between tumor vs normal endothelial cells (n=84) and tumor vs normal fibroblast (n=122) respectively. Gene sets used for signature scoring are listed in FIG. 22 and when necessary, mapped to mouse orthologues using Ensembl BioMart. The visualization of the indicated signature score by Violin Plot and the statistical analysis (T-test) was performed using the ggplot2 package in R software.

### Single-Cell CNV Analysis

Single-cell CNV profiles were inferred using the inferCNV R package (v1.2.1), which computes gene expression intensities across genomic positions from malignant cells as compared to a set of reference cells (goblet cells from non-tumor epithelial scRNAseq data). Input data were prepared as recommended by inferCNV authors. The algorithm was run with the following arguments: cutoff=0.1, cluster_by_groups=TRUE, denoise=TRUE, HMM=TRUE. The feature vectors from inferCNV were exported and visualized on the matching UMAP in Seurat using the add_to_seurat ( ) function. Normal epithelial cells were separated from tumor epithelial cells based on sample origin and CNVs. A small number of tumor cells clustered with normal cells and vice versa were eliminated.

### CellPhoneDB Analysis

CellPhoneDB was used to identify ligand-receptor interactions in scRNAseq data. After identifying different cell types in the scRNA-seq as described above, recommended procedures for the preparation of input files were followed using CellPhoneDB v.2.0.0 (Efremova et al., 2020). The original CellPhoneDB repository was updated with unique interactions and complexes curated from literature using ‘cellphonedb database generate’ command. All CellPhoneDB statistical analysis were performed with this updated database and percentage cell expression threshold of 5%. Cell-cell interactions heatmaps and Dot plots showing number of interactions, Log 2 mean (Molecule 1, Molecule 2) and log 10 (p value), were generated using CellphoneDB.

scRNA-Seq Trajectory Analysis

For RNA velocity analysis (La Manno et al., 2018), loom files were generated using kb_python (Bray et al., 2016; Melsted et al., 2021). The velocity index (Mus Musculus.GRCm38.98) was built using kb ref and the flag ‘lamanno’. Pseudo alignment and quantification was performed using kb count and the following parameters: technology: 10xv3 and workflow lamanno. Kb count uses bustools for quantification and return unfiltered and filtered loom files which were used for downstream analysis using scVelo (Bergen et al., 2020). Briefly, genes were filtered and normalized using the filter_and_normalize function with the following parameters: min_shared_counts=20 and n_top_genes=2000. First and second-order moments (means and uncentered variances) were computed using the moments function, with n_pcs=30 and n_neighbors=30. RNA velocities were calculated with the velocity function setting mode=“stochastic”.

### Bulk RNAseg Analysis

For 3′RNA-Seq, read data was processed with the BlueBee Genomics Platform (BlueBee, San Mateo, CA). RNA-seq data of The Cancer Genome Atlas (TCGA) Colorectal Adenocarcinoma (Tumor Samples with mRNA data (RNA Seq V2 RSEM), 592 tumor samples from 592 patients) was downloaded through cBioportal (http://www.cbioportal.org/indexxdo). GenePattern (https://genepattern.broadinstitute.org/gp/pages/index.jsf) was used to collapse gene matrix files (CollapseDataset module) or to assess the statistical significance of differential gene expression (DESeq2). Genes were sorted by log 2 FC>0.3 and adj>0.05. Volcano plot representation for differentially expressed genes was generated using VolcaNoseR (https://buygens.science.uva.nl/VolcaNoseR/). Gene Set Enrichment Analysis (GSEA) was performed using GSEA 4.0 software (http://www.broadinstitute.org/gsea/index.jsp) with 1000 gene-set permutations using the gene-ranking metric t-test with the collections h.all.v6.1.symbols (H), c2.all.v6.1.symbols (C2), c5.all.v6.1.symbols (C5), or customized gene signatures (Table 4). For ASHIDA_CAF signature, differential gene expression analysis was performed as indicated above between CAF and NAF from GSE34312. Genes were sorted by log 2 FC>0.5 and adj>0.05. The classification of TCGA CRC patients based on the consensus molecular subtype (Guinney et al., 2015) was performed using CMScaller (Eide et al., 2017) R package with default parameters (false discovery rate (FDR)=0.5, seed=1, RNAseq=TRUE).

### Patient Segregation and Survival Analyses

TCGA CRC patients were separated into two groups using the top 25 percentile and bottom 25 percentile of PRKCI and PRKCZ expression. The Microenvironment Cell Populations (MCP)-counter algorithm (webMCP-counter) was used as an independent bioinformatics tool to assess stromal cell enrichment between groups. HAS score for each patient was calculated by computing the average of HAS1, HAS2, and HAS3 expression and the gene set variation analysis (GSVA) algorithm, using standard settings. TCGA CRC patients were separated into two groups according to the median for the HAS score and compared by GSEA (see Bulk RNAseq analysis and TCGA data). The “TcTA”, “TRSC”, “TFSC” and “TGC” signatures (Table 4) were generated based on a list of the Top 50 upregulated genes for each tumoral population using FindAllMarkers( ) function in Seurat. Overlapping genes between populations were removed and mapped to human orthologues. The signature score for each CRC patient was calculated using GSVA as indicated above and separated into two groups using the top 25 percentile and bottom 25 percentile. To generate Kaplan-Meier curves, TCGA patients with available clinical data (n=589) were processed, and the disease-free survival (DFS) rate was compared between groups.

### Quantification and Statistical Analysis

All the statistical tests were justified for every figure. All samples represent biological replicates. Data are presented as the mean±SEM. Statistical analysis was performed using GraphPad Prism 8 or R software environment (http://www.r-project.org/). Significant differences between groups were determined using a Student's t-test (two-tailed) when the data met the normal distribution tested by D'Agostino test. If the data did not meet this test, a Mann-Whitney U-test was used. Differences between more than 3 groups were determined using one-way ANOVA test (parametric) or Brown-Forsythe and Welch ANOVA tests (nonparametric) followed by Dunnett post hoc test. If the data did not meet this test, a Mann-Whitney test was used. Differences in Kaplan Meier plots were analyzed by the Mantel-Cox test. The chi-square test or Fisher's exact test was used to determine the significance of differences between covariates. Logistic regression analysis was employed to estimate univariate and multivariate odds ratio and 95% confidence interval (CI). Values of p<0.05 were considered significantly different.

TABLE 2
List of primers used, Related to STAR Methods

| Gene | | |
|---|---|---|
| symbol | Forward | Reverse |
| 18s | 5′-GTAACCCGTTGAACCCCAT-3′ | 5′-CCATCCAATCGGTAGTAGCG-3′ |
| | (SEQ ID NO: 1) | (SEQ ID NO: 2) |
| Prkcz | 5′-TGTGTCCTCACAGATGGAGC-3′ | 5′-TCCACGGCGGTAGATGGA-3′ |
| | (SEQ ID NO: 3) | (SEQ ID NO: 4) |
| Prkci | 5′- | 5′-TTCCTCATCTATCCATTTCATGGT-3′ |
| | GCGGGGATATTATGATAACACACTT | (SEQ ID NO: 6) |
| | (SEQ ID NO: 5) | |
| Has1 | 5′-GTGCGAGTGTTGGATGAAGACC-3′ | 5′-CCACATTGAAGGCTACCCAGTATC-3′ |
| | (SEQ ID NO: 7) | (SEQ ID NO: 8) |
| Has2 | 5′-GCCATTTTCCGAATCCAAACAGAC-3′ | 5′-CCTGCCACACTTATTGATGAGAACC-3′ |
| | (SEQ ID NO: 9) | (SEQ ID NO: 10) |
| Has3 | 5′-GCTTCAGTCCAGAAACCAAAGTAGG-3′ | 5′-CCTCGTTCCTCAAGAGAAACAAGG-3′ |
| | (SEQ ID NO: 11) | (SEQ ID NO: 12) |
| Anxa10 | 5′-TAGGCGGAGCACTCCAAGGATT-3′ | 5′-GCCATACATGCTCTGATAGGTCC-3′ |
| | (SEQ ID NO: 13) | (SEQ ID NO: 14) |
| Clu | 5′-CTGTCCACTCAAGGGAGTAGG-3′ | 5′-GTGTCCTCCAGAGCATCCTC-3′ |
| | (SEQ ID NO: 15) | (SEQ ID NO: 126 |
| HAS2 | 5′-AAACTGCTGCAAGAGGTTATTCCT-3′ | 5′-GTCATGTACACAGCCTTCAGAGC-3′ |
| | (SEQ ID NO: 17) | (SEQ ID NO: 18) |

TABLE 3
List of guides, Related to STAR Methods

| Guides | Source | Identifier |
|---|---|---|
| gRNA targeting human PRKC1 | Synthego | N/A |
| 5′-UUAAAUUAUCUUCAUGAGCG-3′ | | |
| (SEQ ID NO: 19) | | |
| gRNA targeting human PRKCZ | | |
| 5′-CACCUGCAGAGAGCGUACUG-3′ | | |
| (SEQ ID NO: 20) | | |
| gRNA targeting mouse Prkcz | | |
| 5′-CGUCCACAGGGAGCGUACUG-3′ | | |
| (SEQ ID NO: 21) | | |

### Results

Reduced Expression of Both aPKCs in Human CRC Correlates with a Mesenchymal Phenotype

Gene Set Enrichment Analysis (GSEA) of the CRC dataset in The Cancer Genome Atlas (TCGA) demonstrated that the transcriptome of patients with low aPKC levels (PRKCI^{low}PRKCZI^{low}) was enriched in transcripts corresponding to the mesenchymal phenotype, including an activated stroma and CAF signatures, as compared to patients with high aPKC levels (PRKCI^{high}PRKCZ^{high}) (FIGS. 7A-7C). Stratification of TCGA patients by CMS subtypes using the CMScaller algorithm revealed that the CMS4 transcriptomic phenotype was predominant in the PRKCI^{low}PRKCZ^{low} group (FIGS. 7D and 14A). Furthermore, the PRKCI^{low}PRKCZ^{low} group had a higher monocytic-myeloid abundance and an augmented expression of endothelial and activated fibroblastic signatures as compared to that of the PRKCI^{high}PRKCZ^{high} cohort, as determined by the Microenvironment Cell Populations (MCP)-counter algorithm (FIG. 7E). These results demonstrated that CRC patients with low aPKC levels have a tumor microenvironment that is inflamed, highly vascularized, and rich in activated CAFs, all characteristics of the mCRC type.

Low aPKC Levels in CRC Patients are Associated with Increased Expression of Markers of Hyaluronan Synthesis

The analysis of the differentially expressed genes in PRKCI^{low}PRKCZ^{low} vs. PRKCI^{high}PRKCZ^{high} groups in the TCGA dataset identified hyaluronan (HA) synthase 1 (HAS1) and hyaluronan synthase 2 (HAS2) as two upregulated genes in PRKCI^{low}PRKCZ^{low} patients (FIGS. 14B and 14C), suggesting that aPKC-deficient tumors would potentially be enriched in HA. To test this hypothesis, both aPKC genes were deleted in a CRC patient-derived organoid (FIGS. 7F and 7G). Human sgPRKCI/sgPRKCZ organoids expressed higher levels of HAS2 and increased accumulation of HA (FIGS. 7H and 7I), demonstrating that aPKC deficiency is sufficient to trigger the HA biosynthetic pathway in a cell-autonomous manner. Consistently, the classification of CRC patients based on a HAS score according to their HAS1, HAS2, and HAS3 levels showed a positive enrichment in TGFβ signaling and EMT signatures in CRC patients with a high HAS score, as compared to those with a low HAS score (FIG. 18H), supporting the notion that increased HA levels are a feature of human mCRC tumors.

Next, the expression levels of aPKC in the tumor epithelium and HA deposition in the stroma were analyzed by double immunofluorescence of a large cohort of surgically resected CRC specimens (FIG. 7J). Among the 390 CRC patients in the tissue microarray (TMA), 343 with available clinical information were stratified according to aPKC/HA expression status, including 21 Stage IV patients with matched liver metastasis (FIG. 18D). Kaplan-Meier curves revealed that patients with aPKC-low expression showed significantly worse overall survival than those with aPKC-high levels (FIG. 7K). HA-positive patients also showed a worse prognosis than HA-negative patients (FIG. 7L). Multivariate logistic regression analysis demonstrated that low aPKC expression was correlated with HA stromal deposition independent of other pathological features (FIGS. 14F and 14G). Survival analyses of these patients categorized into four groups based on their aPKC and HA content indicated that those who were aPKC-low and HA-positive had the worst prognosis (FIGS. 7M and 7N). Consistently, multivariate COX proportional hazards regression analysis demonstrated that aPKC-low/HA-positive expression was significantly associated with worse prognosis compared to aPKC high/HA-positive patients (Hazard ratio: 2.39), whereas there was no significant difference between aPKC high/HA positive patients and aPKC high/HA negative or aPKC low/HA negative patients (FIG. 14H). These findings highlight HA deposition as one of the main contributors to the aggressive phenotype of aPKC-deficient tumors. All stage IV CRC patients and their matched liver metastasis studied were HA-positive, and liver metastasis showed reduced expression of aPKC compared to their matched primary tumor (FIGS. 7O and 7P), suggesting that the loss of both aPKCs is an important event for the systemic spread of late-stage CRC. aPKC deletion makes CRC tumors dependent on HA for enhanced malignancy

To address the potential role of HA stromal accumulation in the tumorigenesis of intestinal mesenchymal tumors in vivo, mouse tumor organoids (MTO) with mutations in Apc, Trp53, Kras, and Tgfbr2 (Tauriello et al., 2018) and with deletion of Prkci and Prkcz by CRISPR/Cas9 were characterized. MTO-sgPrkci/sgPrkcz recapitulated the observed phenotype in human organoids showing in vitro upregulation of Has1, Has2, and Has3 transcripts, increased expression of HA, and enrichment in EMT and TGFβ signaling signatures (FIGS. 8A-8E). The injection of MTO-sgPrkci/sgPrkcz into the colon submucosa of C57BL/6J mice generated larger tumors and displayed increased HA accumulation than MTO controls (FIGS. 15A-15E). Next, the impact of depleting HA on tumorigenesis was tested. To that end, MTO-sgPrkci/sgPrkcz or MTO-sgC were subcutaneously transplanted into syngeneic C57BL/6J mice and treated with a low dose of clinical-grade pegylated hyaluronidase (PEGPH20; 0.0375 mg/Kg), equivalent to a human dose evaluated in clinical trials, or with vehicle (FIG. 8F). Tumors from MTO-sgPrkci/sgPrkcz had a more desmoplastic phenotype characterized by enhanced expression of HA, collagen, and αSMA+, which was reverted by PEGPH20 treatment (FIGS. 8G and 8H). MTO-sgPrkci/sgPrkcz tumors showed higher volume and weight than those from MTO-sgC but were sensitive to PEGPH20 treatment, which did not affect the growth properties of MTO-sgC tumors (FIGS. 81 and 8J). Consistently, transcriptomic interrogation of MTO-sgPrkci/sgPrkcz tumors treated with PEGH20 revealed a decrease in signatures related to invasion, tumor progression, and desmoplasia, such as EMT, myogenesis, angiogenesis, TGFβ signaling, stromal activation and the CMS4 subtype (FIGS. 8K-8M). These data demonstrate that aPKC-deficiency switches CRC tumors to a highly mesenchymal phenotype yet confers vulnerability to HA depletion in the context of WNT-driven tumorigenesis.

### HA Accumulation is a Critical Feature of Serrated Mesenchymal Tumors

To test whether HA is also important in serrated-originating CRC, an endogenous model of mesenchymal serrated MSS tumors driven by aPKC deficiency (Nakanishi et al., 2018) was employed. Upon the inducible and simultaneous ablation of both aPKCs, organoids from inducible Prkci^{f/f} Prkcz^{f/f}; Villin-CreER mice showed increased expression of Has1, Has2, and Has3, concomitant with enhanced HA accumulation (FIGS. 3A-C). Furthermore, Prkci^{fl/fl}Prkcz^{fl/fl}; Villin-Cre intestinal tissues showed a substantial deposition of HA compared to control samples (FIG. 9D). The accumulation of stromal HA was detected even in the non-tumor area of the Prkci^{fl/fl}Prkcz^{fl/fl}; Villin-Cre intestine and further increased in adenomas as they progressed from benign SSL to malignant carcinomas (FIG. 9D). The stromal HA accumulation in Prkci^{fl/fl}Prkcz^{fl/fl}; Villin-Cre tumors was eliminated by PEGPH20 treatment, accompanied by a profound remodeling of the tumor stroma, as evidenced by reduced collagen deposition and αSMA expression (FIGS. 9E-9G). Importantly, PEGH20 treatment resulted in a significant reduction in tumor number, average size, and tumor load and lower cancer incidence in the small intestine, concomitant with fewer SSL and reduced invasive carcinomas (FIGS. 9H-9K, 16A and 16B). As reported previously, this mouse model also gives rise to aggressive desmoplastic tumors in the proximal colon, consistent with the location in human patients with this type of aggressive CRC (Nakanishi et al., 2018). Notably, PEGPH20 treatment reduced colon tumorigenesis in this mouse model (FIGS. 16C-16E). In keeping with these histological observations, GSEA of transcriptomic profiling of Prkci^{fl/fl}Prkcz^{fl/fl}; Villin-Cre tumors revealed that PEGPH20 treatment resulted in a decrease in signatures related to invasion and tumor progression such as EMT, myogenesis, angiogenesis, and TGFβ signaling, as well as those corresponding to stromal activation, serrated tumorigenesis and the CMS4 subtype (FIGS. 9L-9O).

Anti-Angiogenic Effect of PEGPH20 on the Vasculature of mCRC Tumors

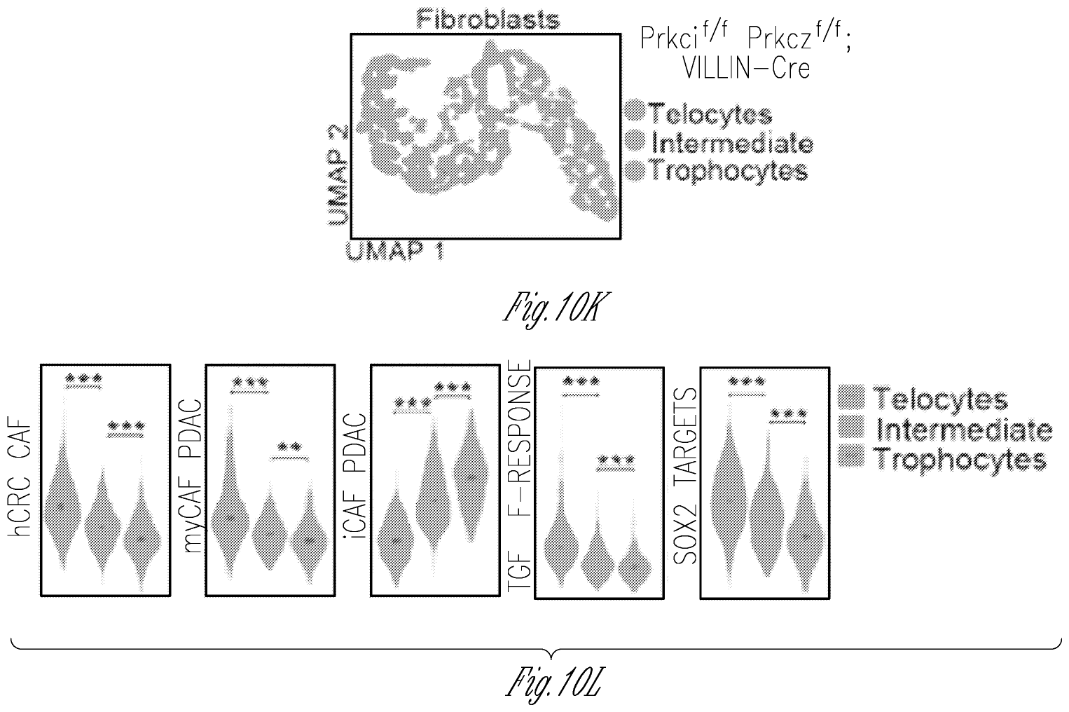

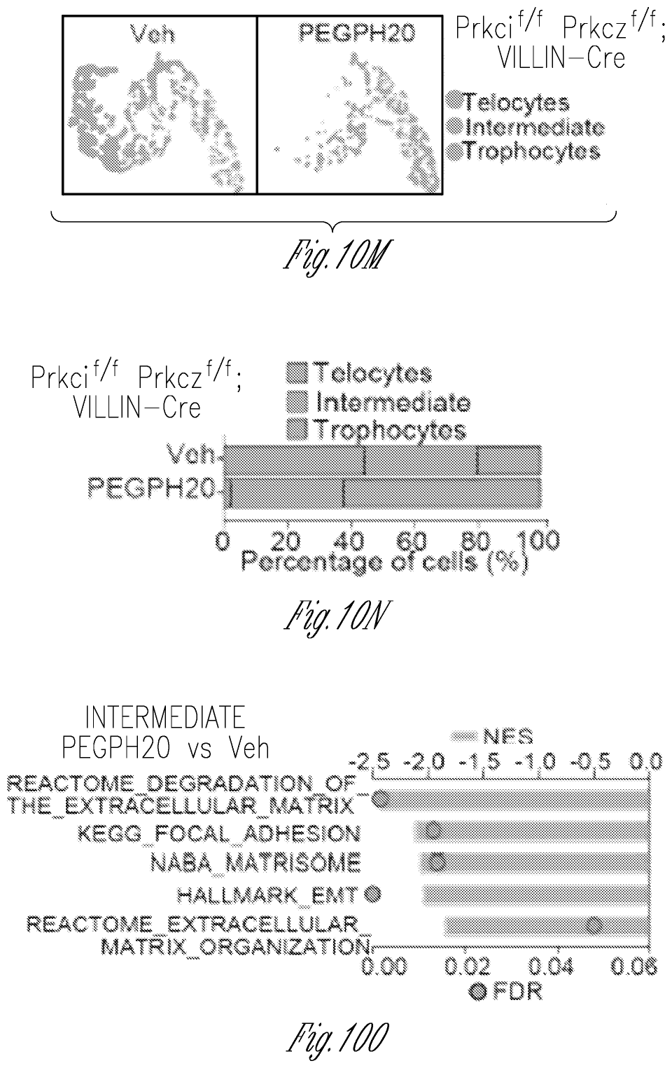

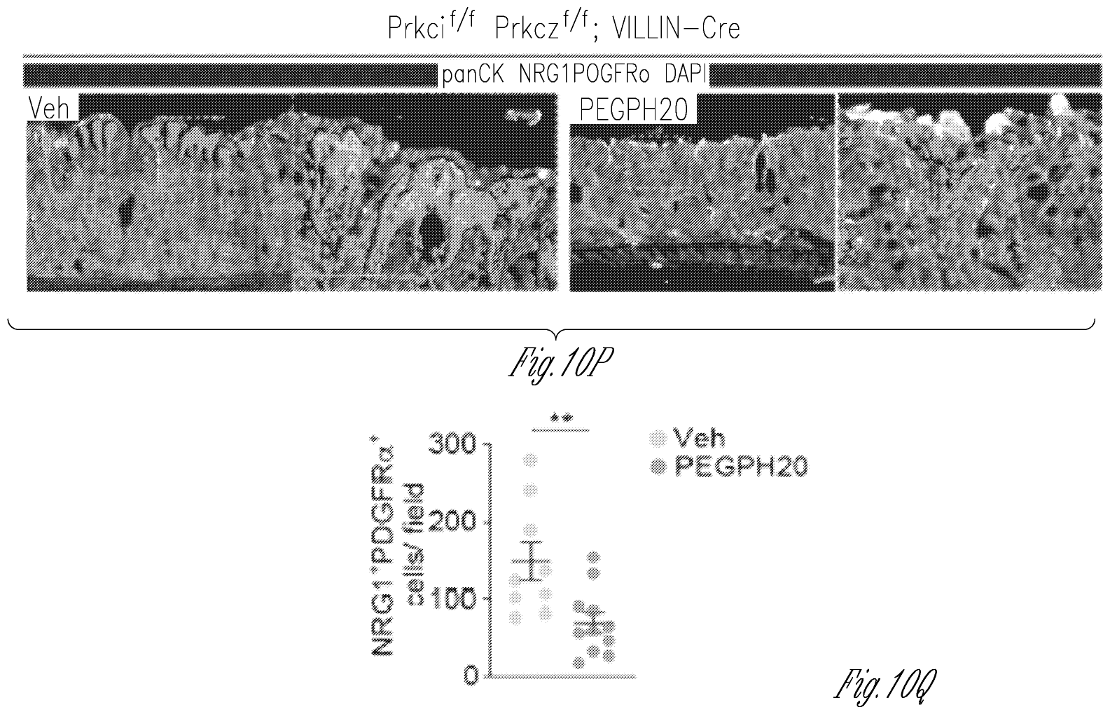

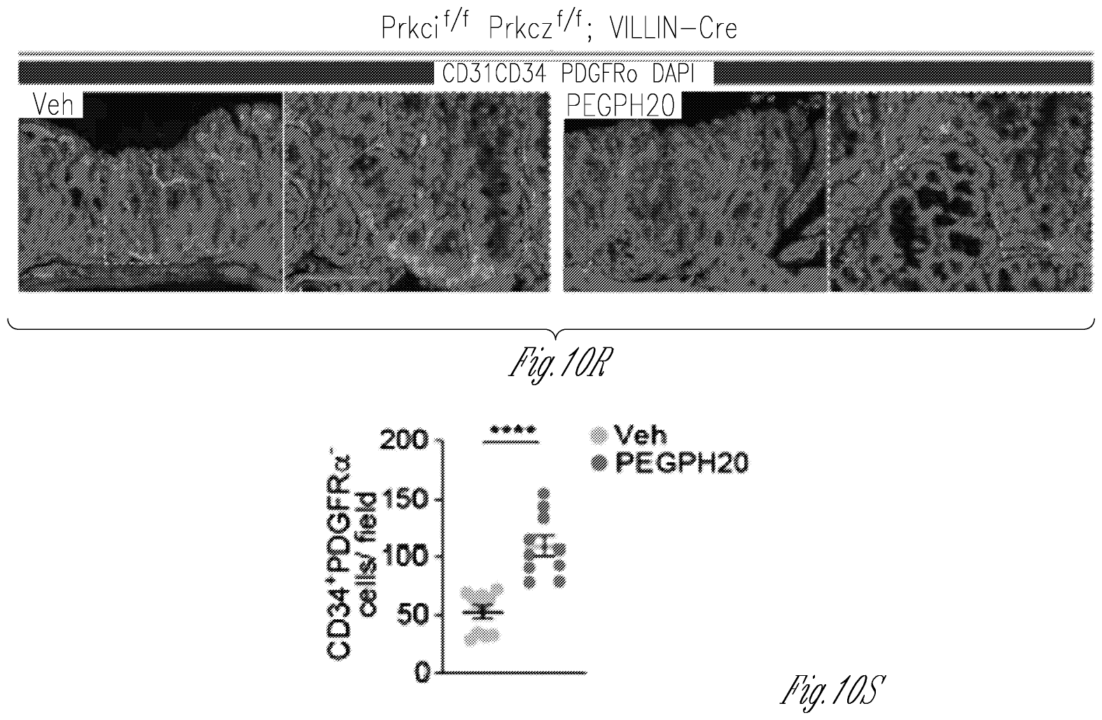

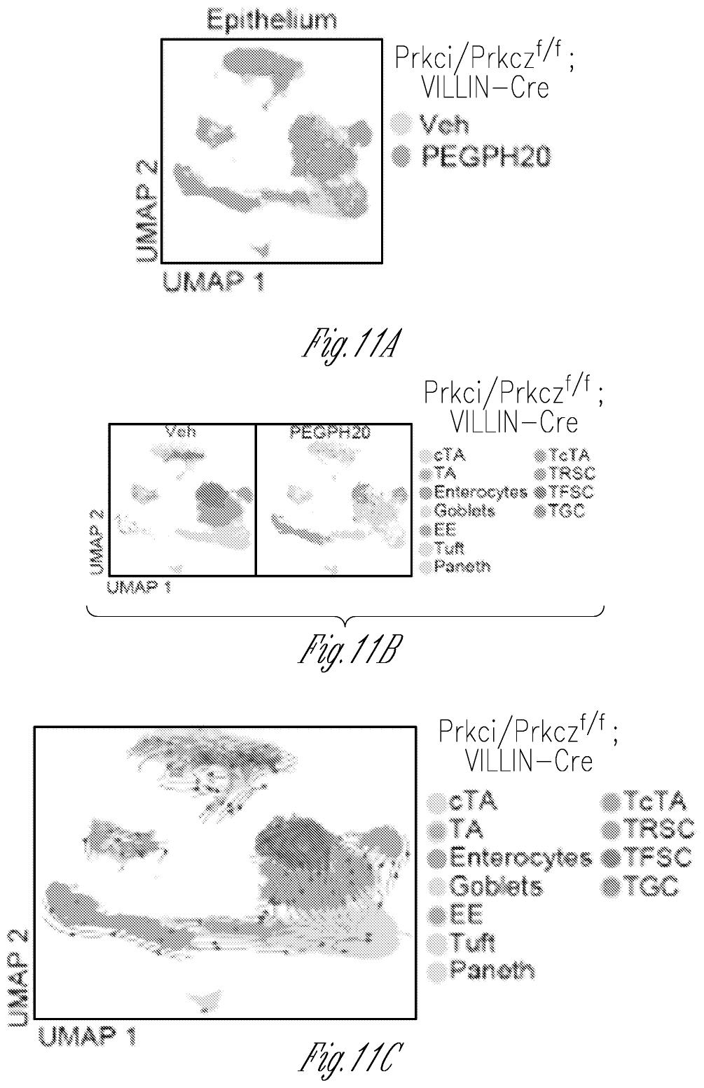

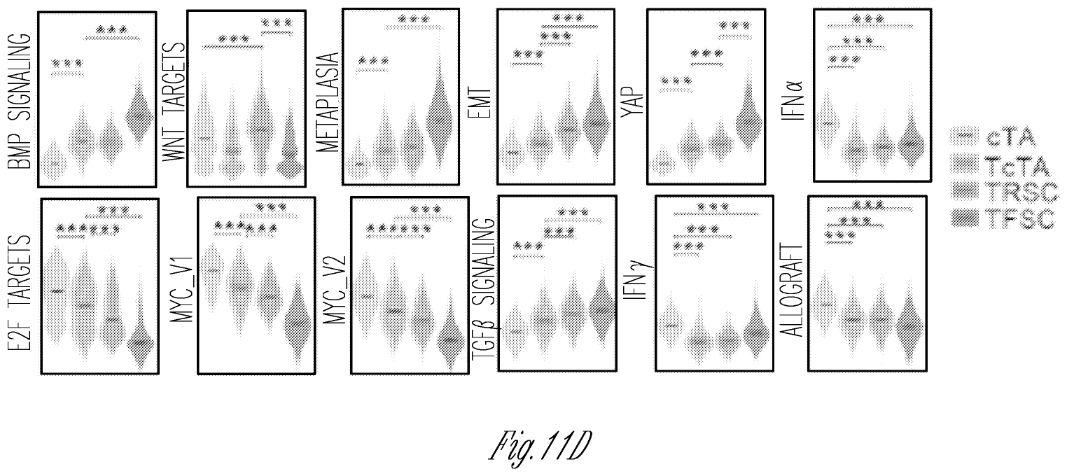

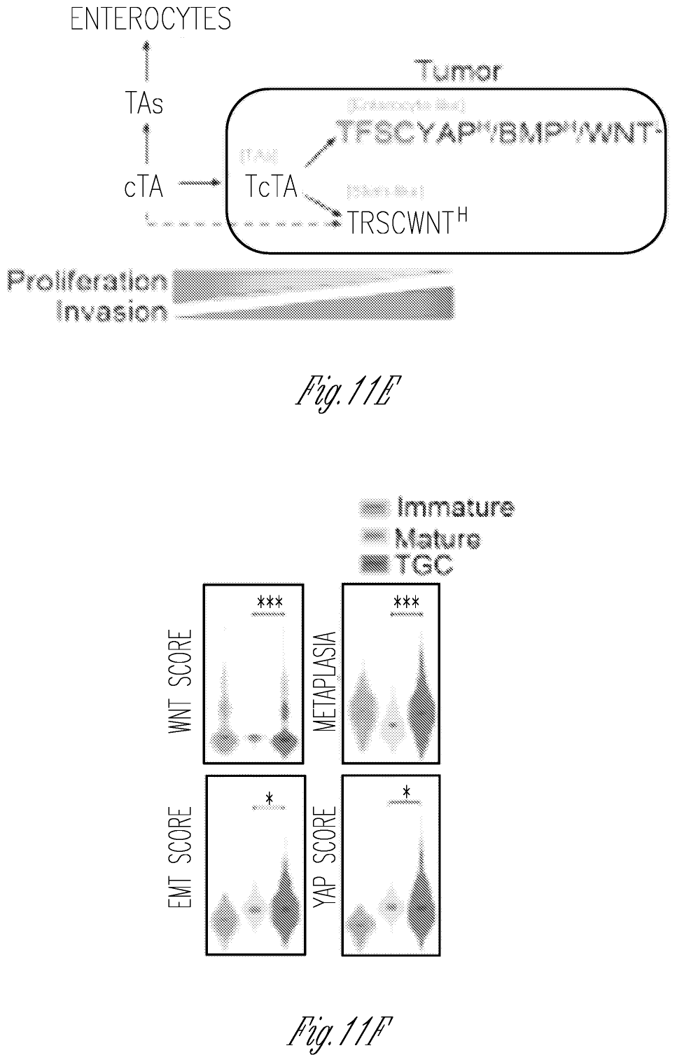

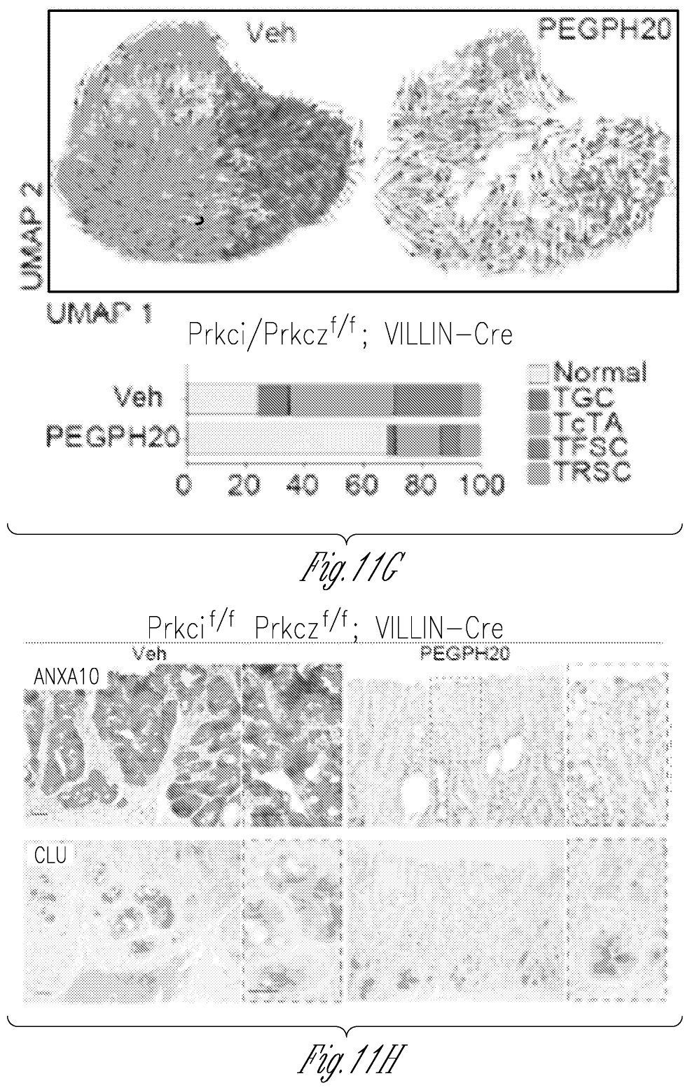

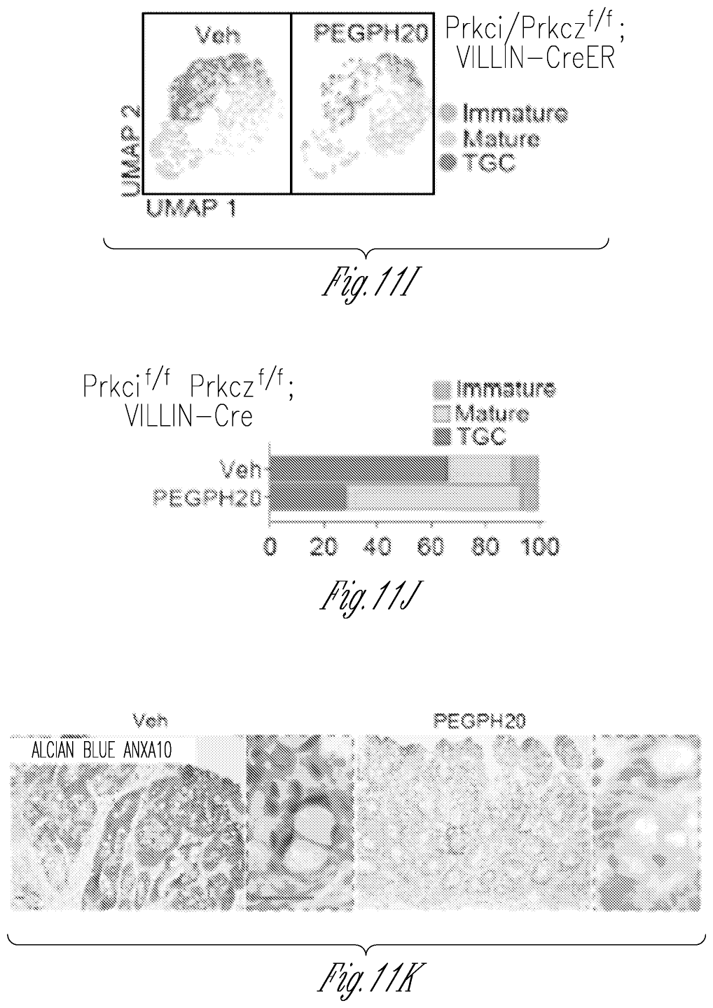

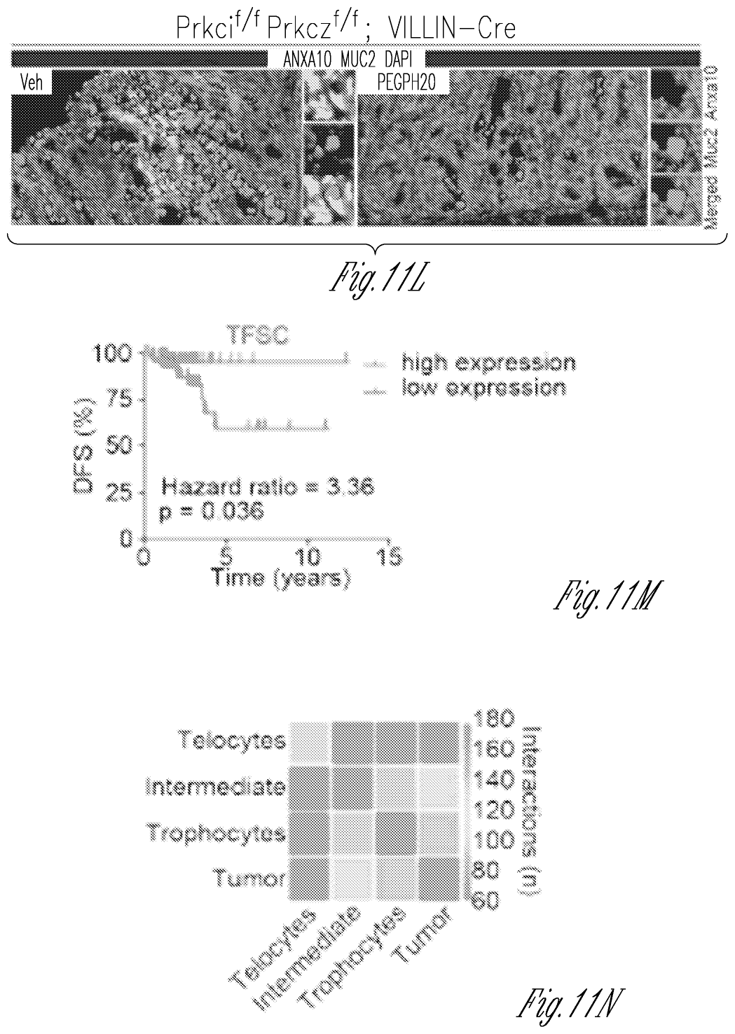

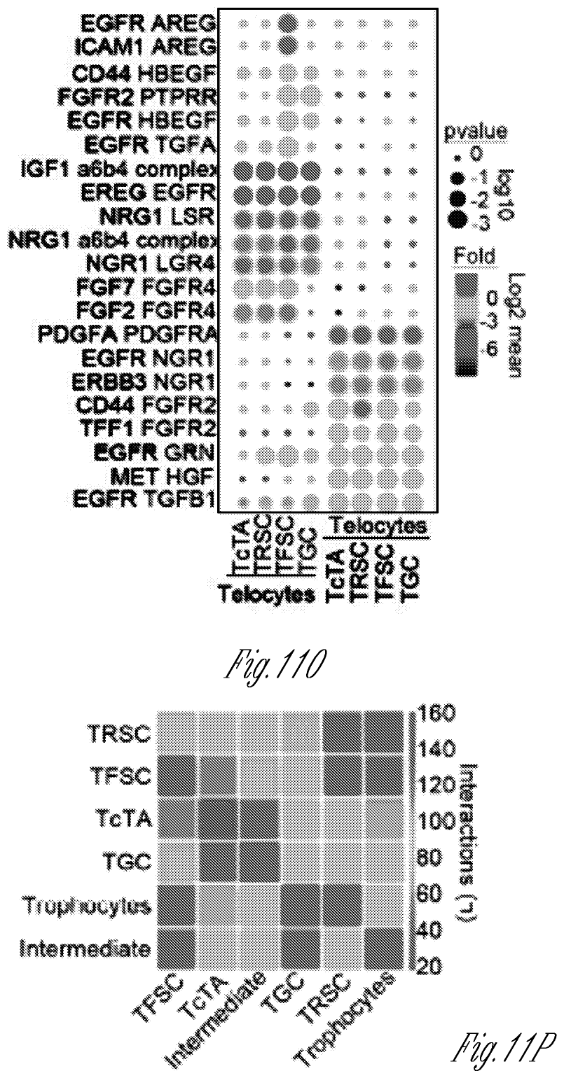

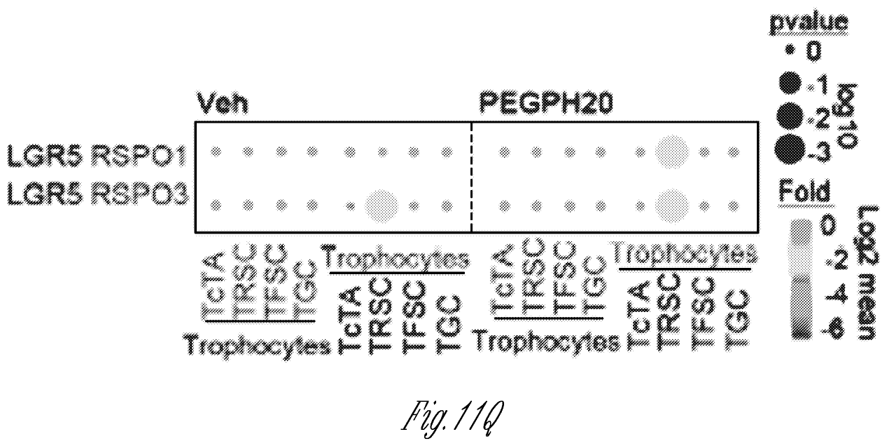

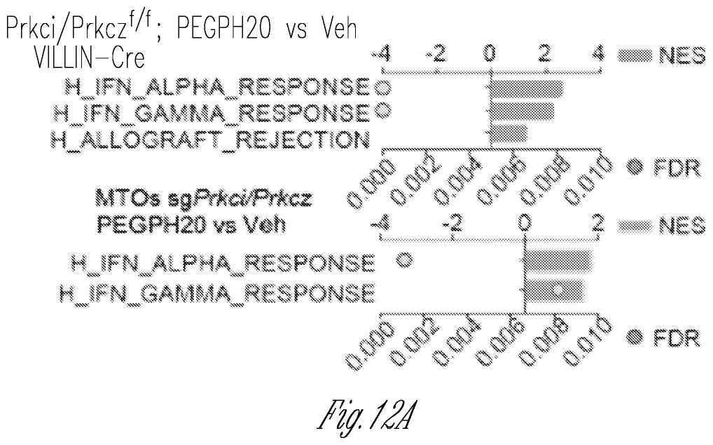

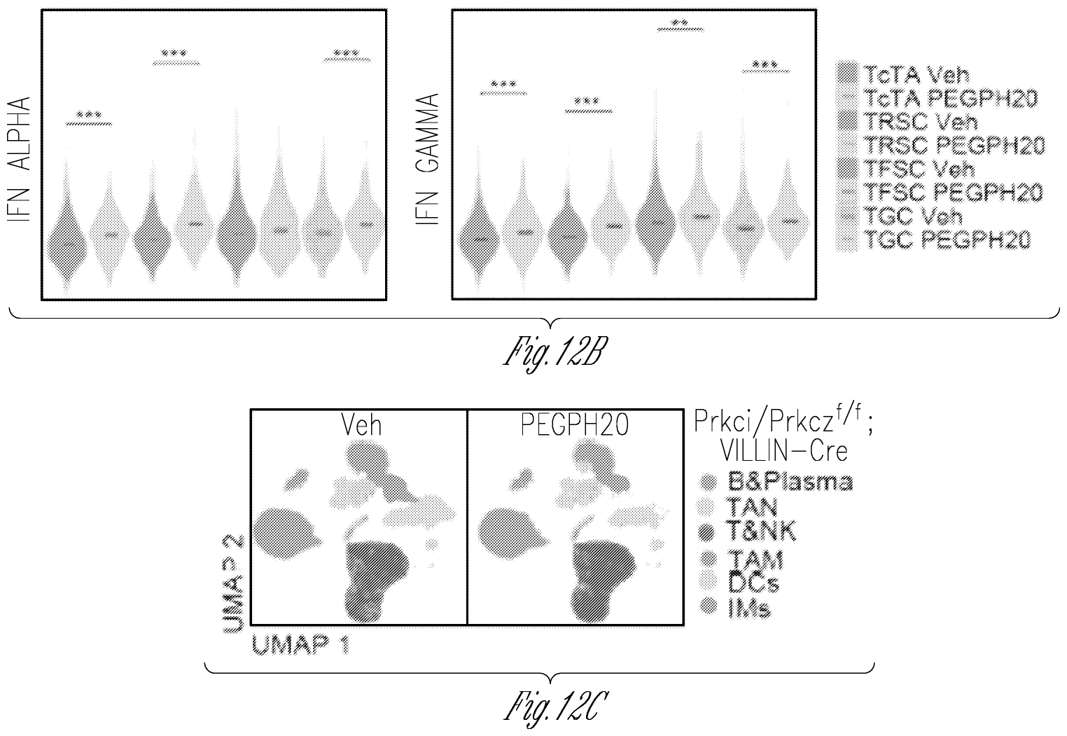

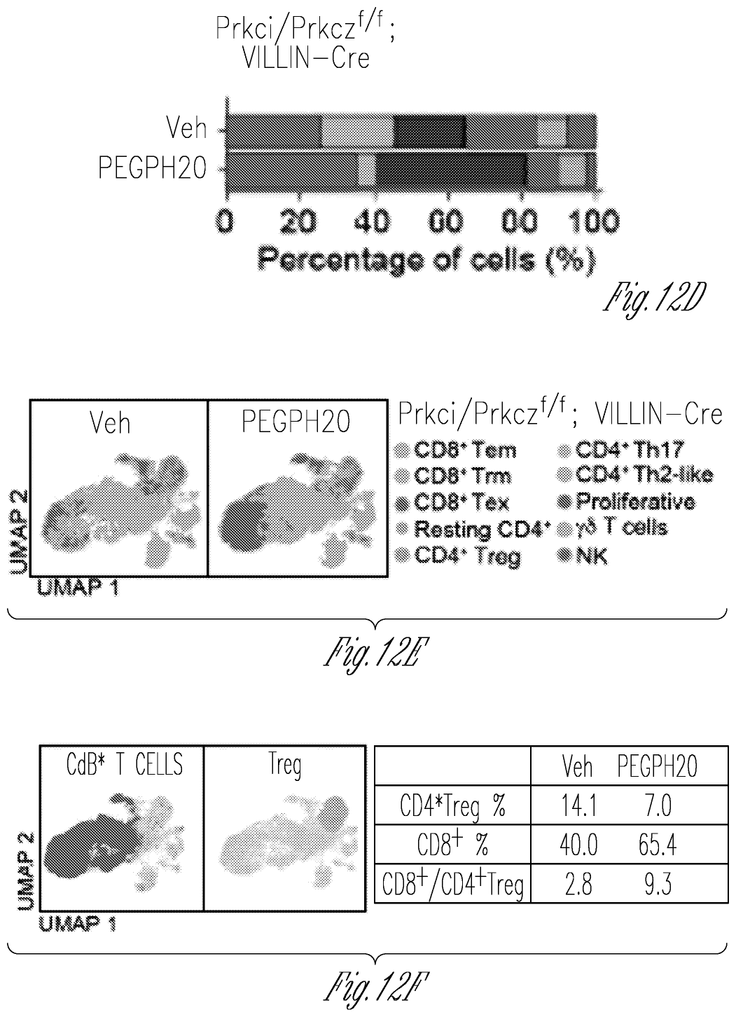

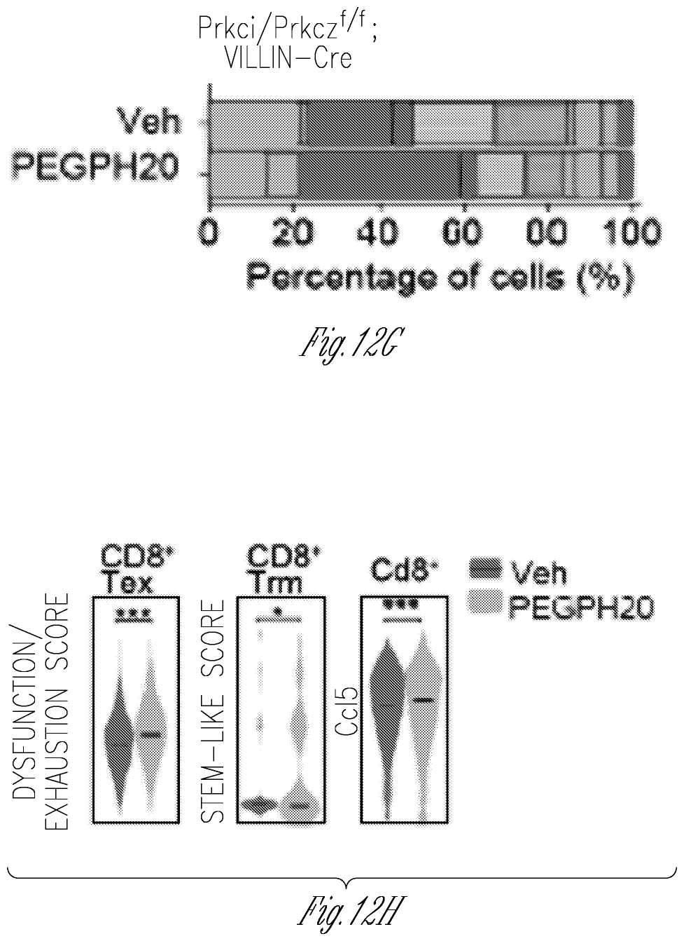

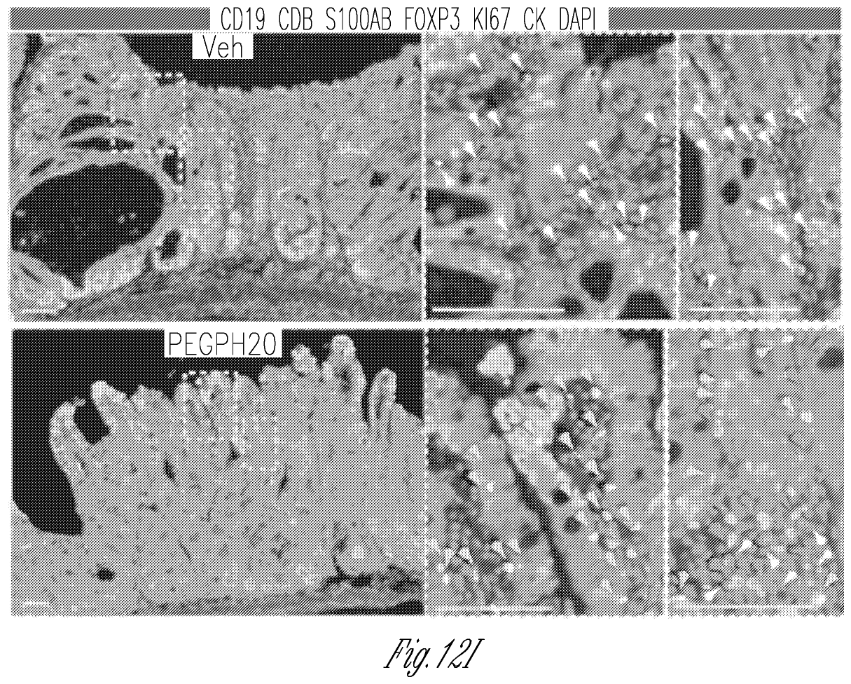

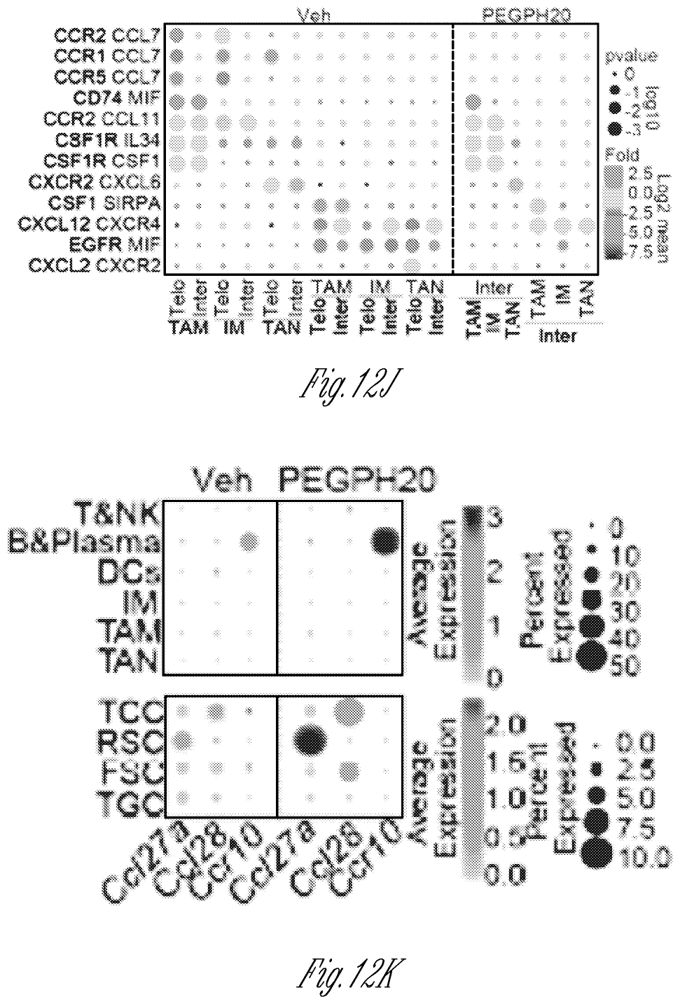

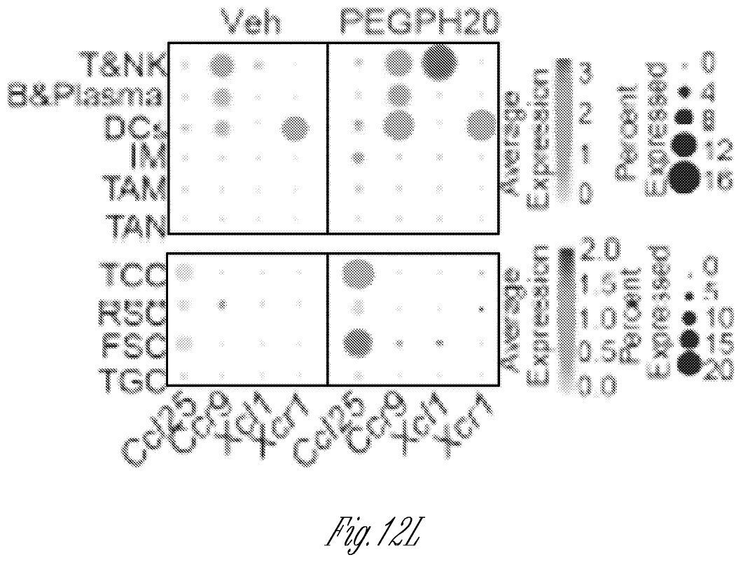

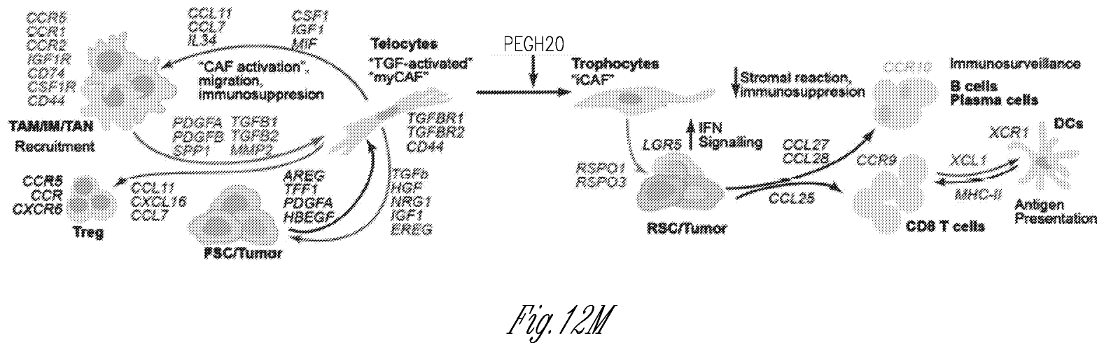

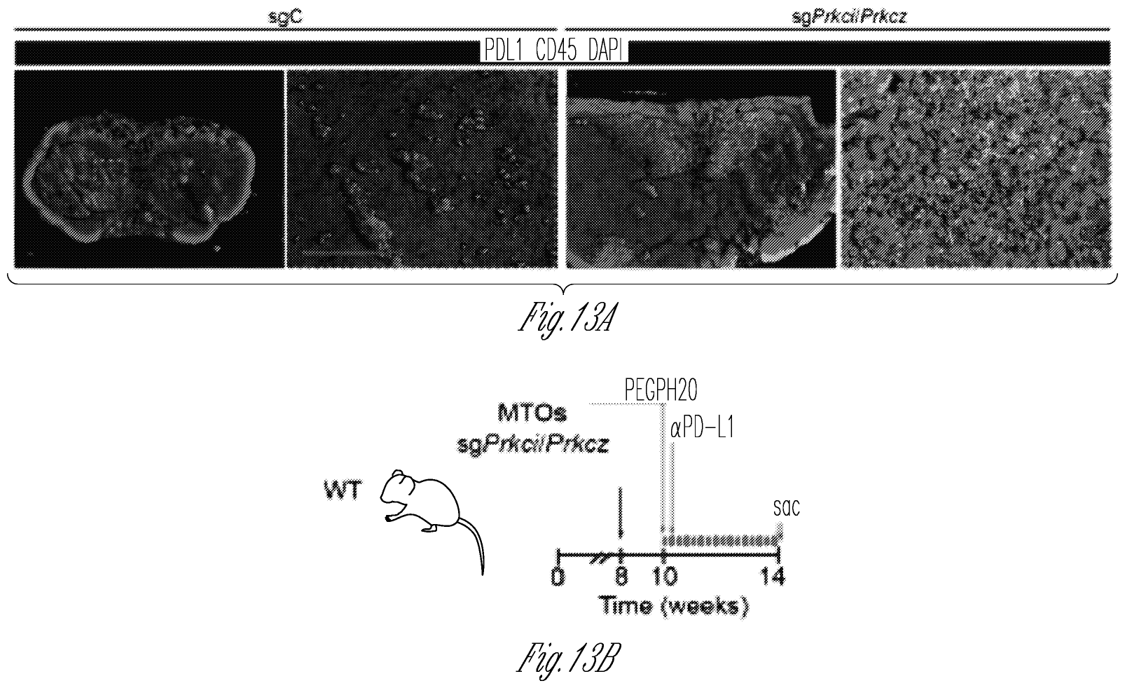

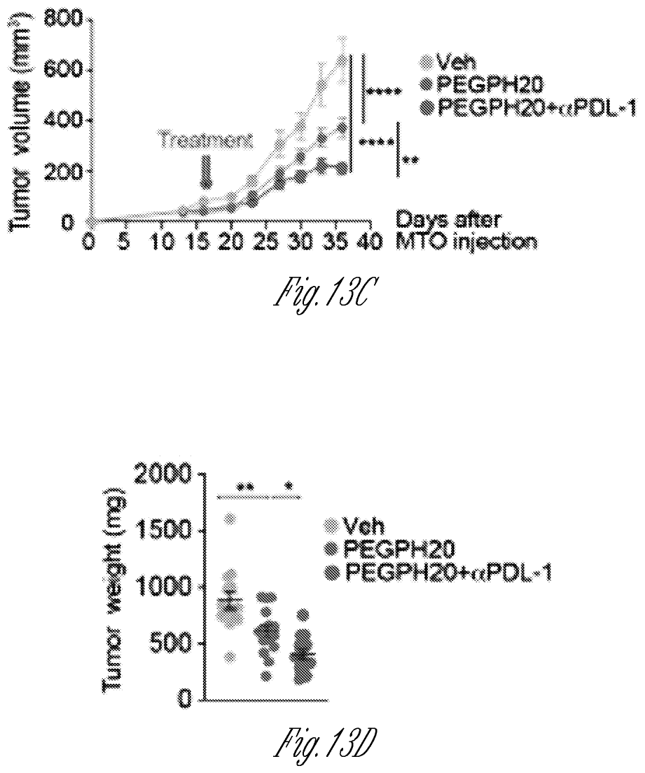

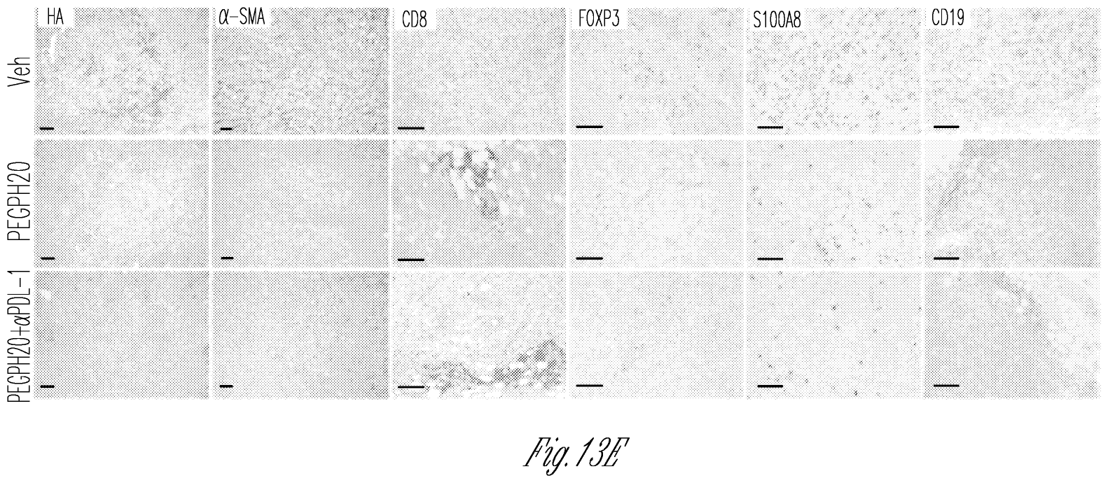

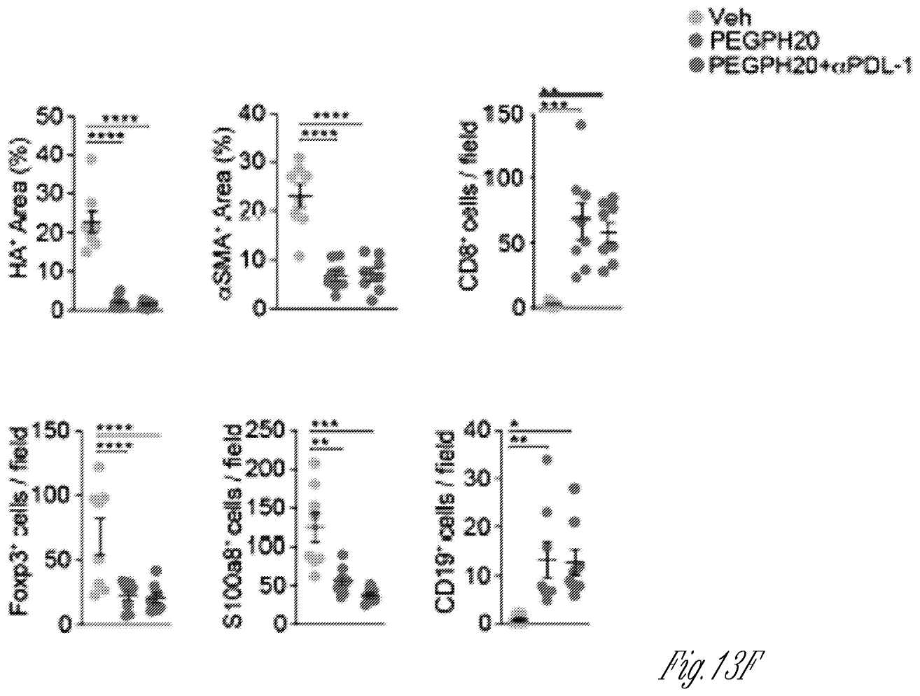

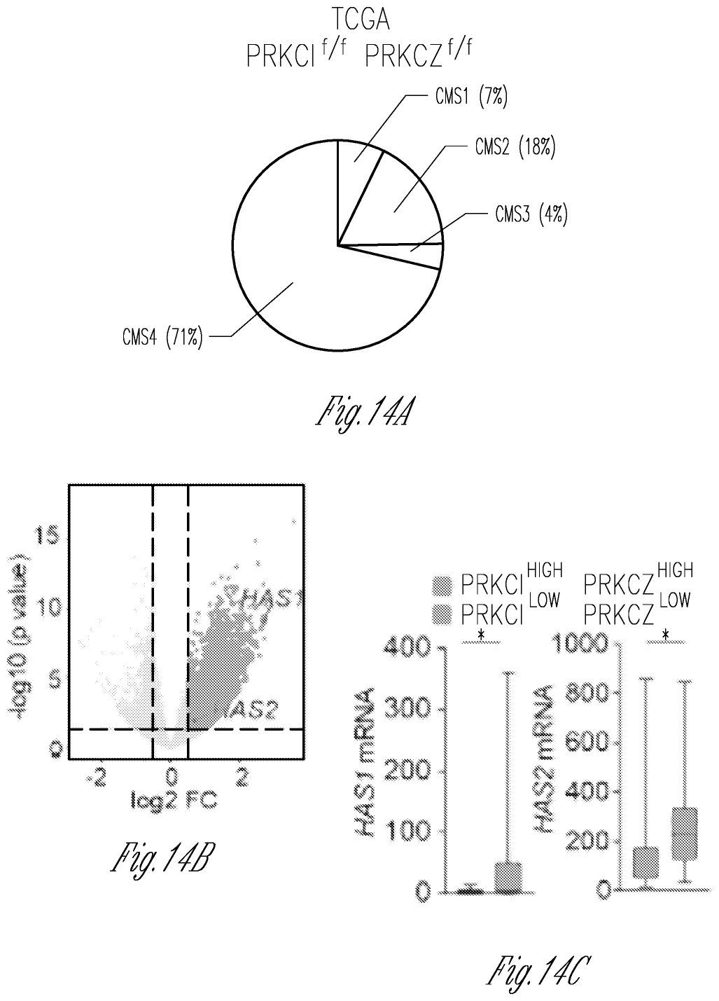

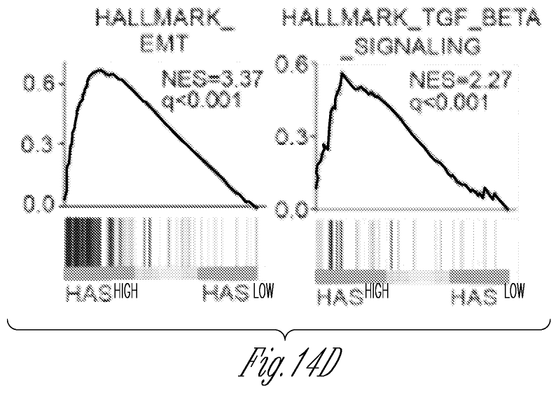

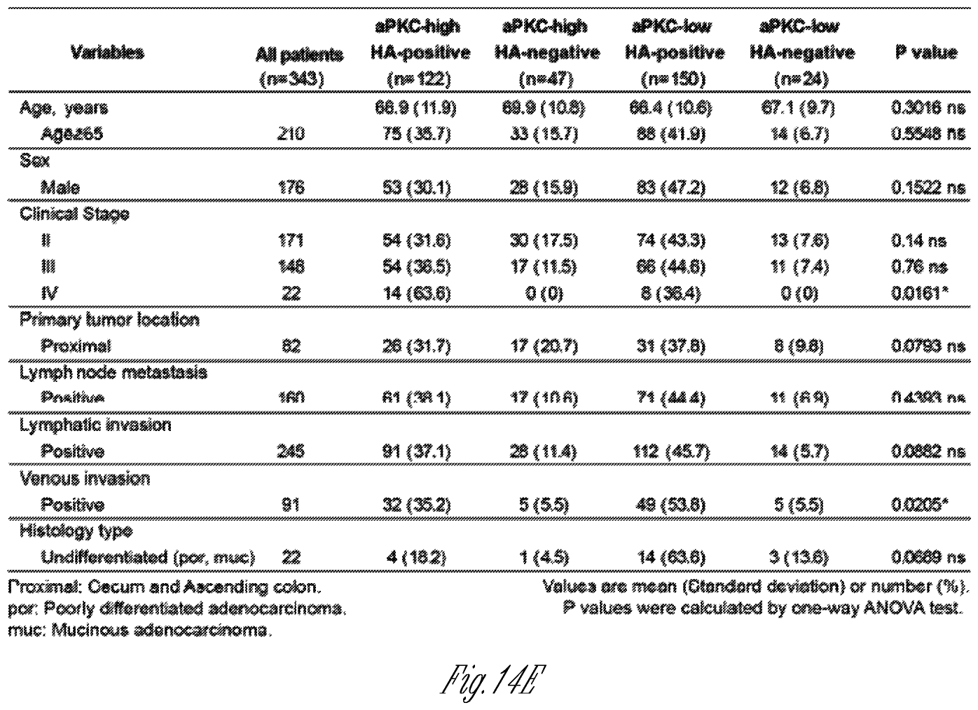

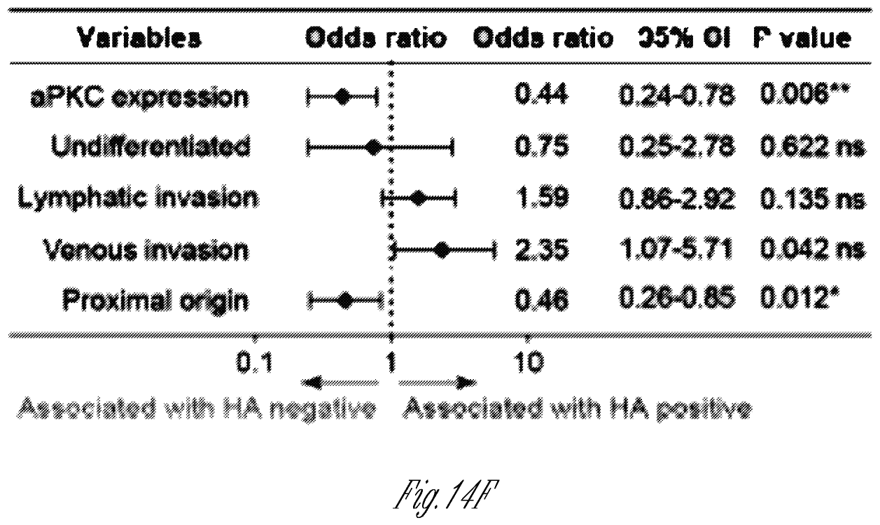

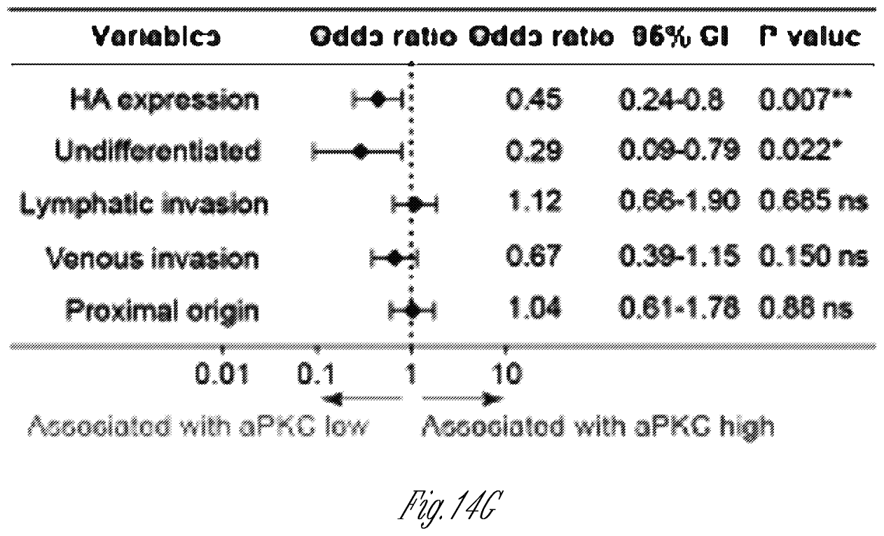

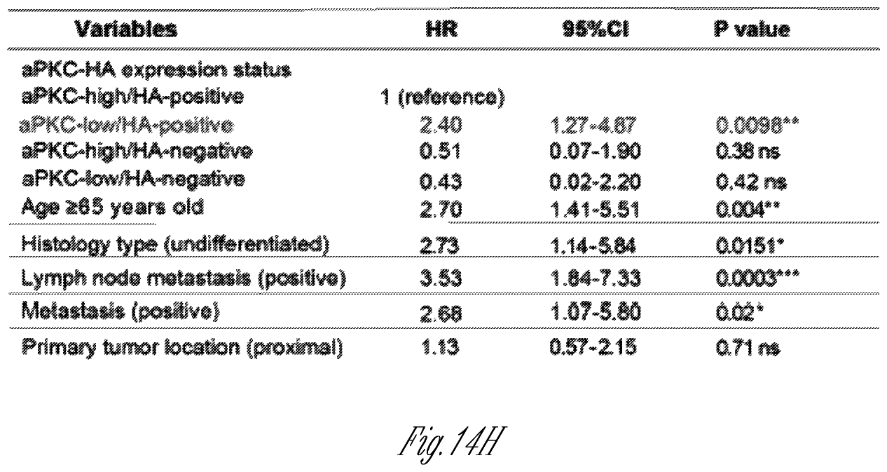

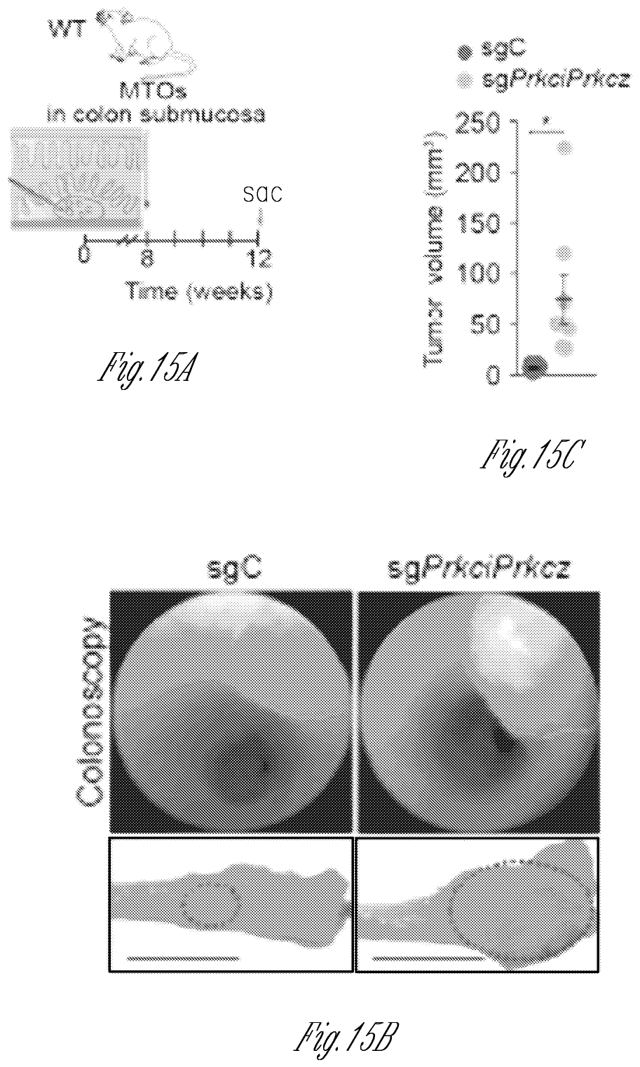

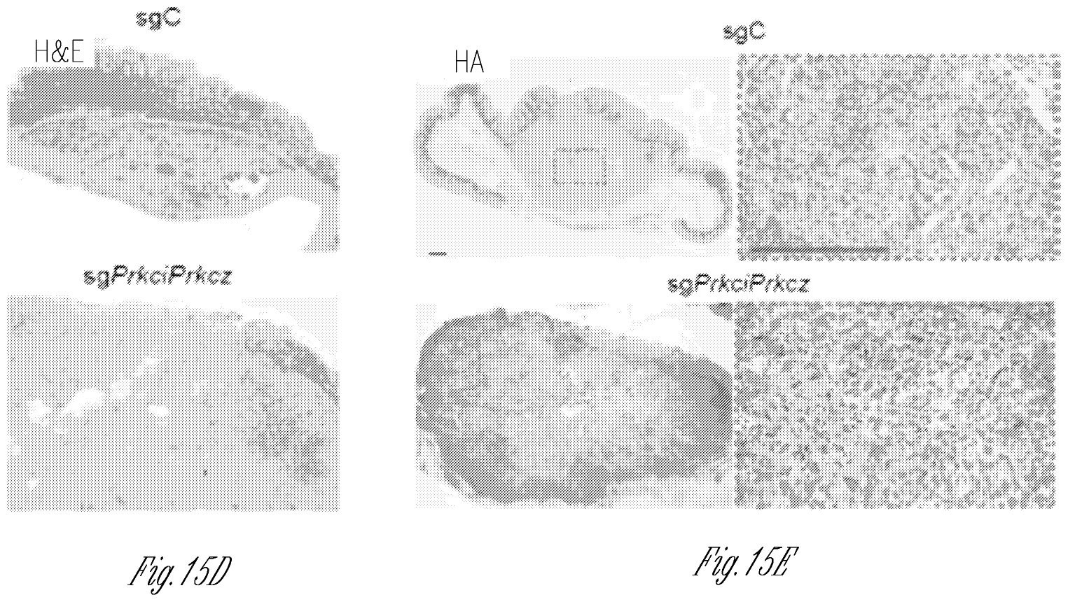

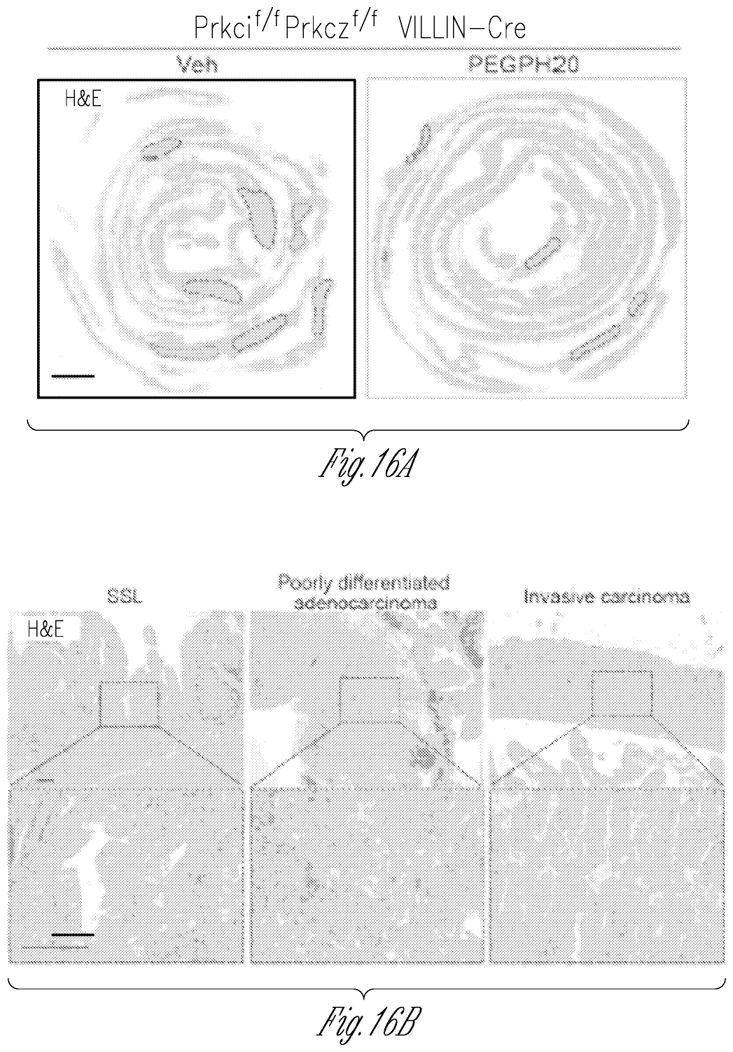

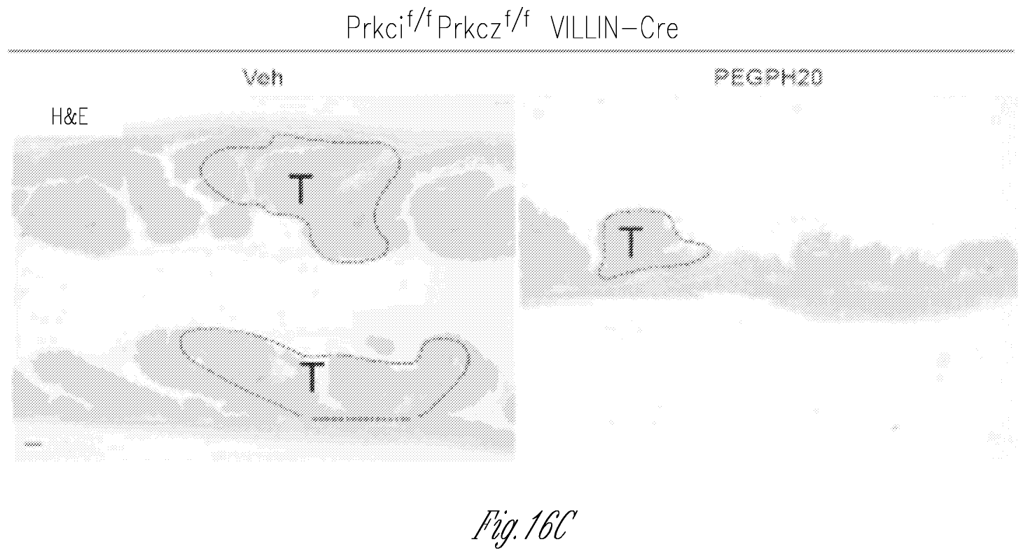

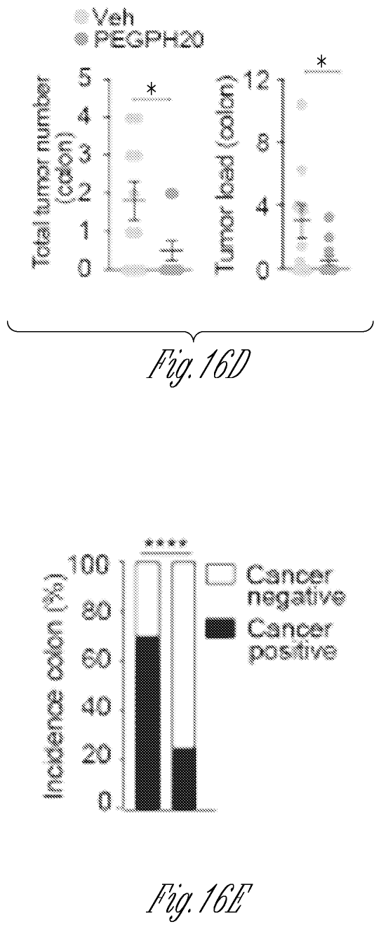

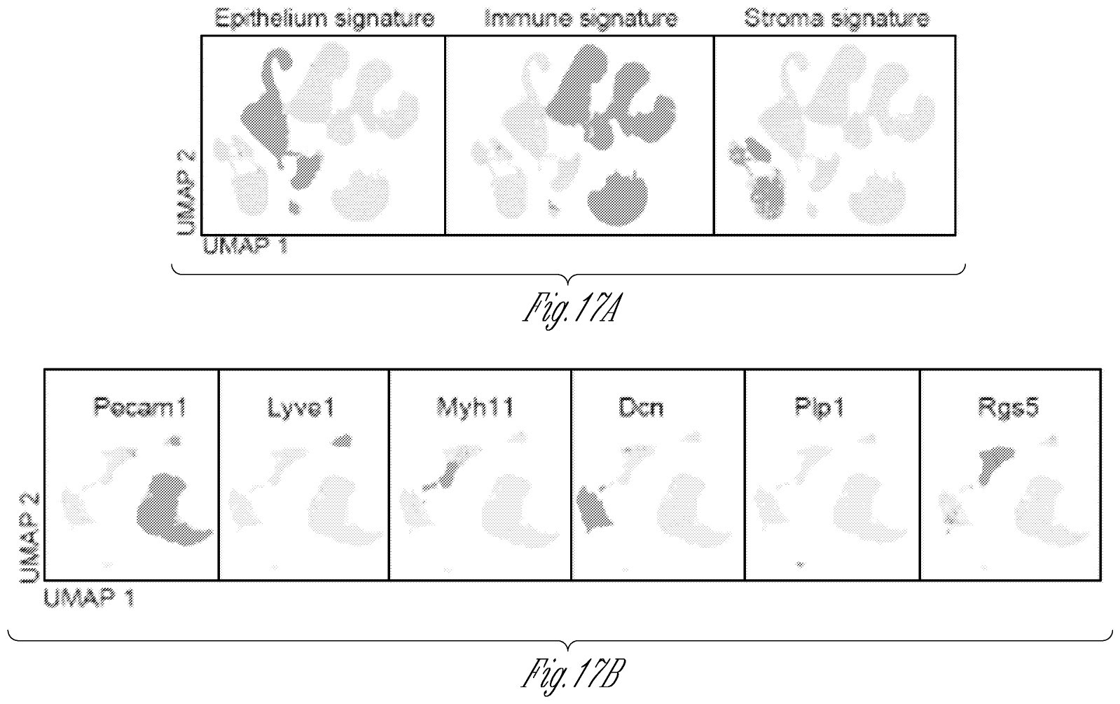

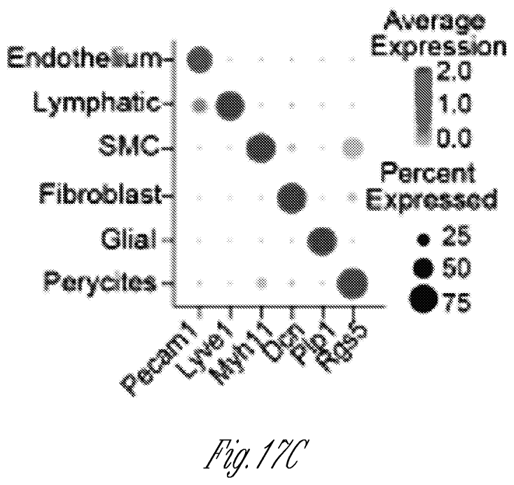

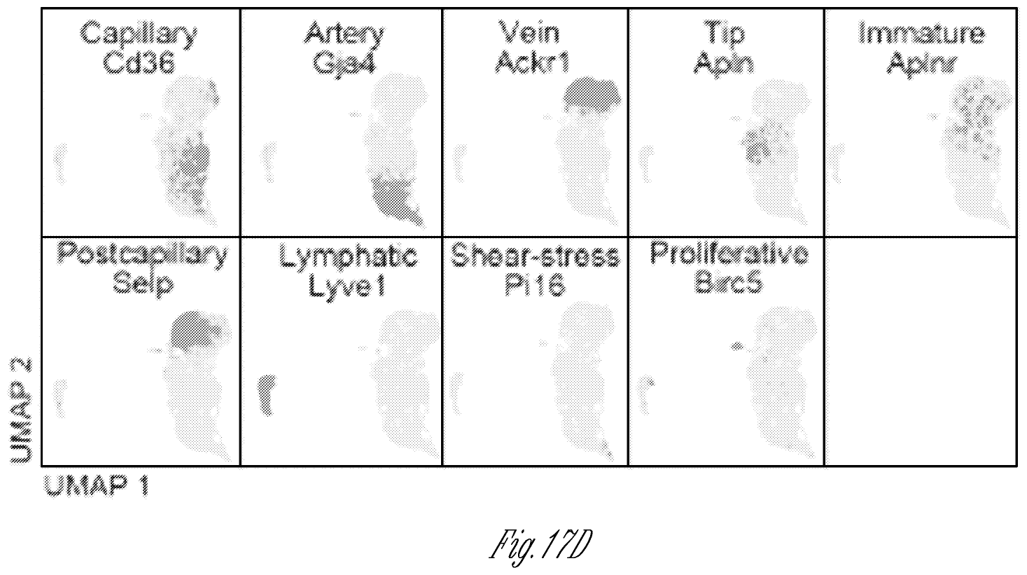

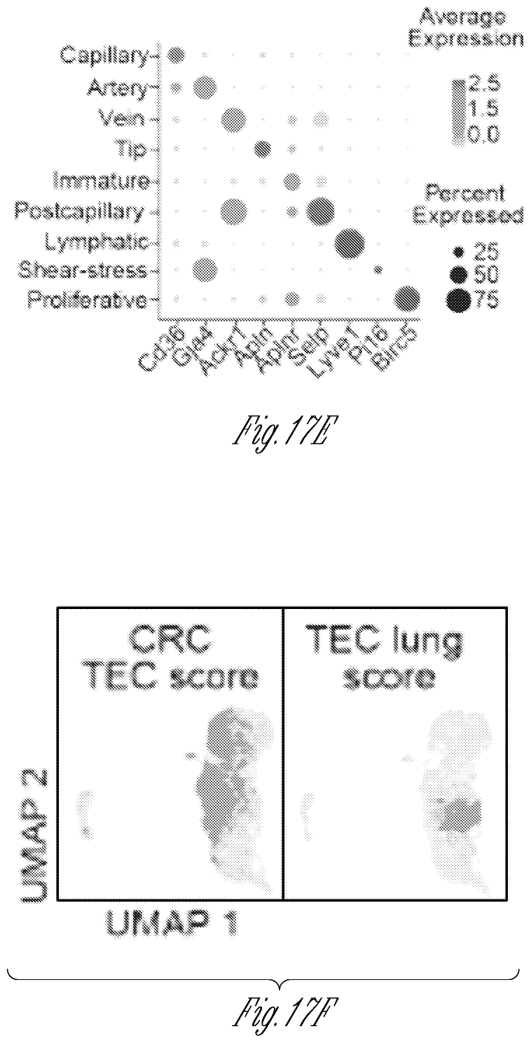

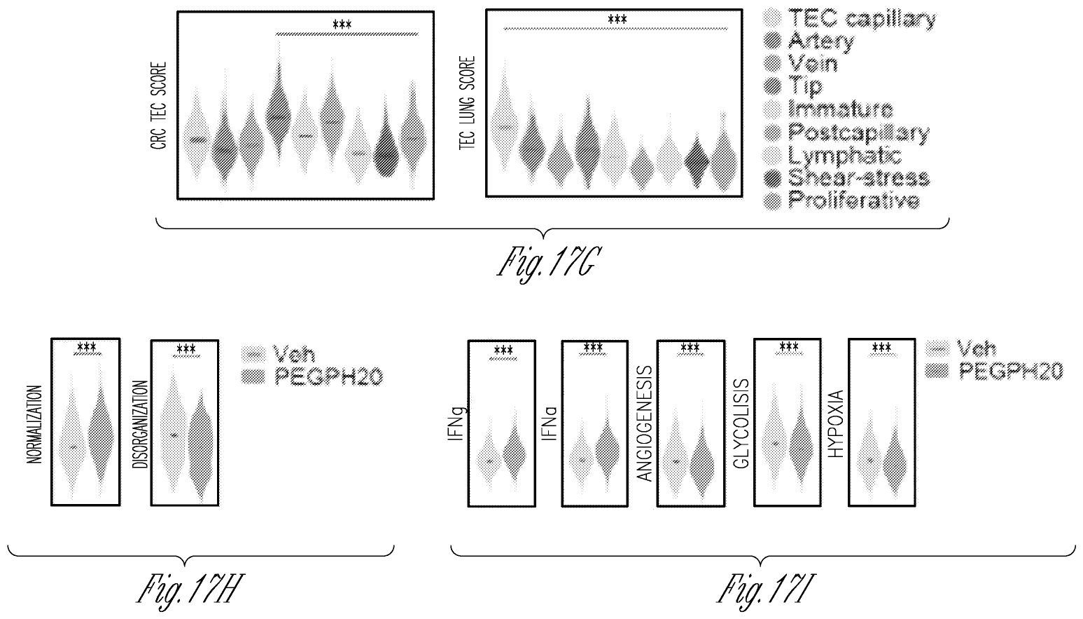

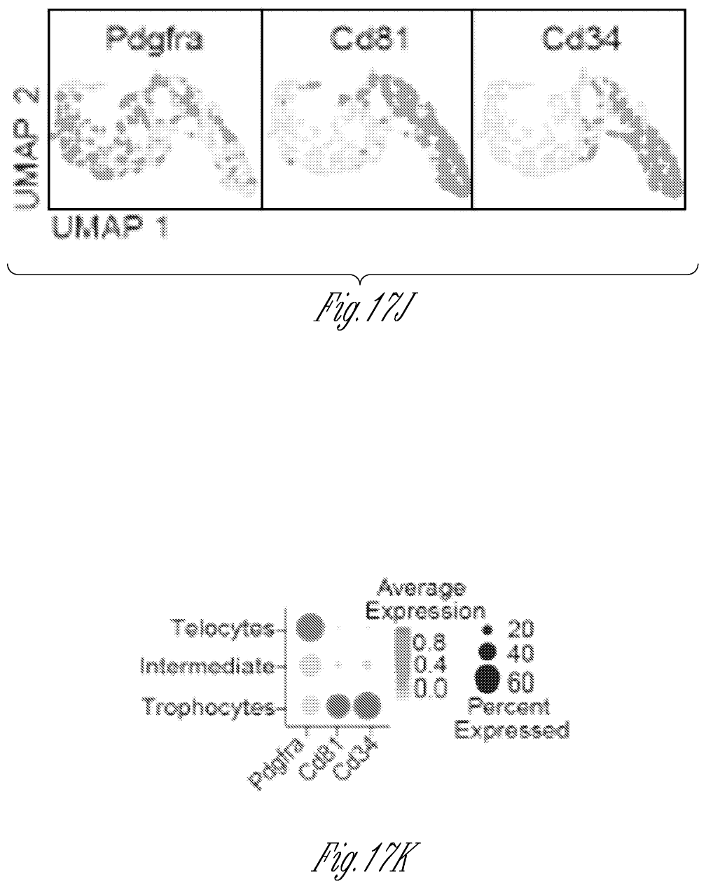

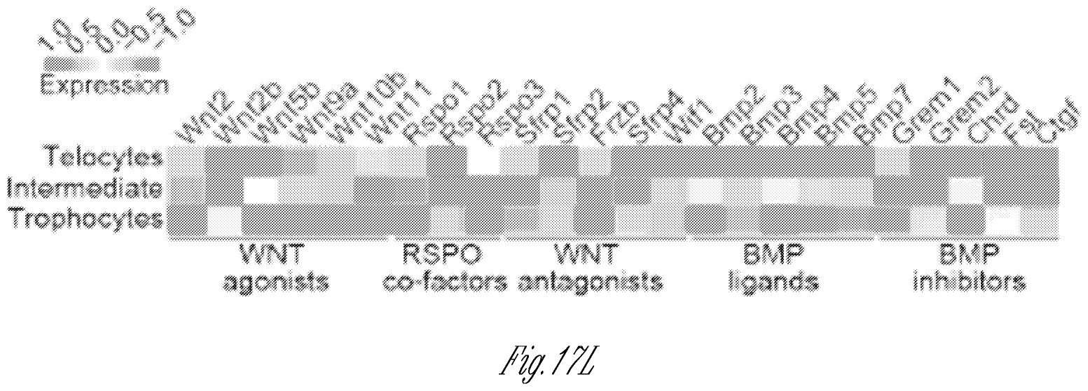

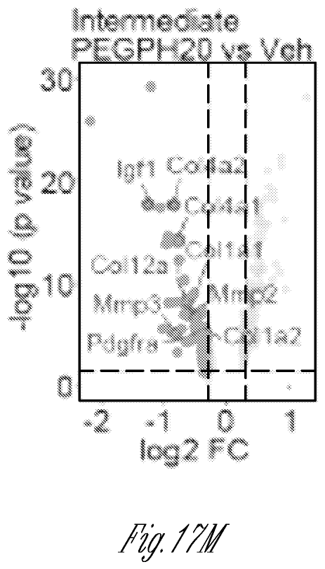

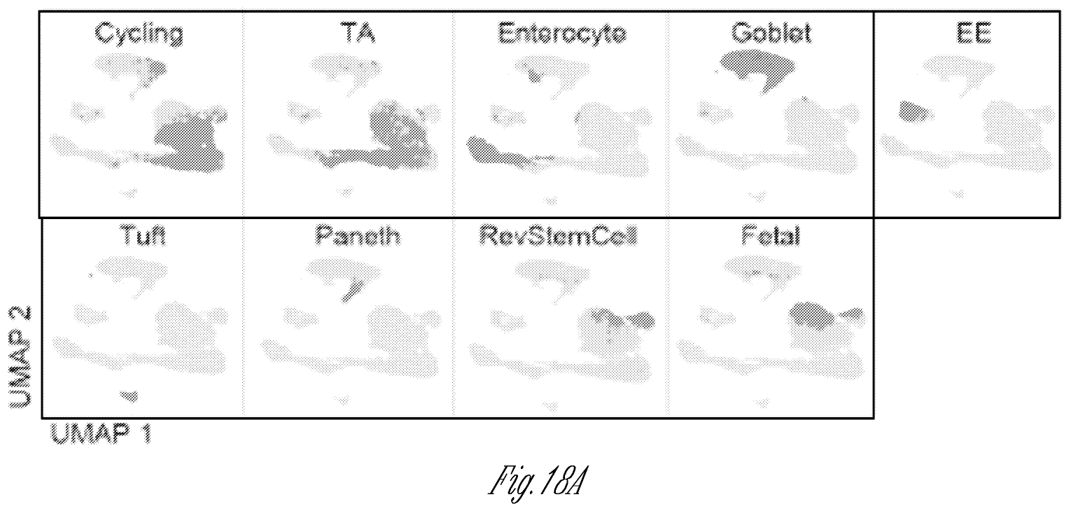

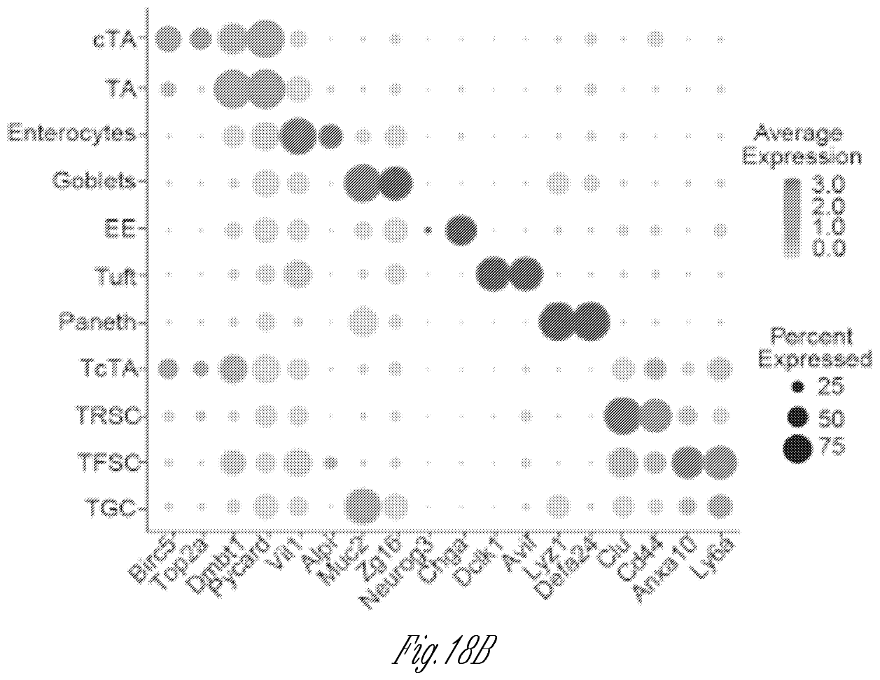

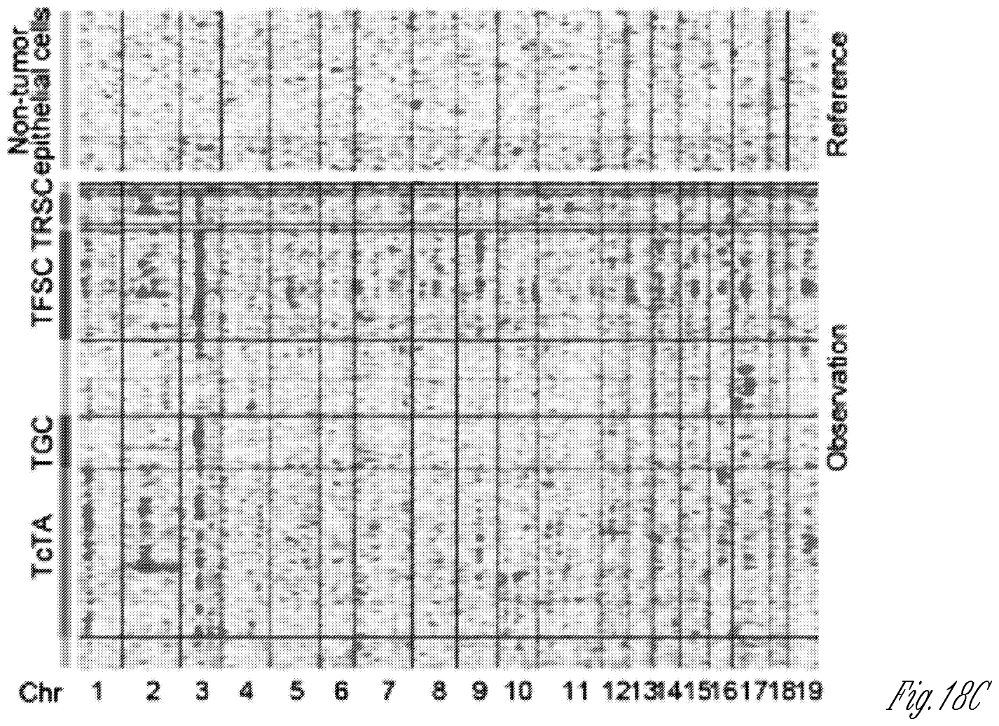

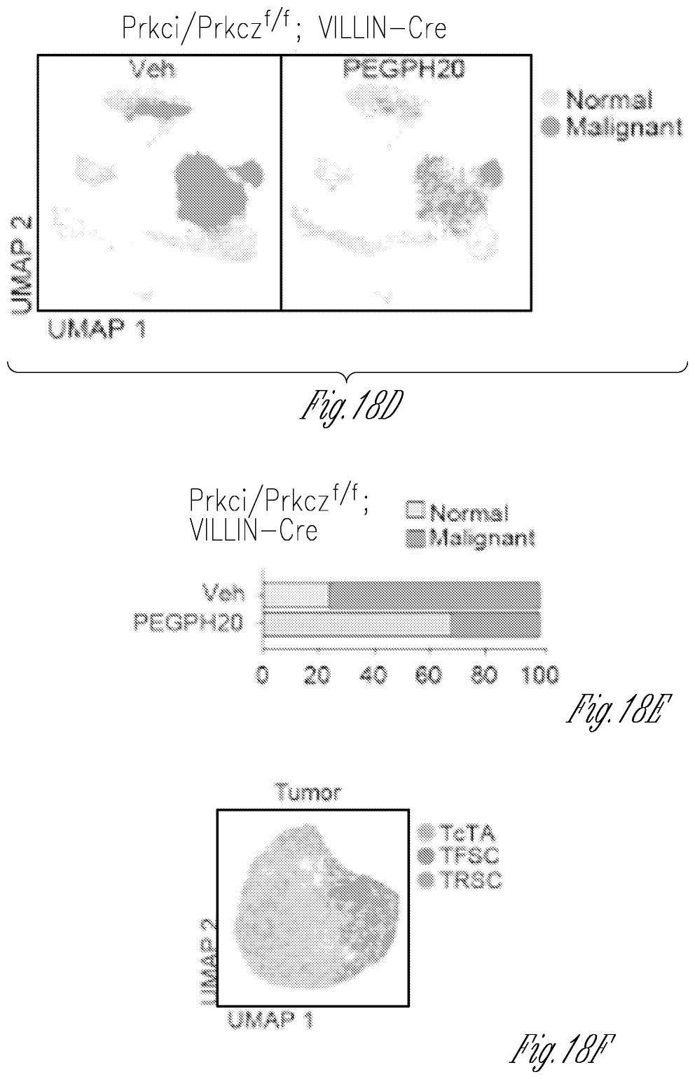

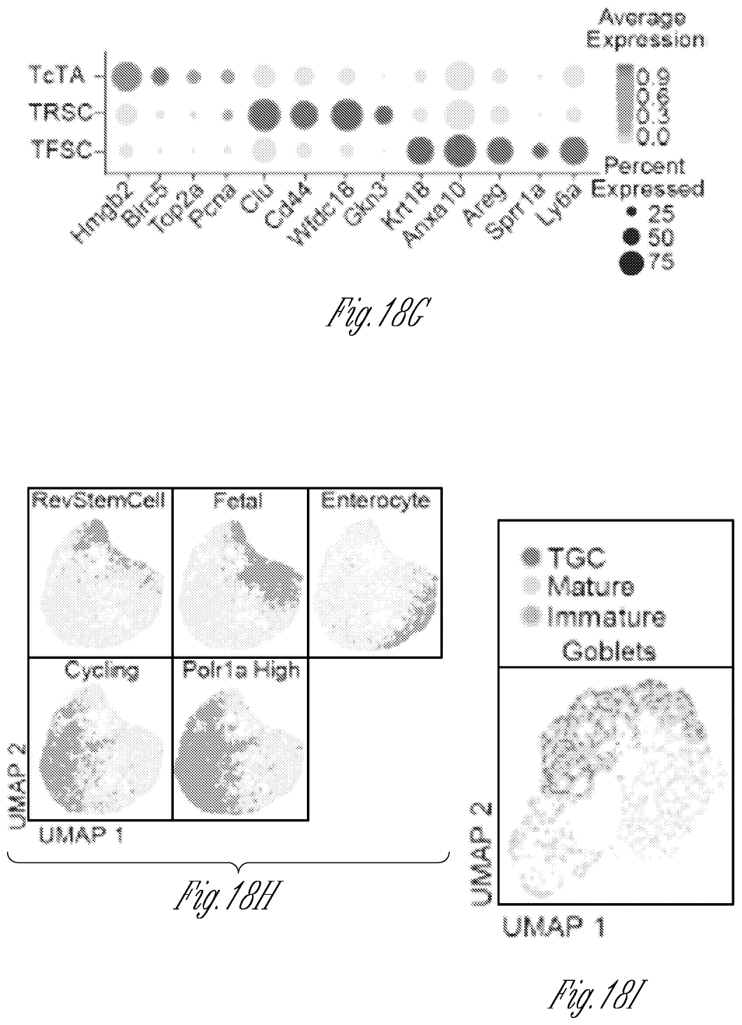

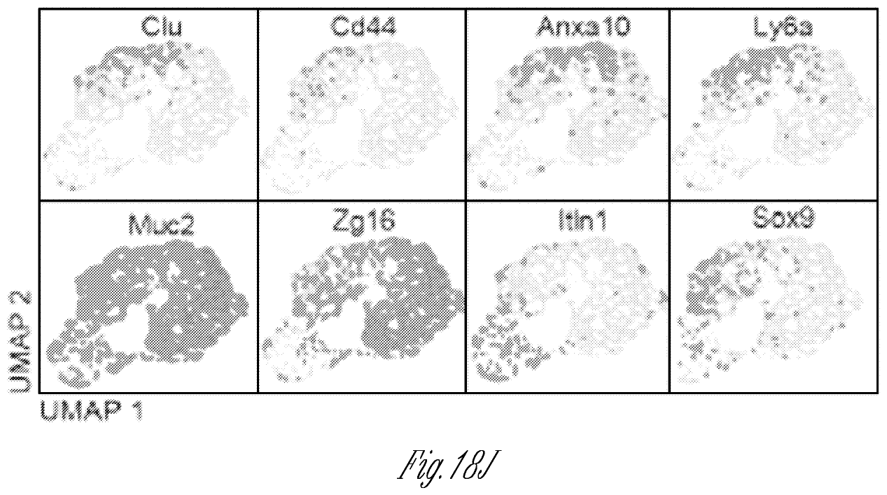

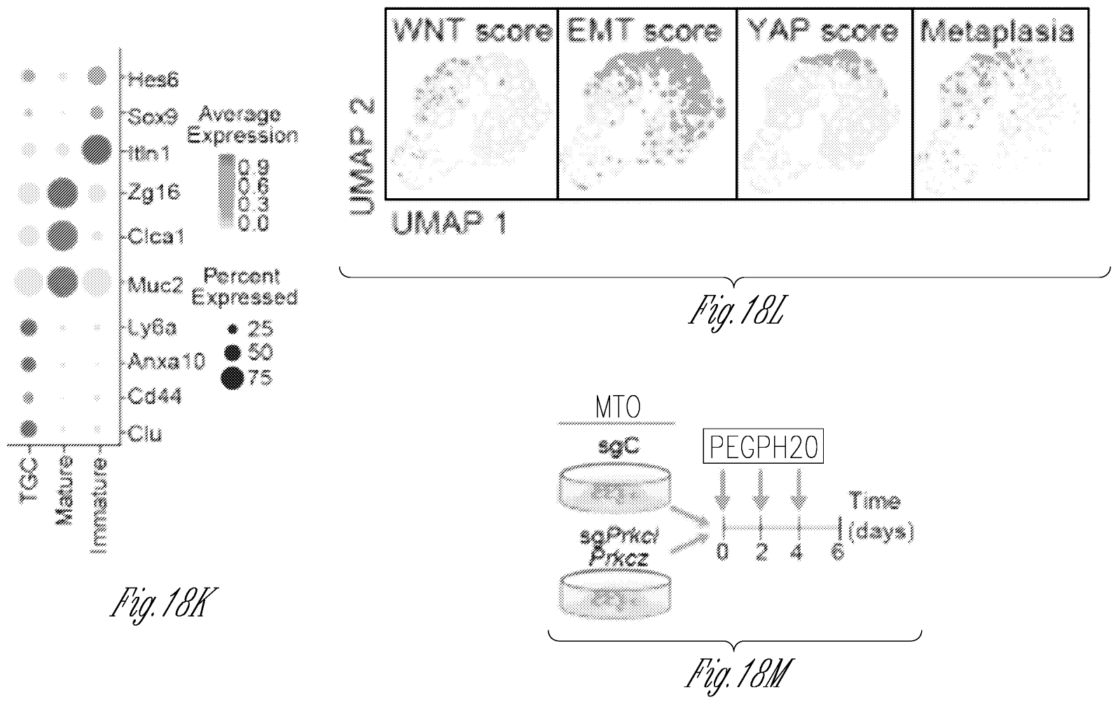

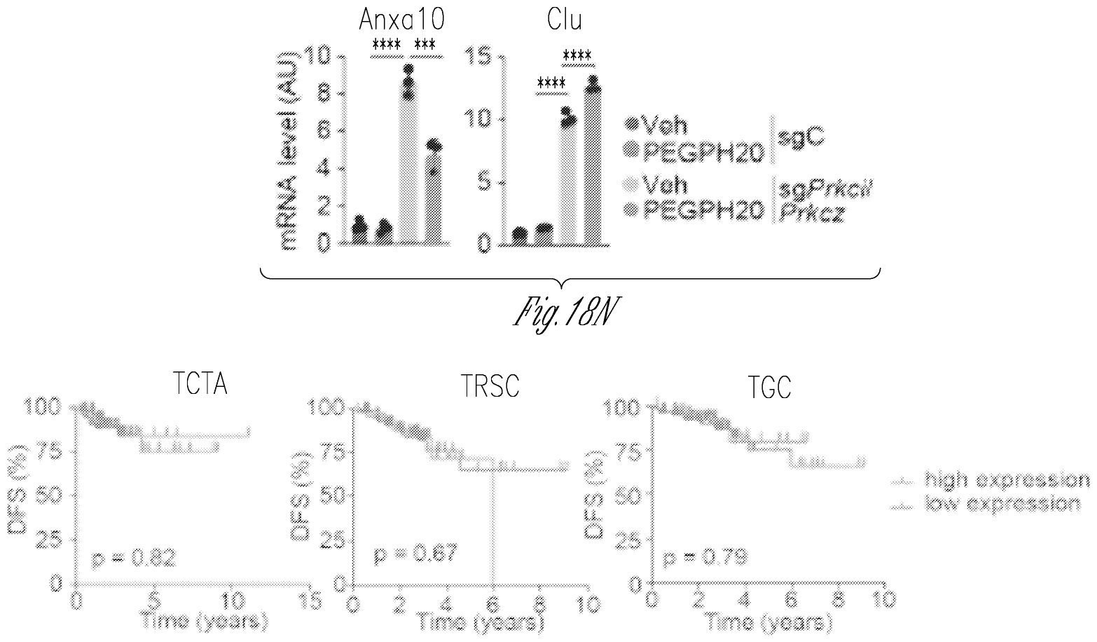

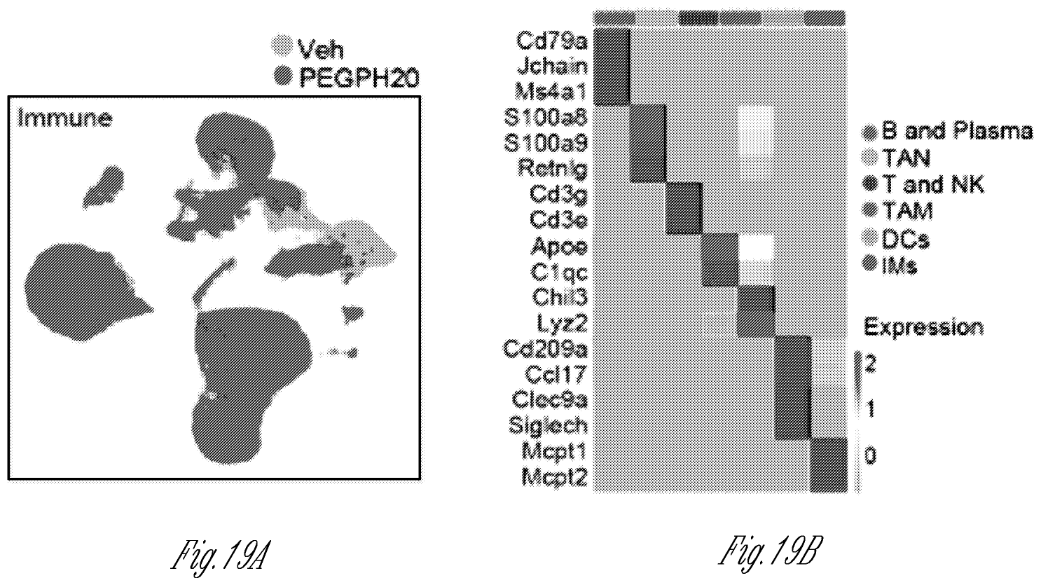

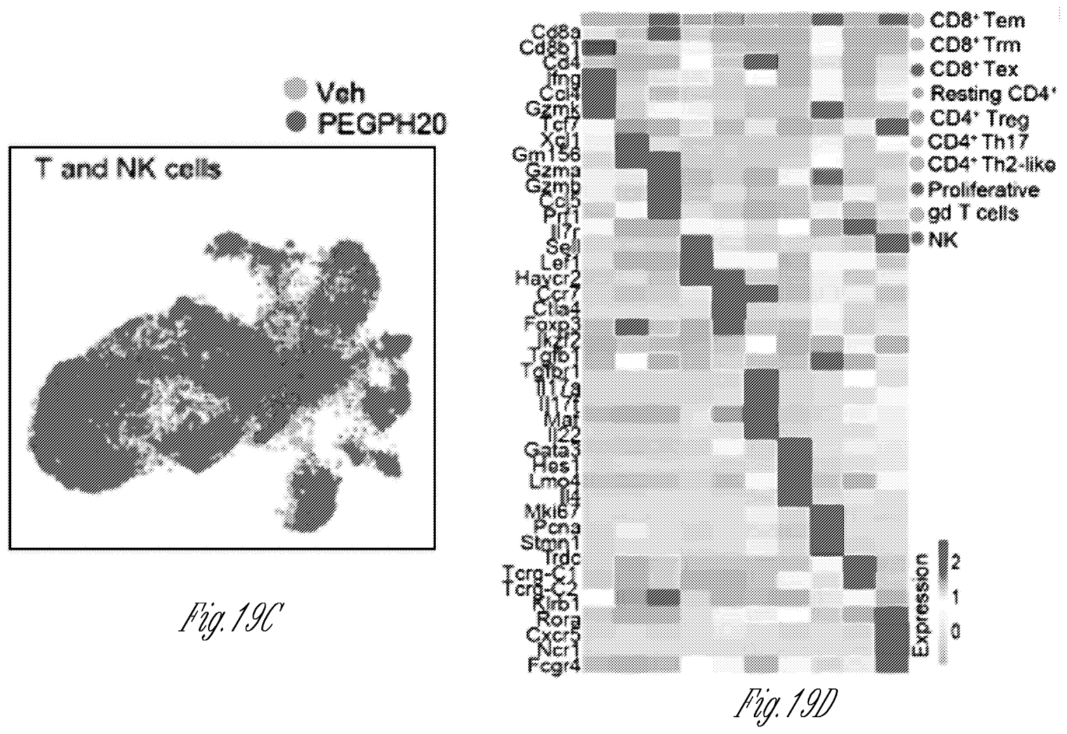

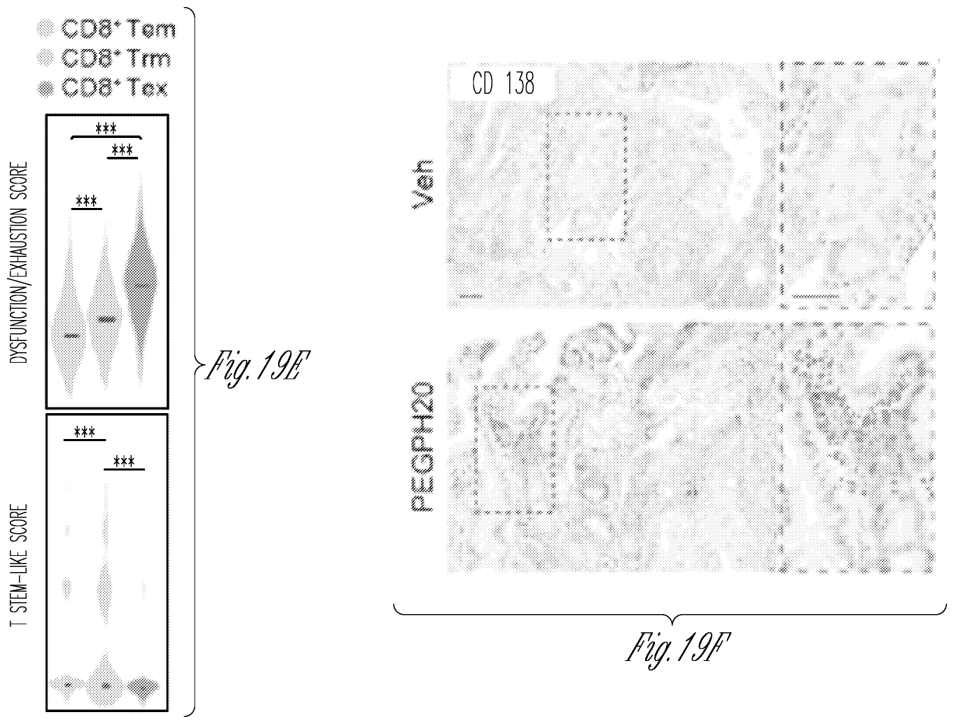

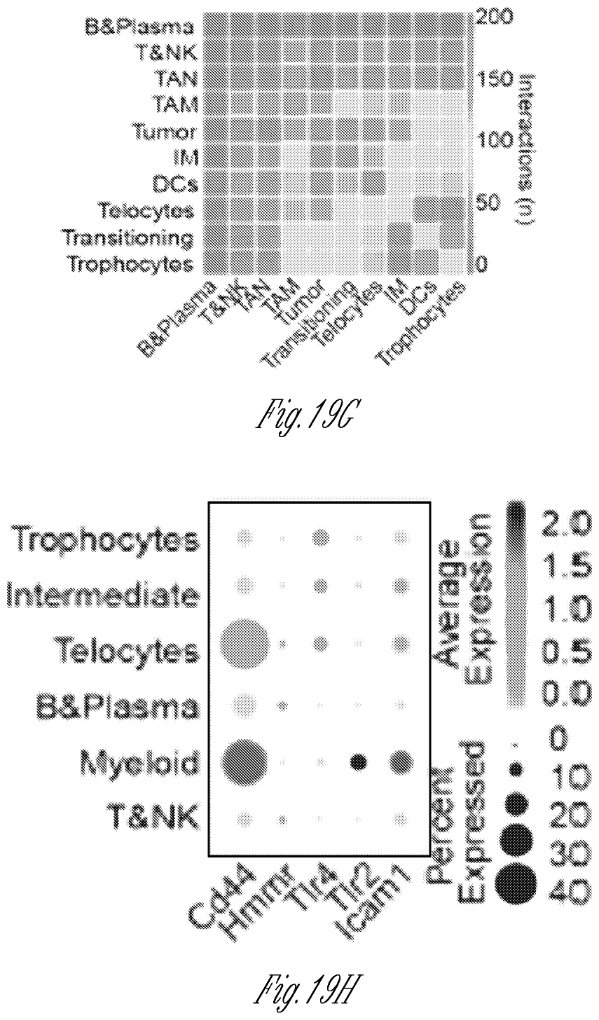

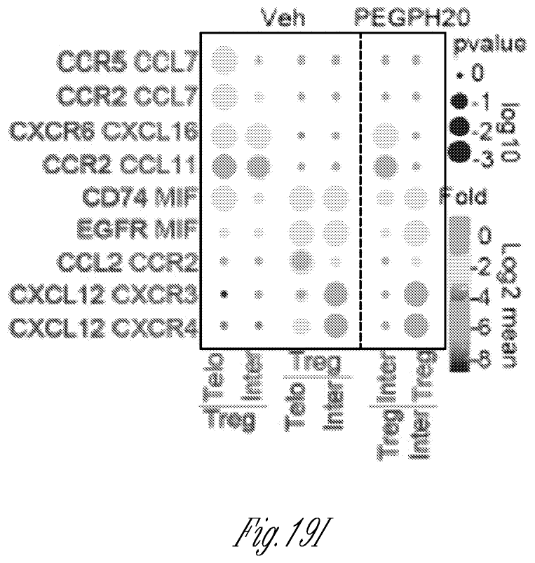

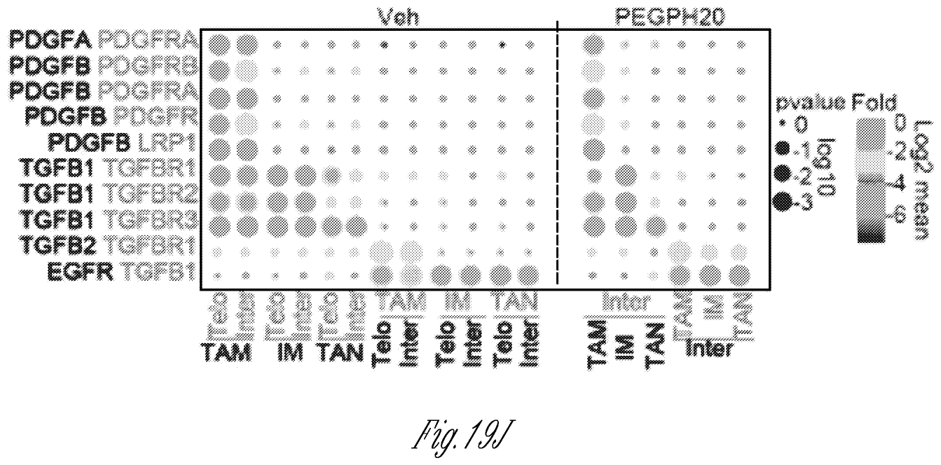

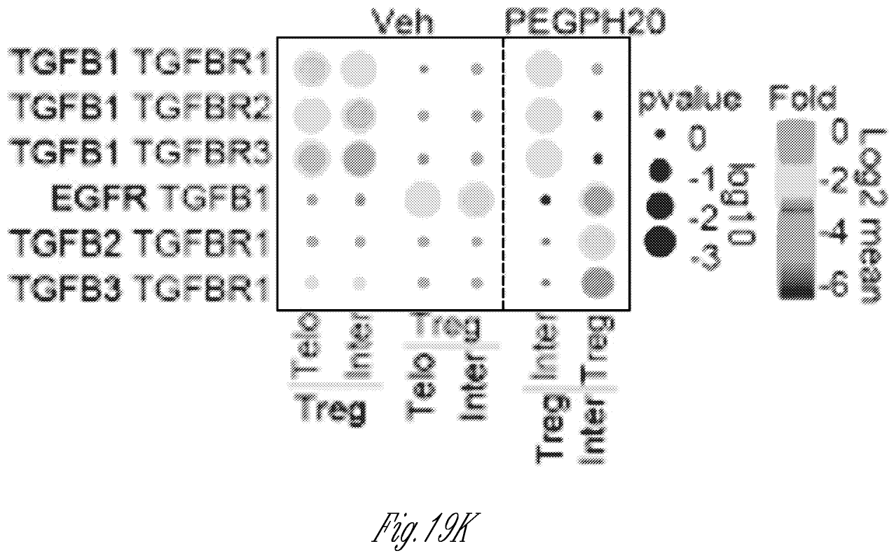

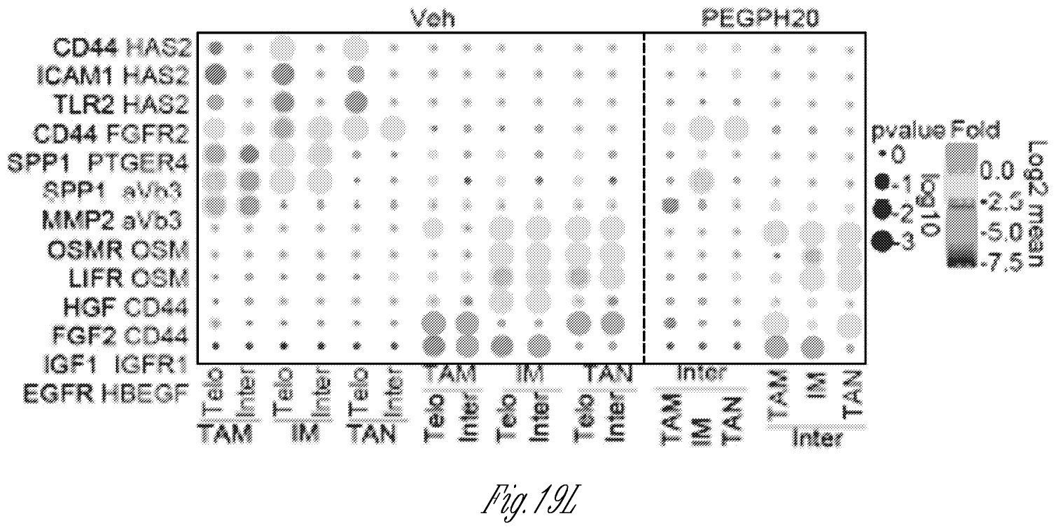

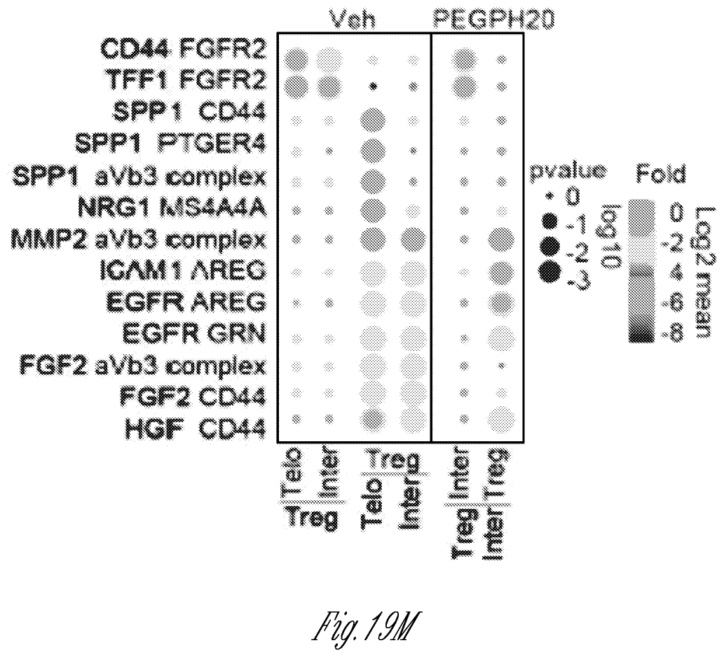

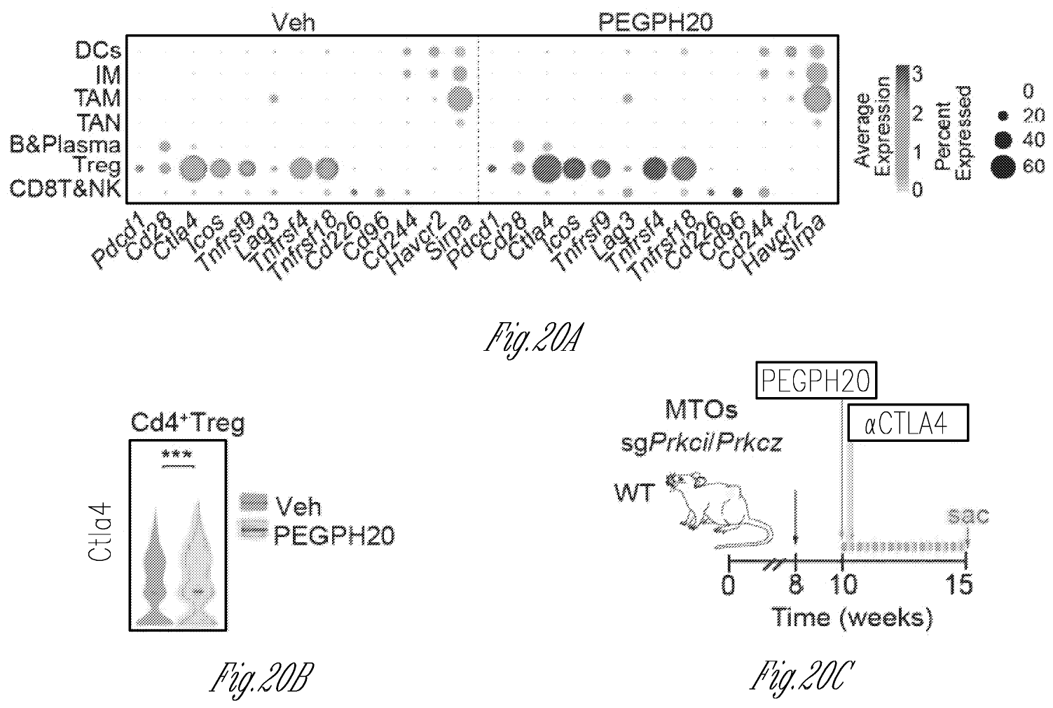

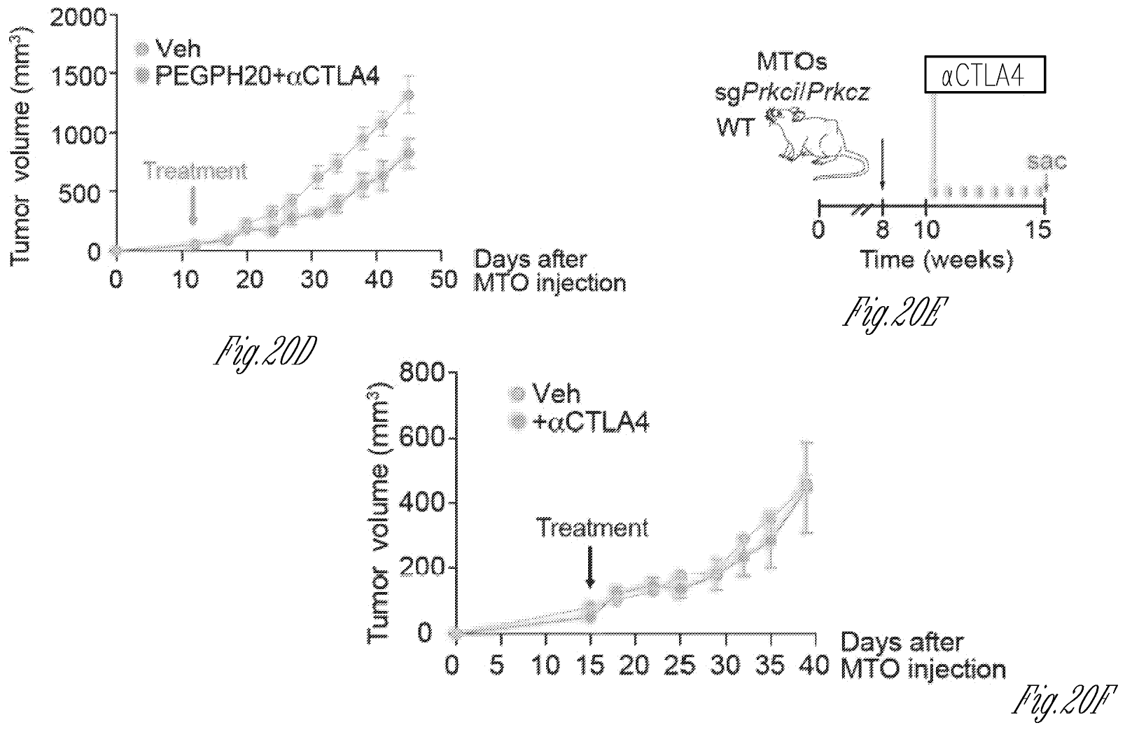

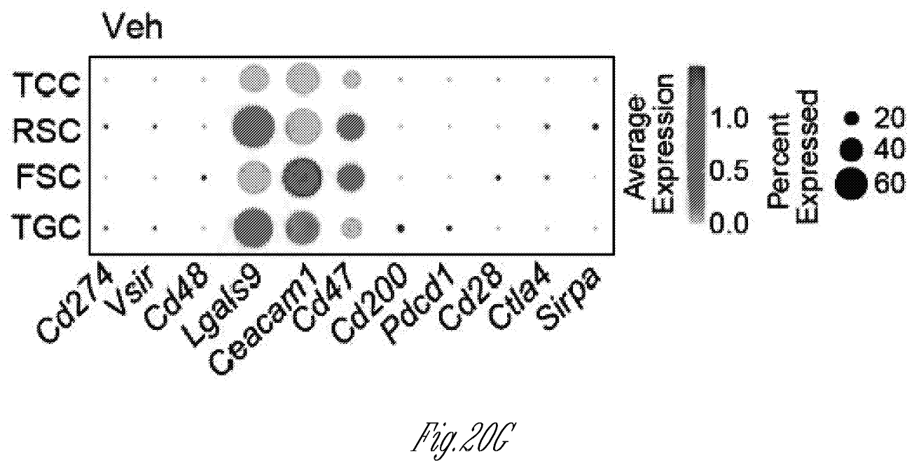

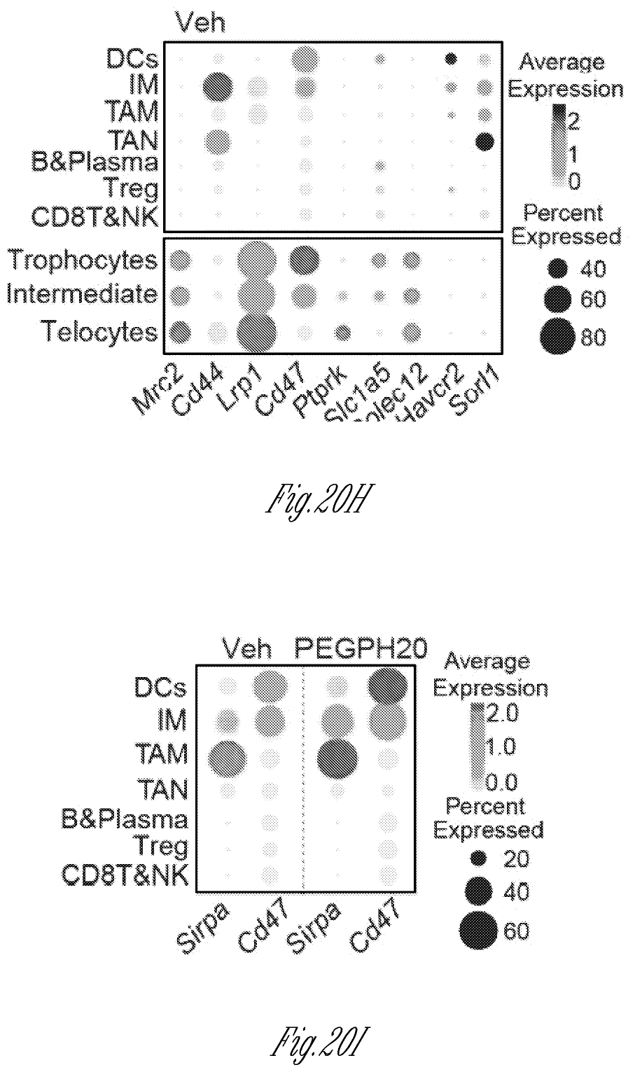

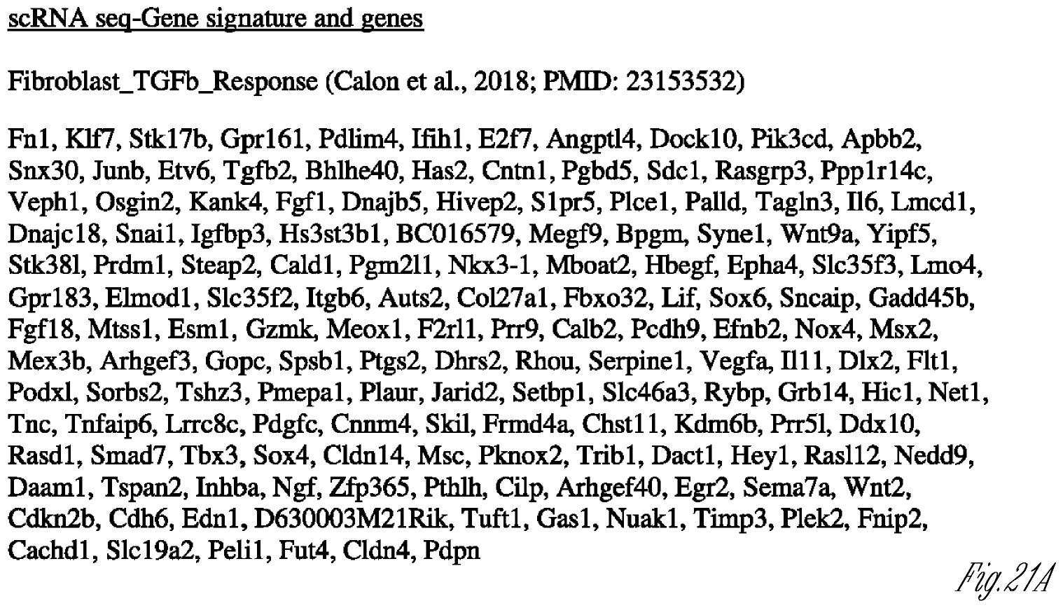

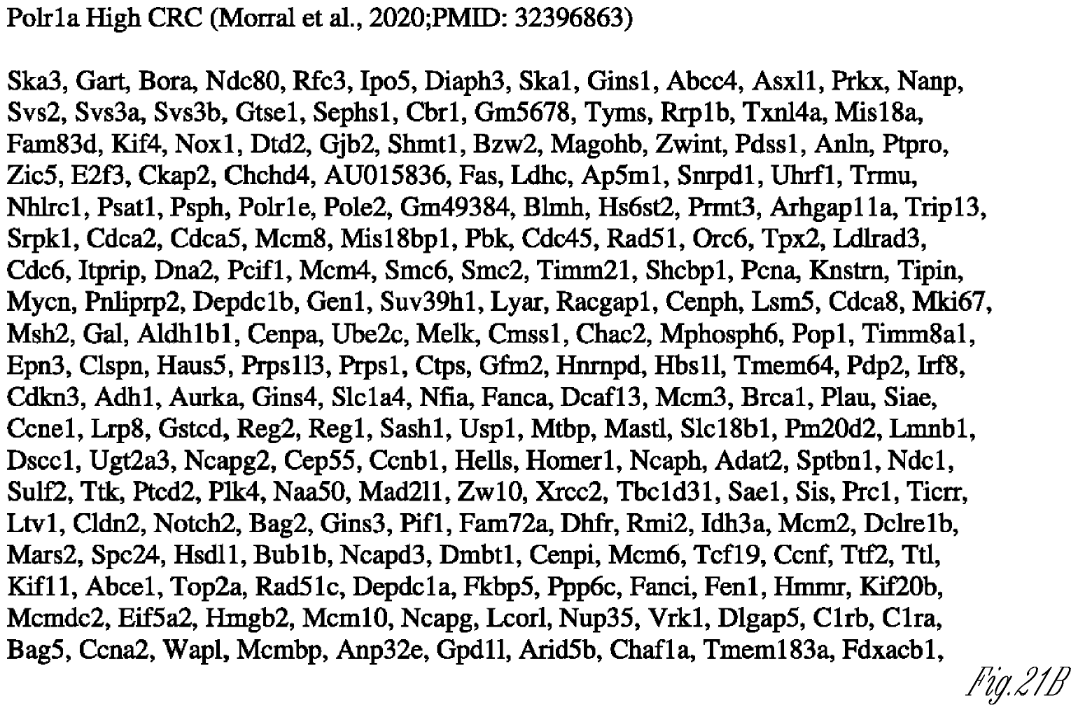

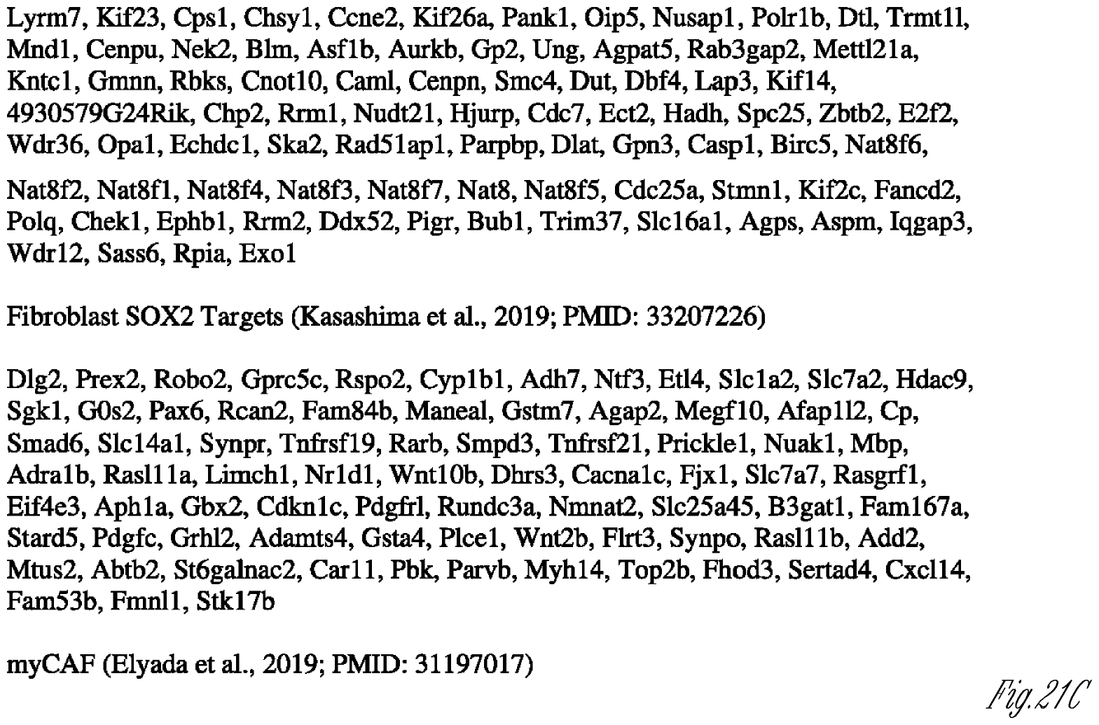

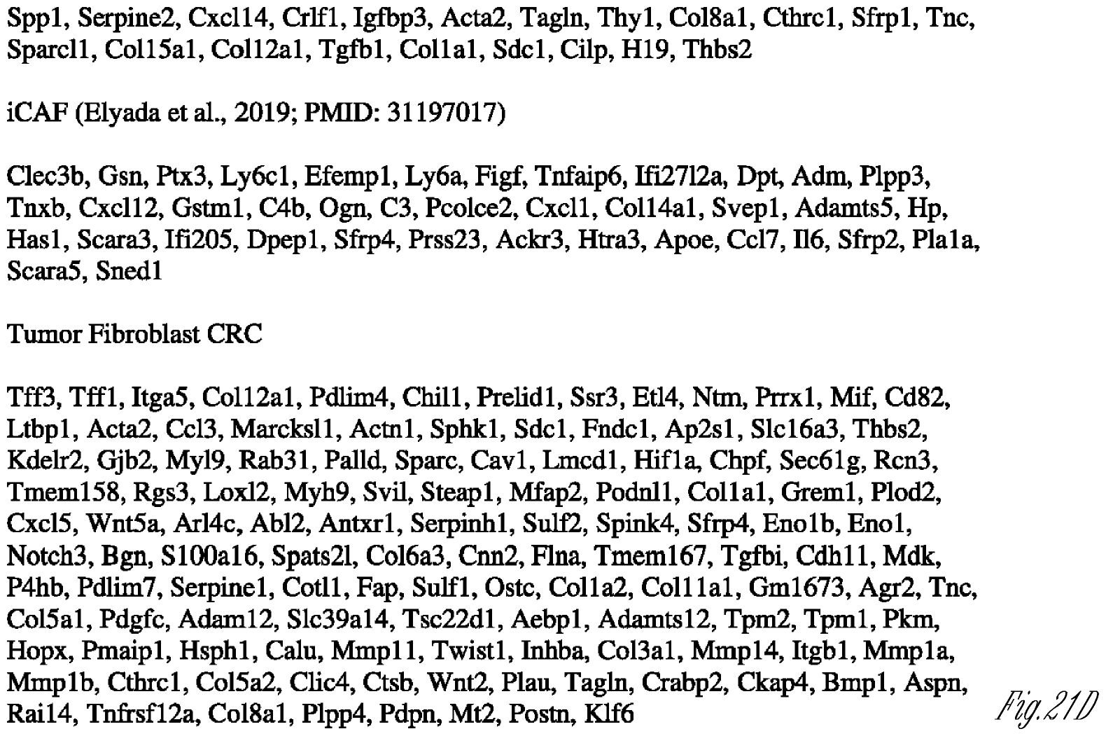

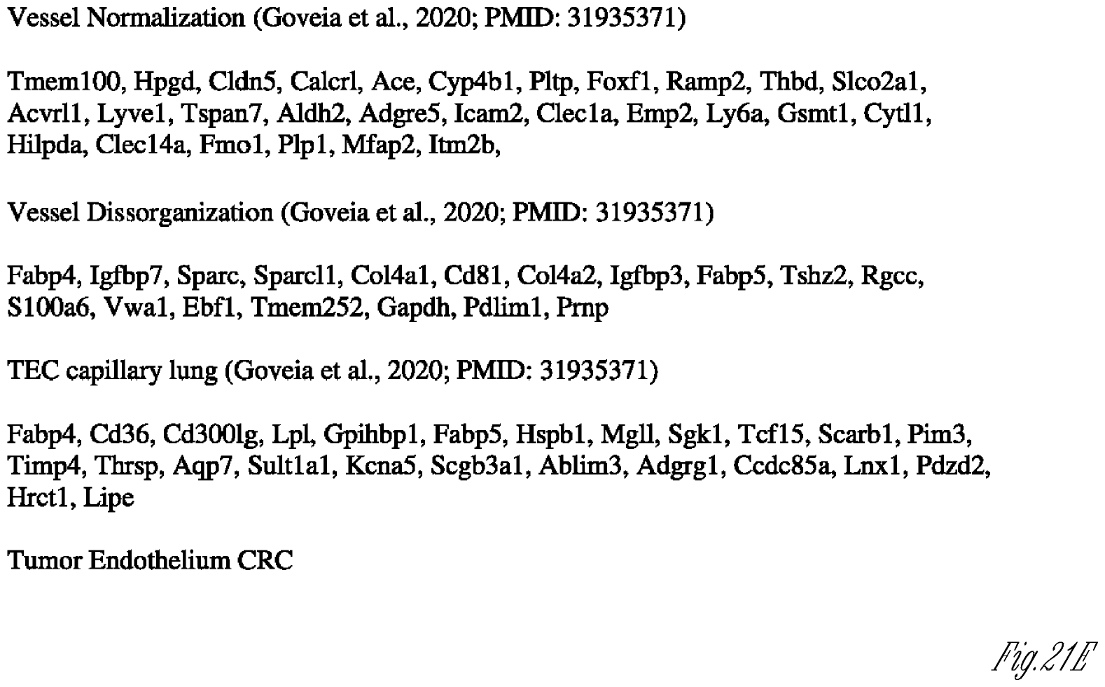

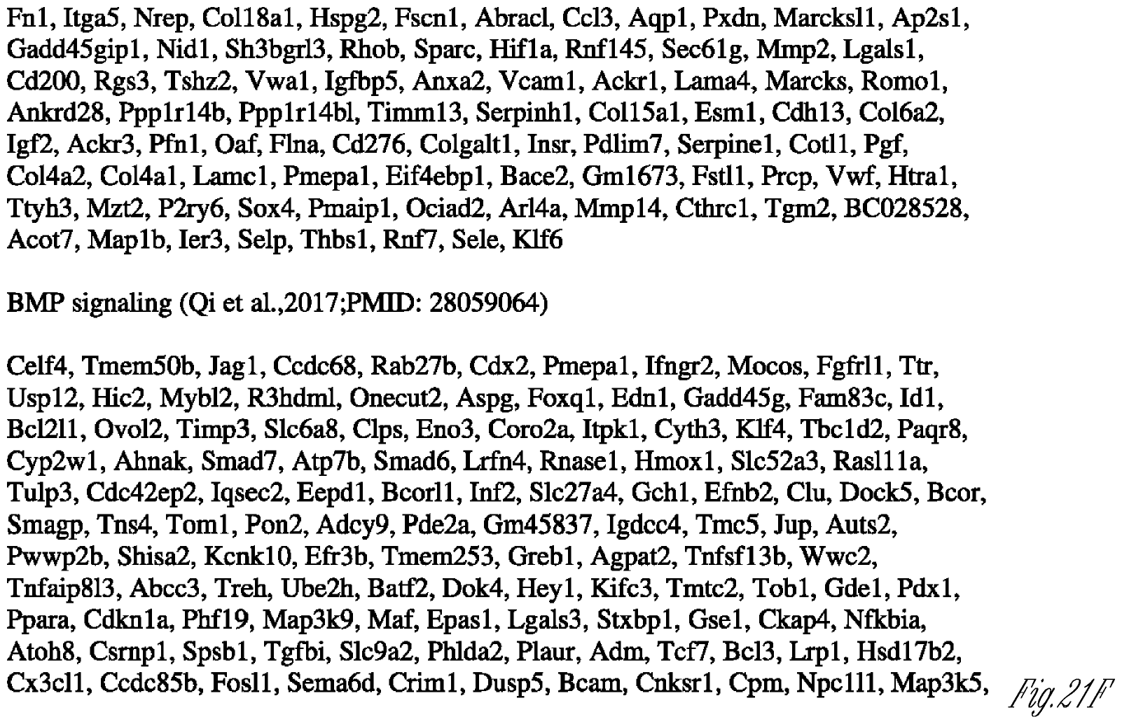

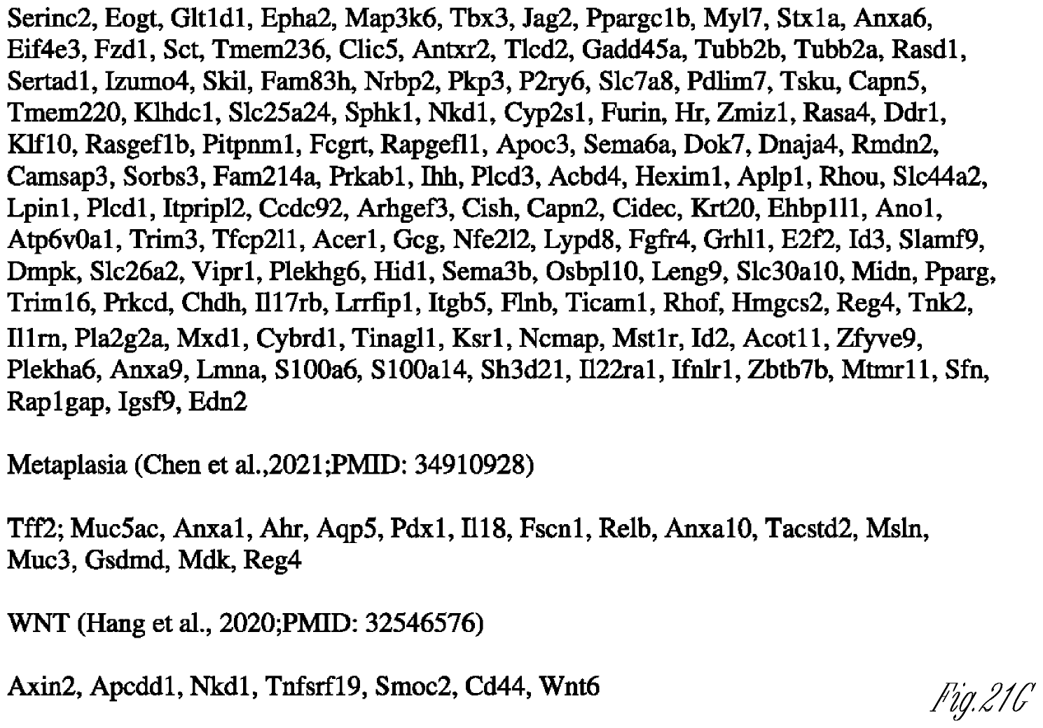

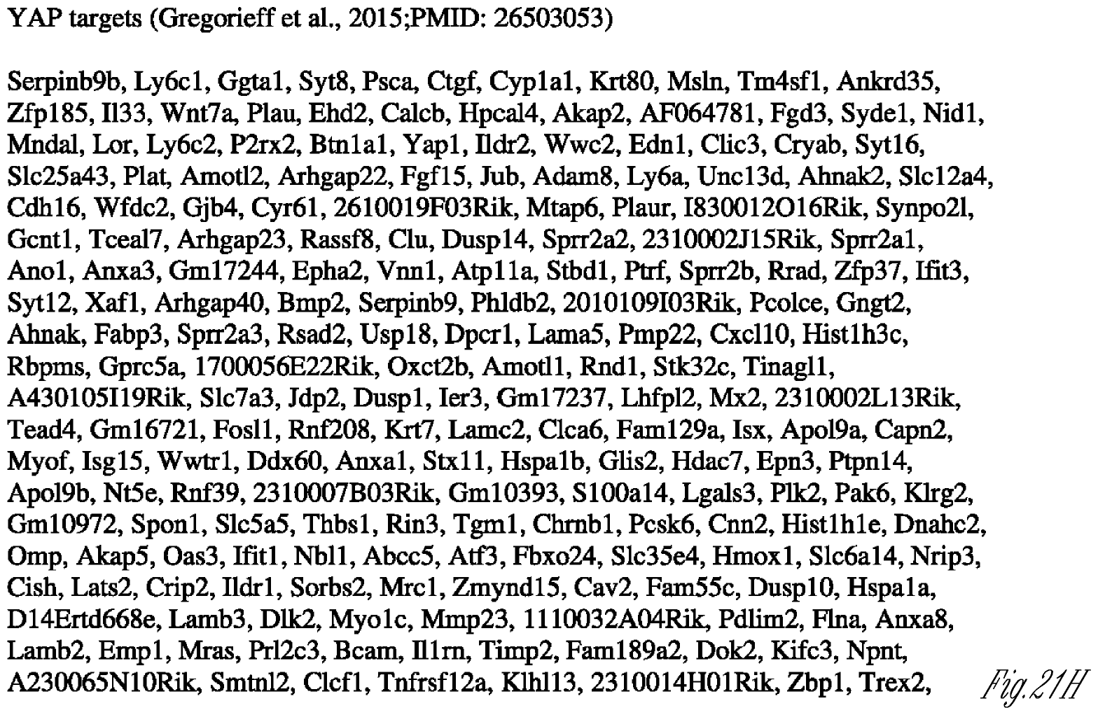

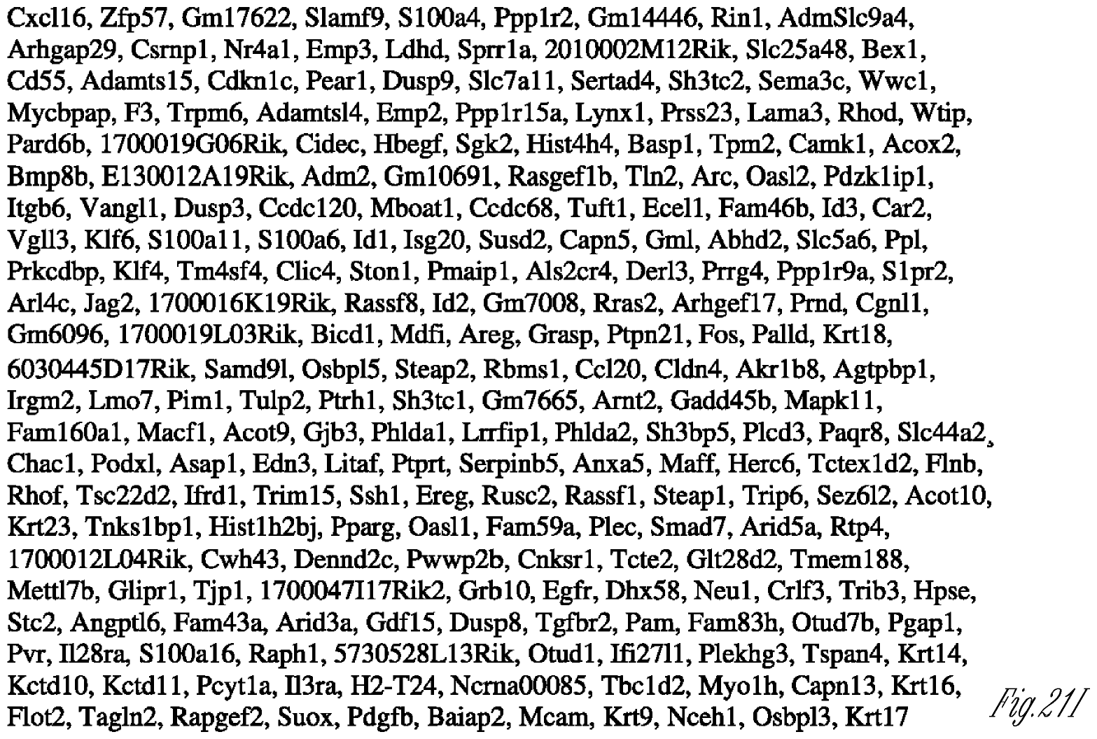

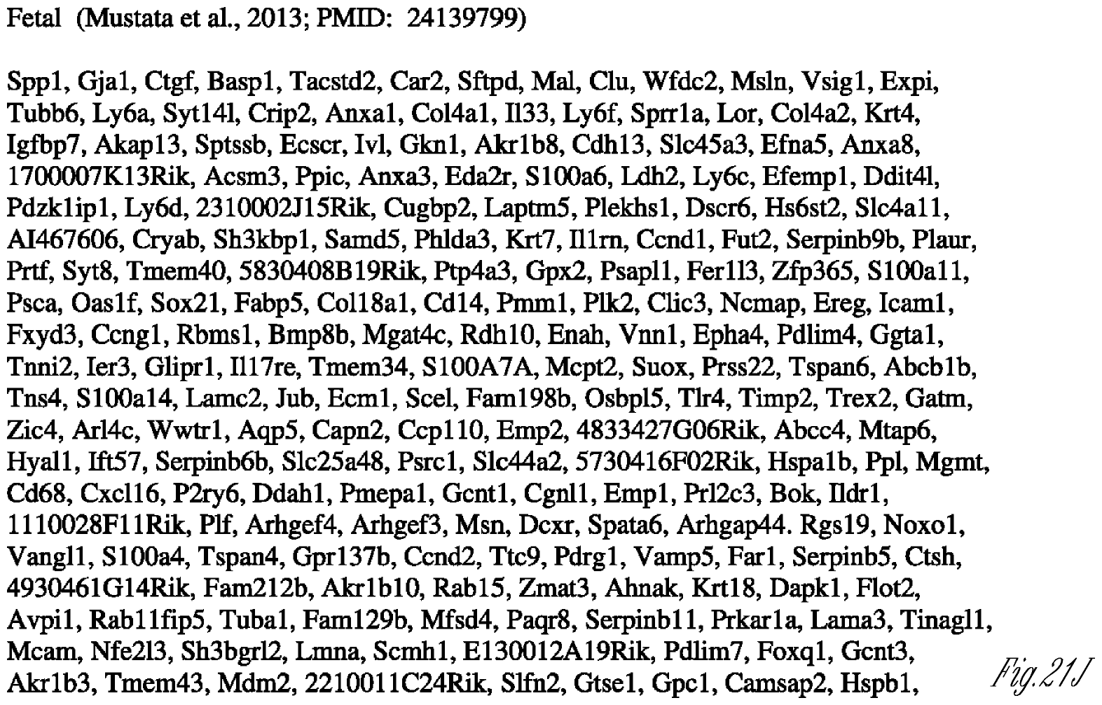

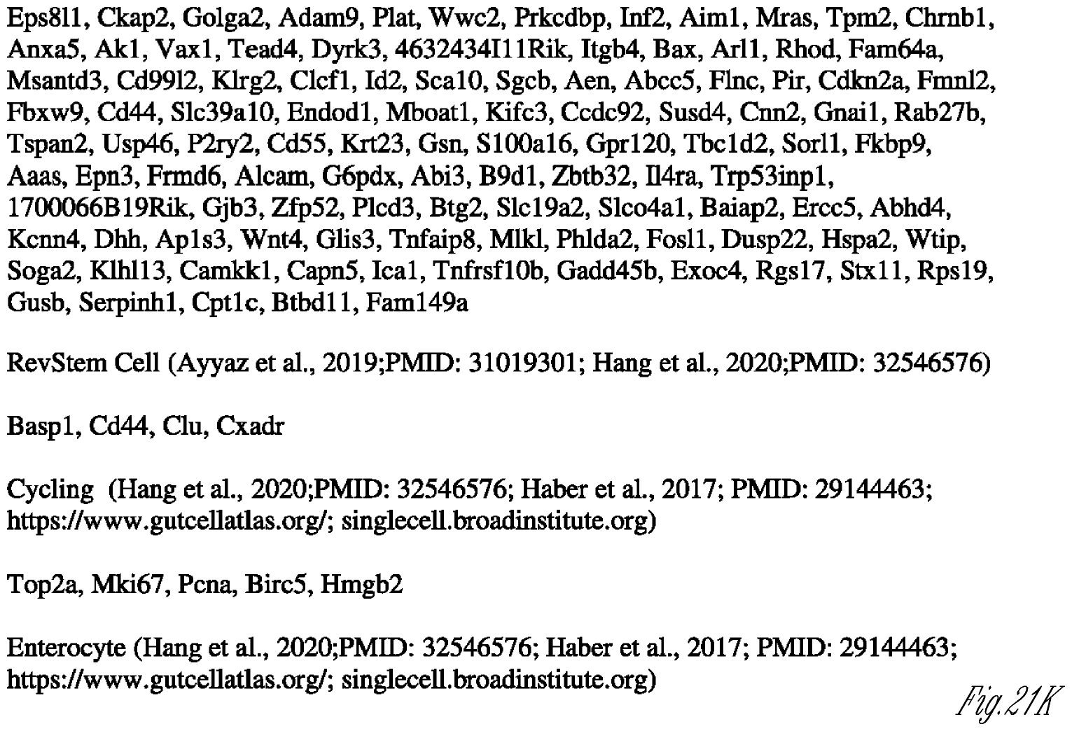

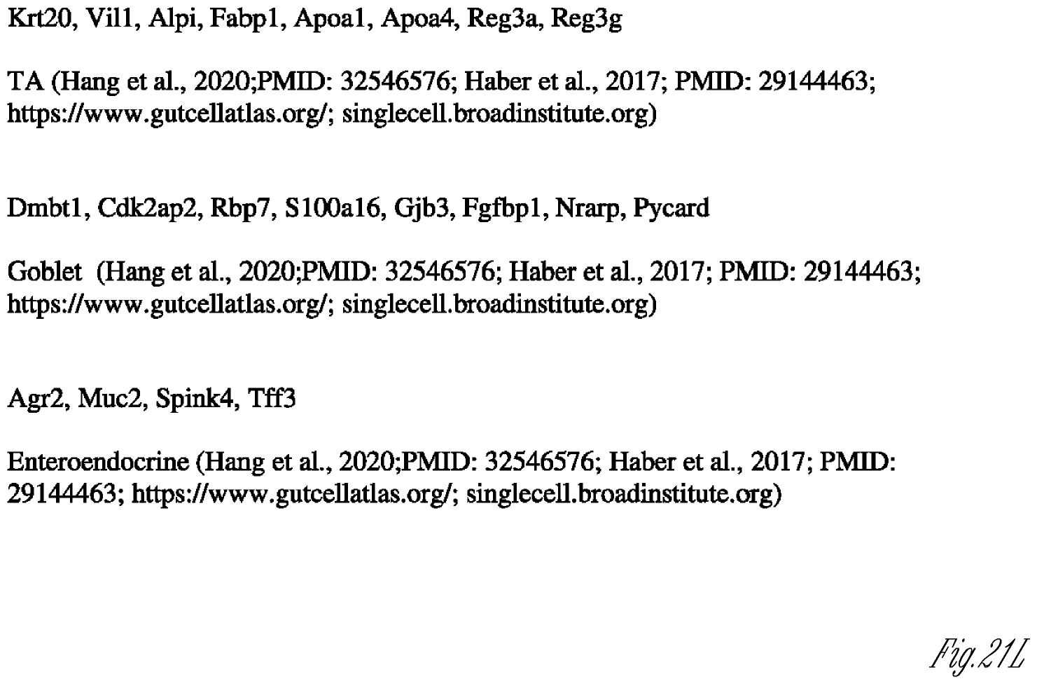

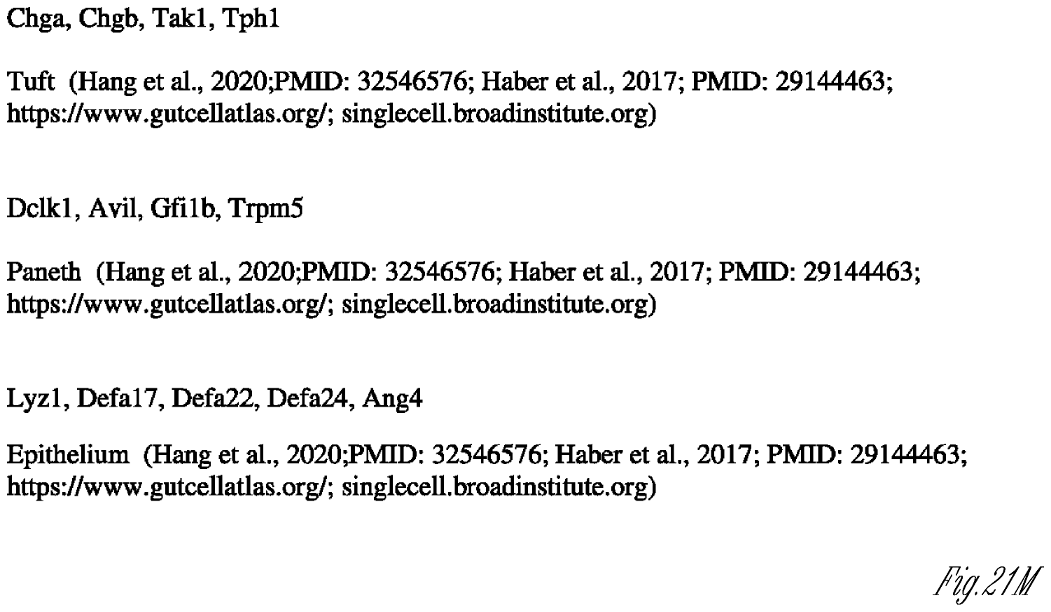

To establish a detailed molecular characterization at a cellular level of the mesenchymal phenotype and to better understand how the extracellular accumulation of HA promotes tumorigenesis and informs the microenvironment in this type of neoplasia, a scRNAseq analysis was carried out of Prkci^{fl/fl}Prkcz^{fl/fl}; Villin-Cre tumors from mice treated or not with PEGPH20 (FIGS. 10A-10B). Unsupervised clustering with selective markers for each population identified epithelial, stromal, and immune cells (FIGS. 10C and 21A). Since PEGPH20 remodels the TME extracellular matrix, first the focus was on the stromal component. Stromal cell re-clustering and mapping of marker gene expression identified six major stromal cell types, including endothelium (Pecam1), lymphatic-endothelial cells (Lyve1), smooth muscle cells (Myh11), fibroblasts (Dcn), glial cells (Plp1), and pericytes (Rgs5) (FIGS. 10D-E and 17B-C). Endothelial cells were the most abundant cell population in the stroma of Prkci^{fl/fl}Prkcz^{fl/fl}; Villin-Cre tumors (FIGS. 10D-F). Analysis of the endothelial cell compartment of Pecam1+ (CD31) and Lyve1+ cells identified nine cell types (FIGS. 7G-H and 17D-E), which were ascribed to the following categories: capillary (Cd36), artery (Gja4), vein (Ackr1), tip (Apln), immature (Aplnr), postcapillary (Selp), lymphatic-endothelial (Lyve1), shear-stress artery (Pi16) and proliferative (Birc5) (FIGS. 17D-E). Previous scRNAseq efforts identified tip and proliferative endothelial cells as specially enriched in the tumor endothelial cell (TEC) population, which have been proposed to be involved in tumor neo-angiogenesis (Goveia et al., 2020; Lee et al., 2020). To rigorously distinguish the clusters corresponding to normal endothelial cells (NEC) from the TEC in the Prkci^{fl/fl}Prkcz^{fl/fl}; Villin-Cre tumors, a TEC-specific signature was created by comparing tumor vs. normal endothelium from a human CRC scRNAseq dataset (GSE132465). This signature labeled Tip, Postcapillary, and Proliferating cells as TEC (FIGS. 17F-G). Also, gene signatures from lung endothelial cells supported that capillary and immature cells belong to the TEC category (FIGS. 17F-G). PEGPH20 reduced the proportion of tip cells and postcapillary and immature cells in Prkci^{fl/fl}Prkcz^{fl/fl}; Villin-Cre tumors (FIGS. 17H-I).

Next, two gene signatures reflecting the transcriptional programs of tumor vessel disorganization and normalization, respectively (Goveia et al., 2020), were characterized. The whole endothelial compartment of Prkci^{fl/fl}Prkcz^{fl/fl}; Villin-Cre tumors were enriched in the vessel disorganization signature, which was reversed upon PEGPH20 treatment, indicating that high HA deposition in mesenchymal tumors promotes endothelial remodeling (FIG. 17H). Furthermore, PEGPH20 also increased the normalization signature (FIG. 17H), demonstrating that HA depletion not only pruned the TECs but also normalized the endothelial landscape of the Prkci^{fl/fl}Prkcz^{fl/fl}; Villin-Cre tumors. Further pathway analysis of the endothelium compartment of these tumors showed that PEGPH20 reduced angiogenesis, glycolysis, and hypoxia signatures.

In contrast, it increased the expression of signatures related to interferon activation (FIG. 17I), suggesting enhanced immunosurveillance mechanisms driven by reprogramming endothelial cells in the tumor vasculature. These results demonstrate that the remodeling of the tumor endothelial vasculature is a hallmark of the high efficacy of PEGPH20 in reducing tumorigenesis.

### Fibroblast Heterogeneity in Endogenous Mesenchymal Tumors

The analysis of the fibroblast compartment by re-clustering the Dcn-expressing cells identified three cell categories (FIGS. 10J-K). Previous studies classified normal intestinal fibroblasts into three cell lineages according to the expression of Pdgfra, Cd81, and Cd34. This determines a dual and compartmentalized fibroblast positioning along the crypt axis and their biological function in intestinal homeostasis (McCarthy et al., 2020b). Thus, Pdgfra^{high} fibroblasts (telocytes) are abundant in the villus region, expressing high levels of BMP ligands, necessary for terminal epithelial cell differentiation (Chen et al., 2019).Conversely, Pdgfra^{low}Cd81^{high} fibroblasts (trophocytes) are CD34⁺, are exclusively located beneath the crypts, and express WNT pathway factors, including RSPO1 and RSPO3, all critical for adult stem cell maintenance (Harnack et al., 2019; Kim et al., 2005). In line with this classification, telocytes and trophocytes were identified as components of the fibroblast compartment of Prkci^{fl/fl}Prkcz^{fl/fl}; Villin-Cre tumors (FIGS. 10K and 17J-L). It was also found that these tumors were enriched in a third fibroblast population (“intermediate”) that shares some trophocyte features, such as the expression of WNT modulators and BMP inhibitors, but also resembles telocytes in that are negative for CD81 and CD34, yet express PDGFRA, albeit at a lower level than telocytes (FIGS. 10K and 17J-L). Telocytes and intermediate fibroblasts showed enrichment of carcinoma-associated fibroblast (CAF) signatures (FIG. 10L). CAFs have been previously described as inflammatory (iCAF) or myofibroblastic (myCAFs), which are strongly activated by TGFβ (Affo et al., 2021; Elyada et al., 2019; Hornburg et al., 2021). Telocytes showed higher myCAF signature expression and TGFβ response than intermediate or trophocytes (FIG. 10L). Conversely, trophocytes showed a higher expression of an iCAF signature than the intermediate and telocytes (FIG. 10L). Telocytes from Prkci^{fl/fl}Prkcz^{fl/fl}; Villin-Cre tumors express high levels of Sfrp2 and Sfrp4 (FIG. 17L), critical targets of SOX2 expression in CAFs to promote the CRC mesenchymal phenotype (Kasashima et al., 2021). Consistently, telocytes in these tumors displayed high expression of SOX2 targets (FIG. 10L), suggesting that telocytes/myCAFs are activated and expanded through the TGFβ/SOX2 axis to drive the progression of Prkci^{fl/fl}Prkcz^{fl/fl}; Villin-Cre tumors.

HA Degradation Remodels the mCRC CAF Compartment

HA degradation in vivo virtually eliminated the telocyte population, the main CAF in Prkci^{fl/fl}Prkcz^{fl/fl}; Villin-Cre tumors, and promoted the concomitant accumulation of trophocytes (FIGS. 10M-10N). In contrast, the proportion of intermediate fibroblast was not affected by PEGPH20 treatment (FIGS. 10M-10N). However, differential gene expression analysis showed a substantial reduction in collagens, matrix metalloproteinases, and signatures related to matrix remodeling in the intermediate fibroblasts from PEGPH20-treated mice (FIGS. 10O and 17M), suggesting that the HA-rich environment is fundamental for the selective maintenance of telocytes and myCAF features. These findings were further validated by multiplex immunofluorescence. Staining of NRG1 (activated telocytes), PDGFRA (telocytes), Pan-CK (epithelial cells), and DAPI (nuclear marker) showed a strong presence of PDGFRA⁺/NRG1⁺ fibroblasts at the luminal border of Prkci^{fl/fl}Prkcz^{fl/fl}; Villin-Cre tumors, consistent with the zonation pattern of telocytes in the normal intestinal epithelium, that was significantly reduced by PEGPH20 treatment (FIGS. 10P-10Q). Furthermore, trophocytes identified as a PDGRA⁻/CD31⁻/CD34⁺ population and normally located beneath the crypts in the normal intestinal epithelium showed a broader distribution in Prkci^{fl/fl}Prkcz^{fl/fl}; Villin-Cre tumors (FIG. 10R). PEGPH20 strongly increased trophocyte levels mainly at the crypt-bottom positions (FIGS. 10R-10S), which suggests a complete remodeling of the trophocyte population upon HA depletion.

Epithelial Cancer Cell Hierarchical Heterogeneity and Dependency on HA in mCRC Tumors

Next it was investigated whether HA depletion modulates the epithelial features of mesenchymal intestinal tumors. Epithelial cell re-clustering and mapping of the scRNAseq data from Prkci^{fl/fl}Prkcz^{fl/fl}; Villin-Cre tumors using marker gene expression classified the non-tumor epithelial cell population of these samples as non-cycling transient amplifying (TA), cycling transient amplifying (cTA), enterocytes, goblet, Paneth, tuft, and enteroendocrine (FIGS. 11A-11B and 18A-18B). Based on sample origin and genome-wide copy-number alteration (CNA) analysis, we classified epithelial cells as malignant or non-malignant (FIGS. 18C-18E). Four tumor epithelial populations were identified. One of the clusters corresponds to the tumor counterpart of the cycling TA population (TcTA) (FIGS. 11B and 18F-18H), which shares similarities with a recently identified cancer cell population seemingly essential for LGR5-independent tumor growth (Morral et al., 2020). Another cluster was enriched in Ly6a and Anxa10, markers of a previously reported fetal stem cell (tumor fetal stem cells; TFSC) type (FIGS. 11B and 18F-18H). A third tumor cell population was characterized by high expression of clusterin and is reminiscent of the “revival stem cell” (tumor revival stem cells; TRSC) that emerges in response to intestinal tissue damage associated with the loss of LGR5⁺ stem cells and activation of regenerative processes in non-tumor intestinal tissues (Ayyaz et al., 2019) (FIGS. 11B and 18F-18H). A fourth cancer cell type corresponded to transformed goblet cells (TGC) and displayed some features of immature goblet cells with high levels of Ly6a, and Anxa10, a transcriptional profile characteristic of the TFSCs found in the non-goblet epithelial tumor cell compartment (FIGS. 11B and 18I-18K).

Trajectory analysis using their estimated RNA velocity established a flow from cTAs to TcTAs, which gives rise to TFSCs and TRSCs albeit some of the latter might originate directly from cTAs (FIG. 11C). Transcriptomic comparison of tumor and normal intestinal epithelial cells shows that TcTAs are related to non-tumor cTAs, whereas TFSCs are more differentiated and related to enterocytes (FIG. 18H). Although there is an increased expression of a BMP signaling signature in the three epithelial cell transformed populations, as compared to cTA, TFSC showed the highest BMP signaling activation while displaying reduced WNT signaling, which was maintained/enhanced in the TRSC population (FIG. 18D). These data demonstrate that TFSCs are the most differentiated transformed cell populations and have features compatible with enterocyte markers (FIG. 18H). The TFSC compartment is enriched in a “metaplasia” gene expression signature (FIG. 11B). This is consistent with recent observations that human serrated tumors are associated with a metaplastic response whereby enterocytes acquire a fetal-gastric lineage as a cell protection mechanism against a chronic cytotoxic response (Chen et al., 2021a). In the diclosed model, toxicity is most likely triggered by constitutive inflammation driven by increased epithelial apoptosis and dysfunctional Paneth cells that promote tumor initiation (Nakanishi et al., 2019; Nakanishi et al., 2018; Nakanishi et al., 2016), which explains why there is a TRSC compartment in these tumors. This scenario accounts for the SSL histology characteristic of the Prkci^{fl/fl}Prkcz^{fl/fl}; Villin-Cre adenomas, which are the precursors of the serrated carcinomas (Chen et al., 2021a).

### Selective Signaling Phenotypes of Mesenchymal Tumor Cells are Maintained by Stromal HA Accumulation

From the signaling point of view, TcTAs are less enriched in proliferative signatures, such as “E2F_TARGETS” or “MYC_TARGETS,” than cTAs, yet these signatures were more enriched in TcTAs than in TFSC or TRSC (FIG. 11D). This indicates that tumor cells are less proliferative than non-tumor cTA cells and become even less proliferative as they differentiate towards the TFSC and TRSC compartments. There is also a progressive upregulation of the EMT and TGFβ gene expression signatures going from cTAs to TcTAs, and from these to TFSC, in keeping with the notion that aPKC-deficient tumor epithelial cells are more invasive yet less proliferative, with TFSC displaying the highest degree of mesenchymal activation (FIG. 11D). Regarding the signaling mechanisms, it was found there was a general increase in the YAP signature in all these transformed cells, which was highest in the TFSC population (FIG. 11D). YAP has been shown to control the production of EGF family growth factors (Gregorieff et al., 2015), suggesting that the strong ERK and EGFR activation responses previously described in Prkci^{fl/fl}Prkcz^{fl/fl}; Villin-Cre tumors can be accounted for by the presence of this more invasive TFSC cell population (Nakanishi et al., 2018). Tumor TRSCs were enriched in WNT signaling, suggesting that a relatively small but significant proportion of the cancer epithelial cells in Prkci^{fl/fl}Prkcz^{fl/fl}; Villin-Cre tumors rely on WNT activation likely to function as a reserve cell type (FIG. 11D). Altogether, these results demonstrated that cTAs is the most probable origin of Prkci^{fl/fl}Prkcz^{fl/fl}; Villin-Cre tumor epithelial cells, which, according to these results, are hierarchically structured reflecting the non-transformed intestinal cell lineage organization but with critical alterations in tumorigenic pathways that equip the serrated tumor cells with differential signaling characteristics (FIG. 11E).

In the secretory compartment, TGCs showed enrichment in EMT, YAP, and WNT signatures, as well as the increased expression of TFSC markers, compared to the non-tumor mature goblet population (FIGS. 11F and 18J-22L). Therefore, the fetal linage transition was not only detected in the enterocytic but also the goblet populations of serrated tumors. Based on the results showing that PEGPH20 treatment reduces TGFβ/EMT signatures in aPKC-deficient tumors (FIGS. 8K and 9L), it can be predicted that targeting HA would preferentially reduce the TFSC population, rendering the remaining tumor cells less invasive. Indeed, the reduction in tumor burden by PEGPH20 (FIGS. 8I-8J and 9G-9J) differentially affected the three tumor cell populations. Thus, while there was a limited proportional reduction of TcTAs, a clear reduction in the proportion of TFSCs was found but with a relative enrichment in the proportion of TRSCs in the persistent tumor cell population (FIG. 11D). This is consistent with the results demonstrating that TFSCs showed the highest upregulation of the TGFβ/EMT signatures and explains why the TcTAs and TRSCs are less sensitive to PEGPH20 treatment. Furthermore, trajectory analysis of the PEGPH20-treated condition showed a total disruption in the flow from TcTA to TFSC, while TFSC gained the ability to differentiate into TRSCs (FIG. 11G). Next, it was determined the localization of TFSCs (ANXA10⁺) and TRSCs (CLU⁺) in Prkci^{fl/fl}Prkcz^{fl/fl}; Villin-Cre tumors. Consistent with their enterocyte features and metaplastic phenotype, TFSCs were more exposed to the cancer luminal surface, whereas TRSCs were distributed at the crypt-bottom areas (FIG. 11H). PEGPH20 strongly reduced the presence of TFSCs (ANXA10⁺ staining), whereas that of TRSCs (CLU⁺ staining) was not reduced, in agreement with our scRNAseq findings (FIG. 11H). Furthermore, MTO-sgPrkci/sgPrkcz recapitulated the observed phenotype in Prkci^{fl/fl}Prkcz^{fl/fl}; Villin-Cre tumors showing in vitro upregulation of Anxa10 and Clu as compared with MTO-sgC (FIGS. 18M-22N). PEGPH20 treatment reduced Anxa10 expression without changes on Clu levels in MTO-sgPrkci/sgPrkcz (FIGS. 22M-22N). These results support a model whereby HA depletion in Prkci^{fl/fl}Prkcz^{fl/fl}; Villin-Cre tumors induced a lineage switch from TFSC to TRSC, suggesting that the WNT-independent TFSC population, which is likely maintained by YAP, can acquire a TRSC phenotype most probably supported by a WNT-dependent state. Regarding the goblet cell compartment, scRNAseq data demonstrated that PEGPH20 treatment strongly reduced the proportion of TGCs in Prkci^{fl/fl}Prkcz^{fl/fl}; Villin-Cre tumors (FIGS. 11I-11J). This was also supported by IHC and multiplex imaging showing ANXA10⁺/Alcian Blue and ANXA10⁺/MUC2⁺ TGCs throughout Prkci^{fl/fl}Prkcz^{fl/fl}; Villin-Cre tumors that were reduced by PEGPH20 treatment (FIGS. 5K and 5L), demonstrating that stromal HA accumulation is a crucial event for the maintenance of TGCs.

To establish the human relevance of these findings, the expression of the four tumor epithelial cell populations was correlated with CRC patient survival data by interrogating the bulk RNA-seq of CRC patients of the TCGA dataset with signatures corresponding to differentially expressed genes from the four cell types identified in the scRNAseq analysis of Prkci^{fl/fl}Prkcz^{fl/fl}; Villin-Cre tumors. Interestingly, gene set variation analysis (GSVA) demonstrated that although patients expressing the TcTA, TRSC, or the TGC signatures did not predict clinical outcomes, those expressing the TFSC gene signature had shorter disease-free survival (FIGS. 11M and 18M). These data demonstrate that the TFSC compartment accounts for the aggressive phenotype of mesenchymal tumors, yet they are sensitive to PEGPH20, reinforcing the notion that treatment with PEGPH20 can be an excellent therapeutic approach to mCRC with low aPKC expression but positive for HA.

### HA Depletion Reformats the CAF-Epithelial Tumor Interactions

Since TFSCs colocalize with telocytes at the luminal surface of Prkci^{fl/fl}Prkcz^{fl/fl}; Villin-Cre tumors and both populations were sensitive to PEGPH20 (FIGS. 10N, 10P and 11G-11H), it was posited that their interaction might be critical for the maintenance of tumor growth. Therefore, we investigated the crosstalk between fibroblasts and tumor epithelial populations by CellphoneDB analysis (Efremova et al., 2020). Telocytes showed the highest interactions with tumor cells compared to those of the intermediate or trophocyte populations (FIG. 11N). Telocytes were enriched in growth factors such as Igf1, Ereg, Fgf2/7, Nrg1, and Hgf, while their cognate receptors were broadly expressed by tumor cells (FIG. 11O), which could account for the regulation of TFSCs by telocytes. Areg was identified as a specific growth factor expressed in TFSC, interacting with Egfr and Icam1 in telocytes. Tff1, previously identified as a biomarker for SSL (Khaidakov et al., 2016), was expressed by TFSCs and could support telocytes expressing Fgfr2 (FIG. 11O). This set of interactions highlights the bi-directional telocyte-TFSC crosstalk that determines mesenchymal tumor malignancy. Furthermore, TRSCs and trophocytes were located at the crypt-bottom region in Prkci^{fl/fl}Prkcz^{fl/fl}; Villin-Cre tumors, and both populations were relatively enriched upon PEGPH20 treatment (FIGS. 10R and 11H). PEGPH20 induced a high number of communications between TRSC and trophocytes while switching off the interactions between TFSCs and telocytes (FIG. 11P). PEGPH20 treatment also enriched trophocytes in Rspo1/3, which interacted with their cognate receptor Lgr5 in the TRSC compartment (FIG. 11Q), in keeping with recent evidence on the maintenance by trophocytes of adult stem cell homeostasis through WNT signaling (McCarthy et al., 2020a; McCarthy et al., 2020b).

### Microenvironmental HA is Critical for Cancer Immunosuppression in Mesenchymal Tumors

Previously published data demonstrated that reduced interferon (IFN) signaling, which in turn impeded CD8⁺ T cell-mediated immunosurveillance, was a central event for the initiation of Prkci^{fl/fl}Prkcz^{fl/fl}; Villin-Cre tumors (Linares et al., 2021; Nakanishi et al., 2018). Notably, PEGPH20 treatment rescued the IFN inhibition in these tumors as demonstrated by enrichment in IFN and allograft rejection signatures by GSEA of RNAseq of Prkci^{fl/fl}Prkcz^{fl/fl}; Villin-Cre endogenous tumors and allografts from MTO-sgPrkci/sgPrkcz (FIG. 12A). This enrichment in IFN upon HA depletion was more evident in the TRSCs, which was the epithelial tumor sub-population more resistant to PEGPH20 treatment (FIG. 12B). These results suggest that PEGPH20 could enable ICB therapy through upregulating the IFN pathways in the “persister” epithelial cancer cells by switching them from immunoevasion to immunosurveillance mode. To further explore this possibility, it was determined how PEGPH20 reprogrammed the immune system of intestinal mesenchymal tumors. Unsupervised clustering of the immune component of the scRNAseq data from Prkci^{fl/fl}Prkcz^{fl/fl}; Villin-Cre tumors identified six major cell types: tumor-associated neutrophils (TANs), tumor-associated macrophages (TAMs), dendritic cells (DCs), inflammatory monocytes (IMs), B and plasma cells, T cells, and natural killer (NK) cells (FIGS. 12C and 19A-19B). Prkci^{fl/fl}Prkcz^{fl/fl}; Villin-Cre tumors were rich in TAN, TAM, and IM, and PEGPH20 treatment reduced the levels of these myeloid cell types and increased the proportion of T and B cells (FIGS. 12C-12D). Further unsupervised T and NK cells clustering identified three different CD8⁺ T cells and four CD4⁺ T cell populations, along with proliferative T cells, γδ T cells, and NK cells (FIGS. 12E and 23C). Interestingly, PEGPH20 reduced CD4⁺ Treg cells, which, together with the relative enrichment in CD8⁺ T cells, resulted in an increased CD8⁺/Treg ratio in their TME, a critical anti-tumorigenic state (FIG. 12E). Within the CD8⁺ T cells, three functional states were found: CD8⁺ T effector memory (Tem), CD8⁺ T resident memory (Trm), and CD8⁺ T exhausted (Tex) (FIGS. 12E and 23D). CD8⁺ Tex cells showed the highest degree of dysfunctionality and exhaustion, whereas CD8⁺ Trm were enriched in stemness (FIG. 23E). Furthermore, the lower levels of exhaustion markers and the expression of Gzmk suggested a pre-dysfunctional phenotype as the main characteristic of the CD8⁺ Tem population (FIG. 12D). PEGPH20 treatment increased the proportion of CD8⁺ Trm and CD8⁺ Tex cells and resulted in the enrichment in the signatures corresponding to CD8⁺ T cells exhaustion, stem-like, and expansion, as indicated by the increased expression of Ccl5 (FIGS. 12G-12H), suggesting strong CD8⁺ T cells activity in response to PEGPH20. VECTRA multiplex imaging showed a substantial accumulation of myeloid cells and CD4⁺ Tregs, a minimal presence of B cells, and the exclusion of CD8⁺ T cells from the tumoral areas in Prkci^{fl/fl}Prkcz^{fl/fl}; Villin-Cre tumors (FIG. 12I), which is consistent with the immunosuppressive environment of mCRC. PEGPH20 treatment produced a concomitant reduction of myeloid cells and CD4⁺ Tregs with strong recruitment of B, plasma, and CD8⁺ T cells to infiltrate the tumors (FIGS. 12E and 23F). These results demonstrate that the immune component of Prkci^{fl/fl}Prkcz^{fl/fl}; Villin-Cre tumors resembles the immunosuppressive features and immune cell composition of mCRC, which can be reprogrammed by PEGPH20 treatment.

### The Permissive Immune Environment of Mesenchymal Tumors is Orchestrated by Cross-Compartment Interactions and Maintained by HA

To elucidate the underlying mechanisms of the tumor response to PEGPH20, cell-specific receptor and ligand expression patterns between the three main compartments of the TME were determined. Based on CellphoneDB analysis, the predicted cell-cell communication networks highlighted the central role of telocytes in the recruitment of TAMs, IM, and TANs to the TME driven by mesenchymal aPKC-deficient epithelial cancer cells (FIG. 19G). Analysis of HA-receptors showed a higher expression of Cd44 in telocytes and myeloid cells, consistent with the high sensitivity of these two populations to HA-depletion (FIG. 19H). Focusing on the cytokines and their receptors that mediate myeloid cell recruitment to Prkci^{fl/fl}Prkcz^{fl/fl}; Villin-Cre tumors, we identified stromal-immune interactions between Ccl7- and Ccl11-expressing telocytes with myeloid cells positive for their cognate receptors Ccr2/Ccr1/Ccr5, and Ccr2, respectively. In addition, Cxcl2-expressing telocytes specifically recruit neutrophils positive for Cxcr2 (FIG. 19J), which can also account for the accumulation and recruitment of CD4⁺ Tregs that express Ccr5/Ccr2 (FIG. 19I). Therefore, eliminating telocytes by HA-depletion might explain the reduction of myeloid and CD4⁺ Tregs in mesenchymal intestinal tumors upon PEGPH20 treatment. Furthermore, PEGPH20 reduced the TGFβ signals provided by myeloid cells and CD4⁺ Tregs, contributing to the low amount of activated telocytes, EMT, and invasive characteristics of TFSCs (FIGS. 19J-19K). PEGPH20 also repressed the secretion of growth signals from telocytes to myeloid cells and CD4⁺ Tregs (FIGS. 19L-19M). Interestingly, Spp1, previously identified as a critical marker of myeloid cells in CMS4 CRC (Lee et al., 2020), was expressed by TAMs and might also account for the maintenance of telocytes expressing its receptors, Ptger4 and Cd44, in the mesenchymal stroma of Prkci^{fl/fl}Prkcz^{fl/fl}; Villin-Cre tumors (FIGS. 19L and 19M). Importantly, PEGPH20 treatment eliminates the interaction between Spp1-Ptger4/Cd44 in fibroblasts and the Spp1 interactions found between telocytes and CD4⁺ Tregs (FIG. 19M). PEGPH20 also increased the secretion of Ccl27a in TRSCs and Cc128 in TcTAs and in the residual TFSCs to recruit B cells and that of Ccl25 in TcTAs and TFSCs to attract Ccr9-expressing T cells to the tumor epithelial compartment (FIGS. 12K-12L). Furthermore, there was also a PEGPH20-driven interaction between Xc11 expressed in T cells and Xcr1-expressing DCs (FIG. 6L), shown to be critical for antigen presentation and a cytotoxic immune response (Dorner et al., 2009; Ozga et al., 2021). Collectively, these results support a model whereby the elimination of telocytes by PEGPH20 alleviates the immunosuppression triggered by myeloid cells and CD4⁺ Tregs, which cooperates with the induction of immunosurveillance through IFN activation in the tumor epithelial cell compartment, and the release of cytokines involved in B and CD8⁺ T cell infiltration (FIG. 12M).

HA Depletion Renders aPKC-Deficient Tumors Sensitive to ICB Therapy

The remodeling of the immune microenvironment by PEGPH20 suggested that it might increase responses to ICB therapy. Since draining lymph nodes of MTO-sgPrkci/sgPrkcz tumors showed a significantly increased proportion of immune cells expressing PD-L1 (FIG. 13A), the therapeutic potential of PEGPH20 in combination with anti-PDL1 (FIG. 13B) was tested. Importantly, the administration of anti-PD-L1 with PEGPH20 significantly enhanced the anti-tumor activity of PEGPH20 as determined by the reduced volume and weight of MTO-sgPrkci/sgPrkcz tumors (FIGS. 13C-13D). PEGPH20 treatment also reduced the HA content and intensity of αSMA⁺ stromal staining and was sufficient to bring CD8⁺ T and B cells to the tumor as well as to reduce the amount of immunosuppressive myeloid and Tregs (FIGS. 13E-13F). These results demonstrate that PEGPH20 treatment makes aPKC deficient tumors sensitive to anti-PD-L1 therapy.

### Discussion

Despite its importance, the cellular and molecular interactions that define the TME of mesenchymal tumors and its potential therapeutics are poorly understood. It was recently demonstrated that reduced levels of the two aPKC isoforms correlated with features of mesenchymal tumorigenesis in human CRC as well as that their simultaneous genetic inactivation in the intestinal epithelium is sufficient to induce this type of tumor in mice in the context of a morphologically serrated pathway driven by YAP-ERK and without mutations in BRAF or the APC/WNT cascade (Nakanishi et al., 2019; Nakanishi et al., 2018). Here we show that mesenchymal tumorigenesis, which is characterized in CRC by an EMT epithelium together with a desmoplastic and inflamed immune landscape with the exclusion of the CD8⁺ T cells to the tumor-stromal periphery, can be also induced in the context of tubular adenocarcinomas by the simultaneous inactivation of both aPKCs in organoids driven by mutations in the APC/KRAS/p53/TGFβ cassette. These observations are consistent with a large proportion of tumors harboring APC mutations that also express low aPKC levels and that show a mesenchymal phenotype. Importantly, our data reveal that a common critical feature shared by both mCRCs in humans and mice is the accumulation of HA in their stroma. HA deposition not only contributes to higher levels of malignancy in both the serrated and conventional settings but also makes these tumors vulnerable to the action of PEGPH20. Interestingly, the initial generation of HA can be accounted for by the cell autonomous activation of HAS expression upon the acute simultaneous genetic inactivation of both aPKCs in organoids from an inducible mouse model. Therefore, the present results support the notion that the activation of the HA biosynthetic machinery by the loss of aPKCs in the epithelial cell compartment is a critical step for the initiation of the mCRC preneoplastic lesions.

Treating mesenchymal tumors in mice with PEGPH20 not only results in the cell-autonomous reduction of the epithelial cell population with the most aggressive invasive features (TFSCs) but also promotes the recruitment of CD8⁺ T and B cells with the concomitant reduction in the infiltrating Tregs and immunosuppressive myeloid cells, which underly the reduction in tumor load. This is in contrast with the effects of inactivating the TGFβ stromal cascade, which is insufficient to reactivate the immune system or to reduce the tumor burden (Mariathasan et al., 2018; Nakanishi et al., 2018; Tauriello et al., 2018). Since other strategies to target the ECM, like the genetic inactivation of type I collagen, gave inconsistent results (Bhattacharjee et al., 2021; Chen et al., 2021b), PEGPH20 emerges as a potential new way for the treatment of mCRC tumors as a stroma-targeting monotherapy. Furthermore, the fact that PEGPH20 treatment of mCRC tumors results in the accumulation of CD8⁺ Trm cells explains why these PEGPH20-treated tumors further respond to anti-PD-L1 treatment, establishing HA degradation as an obligated step in the conversion of residual mCRC, PEGPH20-resistant tumor cells, from an ICB refractory state to a sensitive one.

All these results should be considered in the context of previous clinical trials using PEGPH20 for the treatment of pancreatic cancer, another type of desmoplastic neoplasia. The initial rationale was based on the assumption that this treatment would result in improved drug delivery to solid tumors and prolonged survival, as shown in preclinical models (Jacobetz et al., 2013; Provenzano et al., 2012; Thompson et al., 2010). Unfortunately, these clinical studies gave mixed results, and although patients showed a higher response rate in the experimental arm, there was no improvement in duration of response or progression-free or overall survival (Hingorani et al., 2016; Hingorani et al., 2018; Ramanathan et al., 2019; Van Cutsem et al., 2020). However, what is shown here is that PEGPH20 triggers a complete remodeling of the mCRC TME impacting not only the levels of HA but also the CAF populations and the immune compartment. Therefore, PEGPH20 should be considered a valid strategy to treat mCRC as monotherapy and/or as an enabler of ICB. In this regard, it is important to emphasize that the dose of PEGPH20 utilized in the pancreatic cancer clinical trials was approximately 400-fold lower than the one used in preclinical models, indicating that to achieve clinical response in pancreatic cancer patients, much higher hyaluronidase doses would be required, which will be unfeasible due to toxicity. In marked contrast, herein it was shown that mCRC is fully sensitive to the clinical doses of PEGPH20 (0.0375 mg/Kg), which strongly suggests that perhaps pancreatic cancer was not the right type of neoplasia to be treated with PEGPH20 whereas a better response would be obtained in patients with mCRC.

Another critical question to ensure the success of PEGPH20 in the clinic is the identification of better biomarkers for patient selection. In this regard, the data show that HA expression does not predict survival in CRC patients but that low aPKC levels combined with high HA levels define the cohort of CRC patients with the worst prognosis. Evidence presented here in two mCRC mouse models establishes that low aPKC/HA-positive mCRC patients will benefit from PEGPH20-based treatment. The analysis demonstrates that the epithelial compartment of mCRC tumors is organized echoing the hierarchy and cell types of the normal intestine. This includes the existence of tumor progenitor-like and differentiated-like populations (Dalerba et al., 2011; Merlos-Suarez et al., 2011; Vermeulen et al., 2008). Thus, according to the data, aPKC-deficient serrated tumors originate from a highly proliferative cTA population, which evolves into a tumor cycling cell type expressing a signature previously identified in Lgr5-negative CRC tumor cells (Morral et al., 2020). Therefore, the data strongly support the notion that differentiated cells retaining progenitor features can be transformed to generate a serrated mCRC. In this regard, a recent scRNAseq study in human patients established that whereas conventional adenomas originated from the expansion of adult stem cells at the bottom of the intestinal crypt, serrated adenomas emerge from a more differentiated cell state through a metaplastic process whereby intestinal cells trans-differentiate to a gastric-fetal phenotype (Chen et al., 2021a). The TFSC subpopulation identified in mCRC is what accounts for the most invasive and aggressive phenotype of this type of tumor and predicts poor patient survival. Therefore, this signature could be considered as a third potential biomarker to molecularly identify mCRC patients susceptible to PEGPH20 response, as this population is virtually wiped-out by HA degradation in vivo. Interestingly, there were symbiotic crosstalks between TFSCs and telocytes, which is the tumor fibroblast population with the most CAF features. The fact that PEGPH20 treatment completely ablated the telocyte subpopulation contributes to the eradication of the TFSC compartment and the reformatting of the mCRC TME towards a TRSC-trophocyte-dominated condition.

Notably, TRSCs originated from the TCC population but, in contrast to the TFSCs, retain features of adult sternness such as a heightened WNT pathway, which is reduced in the TFSC population that is instead characterized by a YAP-driven signature. TRSCs are reminiscent of the Clu+“revival stem cells” that emerge during non-tumor intestinal injury-regeneration processes (Ayyaz et al., 2019). Interestingly, this cell type is in a cooperative connection with the trophocyte fibroblasts at the bottom of the crypt through a WNT-dependent network, resembling the requirements of adult intestinal stem cells (Barker et al., 2007; van der Flier and Clevers, 2009). Since the non-tumor Clu+“revival stem cells” hold the ability to repopulate the whole normal epithelium, we posited that the TRSCs would be able to recreate the entire mCRC upon the cessation of PEGPH20 treatment. In this regard, it was show here that these cells are resistant to PEGPH20 treatment in vitro and that they account for a large proportion of the whole “persister” population that is not eradicated by PEGPH20 treatment. The fact that PEGPH20 promotes the accumulation of trophocytes in the mCRC TME further helps to feed the TRSC population as a possible source of therapy resistance. Fortunately, the present findings indicate that persistent cancer cells in PEGPH20-treated tumors are enriched in IFN signatures, which contributes to the sensitivity of these PEGPH20-treated tumors to anti-PDL1 therapy and suggests that, although PEGPH20 treatment profoundly represses mCRC tumorigenesis, combination with ICB will help prevent cancer rebound. Therefore, PEGPH20 reformats the mCRC TME from a TFCS-telocyte-driven scenario of aggressive tumorigenesis to a TRSC-trophocyte “persister” paradigm that can be, nonetheless, ablated by ICB treatment thanks to the upregulation of immunosurveillance IFN-driven pathways by PEGPH20.

In summary, the results of the study reveal interactions between stromal, epithelial, and the immune compartments whereby HA accumulation shapes the mCRC cellular landscape, providing mechanistic insights for immunotherapies. aPKC deficiency emerges as a biomarker, along with HA and the TFSC signature, to select mCRC patients where PEGPH20 could be used as a promising therapy.

### Example 4: Hyaluronan (HA) Depletion Renders aPKC-Deficient Tumors Sensitive to Immune Checkpoint Blockade (ICB) Therapy

In recent years, antibody-based immunotherapies re-activating the immune response against tumors have dramatically altered the cancer treatment paradigm. Unfortunately, ICB therapies have shown activity only in a relatively small percentage of cancer patients. Only cancer types with a high mutational burden, like MSI/CMS1 colorectal carcinomas (CRC), respond to ICB therapy. However, most MSS CRC tumors are entirely refractory to immunotherapies. One of the promising approaches to improve the scope and efficacy of these immune-based treatments is combinatorial therapy. Previous data using aPKC-deficient organoids (MTOs) indicated that the treatment with the hyaluronidase PEGPH20 makes ICB-refractory MSS CRC aPKC-deficient and HA-positive tumors sensitive to anti-PD-L1 therapy. Encouraged by these initial findings, the underlying mechanism of action of HA-depletion that results in tumor vulnerabilities to ICB was identified. To this end, it was determined whether PEGPH20 modulates the expression of additional immune checkpoint inhibitors (ICI) by interrogating Prkci^{fl/fl}Prkcz^{fl/fl}; Villin-Cre tumors from mice treated or not with PEGPH20 at a single-cell level. This analysis showed that PEGPH20 treatment resulted in the higher expression of several ICIs, including the cytotoxic T lymphocyte-associated antigen 4 (Ctla4), in CD4+ Tregs (FIGS. 20A and 20B).

Monoclonal antibodies targeting CTLA4, e.g., ipilimumab, PD1, or PD-L1 are the most broadly FDA-approved antibodies to treat several solid tumors. Interestingly, compared with monotherapy, the anti-CTLA4 plus anti-PD-1 combination has enhanced efficacy in advanced melanoma, renal carcinoma, and some MSI CRC. It was hypothesized that Ctla4 upregulation due to HA-depletion of the tumor microenvironment would render aPKC-deficient MTOs sensitive to anti-CTLA4 therapy. To test this possibility, wild-type C57BL/6J mice were subcutaneously transplanted with MTO-sgPrkci/sPrkcz and treated for four weeks with a low clinically relevant dose of PEGPH20 (37.5 g/Kg) in combination with anti-CTLA4 (FIG. 20C). Notably, PEGPH20, in combination with anti-CTLA4 treatment, resulted in a significant reduction in the volume of MTO-sgPrkci/sgPrkcz tumors, but anti-CTLA4 treatment as monotherapy showed no beneficial effects (FIGS. 20E and 20F). These results demonstrate that, although MSS CRC is resistant to ICB, aPKC deficiency renders these tumors sensitive to PEGPH20+ anti-CTLA4. In contrast, anti-CTLA4 treatment as monotherapy showed no beneficial effect on tumor development (FIGS. 20E and 20F).

Although the results demonstrate that ICB-refractory tumors can be rendered ICB-sensitive by treatment with PEGPH20, immune checkpoint inhibitors beyond conventional ICIs were identified intending to improve the immunotherapy treatment of CRC. Recent studies have identified several new immune checkpoint targets expressed by tumoral cells. The expression of additional ICIs in the epithelial compartment of Prkci^{fl/fl}Prkcz^{fl/fl}; Villin-Cre tumors was evaluated. This analysis revealed a remarkable expression of Galectin-9 (Lgals9), especially in TRSCs, a CRC cell population resistant to PEGPH20 monotherapy (FIG. 20G). Additionally, CellphoneDB analysis showed broadly expressed receptors for Galectin-9 and potential interactions with Lrp1- and Cd47-expressing trophocytes, in the so-called “intermediate” fibroblast population, as well as in Havcr2-expressing dendritic cells and Cd44-expressing myeloid cells (FIG. 20H). Of potential functional relevance, among the immune checkpoint inhibitors identified, Cd47 showed a clear increase upon PEGPH20 treatment (FIG. 20I), concomitant with a higher expression of its receptor, SIRPa, in tumor-associated macrophages and inflammatory monocytes. In summary, these observations suggest that the treatment with PEGPH20 combined with the immune targeting of Lgals9 or Cd47 might represent a potential new venue to reinvigorate CD8+ T cell function and synergize with ICB therapy in PEGPH20-treated CRC tumors.

Thus, therapies that include anti-galectin-9 and anti-Cd47 compounds including but not limited to anti-galectin-9 antibodies or fragments thereof, e.g., see Yang et al. (2022) or Barjon et al. (2012), the disclosures of which are incorpraoted by reference herein, including but not limited to AF2045 or ab69630, anti-Cd47 antibodies or fragments thereof, e.g., antibodies from clone REAL250, 2C8, or B6H12.2 (Lin et al., 2021; the disclosure of which is incorporated by refenrece herein), or humanized versions of any non-humanized antibody or fragment thereof, may be employed.

### Example 5: Hyaluronan (HA) Depletion Renders Liver Metastasis Sensitive to Immune Checkpoint Therapy Blockade

CRC predominantly metastasizes to the liver, which is the leading cause of death. Five-year overall survival for metastatic CRC patients is only 14.7%. Furthermore, patients with metastatic liver disease do worse than those without liver disease across different types of cancer and are totally refractory to ICB. Based on data that PEGPH20 remodels the tumor microenvironment and enables ICB in primary MSS CRC, the potential role of targeting HA in liver metastasis was investigated. To directly test therapeutic effects on liver metastatic disease, MTO-sgPrkci/sgPrkcz was inoculated in the portal circulation of wild-type C57BL/6J mice and mice were for two weeks and a half (5 doses) with a low clinically relevant dose of PEGPH20 (37.5 g/Kg) or in combination with anti-PDL1 or/and anti-CTLA4 (FIG. 22A). Notably, PEGPH20 administrated as monotherapy and in combination with anti-PDL1 markedly decreased the metastatic burden of MTO-sgPrkci/sgPrkcz (FIG. 21B), reducing not only the number of established liver tumors but also the volume of those tumors (FIGS. 22B-22D), indicating a dual role of hyaluronan as an inductor of liver metastasis initiation and metastatic disease progression. Of clinical relevance, the co-administration of PEGPH20 with anti-PDL1 and anti-CTLA4 showed a synergistic response and decreased metastatic burden of MTO-sgPrkci/sgPrkcz liver metastases, effectively curing a large proportion of mice (FIGS. 22B-22D). Altogether, these data strongly suggest that TME-remodeling by PEGPH20 enables immune infiltration and confers susceptibility to ICB therapies, suggesting that hyaluronan depletion could be promising as immunotherapy for primary MSS CRC mesenchymal patients and patients with liver metastatic disease.

### Methods

Intrasplenic injection of sgPrkci/Prkcz MTOs in WT mice. Mice were treated twice a week with Veh or PEGPH20 0.0375 mg/kg and/or αPD-L1 (5 mg/kg) and/or aCTLA4 (100 ug/dose) for 2.5 weeks (n: sgPrkci/Prkcz MTOs Veh-treated=7, sgPrkciiPrkcz MTOs PEGPH20-treated=7, sgPrkci/Prkcz MTOs PEGPH20 and αPD-L1-treated=7, and sgPrkci/Prkcz MTOs PEGPH20 and aCTLA4-treated=6).

### REFERENCES

Affo et al., Cancer Cell, 39:866 (2021).
Ashida et al., Clin. Cancer Res., 18:1578 (2012).
Ayyaz et al., Nature, 569:121 (2019).
Bankhead et al., Sci. Rep., 7:16878 (2017).
Barjon et al., Infect. Agent Cancer, 7:16 (2012).
Barker et al., Nature, 449:1003 (2007).
Bergen et al., Nat. Biotechnol., 38:1408 (2020).
Bhattacharjee et al., J. Clin. Invest., 131:_(2021)
Bray et al., Nat. Biotechnol., 34:525 (2016).
Butler et al., Nat. Biotechnol., 36:411 (2018).
Calon et al., A., Lonardo, E., Berenguer-Llergo, A., Espinet, E., Hernando-Momblona, X., Iglesias, M., Sevillano et al., Nat. Genet., 47:320 (2015).
Chen et al., Cell, 184:6262 (2021a).
Chen et al., Nat. Genet., 51:777 (2019).
Chen et al., Cancer Cell, 39:548 (2021b).
Dalerba et al., Nat. Biotechnol., 29:1120 (2011).
De Jaeghere et al., Trends Cancer, 5:704 (2019).
De Sousa et al., Nat. Med., 19:614 (2013).
Dienstmann et al., Nat. Rev. Cancer, 17:268 (2017).
Dorner et al., Immunity, 31:823 (2009).
Efremova et al., Nat. Protoc., 15:1484 (2020).
Eide et al., Sci. Rep., 7:16618 (2017).
el Marjou et al., Genesis, 39:186 (2004).
Elyada et al., Cancer Discov., 9:1102 (2019).
Espejo-Roamn et al., Pharmaceuticals, 14:788 (2022)
Fessler et al., Trends Cancer, 2:505 (2016).
Goveia et al., Cancer Cell, 37:21 (2020).
Gregorieff et al., Nature, 526:715 (2015)
Guinney et al., Nat. Med., 21:1350 (2015).
Harnack et al., C., Berger, H., Antanaviciute, A., Vidal, R., Sauer, S., Simmons, A., Meyer et al., Nat. Commun., 10:4368 (2019).
Hingorani et al., Clin. Cancer Res., 22:2848 (2016).
Hingorani et al., J. Clin. Oncol., 36:359 (2018).
Hornburg et al., Cancer Cell, 39:928 (2021).
Huyghe et al., Gut, 70:1325 (2021).
Jacobetz et al., Gut, 62:112 (2013).
Kasashima et al., Dev. Cell., 56:95 (2021).
Khaidakov et al., Am. J. Clin. Pathol., 146:530 (2016).
Kim et al., Science, 309:1256 (2005).
Korsunsky et al., Nat. Methods, 16:1289 (2019).
La Manno et al., Nature, 560:494 (2018).
Langmead et al., Nat. Methods, 9:357 (2012).
Lee et al., Nat. Genet., 52:594 (2020).
Levental et al., Cell, 139:891 (2009).
Lin et al., Front. Oncol., 5 Jan. 2021.
Linares et al., Mol. Cell.,_:_( ).
Madison et al., J. Biol. Chem., 277:33275 (2002).
Mandal et al., Science, 364:485 (2019).
Mariathasan et al., Nature, 554:544 (2018).
Martini et al., Ther. Adv. Med. Oncol., 12:_(2020).
McCarthy et al., Nat. Cell. Biol., 22:1033 (2020a).
McCarthy et al., Cell. Stem Cell, 26:391 (2020b).
Melsted et al., Nat. Biotechnol., 39:813 (2021).
Merlos-Suarez et al., Cell. Stem Cell, 8:511 (2011).
Missiaglia et al., Ann. Oncol., 25:1995 (2014).
Morral et al., Cell. Stem Cell, 26:845 (2020).
Moscat et al., Trends Cell Biol., __:__(2022),
Nakanishi et al., Trends Cancer, 5:742 (2019).
Nakanishi et al., Immunity, 49:1132 (2018).
Nakanishi et al., Cell Rep., 16:3297 (2016).
Overman et al., Lancet Oncol., 18:1182 (2017).
Ozga et al., Immunity, 54:859 (2021).
Patel et al., Methods Mol. Biol., 2055:455 (2020).
Patel et al., Blood, 134:2059 (2019).
Provenzano et al., Cancer Cell, 21:418 (2012).
Ramanathan et al., J. Clin. Oncol., 37:1062 (2019).
Reina-Campos et al., Cancer Cell, 36:218 (2019).
Siegel et al., CA Cancer J. Clin., 70:145 (2020).
Stuart et al., Cell, 177:1888 (2019).
Tauriello et al., Nature, 554:538 (2018).
Thompson et al., Mol. Cancer Thera., 9:3052 (2010).
Van Cutsem et al., J. Clin. Oncol., 38:3185 (2020).
van der Flier et al., Annu. Rev. Physiol., 71:241 (2009).
van Ineveld et al., JoVE, __:__(2020).
Vermeulen et al., Proc. Natl. Acad. Sci. USA, 105:13427 (2008).
Yang et al., J. Biol. Chem., 298:101821 (2022).

1. A method for determining that a subject has or is at risk of having a subtype of colorectal cancer, comprising:
(a) obtaining an expression level or an amount of (i) hyaluronic acid (HA) and/or of at least one HAR receptor and (ii) at least two atypical protein kinase Cs (PKCs) comprising PKCζ and PKCλ/ι in a biological sample from the subject;
(b) outputting a report indicative of said subject having or being at risk of having said subtype of said colorectal cancer at least based in part on (a); and
(c) administering a therapeutically effective measurement to said subject.

2-3. (canceled)

4. The method of claim 1, wherein said therapeutically effective measurement comprises administering a HA inhibitor.

5. The method of claim 4, wherein said HA inhibitor is selected from the group consisting of pegvorhyaluronidase alfa (PVHA), PEGPH20, a hyaluronidase, a HA synthase inhibitor, a HA receptor inhibitor, a CD44-ligand inhibitor such as JE22 or a THIQ-ester thereof, or a combination thereof.

6. The method of claim 5, wherein said hyaluronidase is a recombinant hyaluronidase.

7. The method of claim 1, wherein said subtype of colorectal cancer is CMS4.

8. The method of claim 1, said obtaining comprises assaying the expression level or the amount of HA, the at least one HA receptor, or the at least two atypical PKCs comprising PKCζ and PKCλ/ι, wherein said assaying comprises performing immunohistochemistry (IHC), enzyme-linked immunosorbent assay (ELISA), single-molecule array (Simoa), hyaluronidase activity assay, or a combination thereof.

9. The method of claim 8, wherein said assaying further comprises determining said biological sample has an elevated expression level of said HA as compared to a reference sample that does not have said subtype of colorectal cancer.

10. The method of any of claim 1, further comprising assaying an expression level or amount of at least one HA synthase.

11. The method of claim 10, wherein said assaying comprises determining said biological sample has an elevated expression level of said at least one HA synthase as compared to a reference sample that does not have said subtype of colorectal cancer.

12. (canceled)

13. The method of claim 8, wherein said assaying further comprises determining said biological sample has an elevated expression level of said at least one HA receptor as compared to a reference sample that does not have said subtype of colorectal cancer.

14. (canceled)

15. The method of claim 1, further comprising assaying an expression level or amount of at least one CD44-ligand.

16. The method of claim 15, wherein said assaying further comprises determining said colorectal cancer has an elevated expression level of said at least one CD44-ligand as compared to a reference sample that does not have said subtype of colorectal cancer.

17. The method of claim 16, wherein said at least one CD44-ligand comprises osteopontin (OPN/Spp1).

18. The method of claim 1, further comprising assaying an expression level or amount of at least one stromal cell marker.

19. The method of claim 18, wherein said assaying further comprises determining said biological sample has an elevated expression level of said at least one stromal cell marker as compared to a reference sample that does not have said subtype of colorectal cancer.

20. The method of claim 19, wherein said at least one stromal cell marker is selected from the group consisting of GREM1, SFRP1, SFRP2, SFRP4, CXCL14, MMP3, IGF1, and cell proliferation makers.

21.-22. (canceled)

23. The method of claim 8, wherein said assaying further comprises determining said biological sample has a lower expression level of said at least two atypical PKCs as compared to a reference sample that does not have said subtype of colorectal cancer.

24. A method for determining that a subject has or is at risk of having a subtype of colorectal cancer, comprising:
(a) obtaining an expression level or an amount of at least one stromal cell marker in a biological sample from the subject, wherein said at least one stromal cell marker comprises SFRP2 or SFRP4;
(b) outputting a report indicative of said subject having or being at risk of having said subtype of said colorectal cancer at least based in part on (a); and
(c) administering a therapeutically effective measurement to the subject.

25-46. (canceled)

47. A method for treating a subject having or is suspected of having a CMS4 colorectal cancer, comprising administering an effective amount of hyaluronic acid (HA) inhibitor to the subject.

48-68. (canceled)

69. A method for treating colorectal cancer, comprising:
preparing a biological sample taken from a patient in potential need of treatment for colorectal cancer;
assaying an expression level or amount of hyaluronic acid (HA) or expression level of at least one HA receptor and at least two atypical PKCs comprising PKCζ and PKCλ/ι in the prepared biological sample;
determining a subtype of colorectal cancer based at least in part on the results of the assay; and treating the subject by administering at least one therapeutically effective measurement to the subject based at least in part on the determined subtype.
